(19) United States
(12) Patent Application Publication
(10) Pub. No.: US 2009/0291938 A1
(43) Pub. Date: Nov. 26, 2009

(54) POLO-LIKE KINASE INHIBITORS

(75) Inventors: Sheldon X. Cao, San Diego, CA (US); Victoria Feher, San Diego, CA (US); Takashi Ichikawa, Tokyo (JP); Benjamin Jones, Cardiff-By-The-Sea, CA (US); Stephen W. Kaldor, Del Mar, CA (US); Andre A. Kiryanov, San Diego, CA (US); Betty Lam, Spring Valley, CA (US); Yan Liu, San Diego, CA (US); Christopher McBride, San Diego, CA (US); Srinivasa Reddy Natala, San Diego, CA (US); Zhe Nie, San Diego, CA (US); Jeffrey A. Stafford, San Diego, CA (US)

Correspondence Address: TAKEDA SAN DIEGO, INC., 10410 SCIENCE CENTER DRIVE, SAN DIEGO, CA, 92121, US

(73) Assignee: TAKEDA PHARMACEUTICAL COMPANY LIMITED, Osaka (JP)

(21) Appl. No.: 12/274,213

(22) Filed: Nov. 19, 2008

Related U.S. Application Data
(60) Provisional application No. 60/989,018, filed on Nov. 19, 2007.

(51) Int. Cl. A61K 31/551; C07D 487/04

(52) U.S. Cl. 514/210.21; 540/502; 514/221; 514/220; 540/495; 514/217.06; 540/496

(57) ABSTRACT

Compounds of the following formula are provided for use with kinases:

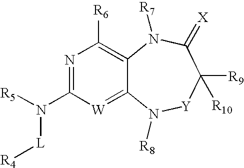

wherein the variables are as defined herein. Also provided are pharmaceutical compositions, kits and articles of manufacture comprising such compounds; methods and intermediates useful for making the compounds; and methods of using said compounds.

### RELATED APPLICATIONS

This application claims the benefit of U.S. Provisional Application No. 60/989,018 filed Nov. 19, 2007, which is incorporated herein by reference.

### FIELD OF THE INVENTION

The present invention relates to compounds that may be used to inhibit kinases, as well as compositions of matter, kits and articles of manufacture comprising these compounds. The invention also relates to methods for inhibiting kinases and treatment methods using compounds according to the present invention. In addition, the invention relates to methods of making the compounds of the present invention, as well as intermediates useful in such methods. In particular, the present invention relates to Polo-like Kinase (PLK) inhibitors, compositions of matter, kits and articles of manufacture comprising these compounds, methods for inhibiting PLK, and methods and intermediates useful for making the inhibitors.

### BACKGROUND OF THE INVENTION

The invention relates to inhibitors of enzymes that catalyze phosphoryl transfer and/or that bind ATP/GTP nucleotides, compositions comprising the inhibitors, and methods of using the inhibitors and inhibitor compositions.

Many diseases states are characterized by the uncontrolled proliferation and differentiation of cells. These diseases states encompass a variety of cell types and maladies such as cancer, atherosclerosis, restenosis, and psoriasis. Uncontrolled signaling due to defective control of protein phosphorylation has been implicated in a number of diseases and disease conditions, including, for example, inflammation, cancer, allergy/asthma, diseases and conditions of the immune system, disease and conditions of the central nervous system (CNS), cardiovascular disease, dermatology, and angiogenesis.

The inhibitors and compositions comprising them are useful for treating or modulating disease in which phosphoryl transferases, including kinases, may be involved, symptoms of such disease, or the effect of other physiological events mediated by phosphoryl transferases, including kinases. The invention also provides for methods of making the inhibitor compounds and methods for treating diseases in which one or more phosphoryl transferase, including kinase, activities is involved.

Phosphoryl transferases are a large family of enzymes that transfer phosphorous-containing groups from one substrate to another. By the conventions set forth by the Nomenclature Committee of the International Union of Biochemistry and Molecular Biology (IUBMB) enzymes of this type have Enzyme Commission (EC) numbers starting with 2.7.-.- (See, Bairoch A., The ENZYME database in Nucleic Acids Res. 28:204-305 (2000)). Kinases are a class of enzymes that function in the catalysis of phosphoryl transfer. The protein kinases constitute the largest subfamily of structurally related phosphoryl transferases and are responsible for the control of a wide variety of signal transduction processes within the cell. (See, Hardie, G. and Hanks, S. (1995) The Protein Kinase Facts Book, I and II, Academic Press, San Diego, Calif.). Protein kinases are thought to have evolved from a common ancestral gene due to the conservation of their structure and catalytic function. Almost all kinases contain a similar 250-300 amino acid catalytic domain. The protein kinases may be categorized into families by the substrates they phosphorylate (e.g., protein-tyrosine, protein-serine/threonine, histidine, etc.). Protein kinase sequence motifs have been identified that generally correspond to each of these kinase families (See, for example, Hanks, S. K.; Hunter, T., FASEB J. 9:576-596 (1995); Kinghton et al., Science, 253:407-414 (1991); Hiles et al., Cell 70:419-429 (1992); Kunz et al., Cell, 73:585-596 (1993); Garcia-Bustos et al., EMBO J., 13:2352-2361 (1994)). Lipid kinases (e.g. PI3K) constitute a separate group of kinases with structural similarity to protein kinases.

Protein and lipid kinases regulate many different cell processes including, but not limited to, proliferation, growth, differentiation, metabolism, cell cycle events, apoptosis, motility, transcription, translation and other signaling processes, by adding phosphate groups to targets such as proteins or lipids. Phosphorylation events catalyzed by kinases act as molecular on/off switches that can modulate or regulate the biological function of the target protein. Phosphorylation of target proteins occurs in response to a variety of extracellular signals (hormones, neurotransmitters, growth and differentiation factors, etc.), cell cycle events, environmental or nutritional stresses, etc. Protein and lipid kinases can function in signaling pathways to activate or inactivate, or modulate the activity of (either directly or indirectly) the targets. These targets may include, for example, metabolic enzymes, regulatory proteins, receptors, cytoskeletal proteins, ion channels or pumps, or transcription factors. Initial interest in protein kinases as pharmacological targets was stimulated by the findings that many viral oncogenes encode structurally modified cellular protein kinases with constitutive enzyme activity. These findings pointed to the potential involvement of oncogene related protein kinases in human proliferatives disorders. Subsequently, deregulated protein kinase activity, resulting from a variety of more subtle mechanisms, has been implicated in the pathophysiology of a number of important human disorders including, for example, cancer, CNS conditions, and immunologically related diseases. The development of selective protein kinase inhibitors that can block the disease pathologies and/or symptoms resulting from aberrant protein kinase activity has therefore generated much interest.

Cancer results from the deregulation of the normal processes that control cell division, differentiation and apoptotic cell death. Protein kinases play a critical role in this regulatory process. A partial non-limiting list of such kinases includes ab1, Aurora-A, Aurora-B, Aurora-C, Akt, bcr-abl, Blk, Brk, Btk, c-Kit, c-Met, c-Src, CDK1, CDK2, CDK4, CDK6, cRaf1, CSF1R, CSK, EGFR, ErbB2, ErbB4, ERK, Fak, fes, FGFR1, FGFR2, FGFR3, FGFR4, FGFR5, Fgr, Flt-4, Flt-1, FER, Frk, Fyn, Hck, IGF-1R, INS-R, Jak, KDR, Lck, Lyn, MEK, p38, PDGFR, PIK, PKC, PLKs, PYK2, Ros, Tie1, Tie2, Trk, Yes and Zap70. In mammalian biology, such protein kinases comprise mitogen activated protein kinase (MAPK) signaling pathways. MAPK signaling pathways are inappropriately activated by a variety of common disease-associated mechanisms such as mutation of ras genes and deregulation of growth factor receptors (Magnuson et al., Seminars in Cancer Biology 5:247-252 (1994)). Therefore the inhibition of protein kinases is an object of the present invention.

Polo-like kinases (PLKs including PLK1, PLK2, PLK3 and PLK4) are serine/threonine protein kinases that have been implicated in human cancer, such as colon, breast and other solid tumors. Polo-like kinases (also referred to as PLKs) are believed to be involved in protein phosphorylation events that regulate the cell cycle. Specifically, PLK1 may play a role in controlling the accurate segregation of chromosomes during mitosis. Misregulation of the cell cycle can lead to cellular proliferation and other abnormalities. In human colon cancer tissue, PLKs have been found to be overexpressed (See, Barr et al in Nat. Rev. Mol. Cell. Biol. 5: 429 (2004); van Vugt et al in Ocogene, 24: 2844 (2005)). PLK1 as an attractive candidate molecule for targeted tumor therapy is reported recently (see Takai et al in Oncogene, 24:287 (2005); McInnes et al in Current Topics in Med. Chem., 5: 181 (2005)).

There is a continued need to find new therapeutic agents to treat human diseases. Protein kinases, specifically but not limited to Polo-like Kinase (PLK), are especially attractive targets for the discovery of new therapeutics due to their important role in hyperproliferative disorders; cancer (e.g., solid tumors, leukemias, lymphomas, non-small cell lung cancers and esophageal carcinomas); inflammatory and autoimmune diseases (e.g., psoriasis, alopecia; multiple sclerosis; colitis, arthritis, Alzheimer's disease, glomerulonephritis and wound healing); chemotherapy agent-induced alopecia and mucositis; cardiovascular diseases (e.g., stenoses, arterioscleroses, restenoses, and hypertrophy); viral, bacterial, fungal and/or parasitic infectious diseases (e.g., cytomegalic infections, herpes, hepatitis B and C, Karposi's sarcoma, HIV diseases); nephrological diseases (e.g., glomerulonephritis); chronic and acute neurodegenerative diseases (e.g., Huntington's disease, amyotrophic lateral sclerosis, Parkinson's disease, AIDS dementia, Alzheimer's disease, ischemias of the brain and neurotraumas); skin diseases (e.g., psoriasis); bone diseases; the protection of proliferating cells (e.g., hair, intestinal, blood and progenitor cells) from DNA damage caused by radiation, UV treatment and/or cytostatic treatment; and other diseases.

### SUMMARY OF THE INVENTION

The present invention relates to compounds that have activity for inhibiting kinases. The present invention also provides compositions, articles of manufacture and kits comprising these compounds. In addition, the invention relates to methods of making the compounds of the present invention, as well as intermediates useful in such methods.

In one embodiment, a pharmaceutical composition is provided that comprises a kinase inhibitor according to the present invention as an active ingredient. Pharmaceutical compositions according to the invention may optionally comprise 0.001%-100% of one or more inhibitors of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

The invention is also directed to kits and other articles of manufacture for treating disease states associated with kinases.

In one embodiment, a kit is provided that comprises a composition comprising at least one kinase inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one kinase inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

Also provided are methods for preparing compounds, compositions and kits according to the present invention. For example, several synthetic schemes are provided herein for synthesizing compounds according to the present invention.

Also provided are methods for using compounds, compositions, kits and articles of manufacture according to the present invention.

In one embodiment, the compounds, compositions, kits and articles of manufacture are used to inhibit kinases. In particular, the compounds, compositions, kits and articles of manufacture can be used to inhibit a PLK.

In another embodiment, the compounds, compositions, kits and articles of manufacture are used to treat a disease state for which kinases possess activity that contributes to the pathology and/or symptomology of the disease state.

In another embodiment, a compound is administered to a subject wherein kinase activity within the subject is altered, preferably reduced.

In another embodiment, a prodrug of a compound is administered to a subject that is converted to the compound in vivo where it inhibits kinase.

In another embodiment, a method of inhibiting kinase is provided that comprises contacting a kinase with a compound according to the present invention.

In another embodiment, a method of inhibiting kinase is provided that comprises causing a compound according to the present invention to be present in a subject in order to inhibit kinase in vivo.

In another embodiment, a method of inhibiting a kinase is provided that comprises administering a first compound to a subject that is converted in vivo to a second compound wherein the second compound inhibits kinase in vivo. It is noted that the compounds of the present invention may be the first or second compounds.

In another embodiment, a therapeutic method is provided that comprises administering a compound according to the present invention.

In another embodiment, a method of treating a condition in a patient that is known to be mediated by kinases, or which is known to be treated by kinase inhibitors, comprising administering to the patient a therapeutically effective amount of a compound according to the present invention.

In another embodiment, a method is provided for treating a disease state for which a kinase possesses activity that contributes to the pathology and/or symptomology of the disease state, the method comprising: causing a compound according to the present invention to be present in a subject in a therapeutically effective amount for the disease state.

In another embodiment, a method is provided for treating a disease state for which a kinase possesses activity that contributes to the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which a kinase possesses activity that contributes to the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by a kinase, or that is known to be treated by kinase inhibitors.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well known in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula,” “compound having the formula” and “compound of the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibiting kinase and thus may themselves be prodrugs for another compound. Such prodrugs of another compound may or may not themselves independently have kinase inhibitory activity.

### BRIEF DESCRIPTION OF THE FIGURES

FIG. 1 illustrates SEQ. ID Nos. 1-6 referred to in this application.

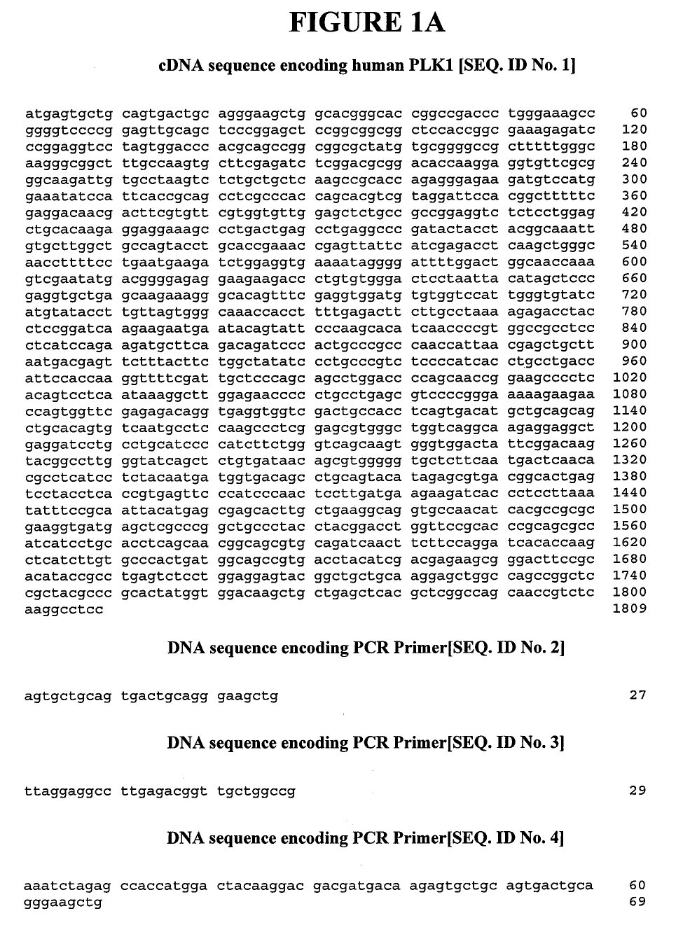

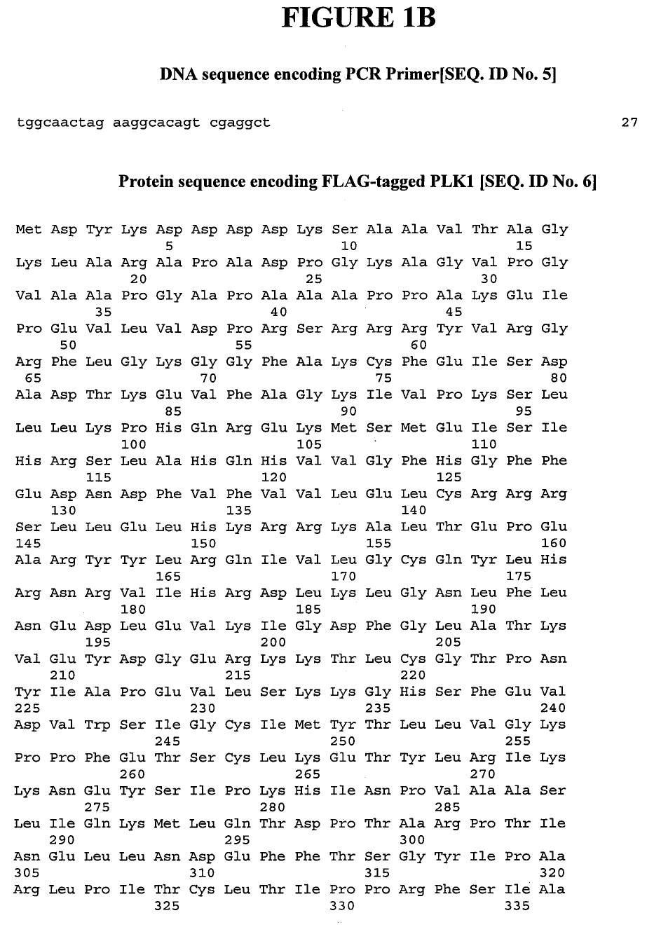

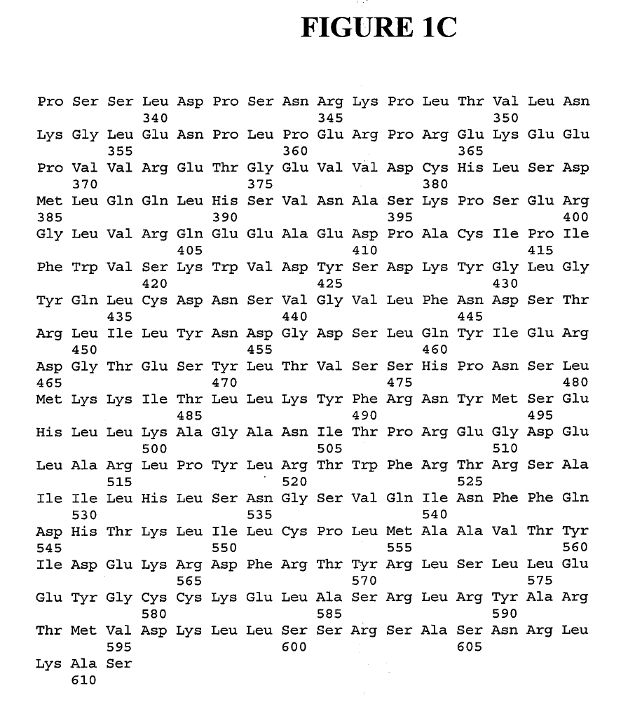

### DEFINITIONS

Unless otherwise stated, the following terms used in the specification and claims shall have the following meanings for the purposes of this application.

It is noted that, as used in the specification and the appended claims, the singular forms “a,” “an” and “the” include plural referents unless the context clearly dictates otherwise. Further, definitions of standard chemistry terms may be found in reference works, including Carey and Sundberg “ADVANCED ORGANIC CHEMISTRY 4^{TH} ED.” Vols. A (2000) and B (2001), Plenum Press, New York. Also, unless otherwise indicated, conventional methods of mass spectroscopy, NMR, HPLC, protein chemistry, biochemistry, recombinant DNA techniques and pharmacology, within the skill of the art are employed.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with (C₃₋₈) rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkenyl” means a straight or branched, carbon chain that contains at least one carbon-carbon double bond (—CR═CR′— or —CR═CR′R″, wherein R, R′ and R″ are each independently hydrogen or further substituents). Examples of alkenyl include vinyl, allyl, isopropenyl, pentenyl, hexenyl, heptenyl, 1-propenyl, 2-butenyl, 2-methyl-2-butenyl, and the like. In particular embodiments, “alkenyl,” either alone or represented along with another radical, can be a (C₂₋₂₀)alkenyl, a (C₂₋₁₅)alkenyl, a (C₂₋₁₀)alkenyl, a (C₂₋₅)alkenyl or a (C₂₋₃)alkenyl. Alternatively, “alkenyl,” either alone or represented along with another radical, can be a (C₂)alkenyl, a (C₃)alkenyl or a (C₄)alkenyl.

“Alkenylene” means a straight or branched, divalent carbon chain having one or more carbon-carbon double bonds (—CR═CR′—, wherein R and R′ are each independently hydrogen or further substituents). Examples of alkenylene include ethene-1,2-diyl, propene-1,3-diyl, methylene-1,1-diyl, and the like. In particular embodiments, “alkenylene,” either alone or represented along with another radical, can be a (C₂₋₂₀) alkenylene, a (C₂₋₁₅) alkenylene, a (C₂₋₁₀) alkenylene, a (C₂₋₅) alkenylene or a (C₂₋₃) alkenylene. Alternatively, “alkenylene,” either alone or represented along with another radical, can be a (C₂) alkenylene, a (C₃) alkenylene or a (C₄) alkenylene.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with one or more of the carbon atoms being replaced with oxygen (See “oxaalkyl”), a carbonyl group (See “oxoalkyl”), sulfur (See “thioalkyl”), and/or nitrogen (See “azaalkyl”). (C_{X})alkyl and (C_{X-Y})alkyl are typically used where X and Y indicate the number of carbon atoms in the chain. For example, (C₁₋₆)alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl and the like) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenethyl, 1-phenylethyl, 3-phenylpropyl, 2-thienylmethyl, 2-pyridinylmethyl and the like). In particular embodiments, “alkyl,” either alone or represented along with another radical, can be a (C₁₋₂₀)alkyl, a (C₁₋₁₅)alkyl, a (C₁₋₁₀)alkyl, a (C₁₋₅)alkyl or a (C₁₋₃)alkyl. Alternatively, “alkyl,” either alone or represented along with another radical, can be a (C₁)alkyl, a (C₂)alkyl or a (C₃)alkyl.

“Alkylene”, unless indicated otherwise, means a straight or branched, saturated or unsaturated, aliphatic, divalent radical. (C_{X})alkylene and (C_{X-Y})alkylene are typically used where X and Y indicate the number of carbon atoms in the chain. For example, (C₁₋₆)alkylene includes methylene (—CH₂—), ethylene (—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like. In particular embodiments, “alkylene,” either alone or represented along with another radical, can be a (C₁₋₂₀)alkylene, a (C₁₋₁₅)alkylene, a (C₁₋₁₀)alkylene, a (C₁₋₅)alkylene or a (C₁₋₃)alkylene. Alternatively, “alkylene,” either alone or represented along with another radical, can be a (C₁)alkylene, a (C₂)alkylene or a (C₃)alkylene.

“Alkylidene” means a straight or branched, saturated or unsaturated, aliphatic radical connected to the parent molecule by a double bond. (C_{X})alkylidene and (C_{X-Y})alkylidene are typically used where X and Y indicate the number of carbon atoms in the chain. For example, (C₁₋₆)alkylidene includes methylene (═CH₂), ethylidene (═CHCH₃), isopropylidene (═C(CH₃)₂), propylidene (═CHCH₂CH₃), allylidene (═CH—CH═CH₂), and the like. In particular embodiments, “alkylidene,” either alone or represented along with another radical, can be a (C₁₋₂₀)alkylidene, a (C₁₋₁₅)alkylidene, a (C₁₋₁₀)alkylidene, a (C₁₋₅)alkylidene or a (C₁₋₃)alkylidene. Alternatively, “alkylidene,” either alone or represented along with another radical, can be a (C₁)alkylidene, a (C₂)alkylidene or a (C₃)alkylidene.

“Alkynyl” means a straight or branched, carbon chain that contains at least one carbon-carbon triple bond (—C≡C— or —C≡CR, wherein R is hydrogen or a further substituent). Examples of alkynyl include ethynyl, propargyl, 3-methyl-1-pentynyl, 2-heptynyl and the like. In particular embodiments, “alkynyl,” either alone or represented along with another radical, can be a (C₂₋₂₀)alkynyl, a (C₂₋₁₅)alkynyl, a (C₂₋₁₀)alkynyl, a (C₂₋₅)alkynyl or a (C₂₋₃)alkynyl. Alternatively, “alkynyl,” either alone or represented along with another radical, can be a (C₂)alkynyl, a (C₃)alkynyl or a (C₄)alkynyl.

“Alkynylene” means a straight or branched, divalent carbon chain having one or more carbon-carbon triple bonds (—CR≡CR′—, wherein R and R′ are each independently hydrogen or further substituents). Examples of alkynylene include ethyne-1,2-diyl, propyne-1,3-diyl, and the like. In particular embodiments, “alkynylene,” either alone or represented along with another radical, can be a (C₂₋₂₀) alkynylene, a (C₂₋₁₅) alkynylene, a (C₂₋₁₀) alkynylene, a (C₂₋₅) alkynylene or a (C₂₋₃) alkynylene. Alternatively, “alkynylene,” either alone or represented along with another radical, can be a (C₂) alkynylene, a (C₃) alkynylene or a (C₄) alkynylene.

“Amido” means the radical —C(═O)—NR—, —C(═O)—NRR′, —NR—C(═O)— and/or —NR—C(═O)R′, wherein each R and R′ are independently hydrogen or a further substituent.

“Amino” means a nitrogen moiety having two further substituents where, for example, a hydrogen or carbon atom is attached to the nitrogen. For example, representative amino groups include —NH₂, —NHCH₃, —N(CH₃)₂, —NH((C₁₋₁₀)alkyl), —N((C₁₋₁₀)alkyl)₂, —NH(aryl), —NH(heteroaryl), —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (See “heteroaryl”).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. (C_{X})aryl and (C_{X-Y})aryl are typically used where X and Y indicate the number of carbon atoms in the ring. In particular embodiments, “aryl,” either alone or represented along with another radical, can be a (C₃₋₁₄)aryl, a (C₃₋₁₀)aryl, a (C₃₋₇)aryl, a (C₈₋₁₀)aryl or a (C₅₋₇)aryl. Alternatively, “aryl,” either alone or represented along with another radical, can be a (C₅)aryl, a (C₆)aryl, a (C₇)aryl, a (C₈)aryl, a (C₉)aryl or a (C₁₀)aryl.

“Azaalkyl” means an alkyl, as defined above, except where one or more of the carbon atoms forming the alkyl chain are replaced with substituted or unsubstituted nitrogen atoms (—NR— or —NRR′, wherein R and R′ are each independently hydrogen or further substituents). For example, a (C₁₋₁₀)azaalkyl refers to a chain comprising between 1 and 10 carbons and one or more nitrogen atoms.

“Bicycloalkyl” means a saturated or partially unsaturated fused, spiro or bridged bicyclic ring assembly. In particular embodiments, “bicycloalkyl,” either alone or represented along with another radical, can be a (C₄₋₁₅)bicycloalkyl, a (C₄₋₁₀)bicycloalkyl, a (C₆₋₁₀)bicycloalkyl or a (C₈₋₁₀)bicycloalkyl. Alternatively, “bicycloalkyl,” either alone or represented along with another radical, can be a (C₈)bicycloalkyl, a (C₉)bicycloalkyl or a (C₁₀)bicycloalkyl.

“Bicycloaryl” means a fused, spiro or bridged bicyclic ring assembly wherein at least one of the rings comprising the assembly is aromatic. (C_{X})bicycloaryl and (C_{X-Y})bicycloaryl are typically used where X and Y indicate the number of carbon atoms in the bicyclic ring assembly and directly attached to the ring. In particular embodiments, “bicycloaryl,” either alone or represented along with another radical, can be a (a (C₄₋₁₅)bicycloaryl, a (C₄₋₁₀)bicycloaryl, a (C₆₋₁₀)bicycloaryl or a (C₈₋₁₀)bicycloaryl. Alternatively, “bicycloalkyl,” either alone or represented along with another radical, can be a (C₈)bicycloaryl, a (C₉)bicycloaryl or a (C₁₀)bicycloaryl.

“Bridging ring” and “bridged ring” as used herein refer to a ring that is bonded to another ring to form a compound having a bicyclic or polycyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRR′, wherein R and R′ are each independently hydrogen or further substituents.

“Carbocycle” means a ring consisting of carbon atoms.

“Carbonyl” means the radical —C(═O)— and/or —C(═O)R, wherein R is hydrogen or a further substituent. It is noted that the carbonyl radical may be further substituted with a variety of substituents to form different carbonyl groups including acids, acid halides, aldehydes, amides, esters, and ketones.

“Carboxy” means the radical —C(═O)—O— and/or —C(═O)—OR, wherein R is hydrogen or a further substituent. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, bicyclic or polycyclic ring assembly. (C_{X})cycloalkyl and (C_{X-Y})cycloalkyl are typically used where X and Y indicate the number of carbon atoms in the ring assembly. For example, (C₃₋₁₀)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like. In particular embodiments, “cycloalkyl,” either alone or represented along with another radical, can be a (C₃₋₁₄)cycloalkyl, a (C₃₋₁₀)cycloalkyl, a (C₃₋₇)cycloalkyl, a (C₈₋₁₀)cycloalkyl or a (C₅₋₇)cycloalkyl. Alternatively, “cycloalkyl,” either alone or represented along with another radical, can be a (C₅)cycloalkyl, a (C₆)cycloalkyl, a (C₇)cycloalkyl, a (C₈)cycloalkyl, a (C₉)cycloalkyl or a (C₁₀)cycloalkyl.

“Cycloalkylene” means a divalent, saturated or partially unsaturated, monocyclic, bicyclic or polycyclic ring assembly. (C_{X})cycloalkylene and (C_{X-Y})cycloalkylene are typically used where X and Y indicate the number of carbon atoms in the ring assembly. In particular embodiments, “cycloalkylene,” either alone or represented along with another radical, can be a (C₃₋₁₄)cycloalkylene, a (C₃₋₁₀)cycloalkylene, a (C₃₋₇)cycloalkylene, a (C₈₋₁₀)cycloalkylene or a (C₅₋₇)cycloalkylene. Alternatively, “cycloalkylene,” either alone or represented along with another radical, can be a (C₅)cycloalkylene, a (C₆)cycloalkylene, a (C₇)cycloalkylene, a (C₈)cycloalkylene, a (C₉)cycloalkylene or a (C₁₀)cycloalkylene.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure where the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” means fluoro, chloro, bromo or iodo.

“Heteroalkyl” means alkyl, as defined in this application, provided that one or more of the atoms within the alkyl chain is a heteroatom. In particular embodiments, “heteroalkyl,” either alone or represented along with another radical, can be a hetero(C₁₋₂₀)alkyl, a hetero(C₁₋₁₅)alkyl, a hetero(C₁₋₁₀)alkyl, a hetero(C₁₋₅)alkyl, a hetero(C₁₋₃)alkyl or a hetero(C₁₋₂)alkyl. Alternatively, “heteroalkyl,” either alone or represented along with another radical, can be a hetero(C₁)alkyl, a hetero(C₂)alkyl or a hetero(C₃)alkyl.

“Heteroaryl” means a monocyclic, bicyclic or polycyclic aromatic group wherein at least one ring atom is a heteroatom and the remaining ring atoms are carbon. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted. In particular embodiments, “heteroaryl,” either alone or represented along with another radical, can be a hetero(C₁₋₁₃)aryl, a hetero(C₂₋₁₃)aryl, a hetero(C₂₋₆)aryl, a hetero(C₃₋₉)aryl or a hetero(C₅₋₉)aryl. Alternatively, “heteroaryl,” either alone or represented along with another radical, can be a hetero(C₃)aryl, a hetero(C₄)aryl, a hetero(C₅)aryl, a hetero(C₆)aryl, a hetero(C₇)aryl, a hetero(C₈)aryl or a hetero(C₉)aryl.

“Heteroatom” refers to an atom that is not a carbon atom. Particular examples of heteroatoms include, but are not limited to, nitrogen, oxygen, and sulfur.

“Heteroatom moiety” includes a moiety where the atom by which the moiety is attached is not a carbon. Examples of heteroatom moieties include —NR—, —N⁺(O⁻)═, —O—, —S— or —S(O)₂—, wherein R is hydrogen or a further substituent.

“Heterobicycloalkyl” means bicycloalkyl, as defined in this application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like. In particular embodiments, “heterobicycloalkyl,” either alone or represented along with another radical, can be a hetero(C₁₋₁₄)bicycloalkyl, a hetero(C₄₋₁₄)bicycloalkyl, a hetero(C₄₋₉)bicycloalkyl or a hetero(C₅₋₉)bicycloalkyl. Alternatively, “heterobicycloalkyl,” either alone or represented along with another radical, can be a hetero(C₅)bicycloalkyl, hetero(C₆)bicycloalkyl, hetero(C₇)bicycloalkyl, hetero(C₈)bicycloalkyl or a hetero(C₉)bicycloalkyl.

“Heterobicycloaryl” means bicycloaryl, as defined in this application, provided that one or more of the atoms within the ring is a heteroatom. For example, hetero(C₄₋₁₂)bicycloaryl as used in this application includes, but is not limited to, 2-amino-4-oxo-3,4-dihydropteridin-6-yl, tetrahydroisoquinolinyl, and the like. In particular embodiments, “heterobicycloaryl,” either alone or represented along with another radical, can be a hetero(C₁₋₁₄)bicycloaryl, a hetero(C₄₋₁₄)bicycloaryl, a hetero(C₄₋₉)bicycloaryl or a hetero(C₅₋₉)bicycloaryl. Alternatively, “heterobicycloaryl,” either alone or represented along with another radical, can be a hetero(C₅)bicycloaryl, hetero(C₆)bicycloaryl, hetero(C₇)bicycloaryl, hetero(C₈)bicycloaryl or a hetero(C₉)bicycloaryl.

“Heterocycloalkyl” means cycloalkyl, as defined in this application, provided that one or more of the atoms forming the ring is a heteroatom selected, independently from N, O, or S, Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like. In particular embodiments, “heterocycloalkyl,” either alone or represented along with another radical, can be a hetero(C₁₋₁₃)cycloalkyl, a hetero(C₁₋₉)cycloalkyl, a hetero(C₁₋₆)cycloalkyl, a hetero(C₅₋₉)cycloalkyl or a hetero(C₂₋₆)cycloalkyl. Alternatively, “heterocycloalkyl,” either alone or represented along with another radical, can be a hetero(C₂)cycloalkyl, a hetero(C₃)cycloalkyl, a hetero(C₄)cycloalkyl, a hetero(C₅)cycloalkyl, a hetero(C₆)cycloalkyl, hetero(C₇)cycloalkyl, hetero(C₈)cycloalkyl or a hetero(C₉)cycloalkyl.

“Heterocycloalkylene” means cycloalkylene, as defined in this application, provided that one or more of the ring member carbon atoms is replaced by a heteroatom. In particular embodiments, “heterocycloalkylene,” either alone or represented along with another radical, can be a hetero(C₁₋₁₃)cycloalkylene, a hetero(C₁₋₉)cycloalkylene, a hetero(C₁₋₆)cycloalkylene, a hetero(C₅₋₉)cycloalkylene or a hetero(C₂₋₆)cycloalkylene. Alternatively, “heterocycloalkylene,” either alone or represented along with another radical, can be a hetero(C₂)cycloalkylene, a hetero(C₃)cycloalkylene, a hetero(C₄)cycloalkylene, a hetero(C₅)cycloalkylene, a hetero(C₆)cycloalkylene, hetero(C₇)cycloalkylene, hetero(C₈)cycloalkylene or a hetero(C₉)cycloalkylene.

“Hydroxy” means the radical —OH.

“IC₅₀” means the molar concentration of an inhibitor that produces 50% inhibition of the target enzyme.

“Imino” means the radical —CR(═NR′) and/or —C(═NR′)—, wherein R and R′ are each independently hydrogen or a further substituent.

“Isomers” means compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Leaving group” means the group with the meaning conventionally associated with it in synthetic organic chemistry, i.e., an atom or group displaceable under reaction (e.g., alkylating) conditions. Examples of leaving groups include, but are not limited to, halo (e.g., F, Cl, Br and I), alkyl (e.g., methyl and ethyl) and sulfonyloxy (e.g., mesyloxy, ethanesulfonyloxy, benzenesulfonyloxy and tosyloxy), thiomethyl, thienyloxy, dihalophosphinoyloxy, tetrahalophosphoxy, benzyloxy, isopropyloxy, acyloxy, and the like.

“Moiety providing X atom separation” and “linker providing X atom separation” between two other moieties mean that the chain of atoms directly linking the two other moieties is X atoms in length. When X is given as a range (e.g., X₁-X₂), then the chain of atoms is at least X₁ and not more than X₂ atoms in length. It is understood that the chain of atoms can be formed from a combination of atoms including, for example, carbon, nitrogen, sulfur and oxygen atoms. Further, each atom can optionally be bound to one or more substituents, as valencies allow. In addition, the chain of atoms can form part of a ring. Accordingly, in one embodiment, a moiety providing X atom separation between two other moieties (R and R′) can be represented by R-(L)_{X}-R′ where each L is independently selected from the group consisting of CR″R′″, NR″″, O, S, CO, CS, C═NR′″″, SO, SO₂, and the like, where any two or more of R″, R′″, R″″ and R′″″ can be taken together to form a substituted or unsubstituted ring.

“Nitro” means the radical —NO₂.

“Oxaalkyl” means an alkyl, as defined above, except where one or more of the carbon atoms forming the alkyl chain are replaced with oxygen atoms (—O— or —OR, wherein R is hydrogen or a further substituent). For example, an oxa(C₁₋₁₀)alkyl refers to a chain comprising between 1 and 10 carbons and one or more oxygen atoms.

“Oxoalkyl” means an alkyl, as defined above, except where one or more of the carbon atoms forming the alkyl chain are replaced with carbonyl groups (—C(═O)— or —C(═O)—R, wherein R is hydrogen or a further substituent). The carbonyl group may be an aldehyde, ketone, ester, amide, acid or acid halide. For example, an oxo(C₁₋₁₀)alkyl refers to a chain comprising between 1 and 10 carbon atoms and one or more carbonyl groups.

“Oxy” means the radical —O— or —OR, wherein R is hydrogen or a further substituent. Accordingly, it is noted that the oxy radical may be further substituted with a variety of substituents to form different oxy groups including hydroxy, alkoxy, aryloxy, heteroaryloxy or carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Polycyclic ring” includes bicyclic and multi-cyclic rings. The individual rings comprising the polycyclic ring can be fused, spiro or bridging rings.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

“Protected derivatives” means derivatives of inhibitors in which a reactive site or sites are blocked with protecting groups. Protected derivatives are useful in the preparation of inhibitors or in themselves may be active as inhibitors. A comprehensive list of suitable protecting groups can be found in T. W. Greene, Protecting Groups in Organic Synthesis, 3rd edition, John Wiley & Sons, Inc. 1999.

“Ring” and “ring assembly” means a carbocyclic or a heterocyclic system and includes aromatic and non-aromatic systems. The system can be monocyclic, bicyclic or polycyclic. In addition, for bicyclic and polycyclic systems, the individual rings comprising the polycyclic ring can be fused, spiro or bridging rings.

“Subject” and “patient” includes humans, non-human mammals (e.g., dogs, cats, rabbits, cattle, horses, sheep, goats, swine, deer, and the like) and non-mammals (e.g., birds, and the like).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [(CH₃)₃C—OCO—], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; CH₃CH(NH₂)CO—), Arg (arginine), Asn (asparagine), Asp (aspartic acid), Cys (cysteine), Glu (glutamic acid), His (histidine), Ile (isoleucine), Leu (leucine; (CH₃)₂CHCH₂CH(NH₂)CO—), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Suitable peptide residues include peptide residues comprising two to five, and optionally two to three, of the aforesaid amino acid residues. Examples of such peptide residues include, but are not limited to, residues of such peptides as Ala-Ala [CH₃CH(NH₂)CO—NHCH(CH₃)CO—], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl).

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —SO— and/or —SO—R, wherein R is hydrogen or a further substituent. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂— and/or —SO₂—R, wherein R is hydrogen or a further substituent. It is noted that the sulfonyl radical may be further substituted with a variety of substituents to form different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thio” denotes replacement of an oxygen by a sulfur and includes, but is not limited to, —SR—, —S— and ═S containing groups.

“Thioalkyl” means an alkyl, as defined above, except where one or more of the carbon atoms forming the alkyl chain are replaced with sulfur atoms (—S— or —S—R, wherein R is hydrogen or a further substituent). For example, a thio(C₁₋₁₀)alkyl refers to a chain comprising between 1 and 10 carbons and one or more sulfur atoms.

“Thiocarbonyl” means the radical —C(═S)— and/or —C(═S)—R, wherein R is hydrogen or a further substituent. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a (C₁)alkyl comprises methyl (i.e., —CH₃) as well as —CRR′R″ where R, R′, and R″ may each independently be hydrogen or a further substituent where the atom attached to the carbon is a heteroatom or cyano. Hence, CF₃, CH₂OH and CH₂CN, for example, are all (C₁)alkyls. Similarly, terms such as alkylamino and the like comprise dialkylamino and the like.

A compound having a formula that is represented with a dashed bond is intended to include the formulae optionally having zero, one or more double bonds, as exemplified and shown below:

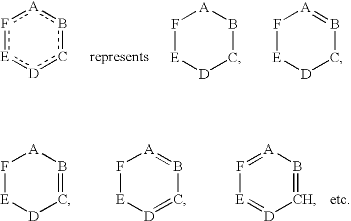

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

### DETAILED DESCRIPTION OF THE INVENTION

The present invention relates to compounds that may be used to inhibit kinases and, in particular, Polo-like Kinases (referred to herein as PLKs). The present invention also relates to pharmaceutical compositions, kits and articles of manufacture comprising such compounds. In addition, the present invention relates to methods and intermediates useful for making the compounds. Further, the present invention relates to methods of using said compounds. It is noted that the compounds of the present invention may also possess activity for other members of the same protein family and thus may be used to address disease states associated with these other family members.

Disregulation of PLKs is implicated in such areas as hyperproliferative disorders; cancer (e.g., solid tumors, leukemias, lymphomas, non-small cell lung cancers and esophageal carcinomas); inflammatory and autoimmune diseases (e.g., psoriasis, alopecia; multiple sclerosis; colitis, arthritis, Alzheimer's disease, glomerulonephritis and wound healing); chemotherapy agent-induced alopecia and mucositis; cardiovascular diseases (e.g., stenoses, arterioscleroses, restenoses, and hypertrophy); viral, bacterial, fungal and/or parasitic infectious diseases (e.g., cytomegalic infections, herpes, hepatitis B and C, Karposi's sarcoma, HIV diseases); nephrological diseases (e.g., glomerulonephritis); chronic and acute neurodegenerative diseases (e.g., Huntington's disease, amyotrophic lateral sclerosis, Parkinson's disease, AIDS dementia, Alzheimer's disease, ischemias of the brain and neurotraumas); skin diseases (e.g., psoriasis); bone diseases; the protection of proliferating cells (e.g., hair, intestinal, blood and progenitor cells) from DNA damage caused by radiation, UV treatment and/or cytostatic treatment.

It is noted that the compounds of the present invention may also possess inhibitory activity for other protein kinase family members and thus may be used to address disease states associated with these other family members.

### Polo-Like Kinase Inhibitors

In one embodiment, PLK inhibitors of the present invention comprise:

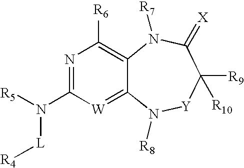

wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
L is absent or a linker providing 1, 2, 3, 4, 5 or 6 atom separation between R₄ and the nitrogen to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₄ is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₅ is hydrogen or a substituent convertible in vivo to hydrogen;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

In another embodiment, PLK inhibitors of the present invention comprise:

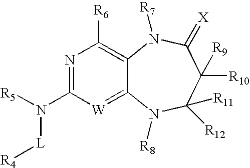

wherein
R₁₁ and R₁₂ are each independently selected from the group consisting of hydrogen, cyano, carbonyl, oxycarbonyl, aminocarbonyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₁ and R₁₂ are taken together with the atom to which they are bound to form a carbonyl or imino group,
or any two R₈, R₉, R₁₀, R₁₁ and R₁₂ are taken together to form a substituted or unsubstituted ring.

In still another embodiment, PLK inhibitors of the present invention comprise:

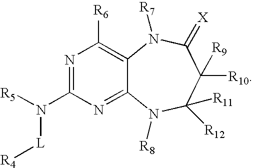

In yet another embodiment, PLK inhibitors of the present invention comprise:

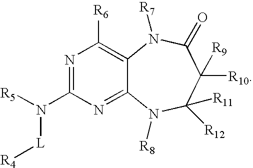

In a further embodiment, PLK inhibitors of the present invention comprise:

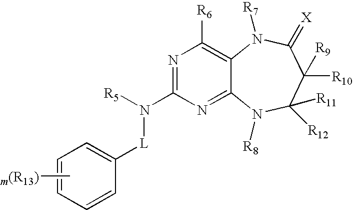

wherein
m is selected from the group consisting of 0, 1, 2, 3, 4 and 5; and
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring.

In still a further embodiment, PLK inhibitors of the present invention comprise:

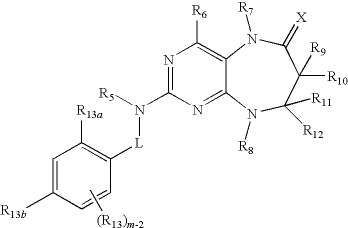

wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ and R_{13b} are each independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In yet a further embodiment, PLK inhibitors of the present invention comprise:

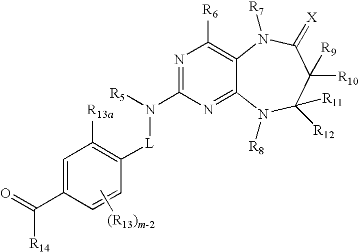

wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ is selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₁₄ is selected from the group consisting of hydrogen, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In another embodiment, PLK inhibitors of the present invention comprise:

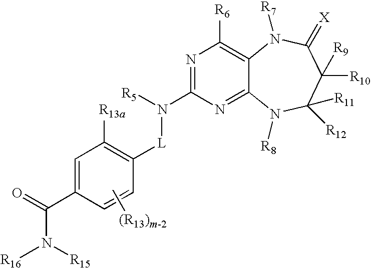

wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ is selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₁₅ are R₁₆ are each independently selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form a substituted or unsubstituted ring.

In still another embodiment, PLK inhibitors of the present invention comprise:

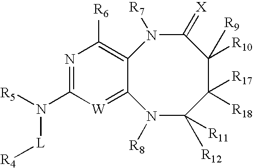

wherein
R₁₁ and R₁₂ are each independently selected from the group consisting of hydrogen, cyano, carbonyl, oxycarbonyl, aminocarbonyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₁ and R₁₂ are taken together with the atom to which they are bound to form a carbonyl or imino group; and
R₁₇ and R₁₈ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted,
or any two R₈, R₉, R₁₀, R₁₁, R₁₂, R₁₇ and R₁₈ are taken together to form a substituted or unsubstituted ring.

In yet another embodiment, the present invention relates to a process comprising:
reacting a compound comprising the formula

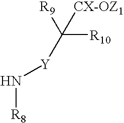

with a compound comprising the formula

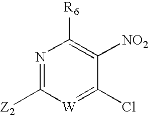

under conditions that form a first reaction product comprising the formula

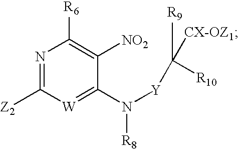

treating the first reaction product under conditions that form a second reaction product comprising the formula

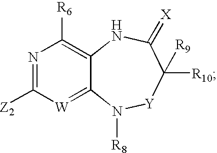

reacting the second reaction product with a compound comprising the formula

R₇-Z₃
under conditions that form a third reaction product comprising the formula

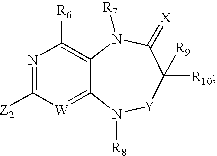

reacting the third reaction product with a compound comprising the formula

R₄-L-NR₅H
under conditions that form a compound comprising the formula

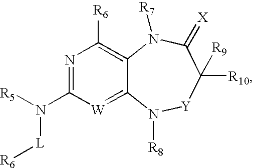

wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₁, Z₂ and Z₃ are each independently a leaving group;
L is absent or a linker providing 1, 2, 3, 4, 5 or 6 atom separation between R₄ and the nitrogen to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₄ is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₅ is hydrogen or a substituent convertible in vivo to hydrogen;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

In a further embodiment, the present invention relates to compounds comprising:

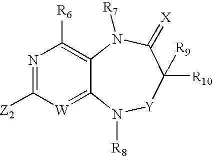

wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₂ is a leaving group;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

In still a further embodiment, the present invention relates to compounds comprising:

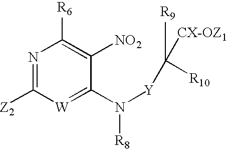

wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₁ and Z₂ are each independently a leaving group;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

In one variation of each of the above embodiments, W is —CH═.

In another variation of each of the above embodiments and variations, X is O.

In another variation of each of the above embodiments and variations, n is 2. In still another variation of each of the above embodiments and variations, n is 3. In yet another variation of each of the above embodiments and variations, n is 4. In a further variation of each of the above embodiments and variations, n is 5.

In still another variation of each of the above embodiments and variations, L is a substituted or unsubstituted (C₁₋₁₃)alkyl.

In yet another variation of each of the above embodiments and variations, L is —CHR₁₉—; and R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In a further variation of each of the above embodiments and variations, L is absent.

In the described definitions of the linker L, the 1-6 atom separation refers to the direct linear linkage between R₄ and the nitrogen to which L is attached. As such, branching along the 1-6 atom separation is permitted, as is clear from the variations described.

In still a further variation of each of the above embodiments and variations, R₁ is selected from the group consisting of hydrogen and a substituted or unsubstituted (C₁₋₅)alkyl. In another variation of each of the above embodiments and variations, R₁ is hydrogen.

In yet a further variation of each of the above embodiments and variations, R₄ is a substituted or unsubstituted hetero(C₁₋₁₀)aryl.

In another variation of each of the above embodiments and variations, R₅ is hydrogen. In still another variation of each of the above embodiments and variations, R₅ is a substituent convertible in vivo to hydrogen. In yet another variation of each of the above embodiments and variations, R₅ is selected from the group consisting of hydrolyzable groups, groups having an oxycarbonyl group, amino acid residues, peptide residues, o-nitrophenylsulfenyl, trimethylsilyl, tetrahydro-pyranyl, diphenylphosphinyl, arylsulfonyl groups, methyl groups substituted with phenyl or benzyloxy, arylmethoxycarbonyl groups, and halogenoethoxycarbonyl groups.

In a further variation of each of the above embodiments and variations, R₆ is hydrogen.

In still a further variation of each of the above embodiments and variations, R₇ is selected from the group consisting of hydrogen and a substituted or unsubstituted (C₁₋₃)alkyl.

In yet a further variation of each of the above embodiments and variations, R₈ is selected from the group consisting of (C₁₋₅)alkyl and (C₃₋₁₂)cycloalkyl, each substituted or unsubstituted. In another variation of each of the above embodiments and variations, R₈ is selected from the group consisting of isopropyl, cyclopropyl, cyclopentyl and cyclohexyl, each substituted or unsubstituted. In a further variation of each of the above embodiments and variations, R₈ is a substituted or unsubstituted cyclohexyl.

In still another variation of each of the above embodiments and variations, R₉ is selected from the group consisting of hydrogen and a substituted or unsubstituted (C₁₋₃)alkyl. In a further variation of each of the above embodiments and variations, R₉ is ethyl. In still a further variation of each of the above embodiments and variations, R₉ is propyl. In yet a further variation of each of the above embodiments and variations, R₉ is —CH₂—CN. In another variation of each of the above embodiments and variations, R₉ is alkoxy. In still another variation of each of the above embodiments and variations, R₉ is hydroxyl.

In yet another variation of each of the above embodiments and variations, R₉ is a substituted or unsubstituted (C₁₋₅)alkenyl. In a further variation of each of the above embodiments and variations, R₉ has the formula

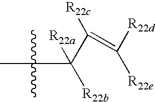

wherein
each R₂₂ₐ, R_{22b}, R_{22c}, R_{22d} and R₂₂ₑ are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In a further variation of each of the above embodiments and variations, R₉ has the formula

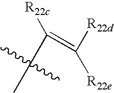

wherein
each R_{22c}, R_{22d} and R₂₂ₑ are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still a further variation of each of the above embodiments and variations, R₉ is a substituted or unsubstituted (C₁₋₅)alkynyl. In yet a further variation of each of the above embodiments and variations, R₉ has the formula

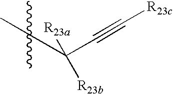

wherein
each R₂₃ₐ, R_{23b} and R_{23c} are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In yet a further variation of each of the above embodiments and variations, R₉ has the formula

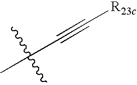

wherein
R_{23c} is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ is selected from the group consisting of hydrogen and a substituted or unsubstituted (C₁₋₃)alkyl. In still another variation of each of the above embodiments and variations, R₁₀ is methyl.

In a further variation of each of the above embodiments and variations, R₁₁ is selected from the group consisting of hydrogen and a substituted or unsubstituted (C₁₋₃)alkyl.

In still a further variation of each of the above embodiments and variations, R₁₂ is selected from the group consisting of hydrogen and a substituted or unsubstituted (C₁₋₃)alkyl.

In yet a further variation of each of the above embodiments and variations, at least one R₁₃ is a substituted or unsubstituted alkoxy.

In another variation of each of the above embodiments and variations, at least one R₁₃, R₁₃ₐ or R_{13b} comprises —C(O)NR₁₅R₁₆, wherein R₁₅ and R₁₆ are each independently selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still another variation of each of the above embodiments and variations, at least one R₁₃, R₁₃ₐ or R_{13b} comprises —C(O)OR₂₀, wherein R₂₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In yet another variation of each of the above embodiments and variations, m is 2.

In a further variation of each of the above embodiments and variations, R₁₃ₐ is a substituted or unsubstituted alkoxy.

In still a further variation of each of the above embodiments and variations, R₁₄ is hydroxy.

In yet a further variation of each of the above embodiments and variations, R₂₀ is hydroxy.

In another variation of each of the above embodiments and variations, R₉ and R₁₇ are taken together to form a substituted or unsubstituted 3-, 4-, 5-, 6-, 7- or 8-membered ring. In still another variation of each of the above embodiments and variations, R₉ and R₁₇ are taken together to form a substituted or unsubstituted (C₃₋₁₀)cycloalkyl ring. In yet another variation of each of the above embodiments and variations, R₉ and R₁₇ are taken together to form a substituted or unsubstituted ring selected from the group consisting of cyclopropane, cyclobutane, cyclopentane and cyclohexane.

In a further variation of each of the above embodiments and variations, the ring formed by R₉ and R₁₇ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still a further variation of each of the above embodiments and variations, R₈ and R₁₁ are taken together to form a substituted or unsubstituted 5-, 6-, 7- or 8-membered ring. In yet a further variation of each of the above embodiments and variations, R₈ and R₁₁ are taken together to form a substituted or unsubstituted pyrrolidine.

In another variation of each of the above embodiments and variations, the ring formed by R₈ and R₁₁ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still another variation of each of the above embodiments and variations, R₁₁ and R₁₇ are taken together to form a substituted or unsubstituted 5-, 6-, 7- or 8-membered ring. In yet another variation of each of the above embodiments and variations, R₁₁ and R₁₇ are taken together to form a substituted or unsubstituted pyrrolidine.

In another variation of each of the above embodiments and variations, R₁₅ is 1-methylpiperidin-4-yl.

In still another variation of each of the above embodiments and variations, R₁₅ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₉₋₁₂)bicycloaryl and hetero(C₄₋₁₂)bicycloaryl, each substituted or unsubstituted.

In a further variation of each of the above embodiments and variations, the ring formed by R₁₁ and R₁₇ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still a further variation of each of the above embodiments and variations, R₉ is ethyl; and R₁₀ is methyl.

In yet a further variation of each of the above embodiments and variations, R₉ is selected from the group consisting of cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and R₁₀ is methyl.

In another variation of each of the above embodiments and variations, L is absent; R₁₃ₐ is —OCH₃; R₁₅ is 1-methylpiperidin-4-yl; and R₁₆ is hydrogen.

In still a further variation of each of the above embodiments and variations, Z₁ is halo.

In yet a further variation of each of the above embodiments and variations, Z₂ is halo.

In another variation of each of the above embodiments and variations, Z₃ is halo.

Particular examples of compounds according to the present invention include, but are not limited to:
4-(9-cyclopentyl-5-methyl-6-oxo-6,7,8,9-tetrahydro-5H-pyrimido[4,5-b][1,4]diazepin-2-ylamino)-3-methoxybenzoic acid;
4-[(9-cyclopentyl-5-methyl-6-oxo-6,7,8,9-tetrahydro-5H-pyrimido[4,5-b][1,4]diazepin-2-yl)amino]-3-methoxy-N-methylbenzamide;
4-[(9-cyclopentyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopentyl-5,8-dimethyl-6-oxo-6,7,8,9-tetrahydro-5H-pyrimido[4,5-b][1,4]diazepin-2-ylamino)-3-methoxybenzoic acid;
4-[(9-cyclopentyl-5,8-dimethyl-6-oxo-6,7,8,9-tetrahydro-5H-pyrimido[4,5-b][1,4]diazepin-2-yl)amino]-3-methoxy-N-methylbenzamide;
4-{[(cis)-10-cyclopentyl-5-methyl-6-oxo-5,6,6a,7,8,9,9a,10-octahydrocyclopenta[e]pyrimido[5,4-b][1,4]diazepin-2-yl]amino}-3-methoxy-N-methylbenzamide;
4-(9-cyclopentyl-8-ethyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-{[(7aR)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
3-methoxy-4-{[(7aS)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
4-[(9-isopropyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopropyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-[(9-cyclohexyll-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-isobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-benzyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-(pentan-3-yl)-6,7,8,9-tetrahydro-5H-pyrimido[4,5-b][1,4]diazepin-2-ylamino)-N-(1-methylpiperidin-4-yl)benzamide;
4-(9-sec-butyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-allyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-phenyl-6,7,8,9-tetrahydro-5H-pyrimido[4,5-b][1,4]diazepin-2-ylamino)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(10-Cyclopentyl-5-methyl-6-oxo-5,6,7,8,9,10-hexahydro-pyrimido[4,5-b][1,4]diazocin-2-ylamino)-3-methoxy-N-(1-methyl-piperidin-4-yl)-benzamide;
(R)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
(S)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
4-(9-cyclopentyl-5-methyl-6-oxo-6,7,8,9-tetrahydro-5H-pyrimido[4,5-b][1,4]diazepin-2-ylamino)-3-methoxy N-(1-(methylsulfonyl)piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(3-(2-oxopyrrolidin-1-yl)propyl)benzamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
N-(2-acetamidoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxoimidazolidin-1-yl)ethyl)benzamide;
N-(2-aminoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxopyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(6-oxopiperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperazin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(methylamino)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-2-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-ylmethyl)benzamide;
4-(9-cyclopentyl-5-methyl-6-oxo-6,7,8,9-tetrahydro-5H-pyrimido[4,5-b][1,4]diazepin-2-ylamino)-N-cyclopropyl-3-methoxy-N-(methylpyridin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-(cyclopropanecarbonyl)pyridin-4-yl)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-N-((1-(cyclopropanecarbonyl)piperidin-4-yl)methyl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(1-isopropylpiperidin-4-yl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(dimethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(diethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(1,2,2,6,6-pentamethylpiperidin-4-yl)benzamide;
9-cyclopentyl-5-methyl-2-(pyridin-3-ylamino)-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
3-Methoxy-4-(5-methyl-6-oxo-9-(tetrahydro-2H-pyran-4-yl)-6,7,8,9-tetrahydro-5H-pyrimido[4,5-b][1,4]diazepin-2-ylamino)-N-(1-methylpiperidin-4-yl)benzamide;
4-(9-(4,4-Difluorocyclohexyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclopentyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(3-(dimethylamino)propyl)-3-methoxybenzamide;
N-(1-acetylpiperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-(methylsulfonyl)piperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(2-(dimethylamino)ethyl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclopentyl-5,7,7-trimethyl-6-oxo-6,7,8,9-tetrahydro-5H-pyrimido[4,5-b][1,4]diazepin-2-ylamino)-N, 3-dimethoxybenzamide;
4-(9-cyclopentyl-5,7,7-trimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-morpholinobenzamide;
4-(9-cyclopentyl-5,7,7-trimethyl-6-oxo-6,7,8,9-tetrahydro-5H-pyrimido[4,5-b][1,4]diazepin-2-ylamino)-N-(4-(2-hydroxyethyl)piperazin-1-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(4-cyclopentylpiperazin-1-yl)-3-methoxybenzamide;
N-(azetidin-3-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
N-(azepan-4-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-hydroxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-ethoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-methylpiperidin-4-yl)-3-propoxybenzamide;
4-(9-cyclopentyl-5,7,7-trimethyl-6-oxo-6,7,8,9-tetrahydro-5H-pyrimido[4,5-b][1,4]diazepin-2-ylamino)-3-(2,2-difluoroethoxy)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic acid;
4-(9-Cyclopentyl-5,7,7-trimethyl-6-oxo-6,7,8,9-tetrahydro-5H-pyrimido[4,5-b][1,4]diazepin-2-ylamino)-3-(difluoromethoxy)-N-(1-methylpiperidin-4-yl)benzamide;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-isopropyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclopentyl-5′-methyl-6′-oxo-5′,6′,8′,9′-tetrahydrospiro-[cyclopropane-1,7′-pyrimido[4,5-b][1,4]diazepine]-2′-ylamino)-3-methoxy-N-(1-methylpiperidin-3-yl)benzamide;
(R)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)-benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-N-(1-ethylpiperidin-4-yl)-3-methoxybenzamide;
3-methoxy-4-(5′-methyl-9′-(2-methylcyclopentyl)-6′-oxo-5′,6′,8′,9′-tetrahydrospiro[cyclopropane-1,7′-pyrimido[4,5-b][1,4]diazepine]-2′-ylamino)-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid;
4-(9′-Cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(4-methylpiperazin-1-yl)benzamide;
(R)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
4-(7-(Cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-(2-amino-2-oxoethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid;
4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(piperidin-4-yl)benzamide;
(S)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(Z)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(pent-2-ynyl)-6,7,8,9-tetrahydro-5H-pyrimido[4,5-b][1,4]diazepin-2-ylamino)-3-methoxy-N-(1-methylpiperidin-4-yl)benzamide;
(E)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9-cyclopentyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxybenzoic acid;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-7-hydroxy-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic acid;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((S)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
(S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
9-cyclopentyl-5-methyl-2-(pyridin-2-ylamino)-8,9-dihydro-5H-pyrimido[4,5-b][1,4]diazepin-6(7H)-one;
9-cyclopentyl-5-methyl-2-(phenylamino)-8,9-dihydro-5H-pyrimido[4,5-b][1,4]diazepin-6(7H)-one;
9-cyclopentyl-5-methyl-2-(2-(trifluoromethyl)phenylamino)-8,9-dihydro-5H-pyrimido[4,5-b][1,4]diazepin-6(7H)-one;
9-cyclopentyl-2-(2-fluoro-6-methoxyphenylamino)-5-methyl-8,9-dihydro-5H-pyrimido[4,5-b][1,4]diazepin-6(7H)-one;
9-cyclopentyl-2-(3-methoxypyrazin-2-ylamino)-5-methyl-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)nicotinonitrile;
5-(9-cyclopentyl-5-methyl-6-oxo-6,7,8,9-tetrahydro-5H-pyrimido[4,5-b][1,4]diazepin-2-ylamino)picolinonitrile;
5-(9-cyclopentyl-5-methyl-6-oxo-6,7,8,9-tetrahydro-5H-pyrimido[4,5-b][1,4]diazepin-2-ylamino)picolinamide;
6-(9-cyclopentyl-5-methyl-6-oxo-6,7,8,9-tetrahydro-5H-pyrimido[4,5-b][1,4]diazepin-2-ylamino)nicotinamide;
9-cyclopentyl-2-(5-fluoro-2-methoxyphenylamino)-5-methyl-8,9-dihydro-5H-pyrimido[4,5-b][1,4]diazepin-6(7H)-one;
9-cyclopentyl-2-(3-fluoro-2-methoxyphenylamino)-5-methyl-8,9-dihydro-5H-pyrimido[4,5-b][1,4]diazepin-6(7H)-one;
9-cyclopentyl-2-(2-methoxyphenylamino)-5-methyl-8,9-dihydro-5H-pyrimido[4,5-b][1,4]diazepin-6(7H)-one;
9-cyclopentyl-5-methyl-2-(1,2,3,4-tetrahydroisoquinolin-6-ylamino)-8,9-dihydro-5H-pyrimido[4,5-b][1,4]diazepin-6(7H)-one;
2-(4-aminophenylamino)-9-cyclopentyl-5-methyl-8,9-dihydro-5H-pyrimido[4,5-b][1,4]diazepin-6(7H)-one;
N-(4-(9-cyclopentyl-5-methyl-6-oxo-6,7,8,9-tetrahydro-5H-pyrimido[4,5-b][1,4]diazepin-2-ylamino)phenyl)acetamide;
2-(3-aminophenylamino)-9-cyclopentyl-5-methyl-8,9-dihydro-5H-pyrimido[4,5-b][1,4]diazepin-6(7H)-one;
N-(3-(9-cyclopentyl-5-methyl-6-oxo-6,7,8,9-tetrahydro-5H-pyrimido[4,5-b][1,4]diazepin-2-ylamino)phenyl)acetamide;
4-(9-cyclobutyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclopentyl-5-methyl-6-oxo-6,7,8,9-tetrahydro-5H-pyrimido[4,5-b][1,4]diazepin-2-ylamino)-N-(3-(dimethylamino)propyl)-3-methoxybenzamide;
9-cyclopentyl-2-(2-methoxy-4-(4-methylpiperazine-1-carbonyl)phenylamino)-5-methyl-8,9-dihydro-5H-pyrimido[4,5-b][1,4]diazepin-6(7H)-one;
9-cyclopentyl-2-(2-methoxy-4-(morpholine-4-carbonyl)phenylamino)-5-methyl-8,9-dihydro-5H-pyrimido[4,5-b][1,4]diazepin-6(7H)-one;
4-(9-cyclopentyl-5-methyl-6-oxo-6,7,8,9-tetrahydro-5H-pyrimido[4,5-b][1,4]diazepin-2-ylamino)-N-(2-hydroxyethyl)-3-methoxybenzamide;
N-(1-(cyanomethyl)piperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N,N-dimethylpiperidine-1-carboxamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
9-cyclopentyl-2-(isoquinolin-7-ylamino)-5-methyl-8,9-dihydro-5H-pyrimido[4,5-b][1,4]diazepin-6(7H)-one;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
tert-butyl 4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)piperidine-1-carboxylate;
9-cyclopentyl-5-methyl-2-(3,4,5-trimethoxyphenylamino)-8,9-dihydro-5H-pyrimido[4,5-b][1,4]diazepin-6(7H)-one;
isopropyl 4-(9-cyclopentyl-5-methyl-6-oxo-6,7,8,9-tetrahydro-5H-pyrimido[4,5-b][1,4]diazepin-2-ylamino)-3-methoxybenzoate;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
4-(9-cyclopentyl-5,7,7-trimethyl-6-oxo-6,7,8,9-tetrahydro-5H-pyrimido[4,5-b][1,4]diazepin-2-ylamino)-N-(1-(2-(dimethylamino)acetyl)piperidin-4-yl)-3-methoxybenzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N-ethylpiperidine-1-carboxamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-((S)-9-cyclohexyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-4-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
N-(1-(azetidine-3-carbonyl)piperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N-ethylpiperidine-1-carboxamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
N-((1r,4r)-4-aminocyclohexyl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-((1-methylpyrrolidin-3-yl)methyl)benzamide;
(S)-4-(9-cyclopentyl-5,7,7-trimethyl-6-oxo-6,7,8,9-tetrahydro-5H-pyrimid[4,5-b][1,4]diazepin-2-ylamino)-3-methoxy-N-((1-methylpyrrolidin-3-yl)methyl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-((1-methylpiperidin-3-yl)methyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-((1-methylpiperidin-4-yl)methyl)benzamide;
4-(9-cyclopentyl-5-methyl-6-oxo-6,7,8,9-tetrahydro-5H-pyrimido[4,5-b][1,4]diazepin-2-ylamino)-3-hydroxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methyl-N-(1-methylpiperidin-4-yl)benzamide;
3-chloro-4-(9-cyclopentyl-5-methyl-6-oxo-6,7,8,9-tetrahydro-5H-pyrimido[4,5-b][1,4]diazepin-2-ylamino)-N-(1-methylpiperidin-4-yl)benzamide; and
4-(9-cyclopentyl-5-methyl-6-oxo-6,7,8,9-tetrahydro-5H-pyrimido[4,5-b][1,4]diazepin-2-ylamino)-N-(1-methylpiperidin-4-yl)-3-(trifluoromethoxy)benzamide.

Particular examples of compounds according to the present invention also include, but are not limited to:
4-(9-cyclopentyl-5,8-dimethyl-6-oxo-6,7,8,9-tetrahydro-5H-pyrimido[4,5-b][1,4]diazepin-2-ylamino)-3-methoxybenzoic acid;
4-(9-cyclopentyl-8-ethyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-{[(7aR)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
3-methoxy-4-{[(7aS)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
4-[(9-isopropyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopropyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-[(9-cyclohexyll-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-isobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-benzyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-(pentan-3-yl)-6,7,8,9-tetrahydro-5H-pyrimido[4,5-b][1,4]diazepin-2-ylamino)-N-(1-methylpiperidin-4-yl)benzamide;
4-(9-sec-butyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-allyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-phenyl-6,7,8,9-tetrahydro-5H-pyrimido[4,5-b][1,4]diazepin-2-ylamino)-N-(1-methylpiperidin-4-yl)benzamide;
(S)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
4-(9-cyclopentyl-5-methyl-6-oxo-6,7,8,9-tetrahydro-5H-pyrimido[4,5-b][1,4]diazepin-2-ylamino)-3-methoxy N-(1-(methylsulfonyl)piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(3-(2-oxopyrrolidin-1-yl)propyl)benzamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
N-(2-acetamidoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxoimidazolidin-1-yl)ethyl)benzamide;
N-(2-aminoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxopyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(6-oxopiperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperazin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(methylamino)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-2-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-ylmethyl)benzamide;
4-(9-cyclopentyl-5-methyl-6-oxo-6,7,8,9-tetrahydro-5H-pyrimido[4,5-b][1,4]diazepin-2-ylamino)-N-cyclopropyl-3-methoxy-N-(methylpyridin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-(cyclopropanecarbonyl)pyridin-4-yl)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-N-((1-(cyclopropanecarbonyl)piperidin-4-yl)methyl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(1-isopropylpiperidin-4-yl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(dimethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(diethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(1,2,2,6,6-pentamethylpiperidin-4-yl)benzamide;
9-cyclopentyl-5-methyl-2-(pyridin-3-ylamino)-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
3-Methoxy-4-(5-methyl-6-oxo-9-(tetrahydro-2H-pyran-4-yl)-6,7,8,9-tetrahydro-5H-pyrimido[4,5-b][1,4]diazepin-2-ylamino)-N-(1-methylpiperidin-4-yl)benzamide;
4-(9-(4,4-Difluorocyclohexyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(3-(dimethylamino)propyl)-3-methoxybenzamide;
N-(1-acetylpiperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-(methylsulfonyl)piperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(2-(dimethylamino)ethyl)-3-methoxybenzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclopentyl-5,7,7-trimethyl-6-oxo-6,7,8,9-tetrahydro-5H-pyrimido[4,5-b][1,4]diazepin-2-ylamino)-N,3-dimethoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-morpholinobenzamide;
4-(9-cyclopentyl-5,7,7-trimethyl-6-oxo-6,7,8,9-tetrahydro-5H-pyrimido[4,5-b][1,4]diazepin-2-ylamino)-N-(4-(2-hydroxyethyl)piperazin-1-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(4-cyclopentylpiperazin-1-yl)-3-methoxybenzamide;
N-(azetidin-3-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
N-(azepan-4-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-hydroxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-ethoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-methylpiperidin-4-yl)-3-propoxybenzamide;
4-(9-cyclopentyl-5,7,7-trimethyl-6-oxo-6,7,8,9-tetrahydro-5H-pyrimido[4,5-b][1,4]diazepin-2-ylamino)-3-(2,2-difluoroethoxy)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic acid;
4-(9-Cyclopentyl-5,7,7-trimethyl-6-oxo-6,7,8,9-tetrahydro-5H-pyrimido[4,5-b][1,4]diazepin-2-ylamino)-3-(difluoromethoxy)-N-(1-methylpiperidin-4-yl)benzamide;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-isopropyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclopentyl-5′-methyl-6′-oxo-5′,6′,8′,9′-tetrahydrospiro-[cyclopropane-1,7′-pyrimido[4,5-b][1,4]diazepine]-2′-ylamino)-3-methoxy-N-(1-methylpiperidin-3-yl)benzamide;
(R)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)-benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-N-(1-ethylpiperidin-4-yl)-3-methoxybenzamide;
3-methoxy-4-(5′-methyl-9′-(2-methylcyclopentyl)-6′-oxo-5′,6′,8′,9′-tetrahydrospiro[cyclopropane-1,7′-pyrimido[4,5-b][1,4]diazepine]-2′-ylamino)-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(4-methylpiperazin-1-yl)benzamide;
(R)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
4-(7-(Cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-(2-amino-2-oxoethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid;
4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(piperidin-4-yl)benzamide;
(S)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(Z)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(pent-2-ynyl)-6,7,8,9-tetrahydro-5H-pyrimido[4,5-b][1,4]diazepin-2-ylamino)-3-methoxy-N-(1-methylpiperidin-4-yl)benzamide;
(E)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9-cyclopentyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxybenzoic acid;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-7-hydroxy-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic acid;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((S)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
(S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
9-cyclopentyl-5-methyl-2-(pyridin-2-ylamino)-8,9-dihydro-5H-pyrimido[4,5-b][1,4]diazepin-6(7H)-one;
9-cyclopentyl-5-methyl-2-(phenylamino)-8,9-dihydro-5H-pyrimido[4,5-b][1,4]diazepin-6(7H)-one;
9-cyclopentyl-5-methyl-2-(2-(trifluoromethyl)phenylamino)-8,9-dihydro-5H-pyrimido[4,5-b][1,4]diazepin-6(7H)-one;
9-cyclopentyl-2-(2-fluoro-6-methoxyphenylamino)-5-methyl-8,9-dihydro-5H-pyrimido[4,5-b][1,4]diazepin-6(7H)-one;
9-cyclopentyl-2-(3-methoxypyrazin-2-ylamino)-5-methyl-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)nicotinonitrile;
5-(9-cyclopentyl-5-methyl-6-oxo-6,7,8,9-tetrahydro-5H-pyrimido[4,5-b][1,4]diazepin-2-ylamino)picolinonitrile;
5-(9-cyclopentyl-5-methyl-6-oxo-6,7,8,9-tetrahydro-5H-pyrimido[4,5-b][1,4]diazepin-2-ylamino)picolinamide;
6-(9-cyclopentyl-5-methyl-6-oxo-6,7,8,9-tetrahydro-5H-pyrimido[4,5-b][1,4]diazepin-2-ylamino)nicotinamide;
9-cyclopentyl-2-(5-fluoro-2-methoxyphenylamino)-5-methyl-8,9-dihydro-5H-pyrimido[4,5-b][1,4]diazepin-6(7H)-one;
9-cyclopentyl-2-(3-fluoro-2-methoxyphenylamino)-5-methyl-8,9-dihydro-5H-pyrimido[4,5-b][1,4]diazepin-6(7H)-one;
9-cyclopentyl-5-methyl-2-(1,2,3,4-tetrahydroisoquinolin-6-ylamino)-8,9-dihydro-5H-pyrimido[4,5-b][1,4]diazepin-6(7H)-one;
2-(4-aminophenylamino)-9-cyclopentyl-5-methyl-8,9-dihydro-5H-pyrimido[4,5-b][1,4]diazepin-6(7H)-one;
N-(4-(9-cyclopentyl-5-methyl-6-oxo-6,7,8,9-tetrahydro-5H-pyrimido[4,5-b][1,4]diazepin-2-ylamino)phenyl)acetamide;
2-(3-aminophenylamino)-9-cyclopentyl-5-methyl-8,9-dihydro-5H-pyrimido[4,5-b][1,4]diazepin-6(7H)-one; and
N-(3-(9-cyclopentyl-5-methyl-6-oxo-6,7,8,9-tetrahydro-5H-pyrimido[4,5-b][1,4]diazepin-2-ylamino)phenyl)acetamide.

It is noted that the compounds of the present invention may be in the form of a pharmaceutically acceptable salt, biohydrolyzable ester, biohydrolyzable amide, biohydrolyzable carbamate, solvate, hydrate or prodrug thereof. For example, the compound optionally comprises a substituent that is convertible in vivo to a different substituent such as a hydrogen.

It is further noted that the compound may be present in a mixture of stereoisomers, or the compound may comprise a single stereoisomer.

The present invention also relates to the use of a compound according to any one of above embodiments and variations as a medicament.

In addition, the present invention relates to the use of a compound according to any one of the above embodiments and variations in the manufacture of a medicament for inhibiting a polo-like kinase.

Further, the present invention relates to the use of a compound according to any one of the above embodiments and variations in the manufacture of a medicament for treating a disease state for which a polo-like kinase possess activity that contributes to the pathology and/or symptomology of the disease state.

The present invention also relates to the use of a compound according to any one of the above embodiments and variations in the manufacture of a medicament for treating hyperproliferative disorders; cancer; inflammatory diseases; auto-immune diseases; chemotherapy agent-induced alopecia and mucositis; cardiovascular diseases; viral, bacterial, fungal and/or parasitic infectious diseases; nephrological diseases; chronic and acute neurodegenerative diseases; skin diseases; bone diseases; and the protection of proliferating cells.

The present invention also provides a pharmaceutical composition comprising as an active ingredient a compound according to any one of the above embodiments and variations. In one particular variation, the composition is a solid formulation adapted for oral administration. In another particular variation, the composition is a liquid formulation adapted for oral administration. In yet another particular variation, the composition is a tablet. In still another particular variation, the composition is a liquid formulation adapted for parenteral administration.

In another of its aspects, there is provided a pharmaceutical composition comprising a compound according to any one of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another of its aspects, there is provided a kit comprising a compound of any one of the above embodiments and variations; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound of any one of the above embodiments and variations; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound of any one of the above embodiments and variations to a subject.

In another of its aspects, there is provided a method of inhibiting a kinase comprising contacting the kinase with a compound of any one of the above embodiments and variations.

In yet another of its aspects, there is provided a method of inhibiting a kinase comprising causing a compound of any one of the above embodiments and variations to be present in a subject in order to inhibit the kinase in vivo.

In a further of its aspects, there is provided a method of inhibiting a kinase comprising administering a first compound to a subject that is converted in vivo to a second compound wherein the second compound inhibits the kinase in vivo, the second compound being a compound according to any one of the above embodiments and variations.

In another of its aspects, there is provided a method of treating a disease state for which a kinase possesses activity that contributes to the pathology and/or symptomology of the disease state, the method comprising causing a compound of any one of the above embodiments and variations to be present in a subject in a therapeutically effective amount for the disease state.

In yet another of its aspects, there is provided a method of treating a disease state for which a kinase possesses activity that contributes to the pathology and/or symptomology of the disease state, the method comprising administering a compound of any one of the above embodiments and variations to a subject, wherein the compound is present in the subject in a therapeutically effective amount for the disease state.

In a further of its aspects, there is provided a method of treating a disease state for which a kinase possesses activity that contributes to the pathology and/or symptomology of the disease state, the method comprising administering a first compound to a subject that is converted in vivo to a second compound wherein the second compound inhibits the kinase in vivo, the second compound being a compound according to any one of the above embodiments and variations.

In one variation of each of the above methods the disease state is selected from the group consisting of hyperproliferative disorders; cancer (e.g., solid tumors, leukemias, lymphomas, non-small cell lung cancers and esophageal carcinomas); inflammatory and autoimmune diseases (e.g., psoriasis, alopecia; multiple sclerosis; colitis, arthritis, Alzheimer's disease, glomerulonephritis and wound healing); chemotherapy agent-induced alopecia and mucositis; cardiovascular diseases (e.g., stenoses, arterioscleroses, restenoses, and hypertrophy); viral, bacterial, fungal and/or parasitic infectious diseases (e.g., cytomegalic infections, herpes, hepatitis B and C, Karposi's sarcoma, HIV diseases); nephrological diseases (e.g., glomerulonephritis); chronic and acute neurodegenerative diseases (e.g., Huntington's disease, amyotrophic lateral sclerosis, Parkinson's disease, AIDS dementia, Alzheimer's disease, ischemias of the brain and neurotraumas); skin diseases (e.g., psoriasis); bone diseases; the protection of proliferating cells (e.g., hair, intestinal, blood and progenitor cells) from DNA damage caused by radiation, UV treatment and/or cytostatic treatment.

In another variation of each of the above methods, the kinase is a protein tyrosine kinase. In still another variation of each of the above methods, the kinase is a Polo-like Kinase (PLK). In yet another variation, the PLK is PLK1, PLK2, PLK3, PLK4, TTK, FAK and/or AIK

### Salts, Hydrates, and Prodrugs of Kinase Inhibitors

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

### Compositions Comprising Kinase Inhibitors

A wide variety of compositions and administration methods may be used in conjunction with the compounds of the present invention. Such compositions may include, in addition to the compounds of the present invention, conventional pharmaceutical excipients, and other conventional, pharmaceutically inactive agents. Additionally, the compositions may include active agents in addition to the compounds of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The kinase inhibitors and compositions comprising them may be administered or coadministered in any conventional dosage form. Co-administration in the context of this invention is intended to mean the administration of more than one therapeutic agent, one of which includes a kinase inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding compounds according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of an inhibitor of the present invention to reduce kinase activity in vivo, thereby treating the disease state of the subject.

Dosage forms or compositions may optionally comprise one or more compounds according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more kinase inhibitors, optionally 0.1-95%, and optionally 1-95%.

Salts, preferably sodium salts, of the inhibitors may be prepared with carriers that protect the compound against rapid elimination from the body, such as time release formulations or coatings. The formulations may further include other active compounds to obtain desired combinations of properties.

### Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, compounds according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose, and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol, and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof; and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate, and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The compounds of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising compounds of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate, and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

### Injectables, Solutions, and Emulsions

The present invention is also directed to compositions designed to administer the compounds of the present invention by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an inhibitor in the parenteral formulation may be adjusted so that an injection administers a pharmaceutically effective amount sufficient to produce the desired pharmacological effect. The exact concentration of an inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the kinase inhibitor to the treated tissue(s). The inhibitor may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The kinase inhibitor may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

### Lyophilized Powders

The compounds of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a kinase inhibitor is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the inhibitor.

### Topical Administration

The compounds of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The kinase inhibitors may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the kinase inhibitor alone or in combination with other pharmaceutically acceptable excipients can also be administered.

### Formulations for Other Routes of Administration

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### Examples of Formulations

The following are particular examples of oral, intravenous and tablet formulations that may optionally be used with compounds of the present invention. It is noted that these formulations may be varied depending on the particular compound being used and the indication for which the formulation is going to be used.

ORAL FORMULATION

| | | |
|---|---|---|
| Compound of the Present Invention | 10-100 | mg |
| Citric Acid Monohydrate | 105 | mg |
| Sodium Hydroxide | 18 | mg |
| Flavoring | | |

| | |
|---|---|
| Water | q.s. to 100 mL |

INTRAVENOUS FORMULATION

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |

| | |
|---|---|
| Water for Injection | q.s. to 1.0 mL |

TABLET FORMULATION

| | |
|---|---|
| Compound of the Present Invention | 1% |
| Microcrystalline Cellulose | 73% |
| Stearic Acid | 25% |
| Colloidal Silica | 1%. |

### Kits Comprising Kinase Inhibitors

The invention is also directed to kits and other articles of manufacture for treating diseases associated with kinases. It is noted that diseases are intended to cover all conditions for which the kinase possesses activity that contributes to the pathology and/or symptomology of the condition.

In one embodiment, a kit is provided that comprises a composition comprising at least one inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Dosage, Host and Safety

The compounds of the present invention are stable and can be used safely. In particular, the compounds of the present invention are useful as PLK inhibitors for a variety of subjects (e.g., humans, non-human mammals and non-mammals). The optimal dose may vary depending upon such conditions as, for example, the type of subject, the body weight of the subject, the route of administration, and specific properties of the particular compound being used. In general, the daily dose for oral administration to an adult (body weight of about 60 kg) is about 1 to 1000 mg, about 3 to 300 mg, or about 10 to 200 mg. It will be appreciated that the daily dose can be given in a single administration or in multiple (e.g., 2 or 3) portions a day.

### Combination Therapies

A wide variety of therapeutic agents may have a therapeutic additive or synergistic effect with kinase inhibitors according to the present invention. Combination therapies that comprise one or more compounds of the present invention with one or more other therapeutic agents can be used, for example, to: 1) enhance the therapeutic effect(s) of the one or more compounds of the present invention and/or the one or more other therapeutic agents; 2) reduce the side effects exhibited by the one or more compounds of the present invention and/or the one or more other therapeutic agents; and/or 3) reduce the effective dose of the one or more compounds of the present invention and/or the one or more other therapeutic agents. For example, such other therapeutic agents may additively or synergistically combine with the kinase inhibitors to inhibit undesirable cell growth, such as inappropriate cell growth resulting in undesirable benign conditions or tumor growth.

In one embodiment, a method is provided for treating a cell proliferative disease state comprising treating cells with a compound according to the present invention in combination with an anti-proliferative agent, wherein the cells are treated with the compound according to the present invention before, at the same time, and/or after the cells are treated with the anti-proliferative agent, referred to herein as combination therapy. It is noted that treatment of one agent before another is referred to herein as sequential therapy, even if the agents are also administered together. It is noted that combination therapy is intended to cover when agents are administered before or after each other (sequential therapy) as well as when the agents are administered at the same time.

Examples of therapeutic agents that may be used in combination with kinase inhibitors include, but are not limited to, anticancer agents, alkylating agents, antibiotic agents, antimetabolic agents, hormonal agents, plant-derived agents, and biologic agents.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Combination therapy including a kinase inhibitor and an alkylating agent may have therapeutic synergistic effects on cancer and reduce sides affects associated with these chemotherapeutic agents.

Antibiotic agents are a group of drugs that produced in a manner similar to antibiotics as a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interferes with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including a kinase inhibitor and an antibiotic agent may have therapeutic synergistic effects on cancer and reduce sides affects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including a kinase inhibitor and a antimetabolic agent may have therapeutic synergistic effects on cancer and reduce sides affects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, and flutamide), aromatase inhibitors (e.g., aminoglutethimide, anastrozole and tetrazole), ketoconazole, goserelin acetate, leuprolide, megestrol acetate and mifepristone. Combination therapy including a kinase inhibitor and a hormonal agent may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), and taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including a kinase inhibitor and a plant-derived agent may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including a kinase inhibitor and a biologic agent may have therapeutic synergistic effects on cancer, enhance the patient's immune responses to tumorigenic signals, and reduce potential sides affects associated with this chemotherapeutic agent.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with a kinase inhibitor include, but are not limited to, interleukin 2 (IL-2), and interleukin 4 (IL-4), interleukin 12 (IL-12).

Interferon include more than 23 related subtypes with overlapping activities, all of the IFN subtypes within the scope of the present invention. IFN has demonstrated activity against many solid and hematologic malignancies, the later appearing to be particularly sensitive.

Other cytokines that may be used in conjunction with a kinase inhibitor include those cytokines that exert profound effects on hematopoiesis and immune functions. Examples of such cytokines include, but are not limited to erythropoietin, granulocyte-CSF (filgrastin), and granulocyte, macrophage-CSF (sargramostim). These cytokines may be used in conjunction with a kinase inhibitor to reduce chemotherapy-induced myelopoietic toxicity.

Other immuno-modulating agents other than cytokines may also be used in conjunction with a kinase inhibitor to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including a kinase inhibitor and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺ pre-B and mature B cells. RITUXAN® is used as single agent for the treatment of patients with relapsed or refractory low-grade or follicular, CD20+, B cell non-Hodgkin's lymphoma. Combination therapy including a kinase inhibitor and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth—cancer. Examples of the tumor suppressor genes include, but are not limited to, DPC-4, NF-1, NF-2, RB, p53, WT1, BRCA1, and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. Combination therapy including a kinase inhibitor and a tumor suppressor may have therapeutic synergistic effects on patients suffering from various forms of cancers.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAAs include, but are not limited to gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancers), melanoma associated antigens (MART-1, gp100, MAGE 1,3 tyrosinase), papillomavirus E6 and E7 fragments, whole cells or portions/lysates of antologous tumor cells and allogeneic tumor cells.

An adjuvant may be used to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

### EXAMPLES

### Preparation of Kinase Inhibitors

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| μL (microliters) | Ac (acetyl) |
| atm (atmosphere) | ATP (Adenosine Triphophatase) |
| BOC (tert-butyloxycarbonyl) | BOP (bis(2-oxo-3-oxazolidinyl) |
| | phosphinic chloride) |
| BSA (Bovine Serum Albumin) | CBZ (benzyloxycarbonyl) |
| CDI (1,1-carbonyldiimidazole) | DCC (dicyclohexylcarbodiimide) |
| DCE (dichloroethane) | DCM (dichloromethane) |
| DMAP (4-dimethylaminopyridine) | DME (1,2-dimethoxyethane) |
| DMF (N,N-dimethylformamide) | DMPU (N,N′-dimethylpropyleneurea) |
| DMSO (dimethylsulfoxide) | EDCI (ethylcarbodiimide hydro- |
| | chloride) |
| EDTA (Ethylenediaminetetra- | Et (ethyl) |
| acetic acid) | |
| Et₂O (diethyl ether) | EtOAc (ethyl acetate) |
| FMOC (9-fluorenylmethoxy- | g (grams) |
| carbonyl) | |
| h (hours) | HOAc or AcOH (acetic acid) |
| HOBT (1-hydroxybenzotriazole) | HOSu (N-hydroxysuccinimide) |
| HPLC (high pressure liquid | Hz (Hertz) |
| chromatography) | |
| i.v. (intravenous) | IBCF (isobutyl chloroformate) |
| i-PrOH (isopropanol) | L (liters) |
| M (molar) | mCPBA (meta-chloroperbenzoic |
| | acid) |
| Me (methyl) | MeOH (methanol) |
| mg (milligrams) | MHz (megahertz) |
| min (minutes) | mL (milliliters) |
| mM (millimolar) | mmol (millimoles) |
| mol (moles) | MOPS (Morpholinepropanesulfonic |
| | acid) |
| mp (melting point) | NaOAc (sodium acetate) |
| OMe (methoxy) | psi (pounds per square inch) |
| RP (reverse phase) | RT (ambient temperature) |
| SPA (Scintillation Proximity | TBAF (tetra-n-butylammonium |
| Assay) | fluoride) |
| TBS (t-butyldimethylsilyl) | tBu (tert-butyl) |
| TEA (triethylamine) | TFA (trifluoroacetic acid) |
| TFAA (trifluoroacetic | THF (tetrahydrofuran) |
| anhydride) | |
| TIPS (triisopropylsilyl) | TLC (thin layer chromatography) |
| TMS (trimethylsilyl) | TMSE (2-(trimethylsilyl)ethyl) |
| Tr (retention time) | |

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

### Synthetic Schemes for Compounds of the Present Invention

Compounds according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

General synthetic routes for producing compounds of the present invention are shown in the following schemes.

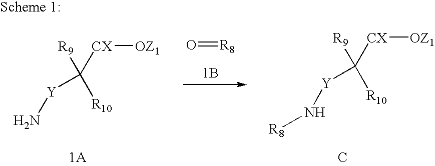

Referring to Scheme 1, Compound C is obtained by reductive amination of Compound 1B by Compound 1A using, for example, sodium triacetoxyborohydride or sodium cyanoborohydride. In particular embodiments, Z₁ is Me or Et; R₈ is cyclopentyl, isopropyl or a 2-6 membered alkyl bridge to R₂ or R₃ of Y; and R₂ and R₃ of Y are each independently H, Me, Et or a 2-6 membered alkyl bridge. In other particular embodiments, Compound 1B is cyclopentanone, cyclohexanone or ketone.

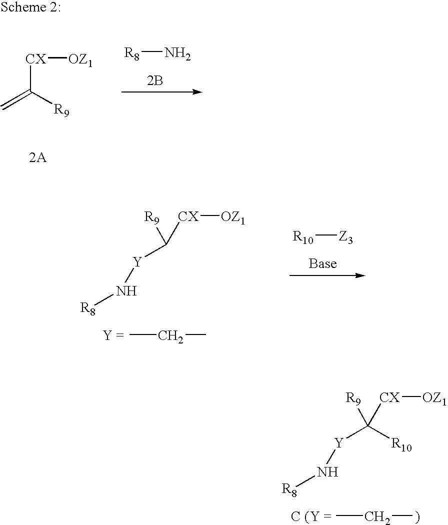

Referring to Scheme 2, Compound C is prepared by Michael addition of Compound 2A (i.e., ethyl acrylate) with Compound 2B (i.e., cyclopropylamine or cyclopentylamine) followed by alkylation with R₁₀-Z₃.

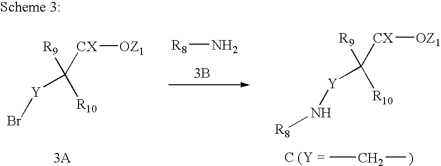

Referring to Scheme 3, Compound 3A is converted to Compound C using Compound 3B (i.e., cyclopropylamine or cyclopentylamine) in the presence of base (i.e., K₂CO₃) and sodium iodide.

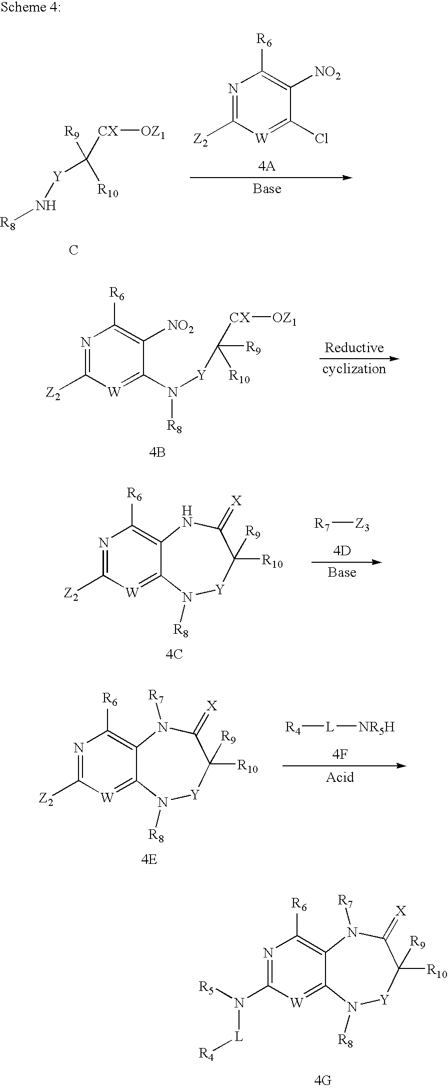

Referring to Scheme 4, Compound C is treated with Compound 4A in the presence of base (i.e., K₂CO₃ or diisopropylethylamine) to give Compound 4B. Compound 4B is transformed by reduction using reduced iron and subsequent cyclization reaction to afford Compound 4C. Compound 4E is prepared by N-alkylation of Compound 4C with Compound 4D (e.g., alkyl halide (e.g., iodomethane)). Compound 4E is treated with Compound 4F in the presence of a catalytic amount of acid (i.e., conc. HCl or pyridinium chloride) to obtain Compound 4G.

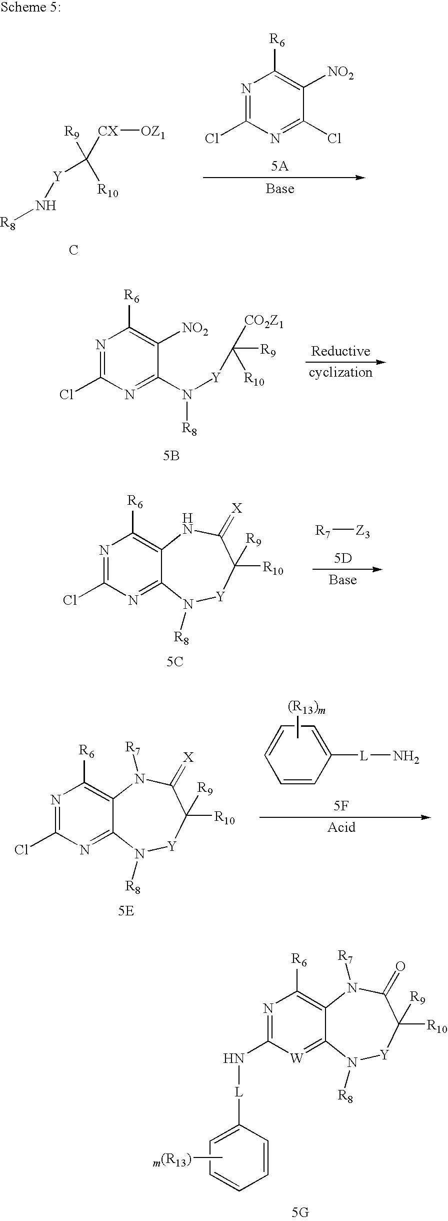

Referring to Scheme 5, Compound C is treated with Compound 5A in the presence of base (i.e., K₂CO₃ or diisopropylethylamine) to give Compound 5B. Compound 5B is transformed by reduction using reduced iron and subsequent cyclization reaction to afford Compound 5C. Compound 5E is prepared by N-alkylation of Compound 5C with alkyl halide 5D (i.e., iodomethane). Compound 5E is treated with aniline or benzylamine (Compound 5F, L=CHR₁₄) in the presence of a catalytic amount of acid (i.e., conc. HCl or pyridinium chloride) to obtain Compound 5G.

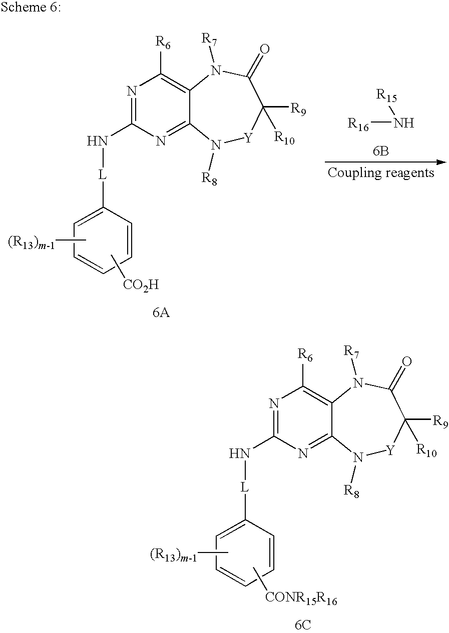

Coupling reaction of Compound 6A with amine (Compound 6B, i.e., methylamine or 1-methylpiperidin-4-amine) using appropriate coupling reagents (i.e., TBTU) is carried out to give Compound 6C.

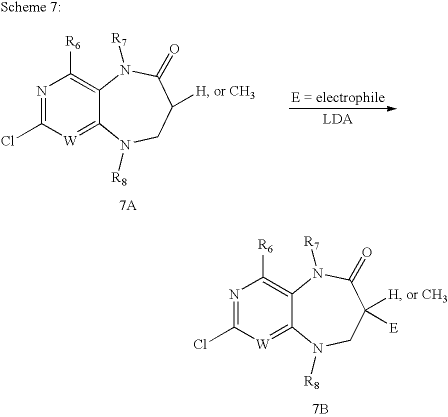

Referring to Scheme 7, 7B can be obtained through deprotonation of 7A using base (i.e. LDA) followed by addition of an electrophile.

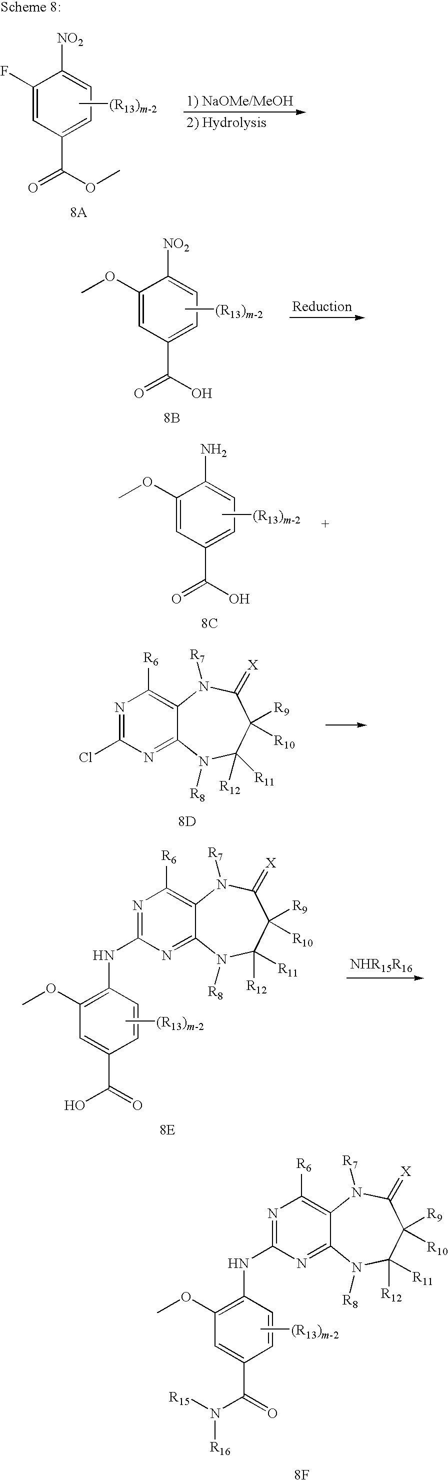

Referring to Scheme 8, Compound 8A is treated with a base (i.e., sodium methoxide) in methanol to afford Compound 8B, which is hydrogenated in presence of a catalyst (i.e, Pd/C) to yield Compound 8C. Compound 8D is refluxed with Compound 8C in presence of acid catalyst (i.e., conc.HCl or TsOH) to give Compound 8E. Compound 8E is condensed with an amine (i.e., methylamine or 1-methylpiperidin-4-amine) using appropriate coupling reagents (i.e., HATU, TBTU etc.) to give the Compound 8F.

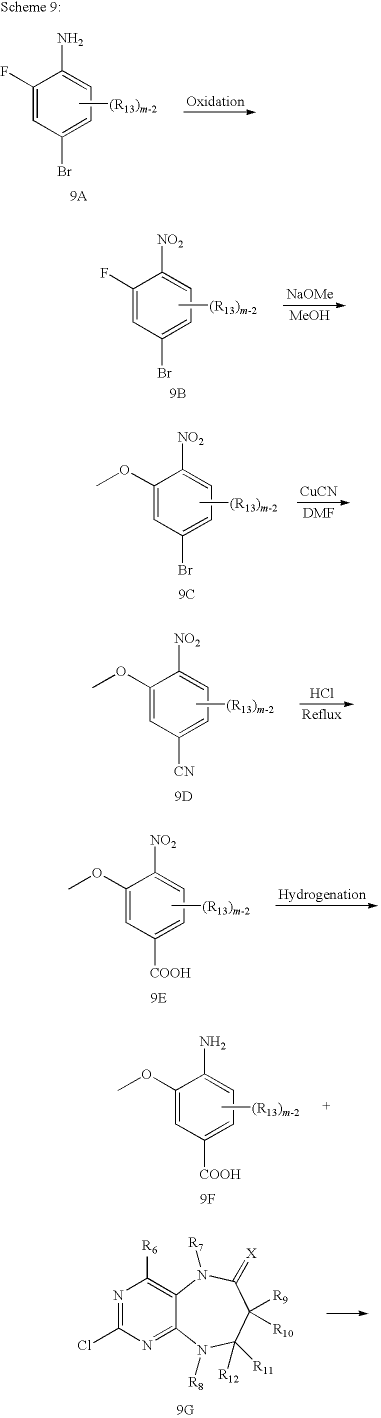

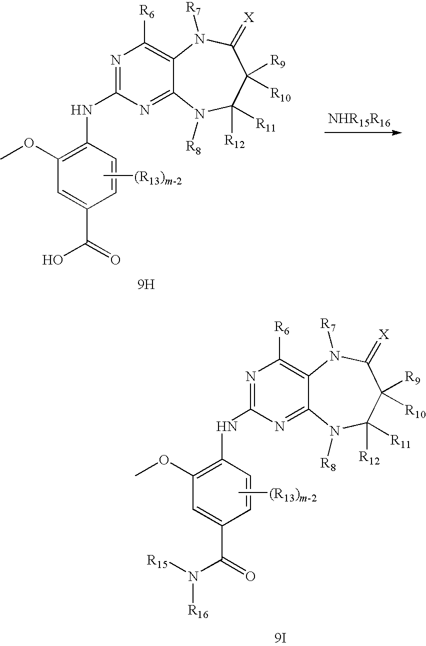

Referring to Scheme 9, Compound 9A is treated with an oxidation agent (e.g., sodium perborate in acetic acid) to afford Compound 9B, which is converted to its methyl ether using a base (i.e., NaOMe) in methanol to give Compound 9C. Compound 9C is subjected to a nucleophilic substitution reaction in presence of CuCN to yield Compound 9D, which is hydrolyzed to its acid 9E in presence of strong acid (i.e., conc.HCl). Compound 9E is hydrogenated in presence of a catalyst (i.e, Pd/C) and acetic acid in alcohol such as methanol to yield Compound 9F. Compound 9F is refluxed with Compound 9G in presence of acid catalyst (i.e., conc.HCl or TsOH) to give Compound 9H. Compound 9H is treated with an amine (i.e., methylamine or 1-methylpiperidin-4-amine) using appropriate coupling reagents (i.e., HATU, TBTU etc.) to give the Compound 9I.

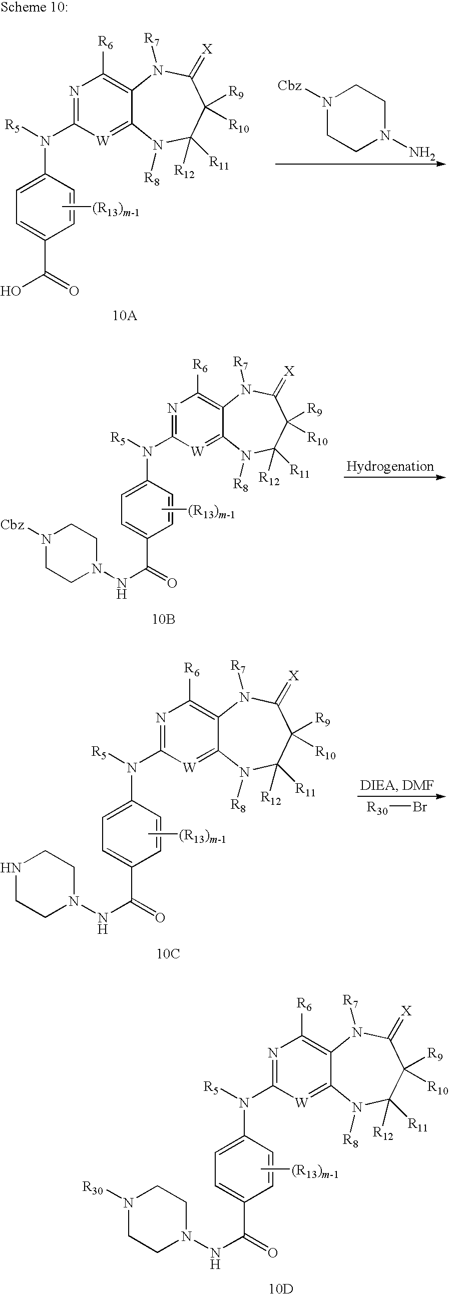

Referring to Scheme 10, Compound 10A is coupled with a piperazine derivative (i.e., benzyl 4-aminopiperazine-1-carboxylate) using appropriate coupling reagents (i.e., HATU, TBTU etc.) to give the Compound 10B. Compound 10B is subjected to hydrogenation for Cbz group deprotection in presence of a catalyst (i.e., Pd—C) in alcohol to get Compound 10C. Compound 10C is alkylated using an alkyl halide (i.e., ethyl bromide or isopropyl bromide) in presence of a base such as diisopropyl ethyl amide to yield Compound 10D.

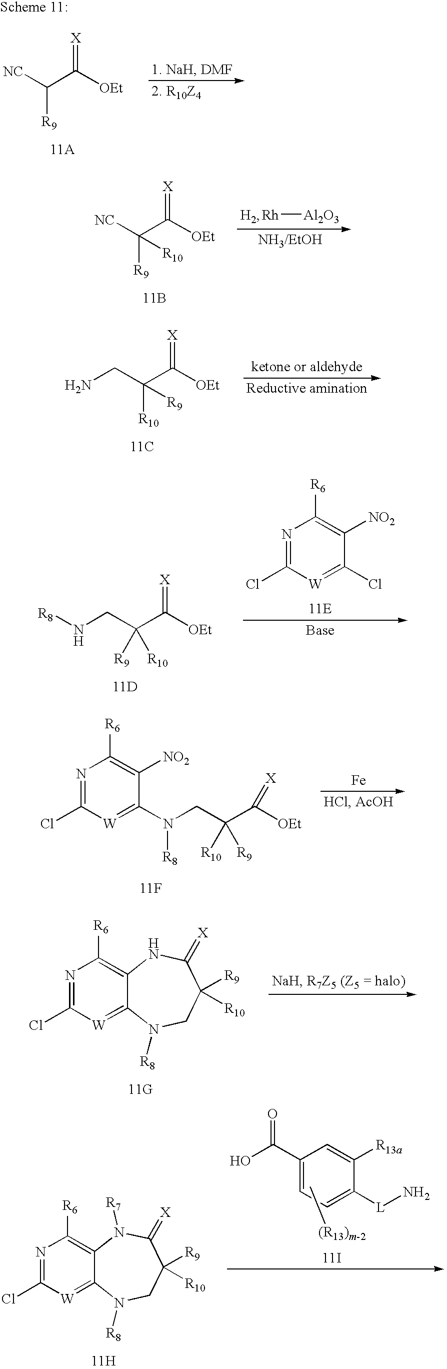

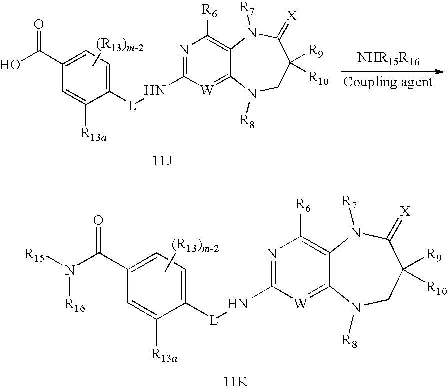

Referring to Scheme 11, Compound 11A is alkylated with an alkylylating agent (i.e., iodomethane) in presence of a strong base (i.e., 60% NaH in mineral oil) to get Compound 11B, which is reduced to its amine 11C by means of hydrogenation in presence of a catalyst (i.e., Rh—Al₂O₃) in basic medium NH₃ in ethanol. Compound 11C is subjected to reductive amination with an aldehyde or a ketone (i.e., cyclopentanone or cyclohexanone) in presence of sodium triacetoxy borohydride and sodium acetate to afford 11D. Compound 11D is subjected to SNAr reaction with 11E (i.e., 2,4-dichloro 5-nitro pyrimidine) in presence of a base (i.e., K₂CO₃) to afford Compound 11F, which on reductive cyclization using a metal/acid catalyst in acetic acid (i.e., Fe/HCl or Sn/HCl) to obtain Compound 11G. Compound 11G is alkylated with an alkyl halide (i.e., iodomethane) to obtain Compound 11H. Compound 11H is refluxed with Compound 11I in presence of an acid catalyst (i.e., conc.HCl or TsOH) in isopropanol to get Compound 11J, which is coupled with an amine (i.e., methylamine or 1-methylpiperidin-4-amine) using appropriate coupling reagents (i.e., HATU, TBTU etc.) to give the Compound 11K.

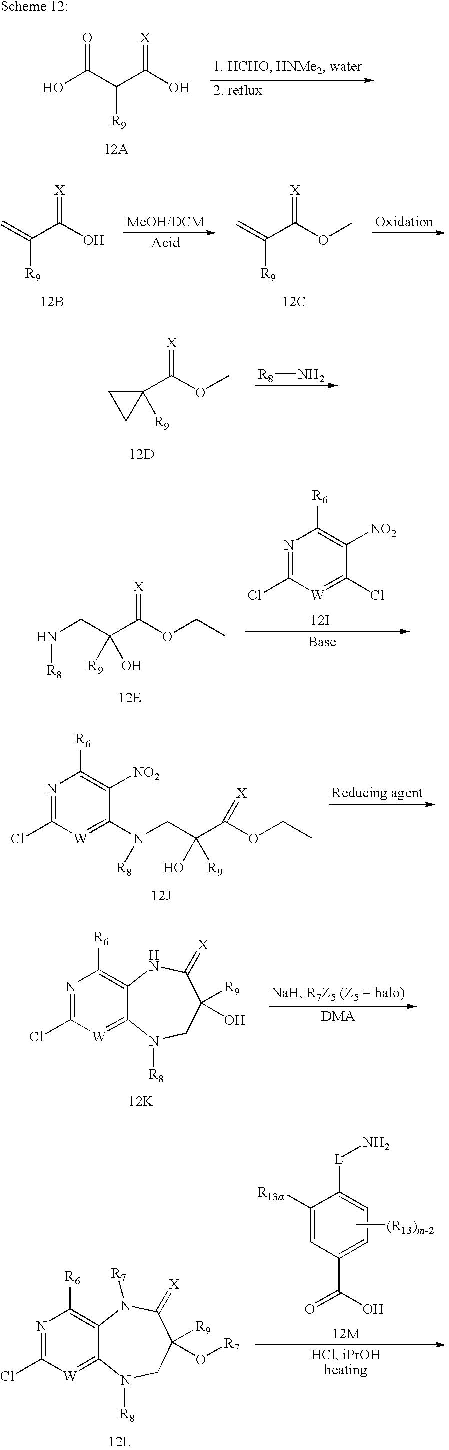

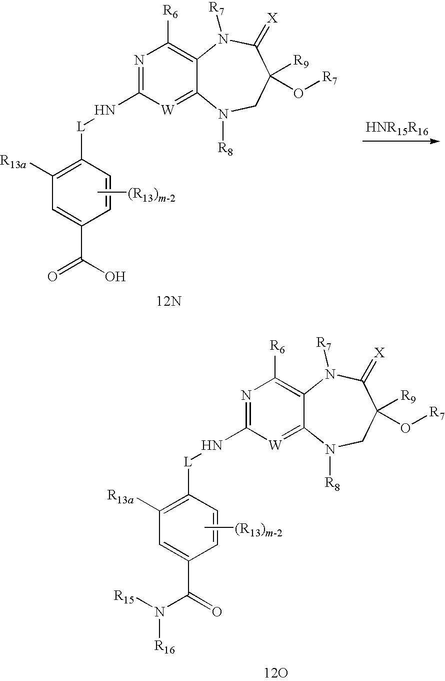

Referring to Scheme 12, 12B can be prepared upon treatment of 12A with formaldehyde and dimethylamine under reflux condition. After conversion of 12B to the methylester 12C, the double bond is oxidized to an epoxide 12D, which is transformed to amino alcohol 12E with alkylamine. Compound 12E is further treat with 121 in the presence of base to give 12J. Upon reduction of the nitro group with a reducing agent under acidic heating condition, Compound 12K can be readily obtained. Compound 12L is prepared by N-alkylation of Compound 12K with R₇-Z₄ (i.e., iodomethane). Compound 12L is treated with aniline or benzylamine in the presence of a catalytic amount of acid (i.e., conc. HCl or pyridinium chloride) to obtain Compound 12N. Coupling reaction of Compound 12N with amine (i.e., methylamine or 1-methylpiperidin-4-amine) using appropriate coupling reagents (i.e., HATU, TBTU etc.) is carried out to give Compound 120.

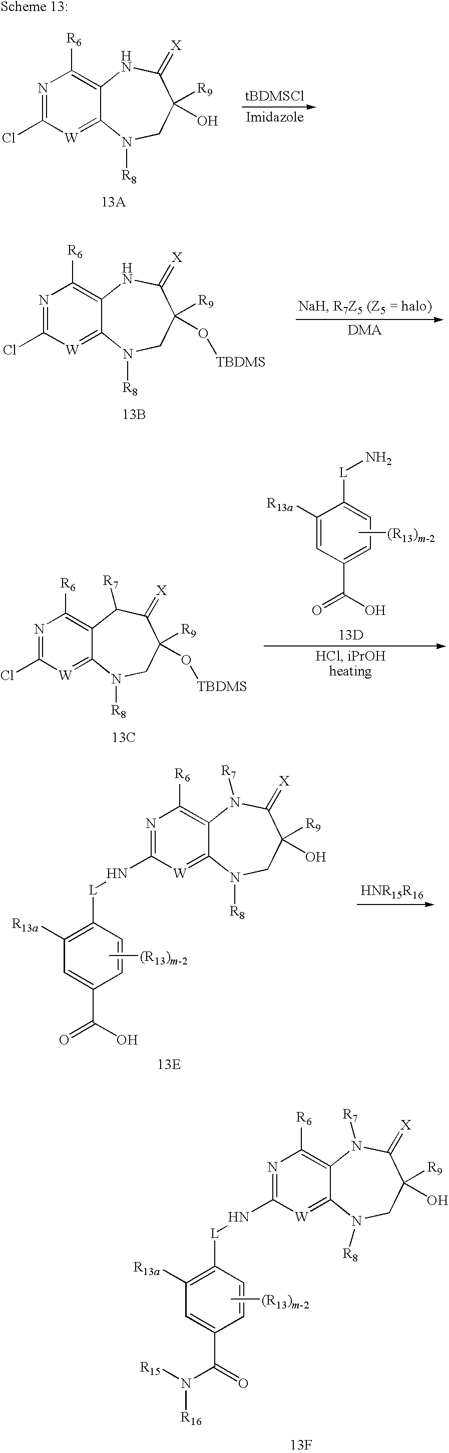

Referring to Scheme 13, the —OH group in 13A can be protected as 13B, which is further alkylated with R₇Z₅ to yield 13C. The final compound 13F can be obtained from 13C through an analogous procedure to that described in connection with the preparation of 120.

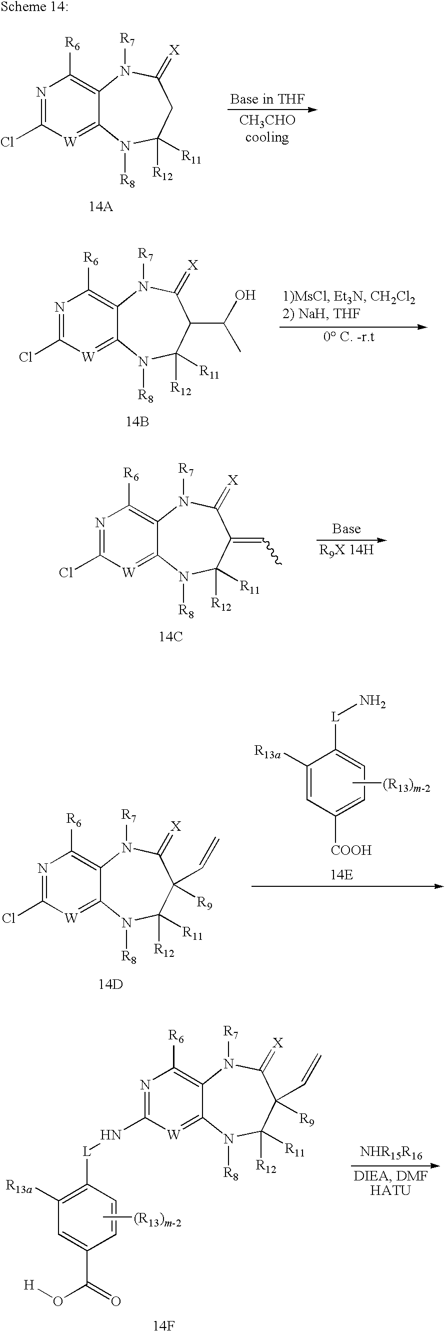

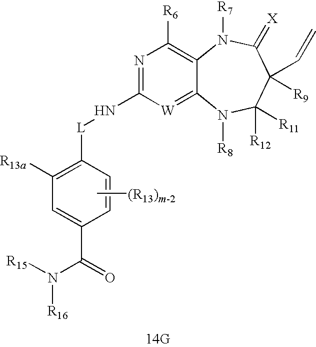

Referring to Scheme 14, Compound 14A is treated with acetaldehyde in the presence of base (i.e. LDA) to give 14B. The hydroxyl group on 14B is then mesylated at low temperature followed by base treatment (i.e. NaH) to yield the eliminated product Compound 14C. Under the cooling condition, Compound 14C is further reacted with halogen source 14H (e.g., N-fluorobenzenesulfonamide) in the presence of base such as LDA to give vinyl fluoro compound 14D. Compound 14D is refluxed with Compound 14E in presence of an acid catalyst (i.e., conc.HCl or TsOH) in isopropanol to get Compound 14F, which is coupled with an amine (i.e., methylamine or 1-methylpiperidin-4-amine) using appropriate coupling reagents (i.e., HATU, TBTU etc.) to give the Compound 14G.

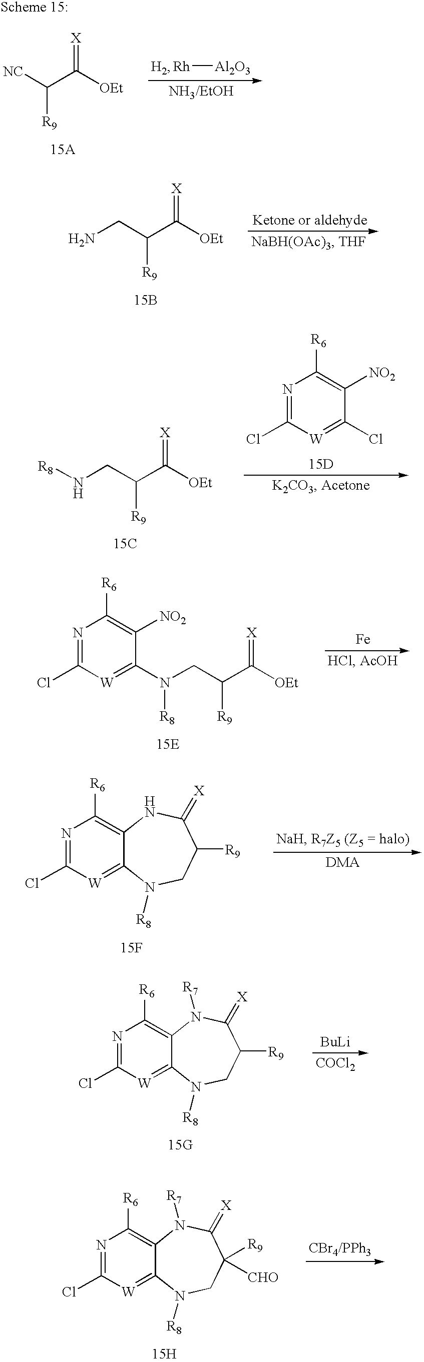

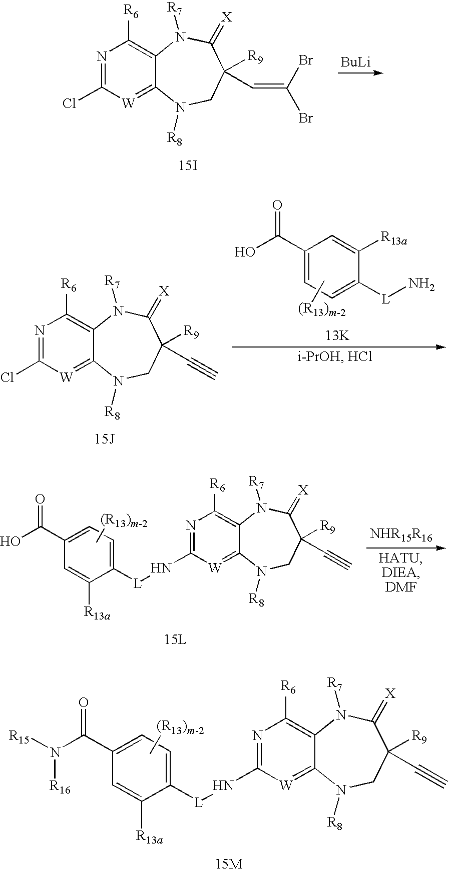

Referring to Scheme 15, hydrogenation of compound 15A in presence of a catalyst (i.e., Rh—Al₂O₃ using basic medium such as NH₃/ethanol to give the compound 15B. Reductive amination of compound 15B with aldehyde or ketone (i.e., cyclopentanone or cyclohexanone) in presence of sodium triacetoxy borohydride to afford 15C. Compound 15C is subjected to SNAr reaction with 15D (i.e., 2,4-dichloro 5-nitro pyrimidine) in presence of a base (i.e., K₂CO₃) to afford compound 15E, which on reductive cyclization using a metal/acid catalyst in acetic acid (i.e., Fe/HCl or Sn/HCl) to obtain compound 15F. Compound 15F is N-alkylated using a base (i.e., NaH or t-BuOK) and alkyl halide (i.e., iodomethane) in DMA to afford compound 15G. Compound 15G is reacted with oxalyl chloride in presence of a base (i.e., BuLi) to yield the compound 15H, which is further reacted with CBr₄/PPh₃ to afford compound 15I. Compound 15I is reacted with a strong base (i.e., BuLi) to yield compound 15J. Compound 15J is refluxed with compound 15K in presence of an acid catalyst (i.e., conc.HCl or TsOH) to get compound 15L, which is coupled with an amine (i.e., methylamine or 1-methylpiperidin-4-amine) using appropriate coupling reagents (i.e., HATU, TBTU etc.) to give the compound 15M. Final compound 15M is subjected to chiral separation to get the pure enantiomers.

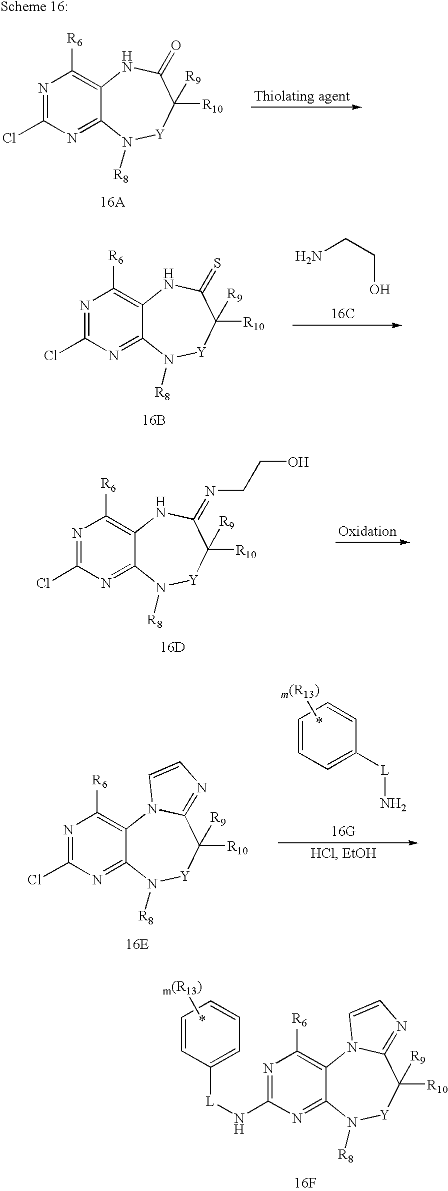

Referring to Scheme 16, Compound 16A is treated with a thiolating reagent to give Compound 16B. Compound 16B is transformed to amidine Compound 16D through reaction with an ethanolic amine represented by 16C. Compound 16E is prepared by oxidation and cyclization of Compound 16D. Compound 16E is treated with aniline or benzylamine (Compound 16G) in the presence of a catalytic amount of acid (i.e., conc. HCl or pyridinium chloride) to obtain Compound 16F.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Watrers ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 μL in methanol at 0.1 mg/mL in concentration.

In another variation, preparative chiral separations are performed using a Berger MultiGram II SFC purification system. For example, samples can be loaded onto a ChiralPak AD column (21×250 mm, 10μ). In particular variations, the flow rate for separation can be 70 mL/min, the injection volume up to 2 mL, and the inlet pressure set at 130 bar. Stacked injections can be applied to increase the efficiency.

In each of the above reaction procedures or schemes, the various substituents may be selected from among the various substituents otherwise taught herein.

Descriptions of the syntheses of particular compounds according to the present invention based on the above reaction scheme are set forth herein.

### Examples of Kinase Inhibitors

The present invention is further exemplified, but not limited by, the following examples that describe the synthesis of particular compounds according to the invention.

### Compound 9

### 4-(9-cyclopentyl-5-methyl-6-oxo-6,7,8,9-tetrahydro-5H-pyrimido[4,5-b][1,4]diazepin-2-ylamino)-3-methoxybenzoic Acid

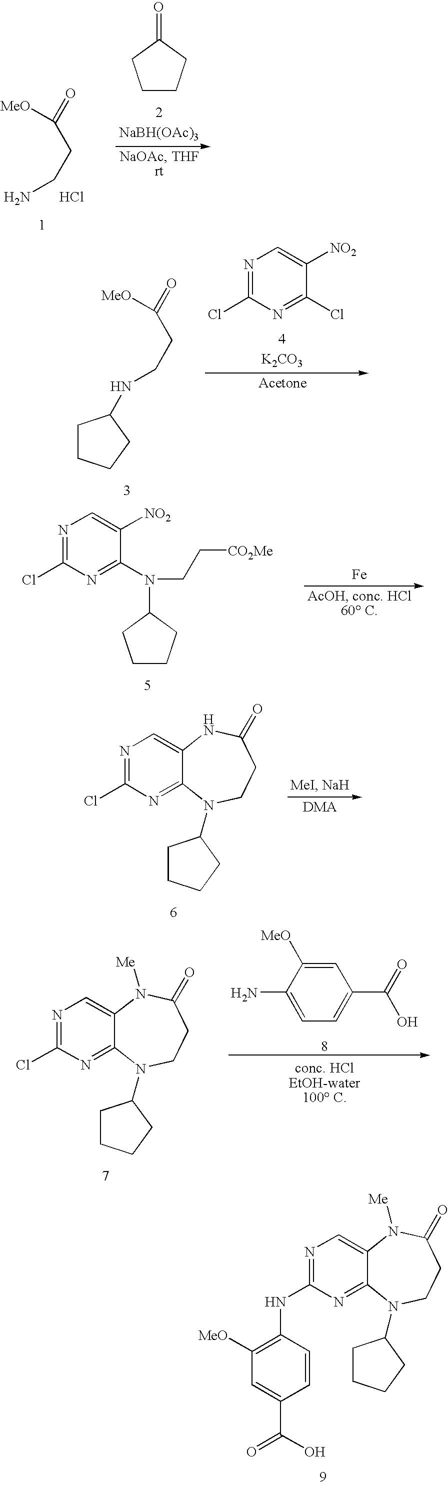

Methyl β-alaninate hydrochloride (Compound 1; 11.0 g) was treated with cyclopentanone (Compound 2; 6.98 ml) in the presence of sodium triacetoxyborohydride (23.4 g) and sodium acetate (6.5 g) in tetrahydrofuran (100 ml) for 18 hours at room temperature. The reaction mixture was diluted with saturated aqueous NaHCO₃ (500 ml). The mixture was stirred for 1 hour at room temperature. The whole was extracted with ethyl acetate (300 ml×2). The organic layer was washed with brine (500 ml), dried over Na₂SO₄ and concentrated in vacuo to obtain Compound 3 (3.6 g, 72% yield) pale brown oil. ¹H NMR (300 MHz, CHLOROFORM-d) δ 1.11 (3H, d, J=6.3 Hz), 1.20-1.37 (2H, m), 1.26 (3H, t, J=7.0 Hz), 1.44-1.74 (4H, m), 1.77-1.94 (2H, m), 2.38 (2H, ddd, J=6.3 Hz), 3.06-3.26 (2H, m), 4.13 (2H, q, J=7.0 Hz).

To the mixture of Compound 3, potassium carbonate (2.85 g) and acetone (80 ml) was added dropwise a solution of 2,4-dichloro-5-nitropyrimidine (Compound 4; 4.0 g) in acetone (10 ml) at 0° C. over 10 minutes and the whole was stirred at room temperature for 18 hours. After evaporation in vacuo, the residue was partitioned with ethyl acetate (200 ml) and water (200 ml). The organic layer was washed with brine (200 ml), dried over Na₂SO₄ and concentrated in vacuo. The residue was purified by silica gel chromatography (ethyl acetate:hexane=1:99 to 15:85) followed by crystallization from diisopropyl ether to obtain Compound 5 (13.7 g, 66% yield) as pale yellow powder. ¹H NMR (300 MHz, CHLOROFORM-d) δ 1.49-1.84 (6H, m), 1.92-2.07 (2H, m), 2.69-2.80 (2H, m), 3.67 (1H, quintet, J=7.9 Hz), 3.72 (3H, s), 3.74-3.81 (2H, m), 8.66 (1H, s). [M+H] calc'd for C₁₃H₁₇ClN₄O₄, 329; found, 329.

To a suspension of Compound 5 (1.5 g), reduced iron (0.64 g) in acetic acid (4.5 ml) was added dropwise concentrated hydrochloric acid (4.5 ml) at 0° C. The mixture was stirred at 60° C. for 1 hour. The mixture was poured into cooled 10% NaOH solution (100 ml) at 0° C., successively ethyl acetate was added and stirred at room temperature for 30 minutes. The whole was filtered through celite. The filtrate was extracted with ethyl acetate (100 ml). The organic layer was washed with brine (100 ml) and dried over Na₂SO₄. The solution was concentrated in vacuo followed by crystallization from diisopropyl ether to afford Compound 6 (1.1 g, 92% yield) as a white powder. ¹H NMR (300 MHz, CHLOROFORM-d) δ 1.38-1.59 (2H, m), 1.59-1.84 (4H, m), 1.91-2.08 (2H, m), 2.69-2.86 (2H, m), 3.56-3.65 (2H, m), 5.21 (1H, quintet, J=8.6 Hz), 7.80 (1H, s), 8.20 (1H, s). [M+H] calc'd for C₁₂H₁₅ClN₄O, 267; found, 267.

To a solution of Compound 6 and methyl iodide (585 mg) in DMA (10 ml) was added sodium hydride (60% dispersion in mineral oil, 159 mg) at −10° C. The mixture was stirred at −10° C. for 30 minutes, and then at room temperature for 4 h. The whole was poured into ice-water (100 ml) and extracted with ethyl acetate (100 ml×2). The organic layer was washed with brine (100 ml) and dried over Na₂SO₄. The solution was concentrated in vacuo followed by crystallization from diisopropyl ether to afford Compound 7 (915 mg, 87% yield) as a white powder. ¹H NMR (300 MHz, CHLOROFORM-d) δ 1.47-1.85 (6H, m), 1.92-2.14 (2H, m), 2.61-2.72 (2H, m), 3.29 (3H, s), 3.63-3.77 (2H, m), 4.89 (1H, quintet, J=8.5 Hz), 7.94 (1H, s). [M+H] calc'd for C₁₃H₁₇ClN₄O, 282; found, 282.

A mixture of Compound 7,1-(4-amino-3-methoxyphenyl)-2-hydroxyethanone (Compound 8), ethanol (4.7 ml), water (18.3 ml) and concentrated hydrochloric acid (0.18 ml) was stirred at 100° C. for 24 hours. The mixture was concentrated in vacuo. The residue was crystallized from ethanol-diethyl ether to give Compound 9 as a white powder. ¹H NMR (300 MHz, DMSO-d₆) δ 1.50-1.76 (6H, m), 1.82-2.01 (2H, m), 2.66-2.81 (2H, m), 3.17 (3H, s), 3.69-3.79 (2H, m), 3.94 (3H, s), 4.88 (1H, quintet, J=8.1 Hz), 7.53-7.70 (2H, m), 8.15 (1H, d, J=8.3 Hz), 8.19 (1H, s), 9.50 (1H, s), 12.94 (1H, br. s.). [M+H] calc'd for C₂₁H₂₅N₅O₄, 412; found, 412.

### Compound 10

### 4-[(9-cyclopentyl-5-methyl-6-oxo-6,7,8,9-tetrahydro-5H-pyrimido[4,5-b][1,4]diazepin-2-yl)amino]-3-methoxy-N-methylbenzamide

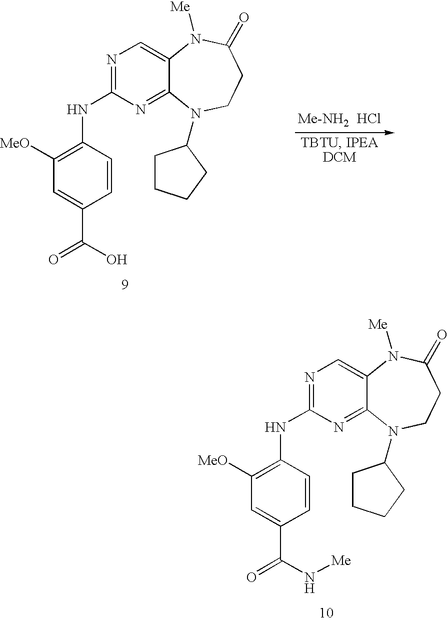

A mixture of Compound 9 (100 mg), O-(Benzotriazol-1-yl)-N,N,N′,N′-tetramethyluronium tetrafluoroborate (TBTU; 85.8 mg), diisopropylethylamine (85 μl) and dichloromethane (5 ml) was stirred at room temperature for 20 minutes. To the mixture added methyl ammonium chloride (19.7 mg) and diisopropylethylamine (42 μl) at room temperature. The whole was stirred at room temperature for 5 hours. The mixture was diluted with ethyl acetate (10 ml), washed with water (10 ml) and brine (10 ml), and dried over Na₂SO₄ followed by filtration. The filtrate was concentrated in vacuo. The residue was purified by NH silica gel (Fuji Silisia Chemical) chromatography (ethyl acetate:hexane=1:1 to ethyl acetate), followed by crystallization from diisopropyl ether to obtain Compound 10 (35.4 mg, 34% yield) as a white powder. ¹H NMR (300 MHz, CHLOROFORM-d) δ 1.55-1.86 (6H, m), 1.97-2.12 (2H, m), 2.59-2.73 (2H, m), 3.02 (3H, d, J=4.9 Hz), 3.29 (3H, s), 3.63-3.73 (2H, m), 3.97 (3H, s), 4.89 (1H, quintet, J=8.4 Hz), 6.10 (1H, d, J=4.9 Hz), 7.20-7.29 (1H, m), 7.43 (1H, d, J=1.9 Hz), 7.66 (1H, s), 7.93 (1H, s), 8.50 (1H, d, J=8.2 Hz). [M+H] calc'd for C₂₂H₂₈N₆O₃ 425; found, 425.

### Compound 11

### 4-[(9-cyclopentyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide

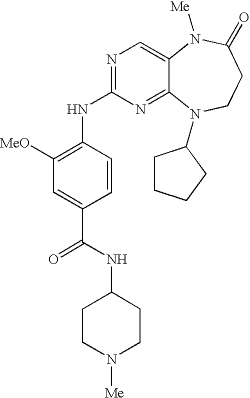

The title compound was synthesized using an analogous procedure to that described in connection with Compound 10 except that 1-methylpiperidin-4-amine was used. ¹H NMR (300 MHz, CHLOROFORM-d) δ 1.51-1.87 (8H, m), 1.96-2.26 (6H, m), 2.31 (3H, s), 2.63-2.73 (2H, m), 2.84 (2H, d, J=11.8 Hz), 3.29 (1H, s), 3.66-3.75 (2H, m), 3.90-4.06 (1H, m), 3.98 (3H, s), 4.90 (1H, quintet, J=8.5 Hz), 5.91 (1H, d, J=7.7 Hz), 7.19-7.28 (2H, m), 7.41 (1H, d, J=1.6 Hz), 7.67 (1H, s), 7.93 (1H, s), 8.50 (1H, d, J=8.5 Hz). [M+H] calc'd for C₂₇H₃₇N₇O₃ 508; found, 508.

### Compound 12

### 4-(9-cyclopentyl-5,8-dimethyl-6-oxo-6,7,8,9-tetrahydro-5H-pyrimido[4,5-b][1,4]diazepin-2-ylamino)-3-methoxybenzoic Acid

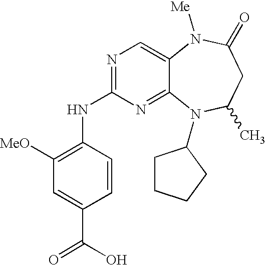

The title compound was synthesized using an analogous procedure to that described in connection with Compound 9 except that methyl ethyl 3-aminobutanoate was used. ¹H NMR (400 MHz, DMSO-d₆) δ 1.23 (d, J=6.4 Hz, 3H), 1.37-1.82 (m, 6H), 1.84-1.98 (m, 2H), 2.53-2.58 (m, 1H), 2.93-3.03 (m, 1H), 3.21 (s, 3H), 3.93 (s, 3H), 4.05-4.19 (m, 1H), 4.66 (quintet, J=7.0 Hz, 1H), 7.54-7.61 (m, 2H), 8.05 (d, J=8.9 Hz, 1H), 8.15 (s, 1H), 9.34 (s, 1H), 12.88 (br. s., 1H). [M+H] calc'd for C₂₂H₂₇N₅O₄ 426; found, 426.

### Compound 13

### 4-[(9-cyclopentyl-5,8-dimethyl-6-oxo-6,7,8,9-tetrahydro-5H-pyrimido[4,5-b][1,4]diazepin-2-yl)amino]-3-methoxy-N-methylbenzamide

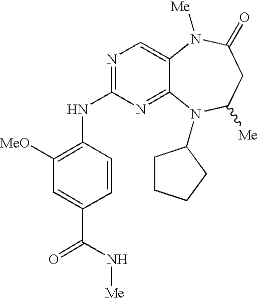

The title compound was synthesized using an analogous procedure to that described in connection with Compound 10 except that ethyl 3-aminobutanoate was used. ¹H NMR (300 MHz, CHLOROFORM-d) δ 1.29 (3H, d, J=6.4 Hz), 1.30-1.43 (1H, m), 1.63-1.89 (5H, m), 1.93-2.06 (1H, m), 2.08-2.21 (1H, m), 2.57 (1H, dd, J=14.1, 7.7 Hz), 2.74 (1H, dd, J=13.9, 2.4 Hz), 3.02 (3H, d, J=4.9 Hz), 3.31 (3H, s), 3.96-4.08 (1H, m), 3.97 (3H, s), 4.61-4.79 (1H, m), 6.11 (1H, q, J=4.5 Hz), 7.20-7.31 (3H, m), 7.44 (2H, d, J=1.7 Hz), 7.67 (1H, s), 7.96 (1H, s), 8.47 (1H, d, J=8.5 Hz). [M+H] calc'd for C₂₃H₃₀N₆O₃ 439; found, 439.

### Compound 14

### 4-{[(cis)-10-cyclopentyl-5-methyl-6-oxo-5,6,6a,7,8,9,9a,10-octahydrocyclopenta[e]pyrimido[5,4-b][1,4]diazepin-2-yl]amino}-3-methoxy-N-methylbenzamide

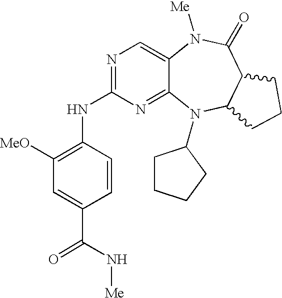

The title compound was synthesized using an analogous procedure to that described in connection with Compound 10 except that ethyl (cis)-2-aminocyclopentanecarboxylate was used. ¹H NMR (300 MHz, CHLOROFORM-d) δ 1.50-2.11 (14H, m), 2.96-3.06 (4H, m), 3.30 (3H, s), 3.99 (3H, s), 4.07-4.21 (1H, m), 7.22-7.26 (1H, m), 7.45-7.48 (2H, m, J=1.5 Hz), 7.72 (1H, s), 8.07 (1H, s), 8.51 (1H, d, J=8.3 Hz). [M+H] calc'd for C₂₅H₃₂N₆O₃ 465; found, 465.

### Compound 15

### 4-(9-cyclopentyl-8-ethyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

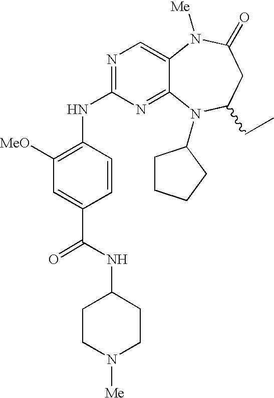

The title compound was synthesized using an analogous procedure to that described in connection with Compound 11 except that methyl 3-aminopentanoate was used and the final compound was purified by reverse phase HPLC as the TFA salt. ¹H NMR (400 MHz, DMSO-d₆) δ 0.92 (t, J=8.0 Hz, 3H), 1.48 (m, 3H), 1.72 (m, 6H), 1.99 (m, 5H), 2.70 (m, 1H), 2.79 (s, 3H), 2.81 (m, 3H), 3.11 (m, 2H), 3.20 (s, 3H), 3.78 (m, 1H), 3.94 (s, 3H), 4.04 (m, 1H), 4.62 (m, 1H), 7.53 (overlap d & s, 2H), 8.02 (d, J=8 Hz, 1H), 8.08 (s, 1H), 8.41 (d, J=4 Hz, 1H), 8.69 (br s, 1H), 9.41 (br s, 1H). [M+H] calc'd for C₂₉H₄₁N₇O₃ 536; found, 536.

### Compound 16

### 4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

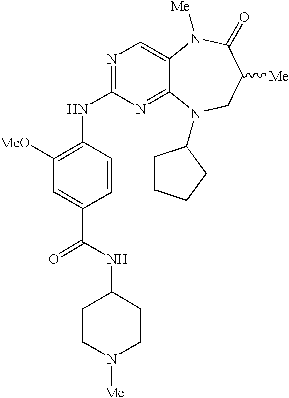

The title compound was synthesized using an analogous procedure to that described in connection with Compound 11 except that methyl 3-amino-2-methylpropanoate was used and the final compound was purified by reverse phase HPLC as the TFA salt. ¹H NMR (400 MHz, DMSO-d₆) δ 1.05 (d, J=8.0 Hz, 3H), 1.57-1.78 (m, 9H), 2.03 (m, 3H) 2.79 (s, 3H), 2.84 (m, 2H), 3.14 (m, 2H), 3.19 (s, 3H), 3.34-3.58 (m, 3H), 3.95 (s, 3H), 4.02 (m, 1H), 4.79 (t, J=8 Hz, 1H), 7.51 (overlap d & s, 2H), 8.12 (s, 1H), 8.23 (m, 1H), 8.39 (d, J=8 Hz, 1H), 8.56 (br s, 1H), 9.40 (br s, 1H). [M+H] calc'd for C₂₈H₃₉N₇O₃ 522; found, 522.

### Compound 17

### 3-methoxy-4-{[(7aR)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide

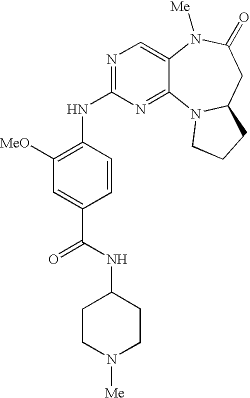

The title compound was synthesized using an analogous procedure to that described in connection with Compound 11 except that (R)-methyl 2-(pyrrolidin-2-yl)acetate was used and the final compound was purified by reverse phase HPLC as the TFA salt. ¹H NMR (400 MHz, DMSO-d₆) δ 1.63 (m, 1H), 1.78 (m, 3H), 2.00 (m, 3H), 2.20 (m, 1H), 2.57 (d, J=12.0 Hz, 1H), 2.71 (d, J=4 Hz, 3H), 2.78 (m, 2H), 3.05 (q, J=8 Hz, 2H), 3.13 (s, 3H), 3.68 (m, 1H), 3.71 (m, 1H), 3.89 (s, 3H), 3.95 (m, 1H), 7.46 (s, 1H), 7.51 (d, J=8 Hz, 1H), 8.04 (s, 1H), 8.27 (d, J=8 Hz, 1H), 8.35 (d, J=8 Hz, 1H), 8.72 (br s, 1H), 9.52 (br s, 1H). [M+H] calc'd for C₂₅H₃₃N₇O₃ 480; found, 480.

### Compound 18

### 3-methoxy-4-{[(7aS)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide

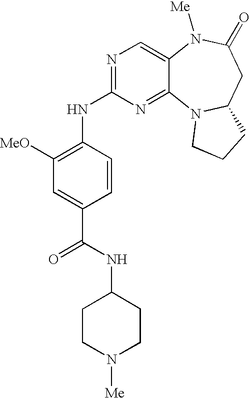

The title compound was synthesized using an analogous procedure to that described in connection with Compound 11 except that (S)-methyl 2-(pyrrolidin-2-yl)acetate was used and the final compound was purified by reverse phase HPLC as the TFA salt. ¹H NMR (400 MHz, DMSO-d₆) δ 1.63 (m, 1H), 1.78 (m, 3H), 2.00 (m, 3H), 2.20 (m, 1H), 2.57 (d, J=12.0 Hz, 1H), 2.71 (d, J=4 Hz, 3H), 2.78 (m, 2H), 3.05 (q, J=8 Hz, 2H), 3.13 (s, 3H), 3.68 (m, 1H), 3.71 (m, 1H), 3.89 (s, 3H), 3.95 (m, 1H), 7.46 (s, 1H), 7.51 (d, J=8 Hz, 1H), 8.04 (s, 1H), 8.27 (d, J=8 Hz, 1H), 8.35 (d, J=8 Hz, 1H), 8.72 (br s, 1H), 9.52 (br s, 1H). [M+H] calc'd for C₂₅H₃₃N₇O₃ 480; found, 480.

### Compound 19

### 4-[(9-isopropyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide

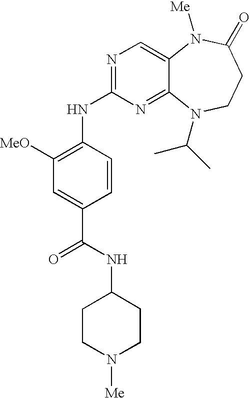

The title compound was synthesized using an analogous procedure to that described in connection with Compound 110 except that methyl 3-(isopropylamino)propanoate was used and the final compound was purified by reverse phase HPLC as the TFA salt. ¹H NMR (400 MHz, DMSO-d₆) δ 1.23 (d, J=4.0 Hz, 6H), 1.78 (m, 2H), 2.01 (m, 2H), 2.67 (m, 2H), 2.79 (s, 3H), 3.12 (m, 1H), 3.16 (s, 3H), 3.46 (m, 2H), 3.68 (m, 2H), 3.95 (s, 3H), 4.03 (m, 2H), 4.83 (m, 1H), 7.54 (overlap d & s, 2H), 8.11 (s, 1H), 8.19 (d, J=8 Hz, 1H), 8.41 (d, J=8 Hz, 1H), 8.85 (br s, 1H), 9.48 (br s, 1H). [M+H] calc'd for C₂₅H₃₅N₇O₃ 481; found, 481.

### Compound 20

### 4-(9-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

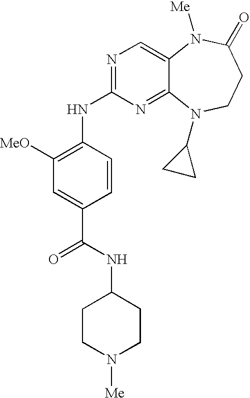

The title compound was synthesized using an analogous procedure to that described in connection with Compound 11 except that methyl 3-(cyclopropylamino)propanolate, prepared by Michael addition of ethyl acrylate (1 equiv.) with cyclopropylamine (1 equiv.) in ethanol overnight (see Scheme 2), was used and the final compound was purified by reverse phase HPLC to yield the TFA salt. ¹H NMR (400 MHz, DMSO-d₆) δ 0.7 (m, 2H), 0.88 (m, 2H), 1.77 (m, 2H), 2.01 (m, 2H), 2.67 (m, 2H), 2.79 (d. J=8 Hz, 3H), 2.93 (m, 1H), 3.12 (overlap m&s, 5H), 3.47 (d, J=12 Hz, 2H), 3.80 (m, 2H), 3.96 (s, 3H), 4.14 (m, 1H), 7.54 (overlap d & s, 2H), 8.18 (s, 1H), 8.35 (d, J=8 Hz, 1H), 8.50 (br s, 1H), 8.64 (d, J=8 Hz, 1H), 9.42 (br s, 1H). [M+H] calc'd for C₂₅H₃₃N₇O₃ 479; found, 479.

### Compound 21

### 4-(9-cyclopropyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

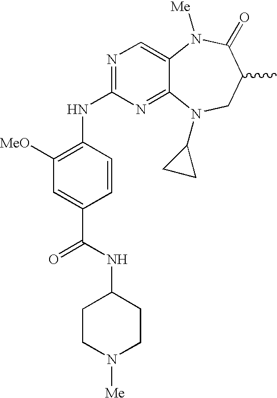

The title compound was synthesized using an analogous procedure to that described in connection with Compound 11 except that methyl 3-(cyclopropylamino)-2-methylpropanoate, prepared by Michael addition of methyl methacrylate (1 equiv.) with cyclopropylamine (1 equiv.) in refluxing ethanol for 3 days, was used and the final compound was purified by reverse phase HPLC to yield the TFA salt. ¹H NMR (400 MHz, DMSO-d₆) δ 0.54 (br. s., 1H) 0.72-0.88 (m, 2H) 1.01 (d, J=6.6 Hz, 3H) 0.88-1.02 (m, 1H) 1.24 (br. s., 1H) 1.75 (m, 2H) 1.93-2.09 (m, 2H) 2.80 (d, J=5.0 Hz, 1H) 2.78 (d, J=4.3 Hz, 3H) 2.94 (dd, J=6.8, 3.3 Hz, 1H) 2.99-3.18 (m, 5H) 3.46-3.54 (m, 2H) 3.67 (t, J=12.0 Hz, 1H) 3.96 (s, 3H), 4.07 (m, 1H) 7.56 (d, J=8.8 Hz, 1H) 7.40-7.60 (m, 1H) 8.20 (s, 1H) 8.37 (d, J=7.6 Hz, 1H) 8.61 (d, J=8.3 Hz, 1H) 8.74 (br. s., 1H) 9.42 (br. s., 1H). [M+H] calc'd for C₂₆H₃₅N₇O₃ 494; found, 494.

### Compound 22

### (S)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

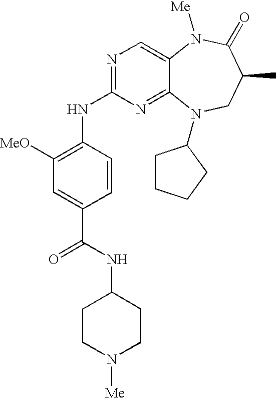

The title compound was synthesized using an analogous procedure to that described in connection with Compound 11 except that (S)-3-amino-2-methylpropionic acid methyl ester was used and the final compound was purified by reverse phase HPLC to yield the TFA salt. ¹H NMR (400 MHz, DMSO-d₆) δ 1.04 (d, J=6.8 Hz, 3H) 1.56 (br. s., 3H) 1.65-1.82 (m, 5H) 2.02 (br. s., 2H) 2.07 (m, 1H) 2.78 (d, J=4.8 Hz, 3H) 2.95 (d, J=7.6 Hz, 1H) 3.11 (m, 2H) 3.19 (s, 3H) 3.34 (m, 1H) 3.42-3.60 (m, 3H) 3.96 (br. s., 1H) 3.95 (s, 3H) 4.02 (m, 1H) 4.79 (t, J=8.1 Hz, 1H) 7.49-7.56 (m, 2H) 8.11 (s, 1H) 8.21 (d, J=8.3 Hz, 1H) 8.39 (d, J=7.3 Hz, 1H) 8.65 (br. s., 1H) 9.38 (br. s., 1H). [M+H] calc'd for C₂₈H₃₉N₇O₃ 522; found, 522.

### Compound 23

### (R)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

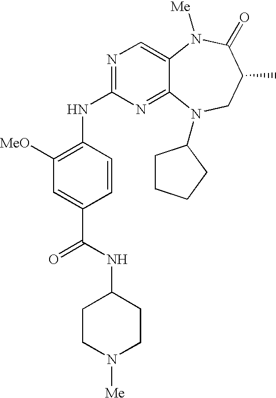

The title compound was synthesized using an analogous procedure to that described in connection with Compound 11 except that (R)-3-amino-2-methylpropionic acid methyl ester was used and the final compound was purified by reverse phase HPLC to yield the TFA salt. ¹H NMR (400 MHz, DMSO-d₆) δ 1.04 (d, J=6.8 Hz, 3H) 1.57 (m, 3H) 1.78 (m, 5H) 2.02 (m., 3H) 2.79 (d, J=4.8 Hz, 3H) 2.93 (m, 1H) 3.11 (m, 2H) 3.19 (s, 3H) 3.34 (m, 1H) 3.42-3.59 (m, 3H) 3.96 (br. s., 1H) 3.95 (s, 3H) 4.04 (br. s., 1H) 4.02 (m, 1H) 4.78 (t, J=8.1 Hz, 1H) 7.48-7.54 (m, 2H) 8.11 (s, 1H) 8.23 (d, J=8.6 Hz, 1H) 8.38 (d, J=7.6 Hz, 1H) 8.54 (br. s., 1H) 9.32 (br. s., 1H). [M+H] calc'd for C₂₈H₃₉N₇O₃ 522; found, 522.

### Compound 24

### 4-[(9-cyclohexyll-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide

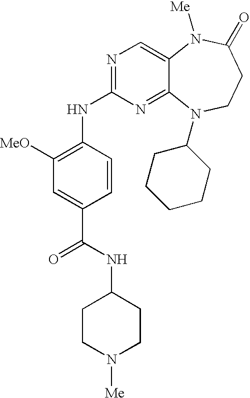

The title compound was synthesized using an analogous procedure to that described in connection with Compound 11 except that ethyl 3-(cyclohexylamino)propanoate, which was prepared by Michael addition of ethyl acrylate (1 equiv.) with cyclohexylamine (1 equiv.) in ethanol overnight (refer to Scheme 2), was used and the final compound was purified by reverse phase HPLC to yield the TFA salt. ¹H NMR (400 MHz, DMSO-d₆) δ 1.14 (m, 1H) 1.30 (m, 2H) 1.58 (d, J=9.8 Hz, 1H) 1.58-1.84 (m, 9H) 2.02 (m, 2H) 2.66 (d, J=8.3 Hz, 2H) 2.66 (br. s., 1H) 2.78 (d, J=4.3 Hz, 3H) 3.02-3.22 (m, 1H) 3.16 (s & m, 6H) 3.95 (s, 3H), 4.02 (m, 1H) 4.39 (t, J=11.7 Hz, 1H) 7.48-7.57 (m, 2H) 8.10 (s, 1H) 8.17 (d, J=8.3 Hz, 1H) 8.43 (d, J=7.3 Hz, 1H) 8.80 (br. s., 1H) 9.47 (br. s., 1H). [M+H] calc'd for C₂₈H₃₉N₇O₃ 522; found, 522.

### Compound 25

### 4-(9-isobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

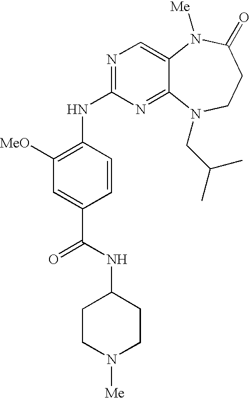

The title compound was synthesized using an analogous procedure to that described in connection with Compound 11 except that ethyl 3-(isobutylamino)propanolate, which was prepared by Michael addition of ethyl acrylate (1 equiv.) with isobutylamine (1.2 equiv.) in ethanol overnight (refer to Scheme 2), was used and the final compound was purified by reverse phase HPLC to yield the TFA salt. ¹H NMR (400 MHz, DMSO-d₆) δ 0.87 (d, J=6.6 Hz, 6H) 1.77 (m, 2H) 1.95-2.15 (m, 3H) 2.73 (m, 2H) 2.79 (d, J=4.6 Hz, 3H) 2.81 (m, 1H) 3.04-3.26 (m, 7H) 3.76 (m, 3H) 3.85-4.07 (m, 5H) 7.48-7.58 (m, 2H) 8.14 (d, J=8.3 Hz, 1H) 8.10 (s, 1H) 8.39 (d, J=7.3 Hz, 1H) 8.66 (br. s., 1H) 9.34 (br. s., 1H). [M+H] calc'd for C₂₆H₃₇N₇O₃ 496; found, 496.

### Compound 26

### 4-(9-benzyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

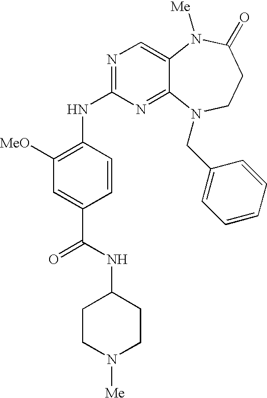

The title compound was synthesized using an analogous procedure to that described in connection with Compound 11 except that methyl 3-(benzylamino)propanoate was used and the final compound was purified by reverse phase HPLC to yield the TFA salt. ¹H NMR (400 MHz, DMSO-d₆) δ 1.17 (t, J=7.2 Hz, 1H) 1.26 (m, 1H) 1.75 (m, 2H) 1.99 (m, 2H) 2.78 (d, J=4.6 Hz, 3H) 3.14 (m, 2H) 3.21 (s, 3H) 3.45 (m, 2H) 3.68 (m, 2H) 3.91 (s, 3H) 4.00 (m, 1H) 4.90 (s, 2H) 7.22 (d, J=8.08 Hz, 1H) 7.26-7.38 (m, 5H) 7.46 (s, 1H) 7.93 (d, J=8.34 Hz, 1H) 8.18 (s, 1H) 8.32 (d, J=7.33 Hz, 1H) 8.56 (br. s., 1H) 9.32 (br. s., 1H). [M+H] calc'd for C₂₉H₃₅N₇O₃ 530; found, 530.

### Compound 27

### 4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

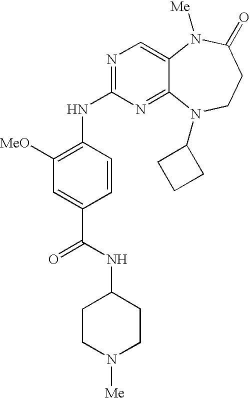

The title compound was synthesized using an analogous procedure to that described in connection with Compound 11 except that ethyl 3-(cyclobutylamino)propanoate, which was prepared by Michael addition of ethyl acrylate (1 equiv.) with cyclobutylamine (1.2 equiv.) in ethanol overnight (refer to Scheme 2), was used and the final compound was purified by reverse phase HPLC to yield the TFA salt. ¹H NMR (400 MHz, DMSO-d₆) δ 1.63-1.83 (m, 4H) 2.00-2.23 (m, 6H) 2.65 (d, J=4.8 Hz, 2H) 2.78 (d, J=4.6 Hz, 3H) 3.05-3.22 (m, 2H) 3.17 (s, 3H) 3.74 (m, 1H) 3.72 (d, J=5.8 Hz, 3H) 3.94 (s, 3H) 4.03 (m, 1H) 4.56 (t, J=8.2 Hz, 1H) 7.54 (q, J=8.2 Hz, 2H) 8.13 (s, 1H) 8.28 (d, J=8.3 Hz, 1H) 8.40 (d, J=7.6 Hz, 1H) 8.57 (br. s., 1H) 9.51 (br. s., 1H). [M+H] calc'd for C₂₆H₃₅N₇O₃ 494; found, 494.

### Compound 28

### 3-methoxy-4-(5-methyl-6-oxo-9-(pentan-3-yl)-6,7,8,9-tetrahydro-5H-pyrimido[4,5-b][1,4]diazepin-2-ylamino)-N-(1-methylpiperidin-4-yl)benzamide

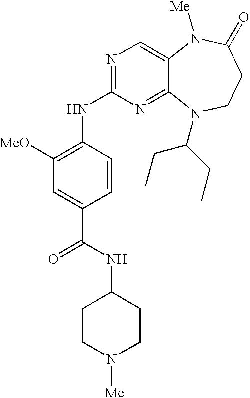

The title compound was synthesized using an analogous procedure to that described in connection with Compound 11 except that ethyl 3-(pentan-3-ylamino)propanolate, which was prepared by Michael addition of ethyl acrylate (1 equiv.) with 1-ethylpropylamine (1.2 equiv.) in ethanol overnight (refer to Scheme 2), was used and the final compound was purified by reverse phase HPLC to yield the TFA salt. ¹H NMR (400 MHz, DMSO-d₆) δ 0.81 (t, J=7.3 Hz, 6H) 1.48-1.67 (m, 4H) 1.68-1.87 (m, 2H) 2.03 (d, J=12.4 Hz, 2H) 2.78 (d, J=4.6 Hz, 3H) 3.15 (m, 2H), 3.18 (s, 3H) 3.48 (d, J=11.6 Hz, 2H) 3.58 (d, J=8.8 Hz, 2H) 3.94 (s, 3H), 4.04 (m, 1H) 4.65 (d, J=6.6 Hz, 2H) 4.63 (m, 1H) 7.43-7.62 (m, 2H) 8.07 (d, J=8.3 Hz, 1H) 8.12 (s, 1H) 8.44 (d, J=7.3 Hz, 1H) 8.99 (br. s., 1H) 9.55 (br. s., 1H). [M+H] calc'd for C₂₇H₃₉N₇O₃ 510; found, 510.

### Compound 29

### 4-(9-sec-butyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

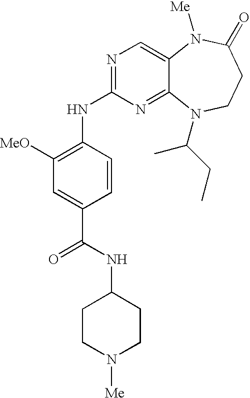

The title compound was synthesized using an analogous procedure to that described in connection with Compound 11 except that ethyl 3-(sec-butylamino)propanoate, which was prepared by Michael addition of ethyl acrylate (1 equiv.) with sec-butylamine (1.2 equiv.) in ethanol overnight (refer to Scheme 2), was used and the final compound was purified by reverse phase HPLC to yield the TFA salt. ¹H NMR (400 MHz, DMSO-d₆) δ 0.83 (t, J=7.3 Hz, 3H) 1.21 (d, J=6.8 Hz, 3H) 1.50-1.68 (m, 2H) 1.71-1.83 (m, 2H) 2.02 (t, J=12.6 Hz, 2H) 2.61-2.75 (m, 2H) 2.78 (d, J=4.3 Hz, 3H) 3.09 (m, 2H) 3.17 (s, 3H) 3.48 (d, J=11.9 Hz, 2H) 3.55-3.76 (m, 2H) 3.94 (s, 3H) 4.03 (m, 1H) 4.66 (m, 2H) 7.41-7.60 (m, 2H) 8.04-8.19 (m, 2H) 8.43 (d, J=7.6 Hz, 1H) 8.93 (br. s., 1H) 9.53 (br. s., 1H). [M+H] calc'd for C₂₆H₃₇N₇O₃ 496; found, 496.

### Compound 30

### 4-(9-allyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

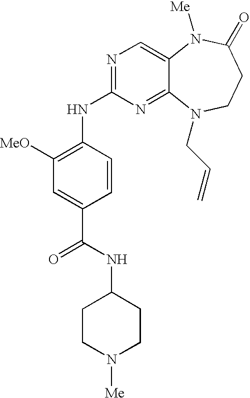

The title compound was synthesized using an analogous procedure to that described in connection with Compound 11 except that methyl 3-(allylamino)propanoate was used and the final compound was purified by reverse phase HPLC to yield the TFA salt. ¹H NMR (400 MHz, DMSO-d₆) δ 1.68-1.90 (m, 1H) 1.77 (d, J=12.6 Hz, 1H) 1.90-2.09 (m, 2H) 2.66-2.84 (m, 2H) 2.78 (d, J=4.3 Hz, 3H) 3.11 (m, 1H) 3.18 (s, 3H) 3.48 (d, J=11.6 Hz, 2H) 3.72 (m, 2H) 3.94 (s, 3H) 4.02 (d, J=6.8 Hz, 1H) 4.24 (d, J=4.0 Hz, 2H) 5.23 (d, J=6.6 Hz, 2H) 5.26 (s, 1H) 5.92 (m, 1H) 7.53 (d, J=3.3 Hz, 2H) 8.16 (m, 2H) 8.41 (d, J=7.3 Hz, 1H) 8.85 (br. s., 1H) 9.54 (br. s., 1H). [M+H] calc'd for C₂₅H₃₃N₇O₃ 480; found, 480.

### Compound 31

### 3-methoxy-4-(5-methyl-6-oxo-9-phenyl-6,7,8,9-tetrahydro-5H-pyrimido[4,5-b][1,4]diazepin-2-ylamino)-N-(1-methylpiperidin-4-yl)benzamide

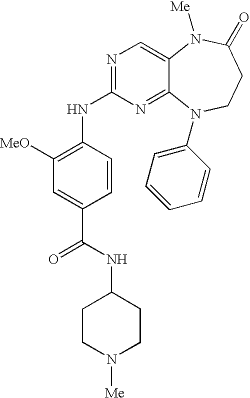

The title compound was synthesized using an analogous procedure to that described in connection with Compound 11 except that methyl 3-(phenylamino)propanoate was used and the final compound was purified by reverse phase HPLC to yield the TFA salt. ¹H NMR (400 MHz, DMSO-d₆) δ 1.75 (m, 1H) 1.90-2.07 (m, 2H) 2.78 (d, J=4.6 Hz, 3H) 2.88 (m, 2H) 3.02-3.19 (m, 2H) 3.27 (s, 3H) 3.46 (m, 2H) 3.89 (s, 3H) 3.95-4.18 (m, 4H) 6.93 (dd, J=8.5, 1.1 Hz, 1H) 7.33-7.42 (m, 4H) 7.51 (t, J=7.7 Hz, 2H) 8.10 (br. s., 1H) 8.27 (d, J=7.6 Hz, 1H) 8.31 (s, 1H) 9.42 (br. s., 1H). [M+H] calc'd for C₂₈H₃₃N₇O₃ 516; found, 516.

### Compound 32

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide

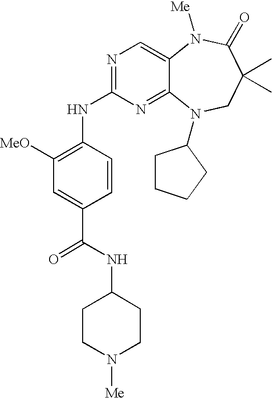

The title compound was synthesized using an analogous procedure to that described in connection with Compound 11 except that 3-amino-2,2-dimethylpropionic acid methyl ester was used and the final compound was purified by reverse phase HPLC to yield the TFA salt. ¹H NMR (400 MHz, DMSO-d₆) δ 1.12 (s, 6H) 1.61-2.02 (m, 12H) 2.67 (dt, J=3.6, 1.9 Hz, 1H) 2.79 (d, J=4.6 Hz, 3H) 3.14 (m, 2H) 3.18 (s, 3H) 3.45 (m, 3H) 3.95 (s, 3H) 4.00 (m, 1H) 5.15 (m, 1H) 7.52 (d, J=2.3 Hz, 1H) 7.50 (d, J=1.8 Hz, 1H) 8.00 (s, 1H) 8.23 (br. s., 1H) 8.36 (d, J=7.6 Hz, 1H) 9.26 (br. s., 1H). [M+H] calc'd for C₂₉H₄₁N₇O₃ 536; found, 536.

### Compound 33

### 4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-4-yl)benzamide

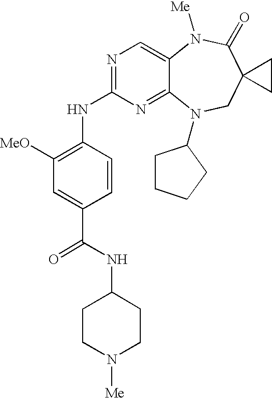

The title compound was synthesized using an analogous procedure to that described in connection with Compound 11 except that 1-aminomethylcyclopropanecarbonic acid methyl ester was used and the final compound was purified by reverse phase HPLC to yield the TFA salt. ¹H NMR (400 MHz, DMSO-d₆) δ 0.68-0.88 (m, 2H) 1.00 (m, 2H) 1.12-1.32 (m, 1H) 1.17 (t, J=7.1 Hz, 1H) 1.53 (br s., 4H) 1.69 (br. s., 2H) 1.77 (m, 2H) 1.83 (br. s., 2H) 2.00 (m, 2H) 2.78 (d, J=4.3 Hz, 3H) 3.01-3.22 (m, 3H) 3.11 (m, 1H) 3.46 (m, 1H) 3.61 (br. s., 1H) 3.95 (s, 3H) 4.03 (m, 1H) 4.86 (q, J=8.3 Hz, 2H) 7.53 (d, J=9.6 Hz, 2H) 7.56 (s, 1H) 8.02 (s, 1H) 8.11 (d, J=8.3 Hz, 1H) 8.44 (d, J=7.3 Hz, 1H) 9.10 (br. s., 1H) 9.55 (br. s., 1H). [M+H] calc'd for C₂₉H₃₉N₇O₃ 534; found, 534.

### Compound 34

### 4-(10-Cyclopentyl-5-methyl-6-oxo-5,6,7,8,9,10-hexahydro-pyrimido[4,5-b][1,4]diazocin-2-ylamino)-3-methoxy-N-(1-methyl-piperidin-4-yl)-benzamide

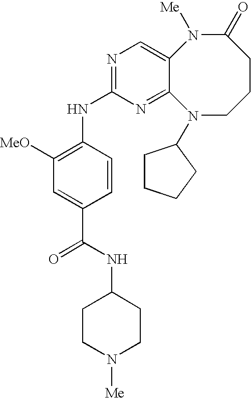

The title compound was synthesized using an analogous procedure to that described in connection with Compound 110 except that 4-cyclopentylamino-butyric acid methyl ester, prepared from reduction of methyl 1-cyanocyclopropanecarboxylate, was used and the final compound was purified by reverse phase HPLC to yield the TFA salt. ¹H NMR (400 MHz, DMSO-d₆) δ 1.45-1.62 (m, 3H), 1.65-1.91 (m, 9H), 1.99-2.09 (m, 2H), 2.14-2.24 (m, 1H), 2.52-2.61 (m, 1H), 2.79 (d, J=4.55 Hz, 3H), 3.06-3.15 (m, 2H), 3.17 (s, 3H), 3.43-3.52 (m, 2H), 3.60 (t, J=13.39 Hz, 2H), 5.18 (quintet, J=8.08 Hz, 1H), 7.50-7.62 (m, 2H), 8.00 (d, J=8.34 Hz, 1H), 8.09 (s, 1H), 8.46 (d, J=7.33 Hz, 1H), 9.19 (br. s., 1H), 9.59 (br. s., 1H). ¹H NMR (400 MHz, DMSO-d₆-D₂O) δ 1.49-1.63 (m, 3H), 1.65-1.94 (m, 9H), 1.98-2.13 (m, 2H), 2.23 (dd, J=11.49, 5.43 Hz, 1H), 2.54-2.63 (m, 1H), 2.80 (s, 3H), 3.03-3.15 (m, 2H), 3.17 (s, 3H), 3.22-3.40 (m, 1H), 3.43-3.55 (m, 3H), 3.58-3.71 (m, 1H), 3.95 (s, 3H), 3.99-4.08 (m, 1H), 5.14 (quintet, J=8.59 Hz, 1H), 7.53 (dd, J=8.34, 1.77 Hz, 1H), 7.58 (d, J=1.77 Hz, 1H), 7.97 (d, J=8.59 Hz, 1H), 8.05 (s, 1H).). [M+H] calc'd for C₂₈H₃₉N₇O₃ 522; found, 522.

### Compound 35

### (R)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide

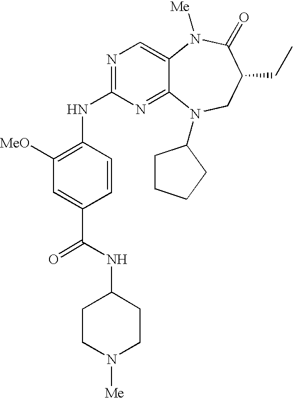

The title compound was synthesized using an analogous procedure to that described in connection with Compound 11 except that (R)-2-aminomethylbutyric acid methyl ester was used and the final compound was purified by reverse phase HPLC to yield the TFA salt. ¹H NMR (400 MHz, DMSO-d₆) δ 0.89 (t, J=7.33 Hz, 3H), 1.34 (septet, J=6.57 Hz, 1H), 1.44-1.64 (m, 3H), 1.47-1.64 (m, 3H), 1.67-1.86 (m, 7H), 1.93-2.11 (m, 3H), 2.66-2.85 (m, 1H), 2.79 (d, J=4.55 Hz, 3H), 3.05-3.17 (m, 2H), 3.19 (s, 3H), 3.38-3.60 (m, 4H), 3.95 (s, 3H), 3.99-4.10 (m, 1H), 4.83 (quintet, J=8.08 Hz, 1H), 7.47-7.64 (m, 2H), 8.07-8.19 (m, 2H), 8.43 (d, J=7.58 Hz, 1H), 8.97 (br. s., 1H), 9.50 (br. s., 1H). [M+H] calc'd for C₂₉H₄₁N₇O₃ 536; found, 536.

### Compound 36

### (S)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide

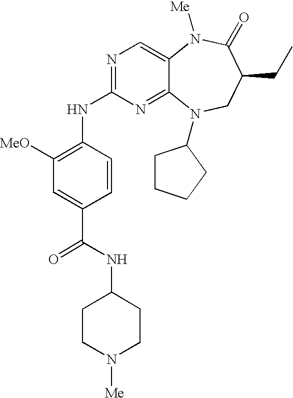

The title compound was synthesized using an analogous procedure to that described in connection with Compound 11 except that (S)-2-aminomethylbutyric acid methyl ester was used and the final compound was purified by reverse phase HPLC to yield the TFA salt. ¹H NMR (400 MHz, DMSO-d₆) δ 0.89 (t, J=7.33 Hz, 3H), 1.34 (septet, J=14.02, 6.95, 6.82 Hz, 1H), 1.48-1.63 (m, 3H), 1.67-1.86 (m, 7H), 1.91-2.09 (m, 3H), 2.71-2.84 (m, 1H), 2.79 (d, J=4.55 Hz, 3H), 3.06-3.16 (m, 2H), 3.19 (s, 3H), 3.40-3.61 (m, 4H), 3.95 (s, 3H), 3.97-4.00 (m, 1H), 4.84 (d, J=8.34 Hz, 1H), 7.49-7.58 (m, 2H), 8.08-8.17 (m, 2H), 8.44 (d, J=7.58 Hz, 1H), 9.09 (br. s., 1H), 9.50 (br. s., 1H).). [M+H] calc'd for C₂₉H₄₁N₇O₃ 536; found, 536.

### Compound 37

### 4-(9-cyclopentyl-5-methyl-6-oxo-6,7,8,9-tetrahydro-5H-pyrimido[4,5-b][1,4]diazepin-2-ylamino)-3-methoxy N-(1-(methylsulfonyl)piperidin-4-yl)benzamide

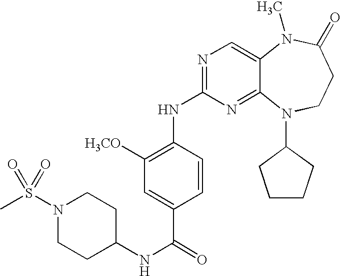

The title compound was synthesized using an analogous procedure to that described in connection with Compound 10 except that except that N-(1-methylsulfonyl)piperidine-4-amine was used. ¹H NMR (400 MHz, DMSO-d₆) δ ppm 1.54-1.76 (m, 1H) 1.61 (td, J=11.87, 8.59 Hz, 4H) 1.91 (d, J=2.53 Hz, 2H) 1.93 (br. s., 1H) 2.67 (d, J=1.77 Hz, 1H) 2.81-2.93 (m, 1H) 2.89 (s, 2H) 3.16 (s, 2H) 3.60 (d, J=12.13 Hz, 2H) 3.70 (d, J=8.84 Hz, 3H) 3.86-4.01 (m, 1H) 3.95 (s, 3H) 4.86 (t, J=8.21 Hz, 1H) 7.55 (s, 1H) 7.52 (d, J=8.59 Hz, 1H) 8.09 (s, 1H) 8.10 (br s, —NH) 8.29 (d, J=8.42 Hz, 1H) 8.89 (br s, —NH). [M+H] calculated for C₂₇H₃₈N₅O₇S, 572; found 572.

### Compound 38

### 4-(9-cyclopentyl-5-methyl-6-oxo-6,7,8,9-tetrahydro-5H-pyrimido[4,5-b][1,4]diazepin-2-ylamino)-3-methoxy-N-(3-(2-oxopyrrolidin-1-yl)propyl)benzamide

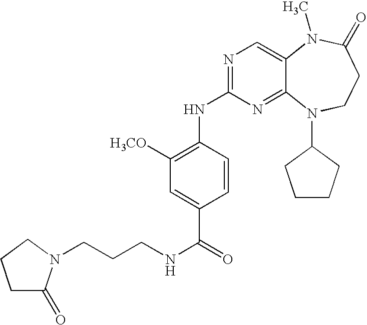

The title compound was synthesized using an analogous procedure to that described in connection with Compound 10 except that 1-(3-aminopropyl)pyrrolidin-2-one was used. ¹H NMR (400 MHz, DMSO-d₆) δ ppm 1.60 (br. s., 2H) 1.64-1.76 (m, J=7.01 Hz, 3H) 1.84-1.97 (m, 1H) 1.91 (d, J=7.83 Hz, 1H) 2.22 (t, J=7.96 Hz, 1H) 2.70 (d, J=4.04 Hz, 1H) 3.16 (s, 2H) 3.24 (q, J=6.99 Hz, 2H) 3.36 (t, J=6.95 Hz, 2H) 3.71 (m, 7H) 3.95 (s, 3H) 4.86 (t, J=8.21 Hz, 1H) 7.52 (d, J=8.0 Hz, 1H) 7.57 (s, 1H) 8.09 (s, 1H) 8.11 (d, J=8.42 Hz, 1H) 8.45 (t, —NH) 8.98 (br s, —NH). [M+H] calculated for C₂₈H₃₈N₇O₄, 536; found 536.

### Compound 39

### N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide

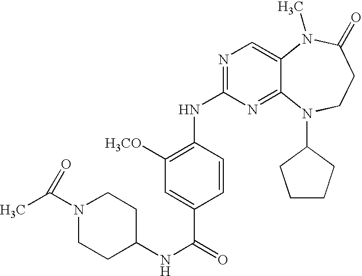

The title compound was synthesized using an analogous procedure to that described in connection with Compound 10 except that 1-N-acetyl 4-aminopiperidine was used. ¹H NMR (400 MHz, DMSO-d₆) δ ppm 1.39-1.89 (m, 12H) 2.02 (s, 3H) 2.67 (dd, J=3.54, 1.77 Hz, 2H) 3.11 (m, 1H) 3.16 (s, 3H) 3.70 (d, J=9.09 Hz, 2H) 3.83 (br. s., 2H) 3.95 (s, 3H) 4.05 (m, 1H) 4.36 (br d., 1H) 4.86 (t, J=8.21 Hz, 1H) 7.52 (d, J=8.0 Hz, 1H) 7.55 (s, 1H) 8.09 (s, 1H) 8.11 (br s, —NH) 8.21 (d, J=8.42 Hz, 1H) 8.88 (br s, —NH). [M+H] calculated for C₂₈H₃₈N₇O₄, 536; found 536.

### Compound 40

### (S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3yl)benzamide

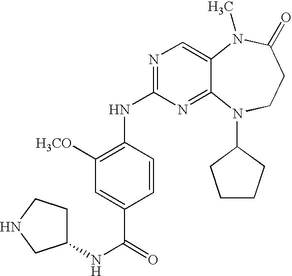

The title compound was synthesized using an analogous procedure to that described in connection with Compound 10 except that (S)-N-Boc-3-aminopyrrolidine was used. Further, after washing with water (10 ml), t-butoxycarbonyl (Boc) protection group was removed using 40% TFA in dichloromethane (6 ml) and purified the product using preparative HPLC. ¹H NMR (400 MHz, DMSO-d₆) δ ppm 1.56-1.75 (m, 4H) 1.73 (dd, J=11.49, 6.95 Hz, 2H) 1.89 (d, J=6.82 Hz, 2H) 2.04 (dd, J=13.50, 8.12 Hz, 1H) 2.23 (dd, J=13.52, 7.20 Hz, 1H) 2.70 (m, 2H) 3.17 (s, 3H) 3.25-3.30 (m, 1H) 3.33-3.54 (m, 2H) 3.66-3.77 (m, 1H) 3.71 (d, J=9.60 Hz, 1H) 3.95 (s, 3H) 4.50 (ddd, J=12.76, 6.57, 6.44 Hz, 1H) 4.86 (dt, J=16.42, 8.21 Hz, 1H) 7.56 (d, J=1.77 Hz, 1H) 7.53 (dd, J=8.46, 1.64 Hz, 1H) 8.12 (s, 1H) 8.15 (d, J=8.40 Hz, 1H) 8.59 (d, J=6.06 Hz, —NH) 8.90 (br. s., 1H). [M+H] calculated for C₂₅H₃₄N₇O₃, 480; found 480.

### Compound 41

### (R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide

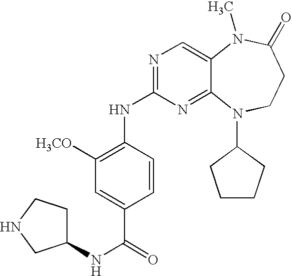

The title compound was synthesized using an analogous procedure to that described in connection with Compound 10 except that (R)-N-Boc-3-aminopyrrolidine was used. N-Boc protection was removed as in Example-4. ¹H NMR (400 MHz, DMSO-d₆) δ ppm 1.54-1.73 (m, 4H) 1.73 (dd, J=11.49, 6.95 Hz, 2H) 1.89 (d, J=6.82 Hz, 2H) 2.04 (dd, J=13.50, 8.12 Hz, 1H) 2.23 (dd, J=13.52, 7.20 Hz, 1H) 2.70 (m, 2H) 3.17 (s, 3H) 3.25-3.30 (m, 1H) 3.33-3.54 (m, 2H) 3.66-3.77 (m, 1H) 3.71 (d, J=9.60 Hz, 1H) 3.95 (s, 3H) 4.50 (ddd, J=12.76, 6.57, 6.44 Hz, 1H) 4.86 (dt, J=16.42, 8.21 Hz, 1H) 7.56 (d, J=1.77 Hz, 1H) 7.53 (dd, J=8.46, 1.64 Hz, 1H) 8.12 (s, 1H) 8.15 (d, J=8.40 Hz, 1H) 8.59 (d, J=6.06 Hz, —NH) 8.90 (br. s., 1H). [M+H] calculated for C₂₅H₃₄N₇O₃, 480; found 480.

### Compound 42

### 4-(9-cyclopentyl-5-methyl-6-oxo-6,7,8,9-tetrahydro-5H-pyrimido[4,5-b][1,4]diazepin-2-ylamino)-3-methoxy-N-(tetrahydro-2H-pyran-4-yl)benzamide

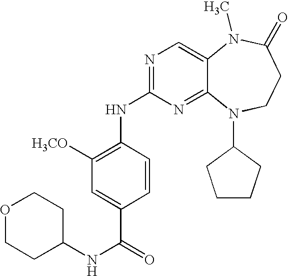

The title compound was synthesized using an analogous procedure to that described in connection with Compound 10 except that tetrahydro-2H-pyran-4-amine was used. ¹H NMR (400 MHz, DMSO-d₆) δ ppm 1.54-89 (m, 12H) 2.65-2.78 (m, 2H) 3.16 (s, 3H) 3.37 (br. s., 1H) 3.41 (dd, J=11.87, 1.77 Hz, 2H) 3.70 (d, J=5.31 Hz, 2H) 3.91-4.04 (m, 3H) 3.95 (s, 3H) 4.87 (dt, J=16.42, 8.21 Hz, 1H) 7.56 (d, J=1.77 Hz, 1H) 7.53 (dd, J=8.46, 1.64 Hz, 1H) 8.09 (s, 1H) 8.12 (br d, —NH) 8.26 (d, J=8.06 Hz, 1H) 8.96 (br. s., —NH). [M+H] calculated for C₂₆H₃₅N₆O₄, 495; found 495.

### Compound 43

### N-(2-acetamidoethyl)-4-(9-cyclopentyl-5-methyl-6-oxo-6,7,8,9-tetrahydro-5H-pyrimido[4,5-b][1,4]diazepin-2-ylamino)-3-methoxybenzamide

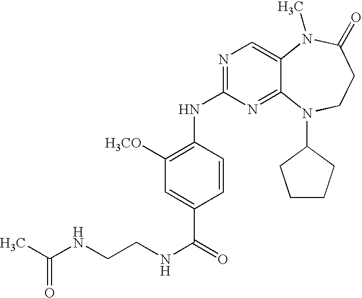

The title compound was synthesized using an analogous procedure to that described in connection with Compound 10 except that N-(2-aminoethyl)acetamide was used. ¹H NMR (400 MHz, DMSO-d₆) δ ppm 1.58-1.91 (m, 8H) 1.82 (s, 3H) 2.72 (d, J=8.34 Hz, 1H) 2.72 (m, 2H) 3.17 (s, 3H) 3.21 (m, 2H) 3.31 (d, J=6.06 Hz, 2H) 3.95 (s, 3H) 4.87 (t, J=8.21 Hz, 1H) 7.52 (dd, J=8.46, 1.64 Hz, 1H) 7.58 (d, J=1.77 Hz, 1H) 8.00-8.15 (m, 2H) 8.56 (t, J=5.43 Hz, 1H) 9.26 (br. s., —NH). [M+H] calculated for C₂₅H₃₄N₇O₄, 496; found 496.

### Compound 44

### 4-(9-cyclopentyl-5-methyl-6-oxo-6,7,8,9-tetrahydro-5H-pyrimido[4,5-b][1,4]diazepin-2-ylamino)-3-methoxy-N-(2-(2-oxoimidazolidin-1-yl)ethyl)benzamide

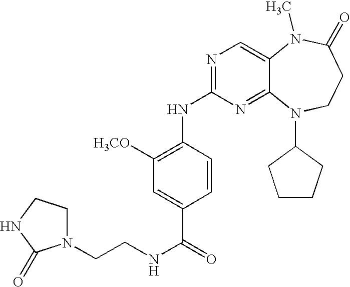

The title compound was synthesized using an analogous procedure to that described in connection with Compound 10 except that 1-(2-aminoethyl) imidazolidin-2-one was used. ¹H NMR (400 MHz, DMSO-d₆) δ ppm 1.58-1.95 (m, 8H) 2.23 (m, 1H), 2.72 (m, 2H) 3.17 (s, 3H) 3.22 (m, 2H) 3.32-3.48 (m, 6H) 3.68 (m, 2H) 3.95 (s, 3H) 4.85 (t, J=8.21 Hz, 1H) 6.30 (br s, 1H) 7.49 (d, J=8.46 Hz, 1H) 7.55 (s, 1H) 8.09 (s, 1H) 8.25 (br d, —NH) 8.56 (br.s, 1H). [M+H] calculated for C₂₆H₃₅N₈O₄, 523; found 523.

### Compound 45

### N-(2-aminoethyl)-4-(9-cyclopentyl-5-methyl-6-oxo-6,7,8,9-tetrahydro-5H-pyrimido[4,5-b][1,4]diazepin-2-ylamino)-3-methoxybenzamide

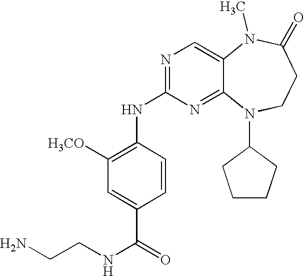

The title compound was synthesized using an analogous procedure to that described in connection with Compound 10 except that N-Boc-ethylenediamine was used. N-Boc protection was removed. ¹H NMR (400 MHz, DMSO-d₆) δ ppm 1.58-1.91 (m, 8H) 2.67 (m, 2H) 2.98 (m, 2H) 3.17 (s, 3H) 3.67 (m, 2H) 3.95 (s, 3H) 4.85 (t, J=8.21 Hz, 1H) 7.52 (d, J=8.46 Hz, 1H) 7.53 (s, 1H) 7.77 (br s, 2H) 8.10 (s, 1H) 8.28 (m, 1H) 8.58 (br s, 1H). [M+H] calculated for C₂₃H₃₂N₇O₃, 454; found 454.

### Compound 46

### 4-(9-cyclopentyl-5-methyl-6-oxo-6,7,8,9-tetrahydro-5H-pyrimido[4,5-b][1,4]diazepin-2-ylamino)-3-methoxy-N-(2-(2-oxopyrrolidin-1-yl)ethyl)benzamide

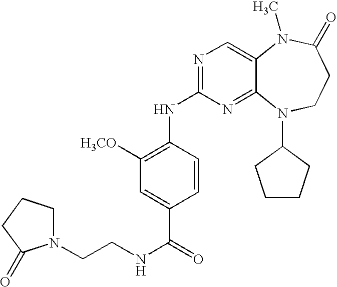

The title compound was synthesized using an analogous procedure to that described in connection with Compound 10 except that 2-amino 1-pyrrolidin-1-yl ethanone hydrochloride was used. ¹H NMR (400 MHz, DMSO-d₆) δ ppm 1.61-1.77 (m, 6H) 1.79 (m, 1H) 1.91 (m, 3H) 2.60-2.74 (m, 1H) 3.17 (s, 3H) 3.31 (t, J=6.82 Hz, 2H) 3.49 (t, J=6.69 Hz, 2H) 3.72 (br. s., 2H) 3.95 (s, 3H) 4.03 (d, J=5.56 Hz, 2H) 4.88 (dt, J=16.42, 8.21 Hz, 1H) 7.53 (dd, J=8.46 Hz, 1H) 7.61 (s, 1H) 8.09 (s, 1H) 8.12 (br s, —NH) 8.62 (br s, 1H). [M+H] calculated for C₂₆H₃₅N₆O₄, 522; found 522.

### Compound 47

### 4-(9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide

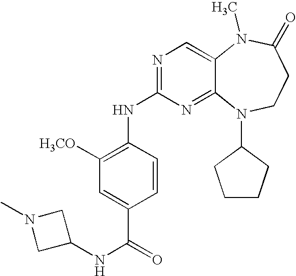

The title compound was synthesized using an analogous procedure to that described in connection with Compound 10 except that 1-methylazetidin-3-amine was used. ¹H NMR (400 MHz, DMSO-d₆) δ ppm 1.60-1.75 (m, 6H) 1.91 (m, 2H) 2.62-2.69 (m, 2H) 2.92 (s, 3H) 3.17 (s, 3H) 3.66 (br. s., 2H) 3.95 (s, 3H) 4.06-4.19 (m, 2H) 4.34-4.50 (m, 2H) 4.70 (m, 1H) 4.88 (dt, J=16.42, 8.21 Hz, 1H) 7.49 (d, J=8.1 Hz, 1H) 7.53 (d, J=8.46 Hz, 1H) 8.11 (s, 1H) 8.32 (br d, —NH) 8.97 (dd, J=8.1 Hz, 1H) 9.64 (br. s, —NH). [M+H] calculated for C₂₅H₃₄N₇O₃, 480; found 480.

### Compound 48

### (S)-4-(9-cyclopentyl-5-methyl-6-oxo-6,7,8,9-tetrahydro-5H-pyrimido[4,5-b][1,4]diazepin-2-ylamino)-3-methoxy-N-(6-oxopiperidin-3-yl)benzamide

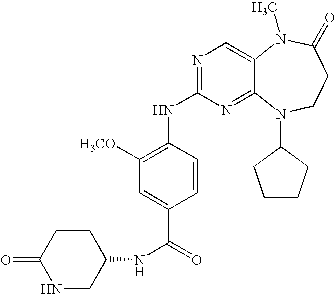

The title compound was synthesized using an analogous procedure to that described in connection with Compound 10 except that (S)-5-aminopiperidin-2-one was used. ¹H NMR (400 MHz, DMSO-d₆) δ ppm 1.60-1.75 (m, 6H) 1.89-1.96 (m, 4H) 2.28-2.33 (m, 2H) 2.62-2.69 (m, 2H) 3.05-3.12 (m, 2H) 3.16 (s, 3H) 3.66 (m., 2H) 3.95 (s, 3H) 4.19 (m, 1H) 4.70 (m, 1H) 4.86 (dt, J=16.42, 8.21 Hz, 1H) 7.49 (d, J=8.1 Hz, 1H) 7.52-7.57 (m, 2H) 8.09 (s, 1H) 8.11 (br s, —NH) 8.32 (br. d., 1H) 9.64 (br. s., 1H). [M+H] calculated for C₂₆H₃₄N₇O₄, 508; found 508.

### Compound 49

### (R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide

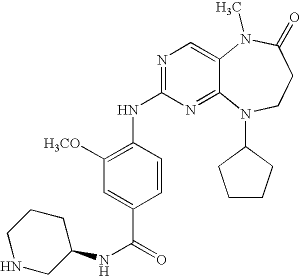

The title compound was synthesized using an analogous procedure to that described in connection with Compound 10 except that (R)-1-Boc-3-aminopiperidine was used. N-Boc protection was removed with TFA in DCM. ¹H NMR (400 MHz, DMSO-d₆) δ ppm 1.50-1.72 (m, 8H) 1.82-1.91 (m, 4H) 2.62-2.69 (m, 2H) 2.79-2.85 (m, 2H) 3.16 (s, 3H) 3.21-3.28 (m, 2H) 3.66 (m, 2H) 3.95 (s, 3H) 4.06-4.19 (m, 1H) 4.83 (dt, J=16.42, 8.21 Hz, 1H) 7.49 (d, J=8.1 Hz, 1H) 7.51 (s, 1H) 8.10 (s, 1H) 8.21 (br. d, 1H) 8.35 (d, J=8.0 Hz, 1H) 8.63 (br. s., 2H). [M+H] calculated for C₂₆H₃₆N₇O₃, 494; found 494.

### Compound 50

### (S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide

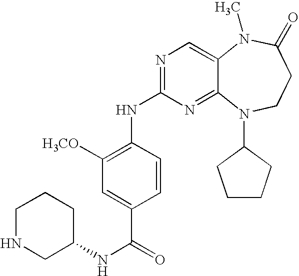

The title compound was synthesized using an analogous procedure to that described in connection with Compound 10 except that (S)-1-Boc-3-aminopiperidine was used. N-Boc protection was removed as in with TFA in DCM. ¹H NMR (400 MHz, DMSO-d₆) δ ppm 1.54-1.76 (m, 8H) 1.84-1.98 (m, 4H) 2.65 (d, J=9.85 Hz, 2H) 2.81 (m, 2H) 3.16 (s, 3H) 3.24-3.41 (m, 2H) 3.67 (m, 2H) 3.95 (s, 3H) 4.15 (br. s., 1H) 4.84 (m, 1H) 7.51 (d, J=8.1 Hz, 1H) 7.52 (s, 1H) 8.10 (s, 1H) 8.25 (d, 1H) 8.36 (d, J=8.1 Hz, 1H) 8.67 (br. s., 2H). [M+H] calculated for C₂₆H₃₆N₇O₃, 494; found 494.

### Compound 51

### (R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide

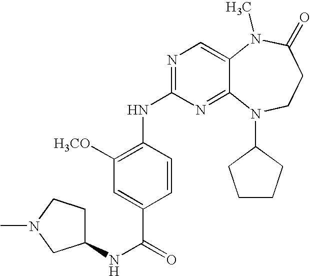

The title compound was synthesized using an analogous procedure to that described in connection with Compound 10 except that 1-methyl-3-(R)-amino pyrrolidine dihydrochloride was used. ¹H NMR (400 MHz, DMSO-d₆) δ ppm 1.56-1.91 (m, 8H) 2.02-2.25 (m, 2H) 2.66 (d, J=9.35 Hz, 2H) 2.89 (dd, J=15.03, 4.42 Hz, 2H) 3.17 (s, 3H) 3.37 (dt, J=11.94, 7.55 Hz, 1H) 3.59 (d, J=11.62 Hz, 2H) 3.69 (d, J=4.80 Hz, 1H) 3.67 (br. s., 3H) 3.95 (s, 3H), 4.55 (m, 1H) 4.84 (m, 1H) 7.51-7.54 (m, 2H) 8.11 (s, 1H) 8.25 (d, J=8.0 Hz, 1H) 8.60 (br. s., 1H) 8.69 (dd, J=14.65, 6.06 Hz, 1H), 10.11 (br. s., 1H). [M+H] calculated for C₂₆H₃₆N₇O₃, 494; found 494.

### Compound 52

### (S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide

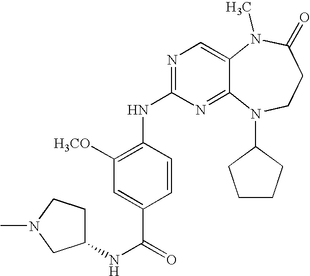

The title compound was synthesized using an analogous procedure to that described in connection with Compound 10 except that 1-methyl-3-(S)-amino pyrrolidine dihydrochloride was used. ¹H NMR (400 MHz, DMSO-d₆) δ ppm 1.57-1.72 (m, 6H) 1.90 (br. s., 2H)) 2.02-2.25 (m, 2H) 2.66 (d, J=9.35 Hz, 2H) 2.89 (dd, J=15.03, 4.67 Hz, 2H) 3.17 (s, 3H) 3.36 (d, J=12.13 Hz, 1H) 3.60 (d, J=11.62 Hz, 2H) 3.67 (br. s., 3H) 3.69 (d, J=4.80 Hz, 1H) 3.95 (s, 5H) 4.55 (m, 1H) 4.84 (m, 1H) 7.52-7.54 (m, 2H) 8.11 (s, 1H) 8.27 (br. s., 1H) 8.45-8.68 (m, 2H) 9.83 (br. s., 1H). [M+H] calculated for C₂₆H₃₆N₇O₃, 494; found 494.

### Compound 53

### (R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide

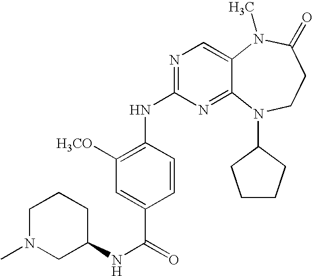

The title compound was synthesized using an analogous procedure to that described in connection with Compound 10 except that 1-methyl-3-(R)-amino piperidine dihydrochloride was used. ¹H NMR (400 MHz, DMSO-d₆) δ ppm 1.57-2.00 (m, 12H) 2.65 (d, J=9.85 Hz, 2H) 2.74 (m, 1H) 2.84 (s, 3H) 3.17 (s, 3H) 3.43-68 (m, 5H) 3.95 (s, 3H) 4.17 (d, J=8.08 Hz, 1H) 4.84 (t, J=8.21 Hz, 1H) 7.49 (d, J=8.0 Hz, 1H) 7.52 (s, 1H) 8.11 (s, 1H) 8.25 (d, J=8.08 Hz, 1H) 8.47 (d, J=7.58 Hz, 1H) 9.67 (br. s., 1H). [M+H] calculated for C₂₇H₃₈N₇O₃, 508; found 508.

### Compound 54

### (S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide

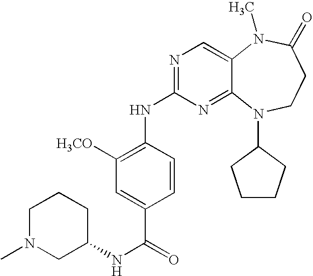

The title compound was synthesized using an analogous procedure to that described in connection with Compound 10 except that 1-methyl-3-(S)-amino piperidine dihydrochloride was used. ¹H NMR (400 MHz, DMSO-d₆) δ ppm 1.57-2.00 (m, 12H) 2.65 (d, J=9.85 Hz, 2H) 2.74 (m, 1H) 2.84 (s, 3H) 3.17 (s, 3H) 3.43-68 (m, 5H) 3.95 (s, 3H) 4.17 (d, J=8.08 Hz, 1H) 4.84 (t, J=8.21 Hz, 1H) 7.49 (d, J=8.0 Hz, 1H) 7.52 (s, 1H) 8.10 (s, 1H) 8.25 (d, J=8.08 Hz, 1H) 8.46 (d, J=7.58 Hz, 1H) 9.62 (br. s., 1H). [M+H] calculated for C₂₇H₃₈N₇O₃, 508; found 508.

### Compound 55

### 4-(9-cyclopentyl-5-methyl-6-oxo-6,7,8,9-tetrahydro-5H-pyrimido[4,5-b][1,4]diazepin-2-ylamino)-3-methoxy-N-(2-(piperazin-1-yl)ethyl)benzamide

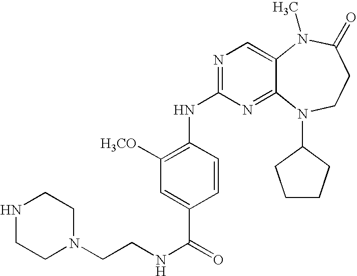

The title compound was synthesized using an analogous procedure to that described in connection with Compound 10 except that 4-N-(2-aminoethyl)-1-N-Boc-piperazine was used. N-Boc protection was then removed with TFA in DCM. ¹H NMR (400 MHz, DMSO-d₆) δ ppm 1.57-2.00 (m, 8H) 2.65 (d, J=9.85 Hz, 2H) 2.90 (m, 1H) 3.17 (s, 3H) 3.19-3.56 (m, 7H) 3.57-3.68 (m, 4H) 3.95 (s, 3H) 4.84 (t, J=8.21 Hz, 1H) 7.49 (d, J=8.0 Hz, 1H) 7.52 (s, 1H) 8.10 (s, 1H) 8.27 (d, J=8.08 Hz, 1H) 8.51-8.64 (br. s., 2H). [M+H] calculated for C₂₇H₃₉N₈O₃, 523; found 523.

### Compound 56

### 4-(9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide

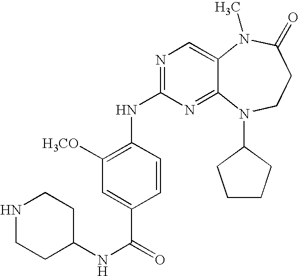

The title compound was synthesized using an analogous procedure to that described in connection with Compound 10 except that 1-N-Boc-4-aminopiperidine was used. N-Boc protection was removed. ¹H NMR (400 MHz, DMSO-d₆) δ ppm 1.52-1.75 (m, 8H) 1.85-2.01 (m, 4H) 2.65 (d, J=9.85 Hz, 2H) 3.06 (m, 2H) 3.17 (s, 3H) 3.33-3.70 (m, 6H) 3.95 (s, 3H) 4.15 (m, 1H) 4.85 (t, J=8.21 Hz, 1H) 7.51 (d, J=8.0 Hz, 1H) 7.52 (s, 1H) 8.10 (s, 1H) 8.21-8.39 (m, 3H) 8.56 (br. s., 1H). [M+H] calculated for C₂₆H₃₆N₇O₃, 494; found 494.

### Compound 57

### 4-(9-cyclopentyl-5-methyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide

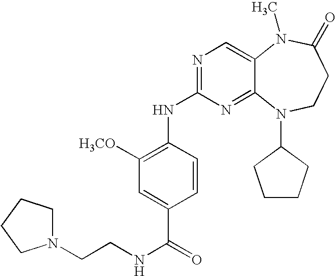

The title compound was synthesized using an analogous procedure to that described in connection with Compound 10 except that 1-(2-aminoethyl)pyrrolidine was used. ¹H NMR (400 MHz, DMSO-d₆) δ ppm 1.58-1.73 (m, 6H) 1.84-2.06 (m, 6H) 2.66 (d, J=9.35 Hz, 2H) 3.08 (m, 2H) 3.17 (s, 2H) 3.34 (q, J=6.15 Hz, 2H) 3.58-3.70 (m, 7H) 3.95 (s, 3H) 4.84 (t, J=8.08 Hz, 1H) 7.53 (d, J=8.0 Hz, 1H) 7.56 (s, 1H) 8.11 (s, 1H) 8.27 (br. s., 1H) 8.69 (br. s., 1H) 9.50 (br. s., 1H). [M+H] calculated for C₂₇H₃₈N₇O₃, 508; found 508.

### Compound 58

### 4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide

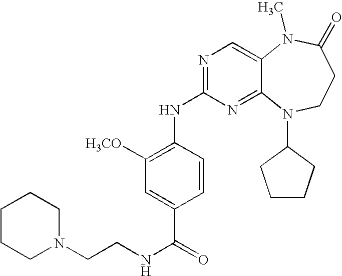

The title compound was synthesized using an analogous procedure to that described in connection with Compound 10 except that 1-(2-aminoethyl)piperidine was used. ¹H NMR (400 MHz, DMSO-d₆) δ ppm 1.37-1.95 (m, 14H) 2.66 (d, J=9.35 Hz, 2H) 2.94 (m, 2H) 3.16 (s, 3H) 3.23 (m, 2H) 3.34-3.70 (m, 7H) 3.94 (s, 3H) 4.83 (m, 1H) 7.51 (d, J=8.0 Hz, 1H) 7.54 (s, 1H) 8.10 (s, 1H) 8.26 (br. s., 1H) 8.52 (br. s., 1H) 8.69 (br. s., 1H) 9.09 (br. s., 1H). [M+H] calculated for C₂₈H₄₀N₇O₃, 522; found 522.

### Compound 59

### 4-(9-cyclopentyl-5-methyl-6-oxo-6,7,8,9-tetrahydro-5H-pyrimido[4,5-b][1,4]diazepin-2-ylamino)-3-methoxy-N-(2-(methylamino)ethyl)benzamide

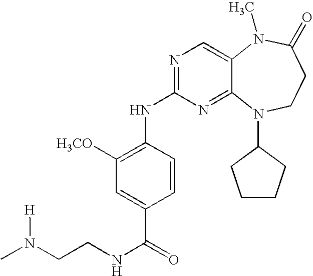

The title compound was synthesized using an analogous procedure to that described in connection with Compound 10 except that N-(2-aminoethyl)-N-methyl carbamic acid tert-butyl ester was used. N-Boc protection was removed. ¹H NMR (400 MHz, DMSO-d₆) δ ppm 1.51-1.75 (m, 6H) 1.84-2.09 (m, 5H) 2.68 (br. s., 2H) 3.17 (s, 6H) 3.25 (m, 1H) 3.45-3.75 (m, 4H) 3.96 (s, 3H) 4.85 (t, J=8.21 Hz, 1H) 7.58 (d, J=4.04 Hz, 1H) 7.54 (d, J=7.83 Hz, 1H) 8.12 (d, J=5.05 Hz, 1H) 8.24 (br. s., 1H) 8.46 (br. s., 1H) 8.80 (br. s., 1H) 9.05 (br. s., 1H). [M+H] calculated for C₂₄H₃₄N₇O₃, 468; found 468.

### Compound 60

### 4-(9-cyclopentyl-5-methyl-6-oxo-6,7,8,9-tetrahydro-5H-pyrimido[4,5-b][1,4]diazepin-2-ylamino)-3-methoxy-N-(piperidin-4-ylmethyl)benzamide

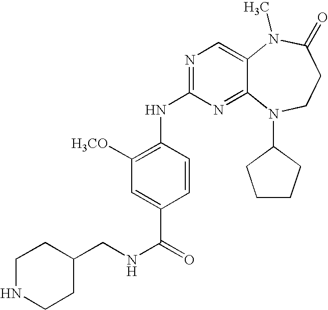

The title compound was synthesized using an analogous procedure to that described in connection with Compound 10 except that 1-Boc-4-(aminomethyl)piperidine was used. N-Boc protection was removed. ¹H NMR (400 MHz, DMSO-d₆) δ ppm 1.30 (m, 1H) 1.50-1.91 (m, 12H) 2.71 (m, 2H) 2.85 (q, J=10.86 Hz, 1H) 3.11-3.24 (m, 3H) 3.17 (s, 3H) 3.29 (d, J=12.13 Hz, 1H) 3.50-3.81 (m, 3H) 3.93 (s, 3H) 4.02 (m, 1H) 4.83 (m, 1H) 7.54 (d, J=8.34 Hz, 1H) 7.59 (s, 1H) 8.14 (d, J=8.34 Hz, 1H) 8.41 (br. s., 1H) 8.62 (d, J=15.66 Hz, 1H). [M+H] calculated for C₂₇H₃₈N₇O₃, 508; found 508.

### Compound 61

### (S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide

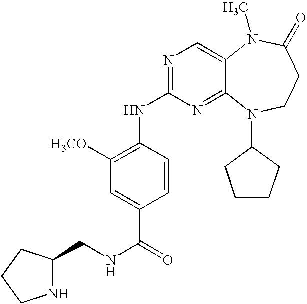

The title compound was synthesized using an analogous procedure to that described in connection with Compound 10 except that (S)-2-aminomethyl-1-N-Boc-pyrrolidine was used. N-Boc protection was then removed with TFA in DCM. ¹H NMR (400 MHz, DMSO-d₆) δ ppm 1.54-1.75 (m, 7H) 1.84-2.09 (m, 5H) 2.68 (br. s., 2H) 3.17 (s, 3H) 3.25 (m, 1H) 3.45-3.70 (m, 5H) 3.96 (s, 3H) 4.85 (m, 2H) 7.54 (d, J=8.0 Hz, 1H) 7.58 (d, J=4.04 Hz, 1H) 8.12 (s, 1H) 8.24 (br. s., 1H) 8.46 (br. s., 1H) 8.80 (br. s., 1H) 9.05 (br. s., 1H). [M+H] calculated for C₂₆H₃₆N₇O₃, 494; found 494.

### Compound 62

### (R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide

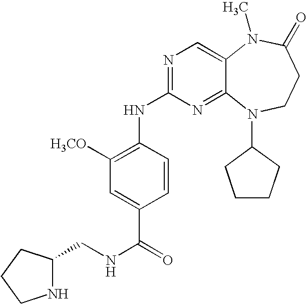

The title compound was synthesized using an analogous procedure to that described in connection with Compound 10 except that (R)-2-aminomethyl-1-N-Boc-pyrrolidine was used. N-Boc protection was then removed with TFA in DCM. ¹H NMR (400 MHz, DMSO-d₆) δ ppm 1.53-1.75 (m, 7H) 1.84-2.09 (m, 5H) 2.68 (br. s., 2H) 3.17 (s, 3H) 3.25 (m, 1H) 3.45-3.70 (m, 5H) 3.96 (s, 3H) 4.85 (m, 2H) 7.54 (d, J=8.0 Hz, 1H) 7.56 (s, 1H) 8.12 (s, 1H) 8.28 (d, J=8.0 Hz, 1H) 8.40 (br. s., 1H) 8.57 (br. s., 1H) 8.78 (br. s., 1H) 9.02 (br. s., 1H). [M+H] calculated for C₂₆H₃₆N₇O₃, 494; found 494.

### Compound 63

### (R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide

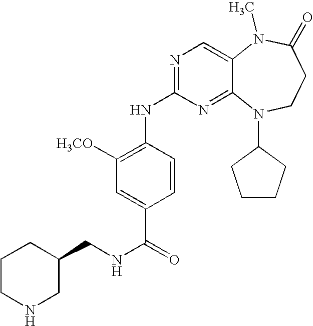

The title compound was synthesized using an analogous procedure to that described in connection with Compound 10 except that (S)-3-aminomethyl-1-N-Boc-piperidine was used. N-Boc protection was then removed with TFA in DCM. ¹H NMR (400 MHz, DMSO-d₆) δ ppm 1.24 (m, 1H) 1.54-1.98 (m, 12H) 2.60 (m, 1H) 2.68 (m, 2H) 2.82 (m, 1H) 3.17 (s, 3H) 3.23 (m, 3H) 3.70 (d, J=9.60 Hz, 2H) 3.95 (s, 3H) 4.84 (m, 2H) 7.52 (dd, J=8.46, 1.64 Hz, 1H) 7.56 (s, 1H) 8.11 (s, 1H) 8.26 (br. s., 1H) 8.60 (br. s., 2H) 8.77 (br. s., 1H). [M+H] calculated for C₂₇H₃₈N₇O₃, 508; found 508.

### Compound 64

### (S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide

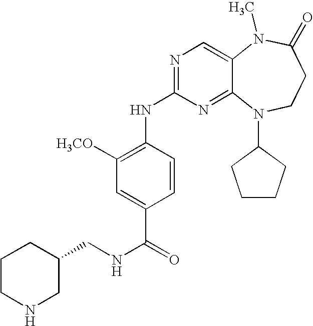

The title compound was synthesized using an analogous procedure to that described in connection with Compound 10 except that (R)-3-aminomethyl-1-N-Boc-piperidine was used. N-Boc protection was then removed with TFA n DCM. ¹H NMR (400 MHz, DMSO-d₆) δ ppm 1.24 (m, 1H) 1.54-1.98 (m, 12H) 2.60 (m, 1H) 2.68 (m, 2H) 2.82 (m, 1H) 3.17 (s, 3H) 3.23 (m, 3H) 3.70 (d, J=9.60 Hz, 2H) 3.95 (s, 3H) 4.84 (m, 2H) 7.53 (dd, J=8.46, 1.64 Hz, 1H) 7.56 (s, 1H) 8.12 (s, 1H) 8.27 (br. s., 1H) 8.60 (br. s., 2H) 9.00 (br. s., 1H). [M+H] calculated for C₂₇H₃₈N₇O₃, 508; found 508.

### Compound 65

### (S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide

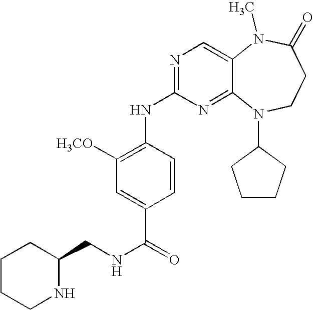

The title compound was synthesized using an analogous procedure to that described in connection with Compound 10 except that (S)-2-aminomethyl-1-N-Boc-piperidine was used. N-Boc protection was then removed with TFA in DCM. ¹H NMR (400 MHz, DMSO-d₆) δ ppm 1.43-1.91 (m, 14H) 2.72 (d, 2H) 2.90 (m, 1H) 3.17 (s, 3H) 3.23 (m, 2H) 3.42 (m, 1H) 3.73 (d, J=9.09 Hz, 2H) 3.96 (s, 3H) 4.87 (t, J=8.21 Hz, 2H) 7.58 (d, J=8.34 Hz, 1H) 7.62 (br. s., 1H) 8.15 (s, 2H) 8.33 (br. s., 1H) 8.64 (br. s., 1H) 8.76 (br. s., 1H) 9.10 (br. s., 1H). [M+H] calculated for C₂₇H₃₈N₇O₃, 508; found 508.

### Compound 66

### (R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide

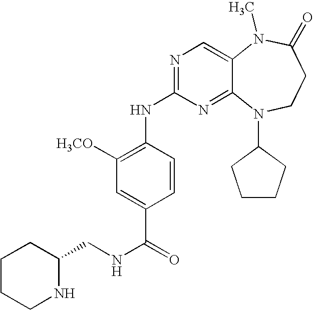

The title compound was synthesized using an analogous procedure to that described in connection with Compound 10 except that (R)-2-aminomethyl-1-N-Boc-piperidine was used. N-Boc protection was then removed with TFA in DCM. ¹H NMR (400 MHz, DMSO-d₆) δ ppm 1.43-1.91 (m, 14H) 2.72 (d, 2H) 2.90 (m, 1H) 3.17 (s, 3H) 3.23 (m, 2H) 3.42 (m, 1H) 3.73 (d, J=9.09 Hz, 2H) 3.96 (s, 3H) 4.86 (t, J=8.21 Hz, 2H) 7.56 (d, J=8.34 Hz, 1H) 7.59 (s 1H) 7.83 (br. s., 1H) 8.07 (s, 1H) 8.12 (s, 2H) 8.24 (br. s., 1H) 8.58 (br. s., 1H) 8.70 (br. s., 1H). [M+H] calculated for C₂₇H₃₈N₇O₃, 508; found 508.

### Compound 67

### 4-(9-cyclopentyl-5-methyl-6-oxo-6,7,8,9-tetrahydro-5H-pyrimido[4,5-b][1,4]diazepin-2-ylamino)-3-methoxy-N-(pyridin-3-yl)benzamide

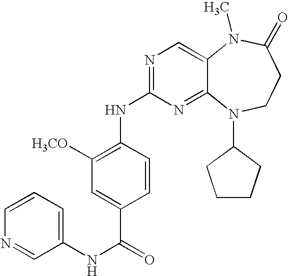

The title compound was synthesized using an analogous procedure to that described in connection with Compound 10 except that 3-aminopyridine was used. ¹H NMR (400 MHz, DMSO-d₆) δ ppm 1.55-1.77 (m, 6H) 1.91-1.94 (m, 2H) 2.70 (d, J=8.59 Hz, 2H) 3.18 (s, 3H) 3.72 (d, J=9.35 Hz, 2H) 4.00 (s, 3H) 4.89 (m, 1H) 7.65-7.75 (m, 3H) 8.14 (s, 1H) 8.27 (d, J=8.84 Hz, 1H) 8.40 (d, J=8.84 Hz, 1H) 8.51 (br. s., 1H) 8.99 (br. s., 1H) 9.12 (br. s., 1H) 10.64 (s, 1H). [M+H] calculated for C₂₆H₃₀N₇O₃, 488; found 488.

### Compound 68

### 4-(9-cyclopentyl-5-methyl-6-oxo-6,7,8,9-tetrahydro-5H-pyrimido[4,5-b][1,4]diazepin-2-ylamino)-3-methoxy-N-(pyridin-4-yl)benzamide

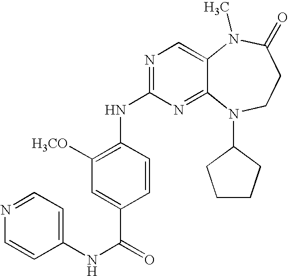

The title compound was synthesized using an analogous procedure to that described in connection with Compound 10 except that 4-aminopyridine was used. ¹H NMR (400 MHz, DMSO-d₆) δ ppm 1.60-1.77 (m, 6H) 1.91-1.94 (m, 2H) 2.68 (br. s., 2H) 3.18 (s, 3H) 3.70 (br. s., 2H) 4.01 (s, 3H) 4.88 (m, 1H) 7.69 (br. s., 1H) 7.74 (d, J=8.59 Hz, 1H) 8.16 (br. s., 1H) 8.30 (d, J=5.05 Hz, 2H) 8.37 (d, J=3.79 Hz, 1H) 8.78 (br. s., 2H) 11.44 (br. s., 1H). [M+H] calculated for C₂₆H₃₀N₇O₃, 488; found 488.

### Compound 69

### 4-(9-cyclopentyl-5-methyl-6-oxo-6,7,8,9-tetrahydro-5H-pyrimido[4,5-b][1,4]diazepin-2-ylamino)-3-methoxy-N-(pyridin-2-ylmethyl)benzamide

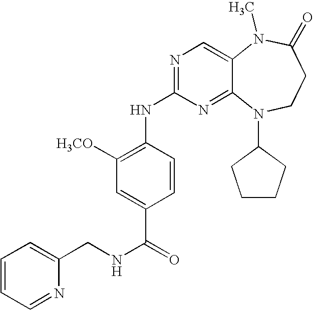

The title compound was synthesized using an analogous procedure to that described in connection with Compound 10 except that 2-(aminomethyl)pyridine was used. ¹H NMR (400 MHz, DMSO-d₆) δ ppm 1.53-1.77 (m, 6H) 1.86-1.92 (m, 2H) 2.71 (br. s., 2H) 0.63 (br. s., 3H) 3.17 (s, 3H) 3.72 (br. s., 2H) 3.95 (s, 3H) 4.67 (br. s., 2H) 4.86 (m, 1H) 7.50-7.66 (m, 4H) 8.01 (m, 1H) 8.13 (br. s., 1H) 8.64 (br. s., 1H) 9.26 (br. s., 2H). [M+H] calculated for C₂₇H₃₂N₇O₃, 502; found 502.

### Compound 70

### 4-(9-cyclopentyl-5-methyl-6-oxo-6,7,8,9-tetrahydro-5H-pyrimido[4,5-b][1,4]diazepin-2-ylamino)-3-methoxy-N-(pyridin-3-ylmethyl)benzamide

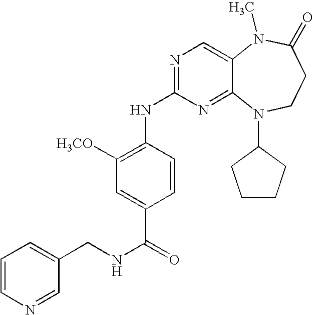

The title compound was synthesized using an analogous procedure to that described in connection with Compound 10 except that 3-(aminomethyl)pyridine was used. ¹H NMR (400 MHz, DMSO-d₆) δ ppm 1.51-1.76 (m, 6H) 1.82-1.90 (m, 2H) 2.70 (d, J=8.84 Hz, 2H) 3.17 (s, 3H) 3.71 (d, J=9.85 Hz, 2H) 3.95 (s, 3H) 4.60 (d, J=5.81 Hz, 2H) 4.85 (m, 1H) 7.57 (d, J=8.0, 1H) 7.62 (d, J=1.77 Hz, 1H) 7.73 (dd, J=7.96, 5.43 Hz, 1H) 8.12 (s, 1H), 8.15 (m, 2H) 8.62 (br. s., 1H) 8.75 (br. s., 1H) 9.05 (br. s., 1H) 9.20 (t, J=5.81 Hz, 1H). [M+H] calculated for C₂₇H₃₂N₇O₃, 502; found 502.

### Compound 71

### 4-(9-cyclopentyl-5-methyl-6-oxo-6,7,8,9-tetrahydro-5H-pyrimido[4,5-b][1,4]diazepin-2-ylamino)-3-methoxy-N-(pyridin-4-ylmethyl)benzamide

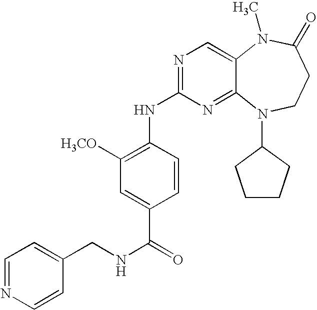

The title compound was synthesized using an analogous procedure to that described in connection with Compound 10 except that 4-(aminomethyl)pyridine was used. ¹H NMR (400 MHz, DMSO-d₆) δ ppm 1.54-1.77 (m, 6H) 1.84-1.92 (m, 2H) 2.73 (d, J=8.59 Hz, 2H) 3.17 (s, 3H) 3.74 (d, J=9.35 Hz, 2H) 3.97 (s, 3H) 4.75 (d, J=5.56 Hz, 2H) 4.88 (m, 1H) 7.64 (dd, J=8.59, 1.77 Hz, 1H) 7.70 (d, J=1.77 Hz, 1H) 7.93 (d, J=6.57 Hz, 2H) 8.12 (d, J=8.34 Hz, 1H) 8.17 (s, 1H) 8.86 (d, J=6.57 Hz, 2H) 9.44 (br. s., 2H). [M+H] calculated for C₂₇H₃₂N₇O₃, 502; found 502.

### Compound 72

### 4-(9-cyclopentyl-5-methyl-6-oxo-6,7,8,9-tetrahydro-5H-pyrimido[4,5-b][1,4]diazepin-2-ylamino)-N-cyclopropyl-3-methoxy-N-(methylpyridin-4-yl)benzamide

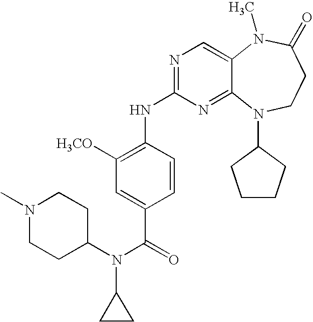

The title compound was synthesized using an analogous procedure to that described in connection with Compound 10 except that 4-cyclopropylamino-1-methylpiperidine was used. ¹H NMR (400 MHz, DMSO-d₆) δ ppm 0.36-0.45 (m, 2H) 0.54 (d, J=6.06 Hz, 2H) 1.13-1.29 (m, 1H) 1.49-1.70 (m, 6H)) 1.82-1.88 (m, 2H) 2.06-2.09 (br. d., 2H) 2.30 (m, 2H) 2.68 (m, 2H) 2.77 (s, 3H) 2.84 (d, J=4.55 Hz, 1H) 3.07-3.19 (m, 4H) 3.49 (d, J=2.53 Hz, 2H) 3.69 (d, J=5.81 Hz, 2H) 3.89 (s, 3H) 4.12 (m, 1H) 4.81 (t, J=8.34 Hz, 1H) 7.14 (dd, J=8.34, 1.26 Hz, 1H) 7.20 (d, J=1.01 Hz, 1H) 7.94 (br. s., 1H) 8.10 (s, 1H) 9.67 (br. s., 1H). [M+H] calculated for C₃₀H₄₂N₇O₃, 548; found 548.

### Compound 73

### 4-(9-cyclopentyl-5-methyl-6-oxo-6,7,8,9-tetrahydro-5H-pyrimido[4,5-b][1,4]diazepin-2-ylamino)-N-(1-(cyclopropanecarbonyl)pyridin-4-yl)-3-methoxybenzamide

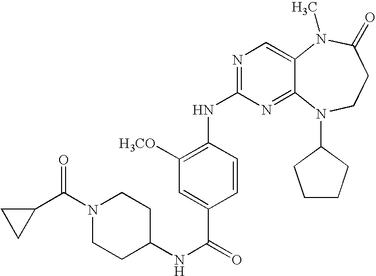

The title compound was synthesized using an analogous procedure to that described in connection with Compound 10 except that 1-N-cyclopropylcarbonyl piperidine-4-amine was used. ¹H NMR (400 MHz, DMSO-d₆) δ ppm 0.72 (m, 4H) 1.25-1.90 (m, 12H) 2.05 (m, 1H) 2.69 (d, J=8.59 Hz, 2H) 3.16 (s, 3H) 3.70 (d, J=9.60 Hz, 2H) 3.95 (s, 2H) 4.10 (m, 1H) 4.36 (dd, 1H) 4.86 (m, 1H) 7.52 (dd, J=8.46, 1.64 Hz, 1H) 7.55 (s, 1H) 8.09 (s, 1H) 8.13 (d, J=7.83 Hz, 1H) 8.22 (d, J=7.83 Hz, 1H) 8.88 (br. s., 1H). [M+H] calculated for C₃₀H₄₀N₇O₄, 562; found 562.

### Compound 74

### (S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide

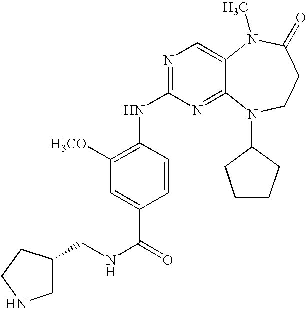

The title compound was synthesized using an analogous procedure to that described in connection with Compound 10 except that (R)-3-(aminomethyl)-1-N-Boc-pyrrolidine was used. N-Boc protection was then removed with TFA in DCM. ¹H NMR (400 MHz, DMSO-d₆) δ 1.54-1.76 (m, 8H) 1.89 (m, 2H) 1.94-2.11 (m, 1H) 2.67 (dt, J=3.79, 1.89 Hz, 2H) 2.92 (m, 1H) 3.17 (s, 3H) 3.27-3.38 (m, 4H) 3.69 (d, J=9.85 Hz, 2H) 3.95 (s, 3H) 4.84 (m, 1H) 7.51 (dd, J=8.59, 1.77 Hz, 1H) 7.55 (s, 1H) 8.10 (s, 1H) 8.19 (d, J=8.59, 1H) 8.62 (m, 2H). [M+H] calculated for C₂₆H₃₆N₇O₃, 494; found 494.

### Compound 75

### (R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide

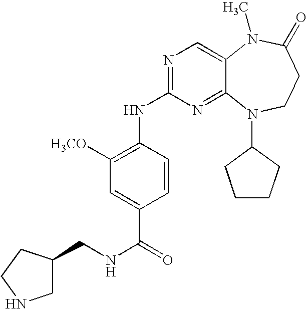

The title compound was synthesized using an analogous procedure to that described in connection with Compound 10 except that (S)-3-(aminomethyl)-1-N-Boc-pyrrolidine was used. N-Boc protection was then removed with TFA in DCM. ¹H NMR (400 MHz, DMSO-d₆) δ 1.54-1.76 (m, 8H) 1.89 (m, 2H) 1.94-2.05 (m, 1H) 2.67 (dt, J=3.79, 1.89 Hz, 2H) 2.92 (m, 1H) 3.17 (s, 3H) 3.27-3.38 (m, 4H) 3.70 (d, J=9.60 Hz, 2H) 3.95 (s, 3H) 4.84 (m, 1H) 7.51 (dd, J=8.59, 1.77 Hz, 1H) 7.55 (s, 1H) 8.10 (s, 1H) 8.19 (d, J=8.59, 1H) 8.63 (m, 2H). [M+H] calculated for C₂₆H₃₆N₇O₃, 494; found 494.

### Compound 76

### 4-(9-cyclopentyl-5-methyl-6-oxo-6,7,8,9-tetrahydro-5H-pyrimido[4,5-b][1,4]diazepin-2-ylamino)-N-((1-(cyclopropanecarbonyl)piperidin-4-yl)methyl)-3-methoxybenzamide

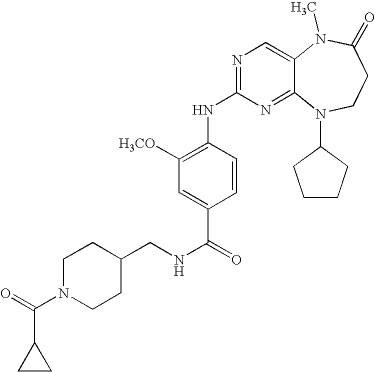

The title compound was synthesized using an analogous procedure to that described in connection with Compound 10 except that 1-N-cyclopropylcarbonyl piperidine-4-methylamine was used. ¹H NMR (400 MHz, DMSO-d₆) δ ppm 0.67 (m, 4H) 1.01 (m, 1H) 1.55-1.77 (m, 8H) 1.84-1.99 (m, 6H) 2.68 (d, J=8.59 Hz, 2H) 3.05 (m, 1H) 3.16 (s, 3H) 3.17 (br. s., 1H) 3.72 (d, J=9.60 Hz, 2H) 3.95 (s, 3H) 4.23 (m, 3H) 4.85 (m, 1H) 7.53 (dd, J=8.34, 1.77 Hz, 1H) 7.58 (s, 1H) 8.09 (s, 1H) 8.13 (d, J=7.83 Hz, 1H) 8.51 (m, 1H) 8.85 (br. s., 1H). [M+H] calculated for C₃₁H₄₂N₇O₄, 576; found 576.

### Compound 77

### 4-(9-cyclopentyl-5-methyl-6-oxo-6,7,8,9-tetrahydro-5H-pyrimido[4,5-b][1,4]diazepin-2-ylamino)-N-(1-isopropylpiperidin-4-yl)-3-methoxybenzamide

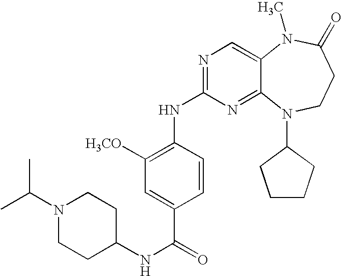

The title compound was synthesized using an analogous procedure to that described in connection with Compound 10 except that except that 1-N-isopropyl piperidine-4-amine was used. ¹H NMR (400 MHz, DMSO-d₆) δ ppm 1.26 (d, J=6.57 Hz, 6H) 1.55-1.96 (m, 10H) 2.01-2.10 (m, 2H) 2.67 (br. s., 2H) 3.13 (m, 2H) 3.17 (s, 3H) 3.41-3.53 (m, 3H) 3.67 (br. s., 2H) 3.95 (s, 3H) 4.05 (m, 1H) 4.84 (m, 1H) 7.49 (d, J=8.09 Hz, 1H) 7.52 (s, 1H) 8.10 (s, 1H) 8.23 (d, J=8.59 Hz, 1H) 8.40 (d, J=7.07 Hz, 1H) 8.53 (br. s., 1H), 9.00 (br. s., 1H). [M+H] calculated for C₂₉H₄₂N₇O₃, 536; found 536.

### Compound 78

### 4-(9-cyclopentyl-5-methyl-6-oxo-6,7,8,9-tetrahydro-5H-pyrimido[4,5-b][1,4]diazepin-2-ylamino)-N-(4-(dimethylamino)cyclohexyl) 3-methoxybenzamide

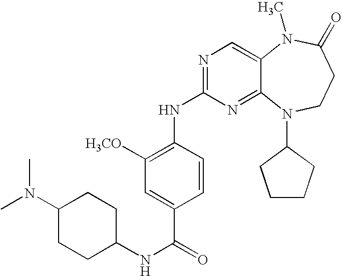

The title compound was synthesized using an analogous procedure to that described in connection with Compound 10 except that N1,N1-dimethylcyclohexane-1,4-diamine was used. ¹H NMR (400 MHz, DMSO-d₆) δ ppm 1.31-2.05 (m, 16H) 2.64 (br. s., 2H) 2.77 (br. s., 6H) 2.93 (m, 1H) 3.17 (s, 3H) 3.66 (br. s., 2H) 3.81 (br. s., 1H) 3.95 (s, 3H) 4.84 (br. s., 1H) 7.52 (s, 1H) 7.48 (d, J=9.09 Hz, 1H) 8.09 (s, 1H) 8.20 (br. s., 2H) 8.46 (br. s., 1H) 9.40 (br. s., 1H). [M+H] calculated for C₂₉H₄₂N₇O₃, 536; found 536.

### Compound 79

### 4-(9-cyclopentyl-5-methyl-6-oxo-6,7,8,9-tetrahydro-5H-pyrimido[4,5-b][1,4]diazepin-2-ylamino)-N-(4-(diethylamino)cyclohexyl) 3-methoxybenzamide

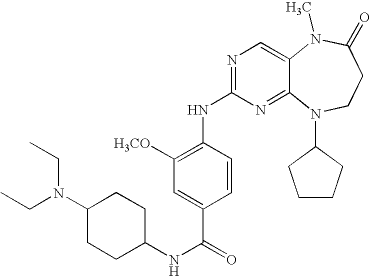

The title compound was synthesized using an analogous procedure to that described in connection with Compound 10 except that N1,N1-diethylcyclohexane-1,4-diamine was used. ¹H NMR (400 MHz, DMSO-d₆) δ ppm 1.25 (t, J=6.82 Hz, 6H) 1.30-2.05 (m, 16H) 2.62-2.67 (m, 2H) 3.10 (m, 2H) 3.17 (s, 3H) 3.24 (m, 2H) 3.22 (m, 1H) 3.66 (d, J=9.60 Hz, 2H) 3.80 (m, 1H) 3.95 (s, 3H) 4.83 (m, 1H) 7.44-7.51 (m, 2H) 8.08 (d, J=1.26 Hz, 1H) 8.14 (d, 1H), 8.25 (m, 2H) 8.72 (br. s., 1H). [M+H] calculated for C₃₁H₄₆N₇O₃, 564; found 564.

### Compound 80

### 4-(9-cyclopentyl-5-methyl-6-oxo-6,7,8,9-tetrahydro-5H-pyrimido[4,5-b][1,4]diazepin-2-ylamino)-3-methoxy-N-(1,2,2,6,6-pentamethylpiperidin-4-yl)benzamide

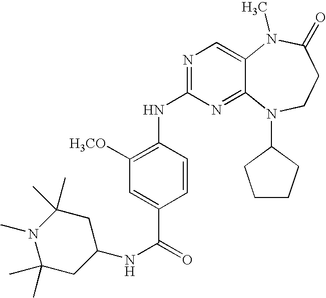

The title compound was synthesized using an analogous procedure to that described in connection with Compound 10 except that 4-amino-1,2,2,6,6-pentamethylpiperidine was used. ¹H NMR (400 MHz, DMSO-d₆) δ ppm 1.43 (s, 12H) 1.52-1.95 (m, 10H) 2.07 (br. d., 2H) 2.67 (m, 2H) 2.76 (s, 3H) 3.17 (s, 3H) 3.68 (br. s., 2H) 3.94 (s, 3H) 4.33 (m, 1H) 4.83 (m, 1H) 7.53 (m, 2H) 8.10 (br. s., 1H) 8.20 (br. s., 1H) 8.41 (br. s., 1H) 8.72 (br. s., 1H). [M+H] calculated for C₃₁H₄₆N₇O₃, 564; found 564.

### Compound 81

### 9-cyclopentyl-5-methyl-2-(pyridin-3-ylamino)-8,9-dihydro-5H-pyrimido[4,5-b][1,4]diazepin-6(7H)-one

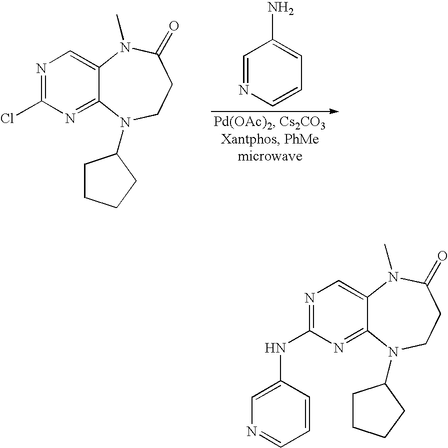

To a mixture of Compound 7 (100 mg, 0.36 mmol), 3-aminopyridine (50 mg, 0.54 mmol), Cs₂CO₃ (234 mg) in 4 mL of anhydrous toluene was added Pd(OAc)₂ (4 mg, 0.05 mmol), xantphos (10 mg, 0.05). The reaction mixture was stirred for 30 min. in a microwave at 140° C. The reaction mixture was then concentrated, diluted with MeOH:water (2:1) and filtered. The filtrand was extracted with EtOAc, the organic layer separated, concentrated and purified by prep HPLC to yield 45.6 mg of the desired product as a TFA salt (27% yield). [M+H] calc'd for C₁₈H₂₂N₆O 339, found 339.

### Compound 82

### 6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide

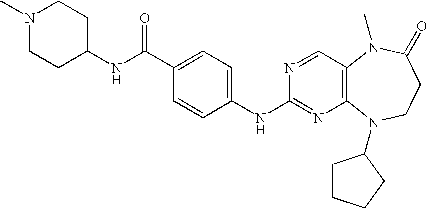

The title compound was synthesized using an analogous procedure to that described in connection with 9-cyclopentyl-5-methyl-2-(pyridin-3-ylamino)-8,9-dihydro-5H-pyrimido[4,5-b][1,4]diazepin-6(7H)-one using methyl 6-nicotinate. The ester was then saponified using 25% NaOH:THF:EtOH (1:1:1) at room temperature overnight. The reaction was then acidified using 1 N HCl and the product extracted into EtOAc. The organic layer was evaporated and the carboxylic acid coupled with 4-amino-N-methyl piperidine in the typical manner. ¹H NMR (400 MHz, DMSO-d₆) δ ppm 1.53-1.81 (m, 8H) 1.91-2.10 (m, 4H) 2.65-2.83 (m, 2H) 2.68-2.81 (m, 7H) 3.19 (s, 3H) 3.75 (d, J=10.1 Hz, 2H) 4.02 (m, 1H) 5.06 (quin., J=8.2 Hz, 1H) 7.51 (br. s., 1H) 8.04 (s, 1H) 8.29 (dd, J=8.8, 2.3 Hz, 1H) 8.62 (d, J=7.3 Hz, 1H) 8.78 (d, J=2.3 Hz, 1H). [M+H] calc'd for C₂₅H₃₄N₈O₂, 479; found, 479.

### Compound 83

### 3-Methoxy-4-(5-methyl-6-oxo-9-(tetrahydro-2H-pyran-4-yl)-6,7,8,9-tetrahydro-5H-pyrimido[4,5-b][1,4]diazepin-2-ylamino)-N-(1-methylpiperidin-4-yl)benzamide

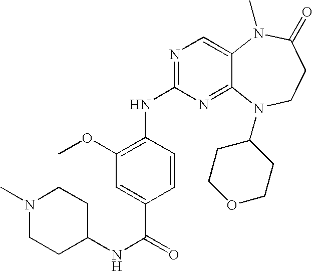

The title compound was synthesized using an analogous procedure to that described in connection with Compound 10 except that ethyl 3-(tetrahydro-2H-pyran-4-ylamino)propanoate, prepared by Michael addition of ethyl acrylate (1 equiv.) with 4-aminotetrahydropyran (1 equiv.) in refluxing ethanol for 24 h, was used and the final compound was purified by reverse phase HPLC to yield the TFA salt. ¹H NMR (400 MHz, DMSO-d₆) δ ppm 1.64 (br. s., 1H) 1.60 (d, J=9.4 Hz, 2H) 1.70-1.79 (m, 4H) 1.85 (d, J=8.3 Hz, 2H) 1.87-2.00 (m, 3H) 2.16 (s, 3H) 2.59 (br. s, 2H) 2.78 (d, J=10.9 Hz, 2H) 3.16 (s, 3H) 3.47 (t, J=11.2 Hz, 2H) 3.64 (br. s., 2H) 3.74 (d, J=7.1 Hz, 1H) 3.94 (s, 3H) 4.00 (d, J=10.9 Hz, 2H) 4.60 (br. s., 1H) 7.44-7.58 (m, 2H) 7.77 (s, 1H) 8.08 (s, 1H) 8.12 (d, J=7.6 Hz, 1H) 8.31 (d, J=8.1 Hz, 1H). [M+H] calc'd for C₂₇H₃₇N₇O₄, 524; found, 524.

### Compound 84

### 4-(9-(4,4-Difluorocyclohexyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

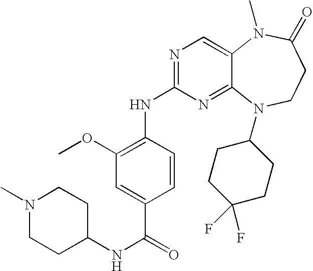

The title compound was synthesized using an analogous procedure to that described in connection with Compound 10 except that ethyl 3-(4,4-difluorocyclohexylamino)propanoate, prepared by Michael addition of ethyl acrylate (1 equiv.) with 4,4-difluorocyclohexylamine hydrochloride (1 equiv.) in refluxing ethanol for 24 h, was used and the final compound was purified by reverse phase HPLC to yield the TFA salt. ¹H NMR (400 MHz, DMSO-d₆) δ ppm 1.76-1.82 (m, 7H) 1.88-2.06 (m, 3H) 2.13 (br. s., 2H) 2.66 (d, J=8.1 Hz, 2H) 2.78 (d, J=4.3 Hz, 3H) 3.11 (m, 2H) 3.16 (s, 3H) 3.48 (d, J=11.4 Hz, 2H) 3.67 (m, 2H) 3.94 (s, 3H) 4.03 (d, J=7.3 Hz, 1H) 4.44 (br. s., 1H) 7.56 (d, J=7.3 Hz, 2H) 8.06 (d, J=8.1 Hz, 1H) 8.13 (s, 1H) 8.43 (d, J=7.3 Hz, 1H) 8.99 (br. s., 1H) 9.57 (br. s., 1H). [M+H] calc'd for C₂₈H₃₇F₂N₇O₃, 558; found, 558.

### Compound 85

### 4-(9-Cyclopentyl-5,7,7-trimethyl-6-oxo-6,7,8,9-tetrahydro-5H-pyrimido[4,5-b][1,4]diazepin-2-ylamino)-3-methoxybenzoic Acid

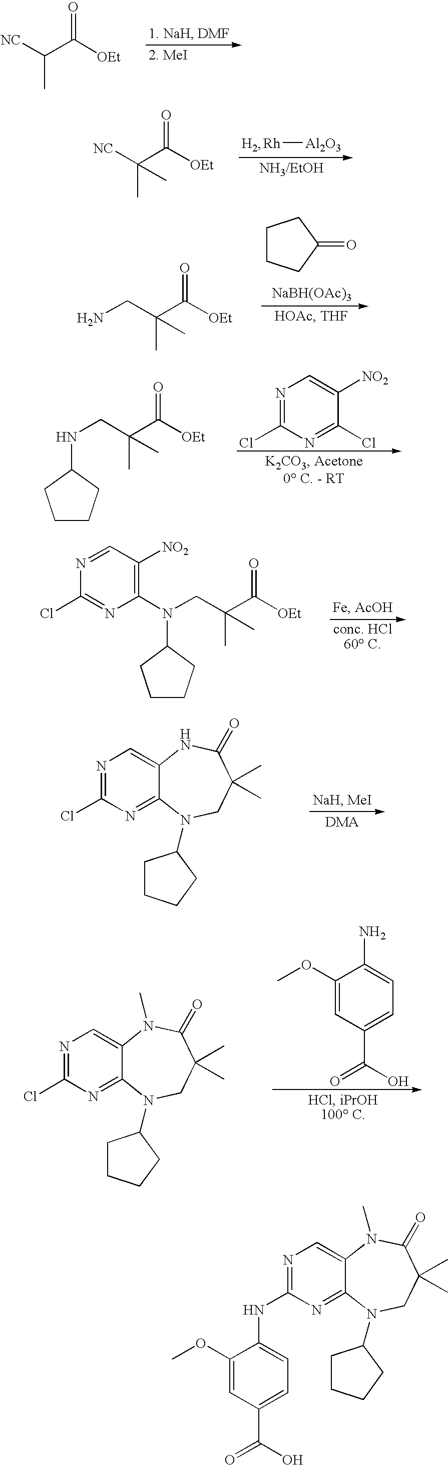

Ethyl 2-cyano-2-methylpropanoate. To a stirred suspension of 4.2 g of NaH (60% dispersion in oil, 105 mmol) in 100 mL of dry DMF was added a solution of ethyl 2-cyanopropinate (12.7 g, 100 mmol). The mixture was stirred for 15 min., then cooled to 0° C., methyl iodide (6.5 mL, 105 mmol) was added drop wise. The reaction mixture was stirred at 0° C. for 3 h and then rt. over night. It was then quenched with saturated ammonium chloride, diluted to ethyl acetate. The organic layer was washed with brine and water, dried over Na₂SO₄ and concentrated in vacuo to obtain ethyl 2-cyano-2-methylpropanoate (14 g, quantitative yield) as light yellow liquid. ¹H NMR (400 MHz, CHLOROFORM-d) δ 1.32 (t, J=7.1 Hz, 3H) 1.60 (s, 6H) 4.26 (q, J=7.2 Hz, 2H).

Ethyl 3-amino-2,2-dimethylpropanoate. A solution of ethyl 2-cyano-2-methylpropanoate (12 g, 85 mmol) in 1% ammonia in ethanol (200 mL) was hydrogenated with 5% rhodium on alumina (3 g) for 20 h, after which, another 3 g of rhodium on alumina was added and the mixture was again hydrogenated for 20 h. When the reaction was done, the reaction mixture was filtered through celite. The filtrate was concentrated and dried to give the product, ethyl 3-amino-2,2-dimethylpropanoate (12.4 g, quantitative yield) as light yellow liquid. ¹H NMR (400 MHz, CHLOROFORM-d) δ 1.12 (s, 6H) 1.20 (t, J=7.1 Hz, 3H) 2.69 (s, 2H) 4.10 (q, J=7.1 Hz, 2H).

Ethyl 3-(cyclopentylamino)-2,2-dimethylpropanoate. Ethyl 3-amino-2,2-dimethylpropanoate (7.5 g, 52 mmol) was treated with cyclopentanone (5.54 ml, 62.4 mmol) in the presence of sodium triacetoxyborohydride (16.5 g, 78 mmol) and glacial acetic acid (15 mL) in tetrahydrofuran (150 ml) for 18 h at rt. The reaction mixture was carefully diluted with saturated aqueous NaHCO₃ (500 ml). The mixture was stirred for 1 hour at room temperature. The whole was extracted with ethyl acetate (300 ml×3). The organic layer was dried over Na₂SO₄ and concentrated in vacuo to obtain ethyl 3-(cyclopentylamino)-2,2-dimethylpropanoate (8.9 g, 80% yield) as light yellow liquid. ¹H NMR (400 MHz, CHLOROFORM-d) δ 1.16 (s, 6H) 1.22-1.32 (m, 5H) 1.43-1.54 (m, 2H) 1.58-1.67 (m, 2H) 1.70-1.80 (m, 2H) 2.62 (s, 2H) 2.95-3.02 (m, 1H) 4.11 (q, J=7.2 Hz, 2H). [M+H] calc'd for C₁₂H₂₃NO₂, 214; found, 214.

Ethyl 3-((2-chloro-5-nitropyrimidin-4-yl)(cyclopentyl)amino)-2,2-dimethyl propanoate. To a solution of 2,4-dichloro-5-nitropyrimidine (5.6 g, 28.8 mmol) in anhydrous acetone (50 ml) at 0° C., was added dropwise a solution of ethyl 3-(cyclopentylamino)-2,2-dimethylpropanoate (5.1 g, 24 mmol) in acetone (10 mL) over 10 min. After which, potassium carbonate (6.9 g, 50 mmol) was added and the whole was stirred at rt for 18 h. After evaporation in vacuo, the residue was partitioned between ethyl acetate (200 ml) and water (200 ml). The organic layer was washed with NaHCO₃, brine and water, dried over Na₂SO₄ and concentrated in vacuo. The residue was purified by silica gel chromatography (ethyl acetate:hexane=1:10 to 1:5) and recrystallized from ether/EtOAc to obtain ethyl 3-((2-chloro-5-nitropyrimidin-4-yl)(cyclopentyl)amino)-2,2-dimethyl propanoate (3 g, 34% yield) as pale yellow powder. ¹H NMR (400 MHz, CHLOROFORM-d) δ 1.18-1.28 (m, 8H) 1.48-1.65 (m, 4H) 1.65-1.78 (m, 2H) 1.92 (m, 2H) 3.50 (m, 1H) 3.81 (s, 2H) 4.13 (q, J=7.2 Hz, 2H) 4.12 (q, J=7.2 Hz, 1H) 8.74 (s, 1H). [M+H] calc'd for C₁₆H₂₃ClN₄O₄, 371; found, 371.

2-Chloro-9-cyclopentyl-7,7-dimethyl-8,9-dihydro-5H-pyrimido[4,5-b][1,4]diazepin-6(7H)-one. To a suspension of ethyl 3-((2-chloro-5-nitropyrimidin-4-yl)(cyclopentyl)amino)-2,2-dimethyl propanoate (3 g, 8.1 mmol), reduced iron (896 mg, 16 mmol) in acetic acid (20 ml) was added dropwise concentrated hydrochloric acid (5 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2-chloro-9-cyclopentyl-7,7-dimethyl-8,9-dihydro-5H-pyrimido[4,5-b][1,4]diazepin-6(7H)-one (1.36 g, 57% yield) as white solid. [M+H] calc'd for C₁₄H₁₉ClN₄O, 295; found, 295.

2-Chloro-9-cyclopentyl-5,7,7-trimethyl-8,9-dihydro-5H-pyrimido[4,5b][1,4]diazepin-6(7H)-one. To a solution of 2-chloro-9-cyclopentyl-7,7-dimethyl-8,9-dihydro-5H-pyrimido[4,5-b][1,4]diazepin-6(7H)-one (2.45 g, 8.3 mmol) in 20 mL of DMA was added sodium hydride (60% dispersion in mineral oil, 366 mg, 9.14 mmol) at 0° C., followed by the dropwise addition of methyl iodide (0.57 mL, 9.14 mmol). The reaction mixture was warmed up to rt and stirred for 1 h. The whole was poured into ice-water, extracted with ethyl acetate. The organic layer was washed with brine and dried over Na₂SO₄. The solution was concentrated in vacuo followed by precipitation from ether/EtOA to afford 2-chloro-9-cyclopentyl-5,7,7-trimethyl-8,9-dihydro-5H-pyrimido[4,5b][1,4]diazepin-6(7H)-one (2 g, 79% yield) as white solid. ¹H NMR (400 MHz, DMSO-d₆) δ ppm 1.08 (s, 6H) 1.57 (d, J=1.8 Hz, 4H) 1.70 (br. s., 2H) 1.82 (d, J=3.0 Hz, 2H) 3.18 (s, 3H) 3.41 (s, 2H) 5.08 (t, J=8.3 Hz, 1H) 8.05 (s, 1H) [M+H] calc'd for C₁₅H₂₁ClN₄O, 309; found, 309.

4-(9-Cyclopentyl-5,7,7-trimethyl-6-oxo-6,7,8,9-tetrahydro-5H-pyrimido[4,5-b][1,4]diazepin-2-ylamino)-3-methoxybenzoic acid. A mixture of 2-chloro-9-cyclopentyl-5,7,7-trimethyl-8,9-dihydro-5H-pyrimido[4,5b][1,4]diazepin-6(7H)-one (2 g, 6.48 mmol), 4-amino-3-methoxybenzoic acid (1 g, 6.5 mmol), isopropanol (30 ml), and concentrated hydrochloric acid (20 drops) was stirred at 100° C. for 20 h. Solid was filtered to give 1.92 g 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid as white solid (70%). ¹H NMR (400 MHz, DMSO-d₆) δ ppm 1.14 (s, 6H) 1.53-1.70 (m, 4H) 1.73 (d, J=5.3 Hz, 2H) 1.79-1.91 (m, 1H) 1.85 (d, J=5.8 Hz, 1H) 3.17 (s, 4H) 3.94 (s, 3H) 5.12 (qd, J=8.4, 8.2 Hz, 1H) 7.60 (d, J=1.5 Hz, 1H) 7.58 (s, 1H) 8.08 (s, 1H) 8.15 (d, J=8.3 Hz, 1H) 9.33 (br. s., 1H) 12.96 (br. s., 1H). [M+H] calc'd for C₂₃H₂₉N₅O₄, 440; found, 440.

### Compound 86

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide

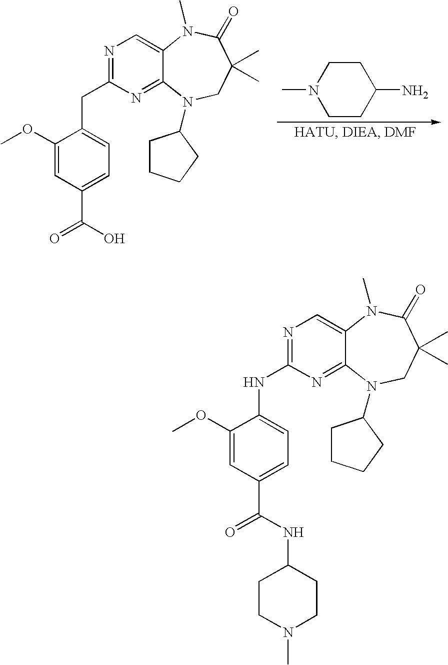

To a mixture of 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid (1 g, 2.28 mmol), 1-methylpiperidin-4-amine (263 mg, 2.3 mmol), DIEA (1 mL, 6 mmol) in 20 mL of anhydrous DMF was added HATU (1.3 g, 3.4 mmol). The reaction mixture was stirred for 30 min. The reaction mixture was then diluted with ethyl acetate, washed with water and brine. The organic layer dried over Na₂SO₄ followed by HPLC purification. The TFA salt was then converted to free base, which was finally crystallized from ether to give 700 mg white crystals (57%) of 4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide. ¹H NMR (400 MHz, DMSO-d₆) δ ppm 1.09 (s, 6H) 1.53-1.67 (m, 6H) 1.67-1.80 (m, 4H) 1.87 (br. s., 2H) 1.97 (d, J=11.4 Hz, 2H) 1.93 (br. s., 1H) 2.18 (s, 3H) 2.79 (d, J=10.9 Hz, 2H) 3.18 (s, 3H) 3.74 (dt, J=7.4, 3.8 Hz, 1H) 3.94 (s, 3H) 5.18 (t, J=8.1 Hz, 1H) 7.41-7.55 (m, 2H) 7.68 (s, 1H) 7.98 (s, 1H) 8.10 (d, J=7.6 Hz, 1H) 8.36 (d, J=8.1 Hz, 1H). [M+H] calc'd for C₂₉H₄₁N₇O₃, 536; found, 536.

### Compound 87

### 4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide

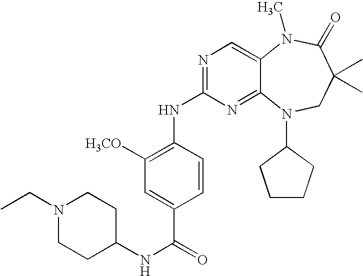

The title compound was synthesized using an analogous procedure described for Compound 86 except that 1-N-ethylpiperidine-4-amine was used. ¹H NMR (400 MHz, DMSO-d₆) δ ppm 1.13 (s, 6H) 1.23 (t, J=7.33 Hz, 3H) 1.53-1.80 (m, 10H) 2.05 (m, 2H) 3.00-3.19 (m, 5H) 3.17 (s, 3H) 3.50 (br. s, 3H) 3.94 (s, 3H) 4.10 (m, 1H) 5.13 (m, 1H) 7.52 (s, 1H) 7.55 (d, J=8.84 Hz, 1H) 8.03 (s, 1H) 8.07 (d, J=8.34 Hz, 1H) 8.47 (br. s., 1H) 9.44 (br. s., 1H). [M+H] calculated for C₃₀H₄₄N₇O₃, 550; found 550.

### Compound 88

### 4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide

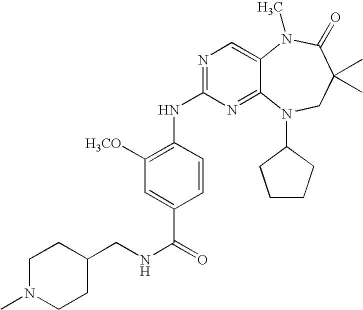

The title compound was synthesized using an analogous procedure described for Compound 86 except that (1-methyl-4-piperidinyl)methanamine was used. ¹H NMR (400 MHz, DMSO-d₆) δ ppm 1.13 (s, 6H) 1.53-2.05 (m, 13H) 2.73 (br. s., 3H) 2.83 (s, 3H) 3.18 (s, 3H) 3.47 (br. s., 5H) 3.91 (s, 3H) 5.12 (m, 1H) 7.02 (d, 1H) 7.11 (s, 1H) 7.99 (s, 1H) 8.09 (br. s., 2H) 9.27 (br. s., 1H). [M+H] calculated for C₃₀H₄₄N₇O₃, 550; found 550.

### Compound 89

### 4-(9-cyclopentyl-5,7,7-trimethyl-6-oxo-6,7,8,9-tetrahydro-5H-pyrimido[4,5-b][1,4]diazepin-2-ylamino)-N-(3-(dimethylamino)propyl)-3-methoxybenzamide

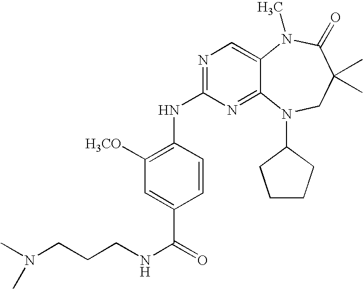

The title compound was synthesized using an analogous procedure described for Compound 86 except that N,N-dimethyl-1,3-propanediamine was used. ¹H NMR (400 MHz, DMSO-d₆) δ ppm 1.14 (s, 6H) 1.52-1.90 (m, 10H) 2.79 (s, 6H) 3.10 (q, J=6.31 Hz, 2H) 3.17 (s, 3H) 3.33 (q, J=6.32 Hz, 2H) 3.51 (br. s., 2H) 3.94 (s, 3H) 5.11 (m, 1H) 7.52 (d, J=8.59 Hz, 1H) 7.58 (s, 1H) 8.04 (br. s., 2H) 8.66 (br. s., 1H) 9.56 (br. s., 1H). [M+H] calculated for C₂₈H₄₂N₇O₃, 524; found 524.

### Compound 90

### N-(1-acetylpiperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide

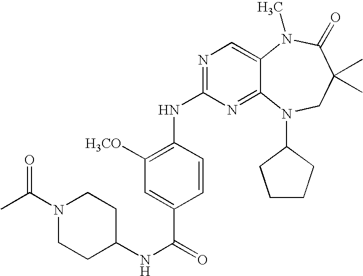

The title compound was synthesized using an analogous procedure described for Compound 86 except that N-1-acetylpiperidin-4-amine was used. ¹H NMR (400 MHz, DMSO-d₆) δ ppm 1.14 (s, 6H) 1.39-1.90 (m, 10H) 2.02 (s, 3H) 2.56-2.73 (m, 1H) 3.17 (s, 4H) 3.50 (br. s., 2H) 3.83 (m, 1H) 3.94 (s, 3H) 4.36 (br. d., 1H) 5.12 (m, 1H) 7.52 (d, J=8.34 Hz, 1H) 7.56 (s, 1H) 8.02 (s, 1H) 8.08 (br. s., 1H) 8.25 (d, J=6.57 Hz, 1H) 9.13 (br. s., 1H). [M+H] calculated for C₃₀H₄₂N₇O₄, 564; found 524.

### Compound 91

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-(methylsulfonyl)piperidin-4-yl)benzamide

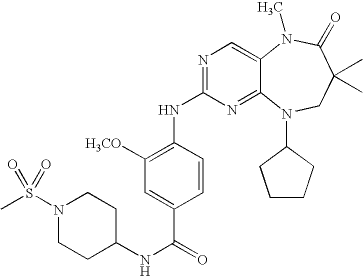

The title compound was synthesized using an analogous procedure described for Compound 86 except that N-1-(methylsulfonyl)piperidin-4-amine was used. ¹H NMR (400 MHz, DMSO-d₆) δ ppm 1.13 (s, 6H) 1.53-1.95 (m, 12H) 2.79-2.93 (m, 5H) 3.17 (s, 3H) 3.49 (br. s., 2H) 3.61 (br. d., 2H) 3.95 (s, 4H) 5.14 (m, 1H) 7.52 (d, J=8.34 Hz, 1H) 7.56 (s, 1H) 8.00 (s, 1H) 8.12 (d, J=1.52 Hz, 1H) 8.30 (d, J=7.58 Hz, 1H) 8.93 (br. s., 1H). [M+H] calculated for C₃₀H₄₂N₇O₄, 600; found 600.

### Compound 92

### 4-(9-cyclopentyl-5,7,7-trimethyl-6-oxo-6,7,8,9-tetrahydro-5H-pyrimido[4,5-b][1,4]diazepin-2-ylamino)-N-(2-(dimethylamino)ethyl)-3-methoxybenzamide

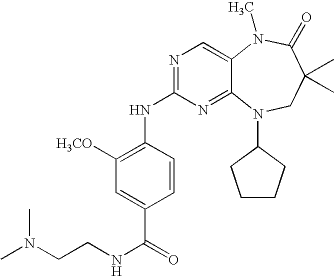

The title compound was synthesized using an analogous procedure described for Compound 86 except that N,N-dimethylethylenediamine was used. ¹H NMR (400 MHz, DMSO-d₆) δ ppm 1.14 (s, 6H) 1.55-1.90 (m, 8H) 2.87 (s, 6H) 3.18 (s, 3H) 3.28 (t, J=5.43 Hz, 2H) 3.50 (s, 2H) 3.62 (q, J=5.98 Hz, 2H) 3.95 (s, 3H) 5.12 (m, 1H) 7.54 (d, J=8.34 Hz, 1H) 7.58 (s, 1H) 8.05 (s, 1H) 8.14 (d, J=8.34 Hz, 1H) 8.76 (br. s., 1H) 9.57 (br. s., 1H). [M+H] calculated for C₂₇H₄₀N₇O₃, 510; found 510.

### Compound 93

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide

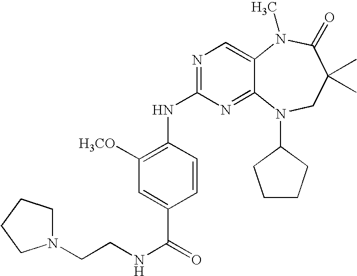

The title compound was synthesized using an analogous procedure described for Compound 86 except that 1-(2-aminoethyl)pyrrolidine was used. ¹H NMR (400 MHz, DMSO-d₆) δ ppm 1.14 (s, 6H) 1.55-2.05 (m, 12H) 3.08 (m, 2H) 3.19 (s, 3H) 3.35 (q, J=6.06 Hz, 2H) 3.50 (s, 2H) 3.63 (m, 4H) 3.96 (s, 3H) 5.14 (m, 1H) 7.54 (d, J=8.34 Hz, 1H) 7.58 (s, 1H) 8.04 (s, 1H) 8.18 (br. s., 1H) 8.74 (br. s., 1H) 9.60 (br. s., 1H). [M+H] calculated for C₂₉H₄₂N₇O₃, 536; found 536.

### Compound 94

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(tetrahydro-2H-pyran-4-yl)benzamide

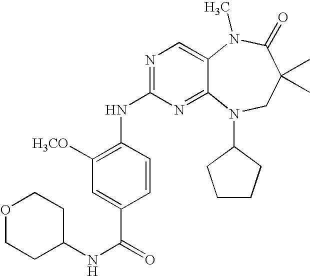

The title compound was synthesized using an analogous procedure described for Compound 86 except that 4-aminotetrahydropyran was used. ¹H NMR (400 MHz, DMSO-d₆) δ ppm 1.14 (s, 6H) 1.55-1.90 (m, 12H) 3.18 (s, 3H) 3.39 (m, 2H) 3.51 (s, 2H) 3.87-4.06 (m, 3H) 3.96 (s, 3H) 5.14 (m, 1H) 7.53 (dd, J=8.46, 1.64 Hz, 1H) 7.57 (d, J=1.52 Hz, 1H) 8.01 (s, 1H) 8.08 (d, J=8.34 Hz, 1H) 8.27 (d, J=7.58 Hz, 1H) 9.06 (br. s., 1H). [M+H] calculated for C₂₈H₃₉N₆O₄, 523; found 523.

### Compound 95

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide

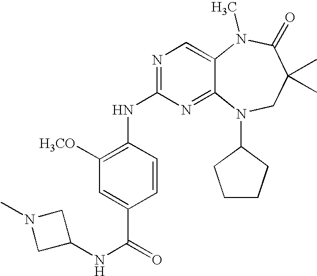

The title compound was synthesized using an analogous procedure described for Compound 86 except that 3-amino 1-N-methylazetidine was used. ¹H NMR (400 MHz, DMSO-d₆) δ ppm 1.13 (s, 6H) 1.51-1.91 (m, 8H) 2.91 (s, 3H) 3.18 (s, 3H) 3.48 (s, 2H) 3.95 (s, 2H) 4.05-4.23 (m, 2H) 4.40 (m, 1H) 4.49 (m, 1H) 4.71 (m, 1H) 5.12 (m, 1H) 7.50-7.59 (m, 2H) 8.04 (s, 1H) 8.19 (d, J=8.34 Hz, 1H) 8.91 (br. s., 1H) 9.91 (br. s., 1H). [M+H] calculated for C₂₇H₃₈N₇O₃, 508; found 508.

### Compound 96

### (R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide

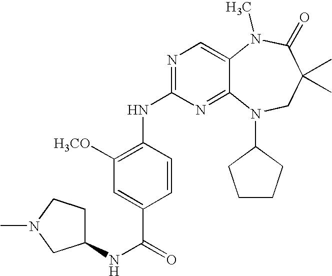

The title compound was synthesized using an analogous procedure described for Compound 86 except that 1-methyl-3-(R)-amino pyrrolidine dihydrochloride was used. ¹H NMR (400 MHz, DMSO-d₆) δ ppm 1.13 (s, 6H) 1.52-1.93 (m, 8H) 2.04-2.30 (m, 2H) 2.88 (d, J=4.80 Hz, 2H) 2.92 (d, J=4.29 Hz, 1H) 3.08 (m, 1H) 3.18 (s, 3H) 3.37 (dt, J=11.87, 7.58 Hz, 1H) 3.48 (br. s., 2H) 3.55-3.75 (m, 2H) 3.95 (s, 3H) 4.58 (m, 1H) 5.15 (m, 1H) 7.52 (s, 1H) 7.55 (d, J=5.05 Hz, 1H) 8.03 (s, 1H) 8.18 (br. s., 1H) 8.69 (br. s., 1H) 10.00 (br. s., 1H). [M+H] calculated for C₂₈H₄₀N₇O₃, 522; found 522.

### Compound 97

### (S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide

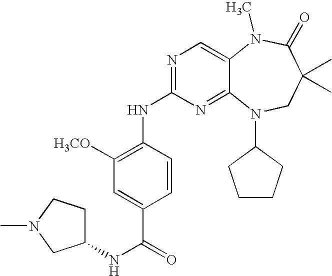

The title compound was synthesized using an analogous procedure described for Compound 86 except that 1-methyl-3-(S)-amino pyrrolidine dihydrochloride was used. ¹H NMR (400 MHz, DMSO-d₆) δ ppm 1.12 (s, 6H) 1.52-1.93 (m, 8H) 2.03-2.30 (m, 2H) 2.88 (d, J=4.80 Hz, 2H) 2.92 (d, J=4.29 Hz, 1H) 3.08 (m, 1H) 3.18 (s, 3H) 3.37 (dt, J=11.87, 7.58 Hz, 1H) 3.47 (br. s., 2H) 3.55-3.73 (m, 2H) 3.95 (s, 3H) 4.54 (m, 1H) 5.15 (m, 1H) 7.52 (s, 1H) 7.55 (d, J=5.05 Hz, 1H) 8.02 (s, 1H) 8.21 (br. s., 1H) 8.64 (br. s., 2H) 9.86 (br. s., 1H). [M+H] calculated for C₂₈H₄₀N₇O₃, 522; found 522.

### Compound 98

### (R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide

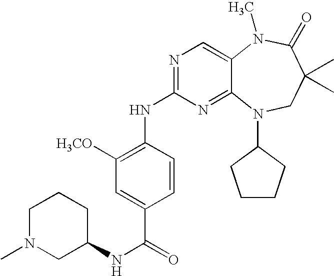

The title compound was synthesized using an analogous procedure described for Compound 86 except that 1-methyl-3-(R)-amino piperidine dihydrochloride was used. ¹H NMR (400 MHz, DMSO-d₆) δ ppm 1.13 (s, 6H) 1.52-1.99 (m, 12H) 2.72-2.80 (m, 2H) 2.85 (s, 3H) 3.18 (s, 3H) 3.48 (dd, 2H) 3.49 (s, 2H) 3.95 (s, 3H) 4.18 (m, 1H) 5.13 (m, 1H) 7.52 (d, 1H) 7.55 (s, 1H) 8.03 (s, 1H) 8.15 (br. d., 1H) 8.52 (d, J=7.33 Hz, 1H) 9.76 (br. s., 1H). [M+H] calculated for C₂₉H₄₂N₇O₃, 536; found 536.

### Compound 99

### (S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide

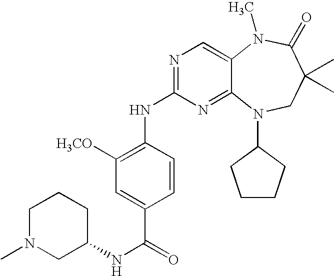

The title compound was synthesized using an analogous procedure described for Compound 86 except that 1-methyl-3-(S)-amino piperidine dihydrochloride was used. ¹H NMR (400 MHz, DMSO-d₆) δ ppm 1.13 (s, 6H) 1.52-1.99 (m, 12H) 2.72-2.80 (m, 2H) 2.85 (s, 3H) 3.18 (s, 3H) 3.48 (dd, 2H) 3.49 (s, 2H) 3.95 (s, 3H) 4.18 (m, 1H) 5.13 (m, 1H) 7.52 (d, 1H) 7.54 (s, 1H) 8.03 (s, 1H) 8.15 (br. d., 1H) 8.52 (d, J=7.33 Hz, 1H) 9.72 (br. s., 1H). [M+H] calculated for C₂₉H₄₂N₇O₃, 536; found 536.

### Compound 100

### (R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide

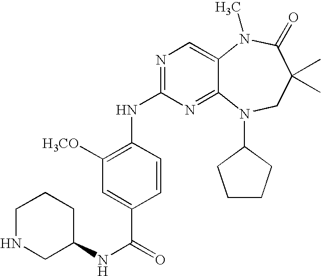

The title compound was synthesized using an analogous procedure described for Compound 86 except that (R)-1-Boc-3-aminopiperidine was used. The Boc was then removed with TFA in DCM. ¹H NMR (400 MHz, DMSO-d₆) δ ppm 1.13 (s, 6H) 1.54-1.98 (m, 12H) 2.83 (m, 2H) 3.18 (s, 3H) 3.25 (br. s., 1H) 3.33 (br. s., 1H) 3.48 (s, 2H) 3.95 (s, 3H) 4.17 (m, 1H) 5.13 (m, 1H) 7.52 (d, J=8.59 Hz, 1H) 7.54 (s, 1H) 8.03 (s, 1H) 8.16 (br. s., 1H) 8.42 (br. s., 1H) 8.68 (br. s., 1H) 8.79 (br. s., 1H). [M+H] calculated for C₂₈H₄₀N₇O₃, 522; found 522.

### Compound 101

### (S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide

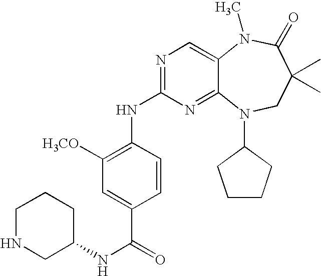

The title compound was synthesized using an analogous procedure described for Compound 86 except that (S)-1-Boc-3-aminopiperidine was used. The Boc was then removed with TFA in DCM. ¹H NMR (400 MHz, DMSO-d₆) δ ppm 1.13 (s, 6H) 1.54-1.98 (m, 12H) 2.83 (m, 2H) 3.18 (s, 3H) 3.25 (br. s., 1H) 3.33 (br. s., 1H) 3.48 (s, 2H) 3.95 (s, 3H) 4.16 (m, 1H) 5.14 (m, 1H) 7.52 (d, J=8.59 Hz, 1H) 7.54 (s, 1H) 8.03 (s, 1H) 8.18 (br. s., 1H) 8.40 (br. s., 1H) 8.67 (br. s., 2H). [M+H] calculated for C₂₈H₄₀N₇O₃, 522; found 522.

### Compound 102

### 4-(9-cyclopentyl-5,7,7-trimethyl-6-oxo-6,7,8,9-tetrahydro-5H-pyrimido[4,5-b][1,4]diazepin-2-ylamino)-N,3-dimethoxybenzamide

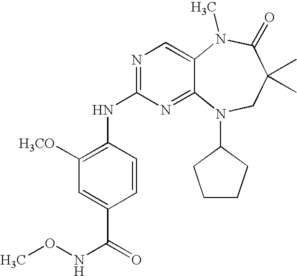

The title compound was synthesized using an analogous procedure described for Compound 86 except that N-methoxylamine hydrochloride was used. ¹H NMR (400 MHz, DMSO-d₆) δ ppm 1.10 (s, 6H) 1.56-1.90 (m, 8H) 3.19 (s, 3H) 3.36 (d, J=18.69 Hz, 2H) 3.71 (s, 3H) 3.93 (s, 3H) 5.18 (m, 1H) 7.39 (d, J=8.34 Hz, 1H) 7.41 (s, 1H) 7.71 (s, 1H) 7.99 (s, 1H) 8.39 (d, J=8.34 Hz, 1H) 11.65 (br. s., 1H). [M+H] calculated for C₂₈H₄₀N₇O₃, 469; found 469.

### Compound 103

### 4-(9-cyclopentyl-5,7,7-trimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

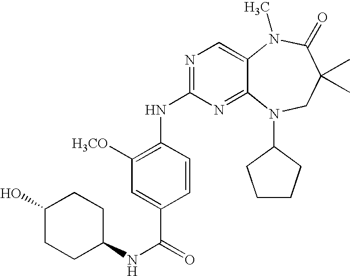

The title compound was synthesized using an analogous procedure described for Compound 86 except that (1R,4R)-4-hydroxycyclohexylamine was used. ¹H NMR (400 MHz, DMSO-d₆) δ ppm 1.13 (s, 6H) 1.24 (m, 1H) 1.37 (m, 1H) 1.53-1.98 (m, 12H) 3.17 (s, 3H) 3.40 (m, 1H) 3.49 (s, 2H) 3.74 (m, 1H) 3.94 (s, 3H) 7.54 (d, J=1.52 Hz, 1H) 7.50 (dd, J=8.34, 1.52 Hz, 1H) 8.00 (s, 1H) 8.08 (br. s., 1H) 8.10 (d, J=8.84 Hz, 1H) 8.88 (br. s., 1H). [M+H] calculated for C₂₉H₄₁N₆O₄, 537; found 537.

### Compound 104

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-morpholinobenzamide

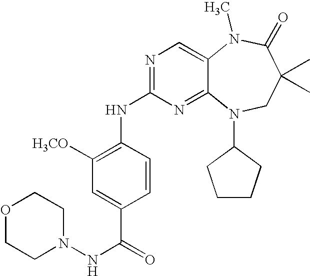

The title compound was synthesized using an analogous procedure described for Compound 86 except that 4-aminomorpholine was used. ¹H NMR (400 MHz, DMSO-d₆) δ ppm 1.09 (s, 6H) 1.55-1.93 (m, 8H) 2.92 (br. s., 4H) 3.18 (s, 3H) 3.26-3.44 (m, 2H) 3.67 (br. s., 4H) 3.94 (s, 3H) 5.18 (m, 1H) 7.41 (d, J=8.34 Hz, 1H) 7.42 (s, 1H) 7.69 (s, 1H) 7.99 (s, 1H) 8.37 (d, J=8.34 Hz, 1H) 9.42 (s, 1H). [M+H] calculated for C₂₇H₃₈N₇O₄, 524; found 524.

### Compound 105

### 4-(9-cyclopentyl-5,7,7-trimethyl-6-oxo-6,7,8,9-tetrahydro-5H-pyrimido[4,5-b][1,4]diazepin-2-ylamino)-N-(4-(2-hydroxyethyl)piperazin-1-yl)-3-methoxybenzamide

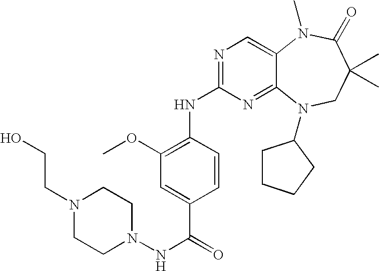

The title compound was synthesized using an analogous procedure described for Compound 86 except that 1-amino 4-(2-hydroxyethyl)piperazine was used. ¹H NMR (400 MHz, DMSO-d₆) δ ppm 1.09 (s, 6H) 1.56-1.91 (m, 8H) 2.40 (t, J=6.06 Hz, 2H) 2.91 (br. s., 4H) 3.18 (s, 3H) 3.31-3.40 (m, 5H) 3.50 (d, J=3.54 Hz, 2H) 3.93 (s, 3H) 4.41 (br. s., 1H) 5.19 (m, 1H) 7.37-7.45 (m, 2H) 7.69 (s, 1H) 7.98 (s, 1H) 8.36 (d, J=8.08 Hz, 1H) 9.32 (s, 1H). [M+H] calculated for C₂₇H₃₈N₇O₄, 567; found 567.

### Compound 106

### 4-(9-cyclopentyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide

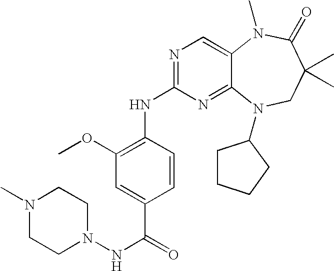

The title compound was synthesized using an analogous procedure described for Compound 86 except that 1-amino 4-methylpiperazine was used. ¹H NMR (400 MHz, DMSO-d₆) δ ppm 1.09 (s, 6H) 1.55-1.90 (m, 8H) 2.19 (s, 3H) 2.92 (s, 4H) 3.19 (s, 3H) 3.28-3.42 (m, 6H) 3.94 (s, 3H) 5.19 (m, 1H) 7.38-7.49 (m, 2H) 7.69 (s, 1H) 7.99 (s, 1H) 8.37 (d, J=8.08 Hz, 1H) 9.32 (s, 1H). [M+H] calculated for C₂₇H₃₈N₇O₄, 537; found 537.

### Compound 107

### 4-(9-cyclopentyl-5,7,7-trimethyl-6-oxo-6,7,8,9-tetrahydro-5H-pyrimido[4,5-b][1,4]diazepin-2-ylamino)-3-methoxybenzamide

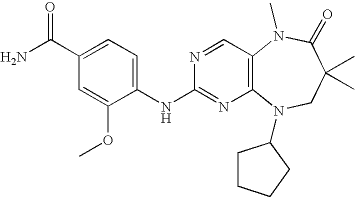

The title compound was synthesized using an analogous procedure described for Compound 86 except that ammonium chloride was used. ¹H NMR (400 MHz, DMSO-d₆) δ ppm 1.14 (s, 6H) 1.53-1.88 (m, 8H) 3.17 (s, 3H) 3.51 (s, 2H) 3.93 (s, 3H) 5.10 (quin., J=8.1 Hz, 1H) 7.37 (br. s., 1H) 7.55 (dd, J=8.3, 1.3 Hz, 1H) 7.61 (s, 1H) 8.01 (s, 2H) 8.04 (d, J=8.3 Hz, 1H). [M+H] calc'd for C₂₃H₃₀F₂N₆O₃ 439; found 439.

### Compound 108

### 4-(9-cyclohexyl-5,7,7-trimethyl-6-oxo-6,7,8,9-tetrahydro-5H-pyrimido[4,5-b][1,4]diazepin-2-ylamino)-3-methoxybenzoic Acid

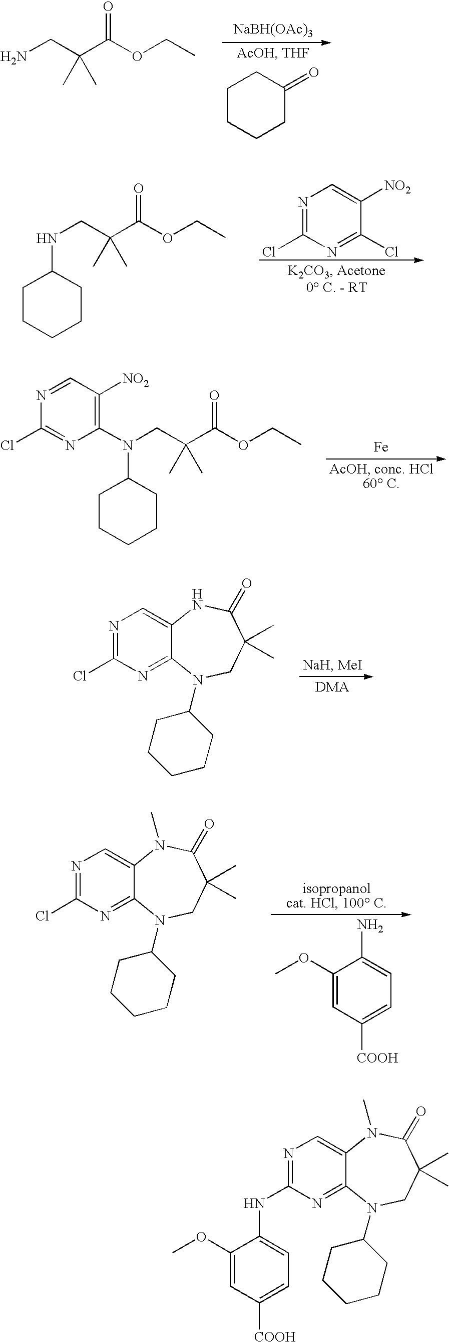

Ethyl 3-(cyclohexylamino)-2,2-dimethylpropanoate. Ethyl 3-amino-2,2-dimethylpropanoate (1.45 g, 10 mmol) was treated with cyclohexanone (1.177 g, 12 mmol) in the presence of sodium triacetoxyborohydride (3.17 g, 15 mmol) and glacial acetic acid (3 mL) in tetrahydrofuran (30 mL) for 18 h at room temperature. The reaction mixture was carefully diluted with saturated aqueous NaHCO₃ (75 mL). The mixture was stirred for 1 hour at room temperature. The whole was extracted with ethyl acetate (100 mL×3). The organic layer was dried over Na₂SO₄ and concentrated in vacuo to obtain ethyl 3-(cyclohexylamino)-2,2-dimethylpropanoate (1.95 g, 86% yield) as light yellow liquid. ¹H NMR (400 MHz, CHLOROFORM-d) δ 1.16 (s, 6H) 1.22-1.32 (m, 6H) 1.39-2.10 (m, 7H) 2.65 (s, 2H) 2.95-3.02 (m, 1H) 4.19 (q, J=7.2 Hz, 2H). [M+H] calculated for C₁₃H₂₆NO₂, 228; found 228.

Ethyl 3-((2-chloro-5-nitropyrimidin-4-yl)(cyclohexyl)amino)-2,2-dimethylpropanoate. To a solution of 2,4-dichloro-5-nitropyrimidine (1.90 g, 9.82 mmol) in anhydrous acetone (25 ml) at 0° C., was added dropwise a solution of ethyl 3-(cyclohexylamino)-2,2-dimethylpropanoate (2.23 g, 9.82 mmol) in acetone (10 mL) over 10 min. After which, potassium carbonate (2.71 g, 19.64 mmol) was added and the whole was stirred at rt for 18 h. After evaporation in vacuo, the residue was partitioned between ethyl acetate (75 ml) and water (50 ml). The organic layer was washed with NaHCO₃, brine and water, dried over Na₂SO₄ and concentrated in vacuo. The residue was purified by silica gel chromatography (ethyl acetate:hexane=1:10 to 1:5) and recrystallized from ether/EtOAc to obtain compound 11-2 (1.29 g, 34% yield) as pale yellow powder. ¹H NMR (400 MHz, CHLOROFORM-d) δ ppm 1.09-1.32 (m, 8H) 1.39-1.55 (m, 1H) 1.68-1.79 (m, 1H) 1.79-1.91 (m, 3H) 2.90 (m, 1H) 4.12 (q, J=7.16 Hz, 3H), 8.73 (s, 1H). [M+H] calculated for C₁₇H₂₆ClN₄O₄, 385; found 385.

2-Chloro-9-cyclohexyl-7,7-dimethyl-8,9-dihydro-5H-pyrimido[4,5-b][1,4]diazepin-6(7H)-one. To a suspension of ethyl 3-((2-chloro-5-nitropyrimidin-4-yl)(cyclohexyl)amino)-2,2-dimethylpropanoate (0.46 g, 1.69 mmol), reduced iron (189.2 mg, 3.38 mmol) in acetic acid (10 ml) was added dropwise concentrated hydrochloric acid (1 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2-chloro-9-cyclohexyl-7,7-dimethyl-8,9-dihydro-5H-pyrimido[4,5-b][1,4]diazepin-6(7H)-one (0.46 g, 89% yield) as white solid. [M+H] calculated for C₁₅H₂₂ClN₄O, 309; found 309.

2-Chloro-9-cyclohexyl-5,7,7-trimethyl-8,9-dihydro-5H-pyrimido[4,5-b][1,4]diazepin-6(7H)-one. To a solution of 2-chloro-9-cyclohexyl-7,7-dimethyl-8,9-dihydro-5H-pyrimido[4,5-b][1,4]diazepin-6(7H)-one (0.46 g, 1.49 mmol) in 10 mL of DMA was added sodium hydride (60% dispersion in mineral oil, 89.6 mg, 2.24 mmol) at 0° C., followed by the drop wise addition of methyl iodide (0.153 mL, 1.79 mmol). The reaction mixture was warmed up to rt and stirred for 1 h. The whole was poured into ice-water, extracted with ethyl acetate. The organic layer was washed with brine and dried over Na₂SO₄. The solution was concentrated in vacuo followed by precipitation from ether/EtOA to afford 2-chloro-9-cyclohexyl-5,7,7-trimethyl-8,9-dihydro-5H-pyrimido[4,5-b][1,4]diazepin-6(7H)-one (0.47 g, 97% yield) as white solid. ¹H NMR (400 MHz, DMSO-d6) δ ppm 1.08 (s, 6H) 1.58-1.90 (m, 10H) 3.18 (s, 3H) 3.41 (s, 2H) 5.08 (t, J=8.3 Hz, 1H) 8.05 (s, 1H). [M+H] calculated for C₁₆H₂₄ClN₄O, 323; found 323.

4-(9-Cyclohexyl-5,7,7-trimethyl-6-oxo-6,7,8,9-tetrahydro-5H-pyrimido[4,5-b][1,4]diazepin-2-ylamino)-3-methoxybenzoic acid. A mixture of 2-chloro-9-cyclohexyl-5,7,7-trimethyl-8,9-dihydro-5H-pyrimido[4,5-b][1,4]diazepin-6(7H)-one (0.47 g, 1.45 mmol), 4-amino-3-methoxybenzoic acid (0.36 g, 2.18 mmol), isopropanol (10 ml), and concentrated hydrochloric acid (4 drops) was stirred at 100° C. for 20 h. Solid was filtered to give 0.43 g product 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid as white solid (65%). ¹H NMR (400 MHz, DMSO-d₆) δ ppm 1.14 (s, 6H) 1.58-1.90 (m, 10H) 3.17 (s, 4H) 3.94 (s, 3H) 5.12 (qd, J=8.4, 8.2 Hz, 1H) 7.60 (d, J=1.5 Hz, 1H) 7.58 (s, 1H) 8.08 (s, 1H) 8.15 (d, J=8.3 Hz, 1H) 9.33 (br. s., 1H) 12.96 (br. s., 1H). [M+H] calculated for C₂₄H₃₂N₅O₄, 454; found 454.

### Compound 109

### 4-(9-cyclohexyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide

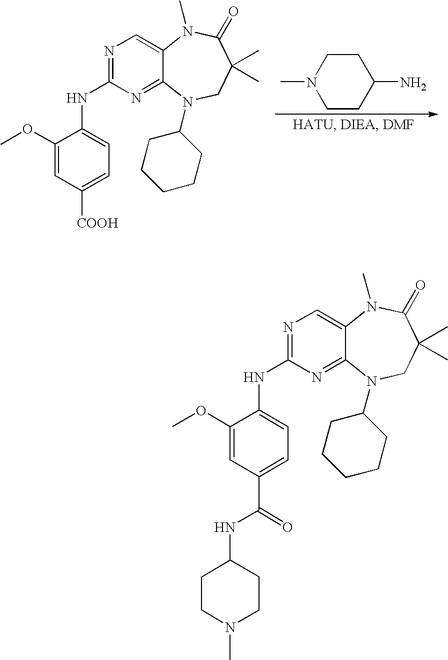

To a mixture of compound 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid (90.6 mg, 0.2 mmol), 1-methylpiperidin-4-amine (34.2 mg, 0.3 mmol), DIEA (106 μL, 0.3 mmol) in 2.0 mL of anhydrous DMF was added HATU (114 mg, 0.3 mmol). The reaction mixture was stirred for 30 min. The reaction mixture was then diluted with ethyl acetate, washed with water and brine. The organic layer dried over Na₂SO₄ followed by HPLC purification. The TFA salt was then converted to free base, which was finally crystallized from ether to give 72 mg (66%) of the title compound. ¹H NMR (400 MHz, DMSO-d₆) δ ppm 1.09 (s, 6H) 1.10-1.26 (m, 1H) 1.41-1.98 (m, 14H) 2.17 (s, 3H) 2.79 (d, J=11.62 Hz, 2H) 3.18 (s, 3H) 3.41 (s, 2H) 3.75 (m, 1H) 3.95 (s, 3H) 4.71 (m, 1H) 7.47 (d, J=8.59 Hz, 1H) 7.50 (s, 1H) 7.68 (s, 1H) 7.97 (s, 1H) 8.12 (d, J=8.03 Hz, 1H) 8.36 (d, J=8.34 Hz, 1H). [M+H] calculated for C₃₀H₄₄N₇O₃, 550; found 550.

### Compound 110

### (R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide

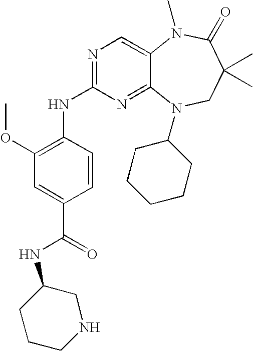

The title compound was synthesized using an analogous procedure described for Compound 109 except that (R)-tert-butyl 3-aminopiperidine-1-carboxylate was used, followed by removal of the Boc with TFA in DCM. The TFA salt was then converted to free base, which was finally crystallized from ether to give the title compound. ¹H NMR (400 MHz, DMSO-d₆) δ ppm 1.09 (t, J=7.07 Hz, 6H) 1.14-1.42 (m, 4H) 1.52-1.77 (m, 6H) 1.79-1.97 (m, 4H) 2.73-2.92 (m, 2H) 3.17 (s, 3H) 3.24 (d, J=14.40 Hz, 1H) 3.49 (s, 2H) 3.96 (br. s., 4H) 4.15 (br. s., 2H) 4.60-4.78 (m, 1H) 7.51 (dd, J=8.59, 1.52 Hz, 2H) 8.01 (s, 1H) 8.23 (d, J=8.34 Hz, 1H) 8.40 (d, J=7.58 Hz, 1H) 8.70 (br. s., 1H). [M+H] C₂₉H₄₁N₇O₃ calc'd for, 536; found 536.

### Compound 111

### (S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide

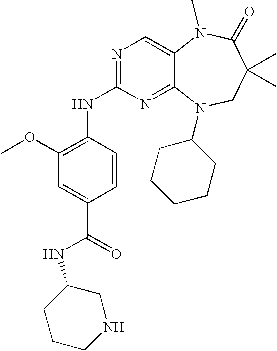

The title compound was synthesized using an analogous procedure described for Compound 109 except that (S)-tert-butyl 3-aminopiperidine-1-carboxylate was used, followed by removal of the Boc with TFA in DCM. The TFA salt was then converted to free base, which was finally crystallized from ether to give the title compound. ¹H NMR (400 MHz, DMSO-d₆) δ ppm 1.09 (s, 6H) 1.14-1.42 (m, 4H) 1.52-1.77 (m, 6H) 1.79-1.97 (m, 4H) 2.73-2.92 (m, 2H) 3.17 (s, 3H) 3.24 (d, J=14.40 Hz, 1H) 3.49 (s, 2H) 3.96 (br. s., 4H) 4.15 (br. s., 2H) 4.60-4.78 (m, 1H) 7.51 (dd, J=8.59, 1.52 Hz, 2H) 8.01 (s, 1H) 8.23 (d, J=8.34 Hz, 1H) 8.40 (d, J=7.58 Hz, 1H) 8.70 (br. s., 1H). [M+H] C₂₉H₄₁N₇O₃ calc'd for, 536; found 536.

### Compound 112

### 4-(9-cyclohexyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide

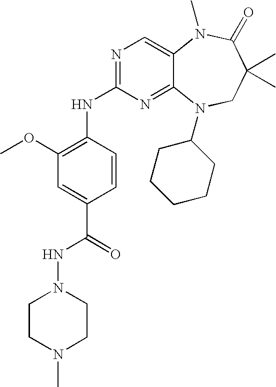

The title compound was synthesized using an analogous procedure described for Compound 109 except that 4-methylpiperazin-1-amine was used. The TFA salt was then converted to free base, which was finally crystallized from ether to give the title compound. ¹H NMR (400 MHz, DMSO-d₆) δ ppm 1.09 (s, 6H) 1.13-1.91 (m, 10H) 2.19 (s, 3H) 2.26-2.44 (m, 4H) 2.94 (br. s., 2H) 3.17 (s, 3H) 3.39 (d, J=6.82 Hz, 2H) 3.51 (br. s, 2H) 3.94 (s, 3H) 4.69 (br. s., 1H) 6.95 (dd, J=8.21, 1.39 Hz, 1H) 7.35-7.47 (m, 1H) 7.67 (d, J=4.80 Hz, 1H) 7.96 (d, J=6.82 Hz, 1H) 8.21-8.44 (m, 1H) 9.37 (s, 1H). [M+H] C₂₉H₄₂N₈O₃ calc'd for, 551; found 551.

### Compound 113

### 4-(9-cyclohexyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide

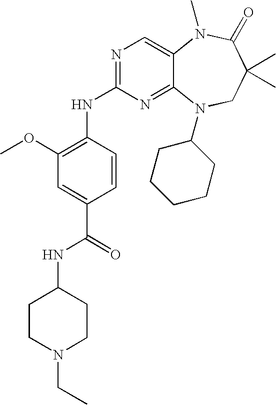

The title compound was synthesized using an analogous procedure described for Compound 109 except that 4-ethylpiperazin-1-amine was used. The TFA salt was then converted to free base, which was finally crystallized from ether to give the title compound. ¹H NMR (400 MHz, DMSO-d₆) δ ppm 1.00 (t, J=7.20 Hz, 3H) 1.09 (s, 6H) 1.13-1.26 (m, 2H) 1.30-2.02 (m, 14H) 2.32 (q, J=7.33 Hz, 2H) 2.89 (d, J=11.62 Hz, 2H) 3.17 (s, 3H) 3.40 (s, 2H) 3.68-3.81 (m, 1H) 3.95 (s, 3H) 4.65-4.77 (m, 1H) 7.40-7.55 (m, 2H) 7.68 (s, 1H) 7.97 (s, 1H) 8.12 (d, J=7.83 Hz, 1H) 8.36 (d, J=8.59 Hz, 1H). [M+H] C₃₁H₄₅N₇O₃ calc'd for, 564; found 564.

### Compound 114

### (R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide

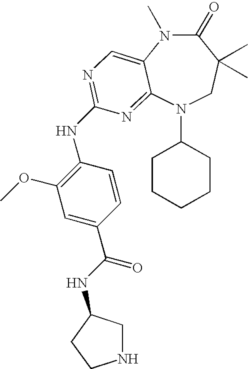

The title compound was synthesized using an analogous procedure described for Compound 109 except that (R)-tert-butyl 3-aminopyrrolidine-1-carboxylate was used, followed by removal of the Boc with TFA in DCM. The TFA salt was then converted to free base, which was finally crystallized from ether to give the title compound. ¹H NMR (400 MHz, DMSO-d₆) δ ppm 1.09 (s, 6H) 1.13-1.27 (m, 2H) 1.30-1.49 (m, 2H) 1.50-1.79 (m, 7H) 1.80-1.91 (m, 2H) 1.91-2.03 (m, 1H) 2.60-2.69 (m, 1H) 2.69-2.82 (m, 1H) 2.84-3.03 (m, 2H) 3.18 (s, 3H) 3.40 (s, 2H) 3.95 (s, 3H) 4.31 (br. s., 1H) 4.62-4.84 (m, 1H) 7.47 (d, J=8.59 Hz, 2H) 7.68 (s, 1H) 7.97 (s, 1H) 8.22 (d, J=6.82 Hz, 1H) 8.36 (d, J=8.59 Hz, 1H) [M+H] C₂₈H₃₉N₇O₃ calc'd for, 522; found 522.

### Compound 115

### (S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide

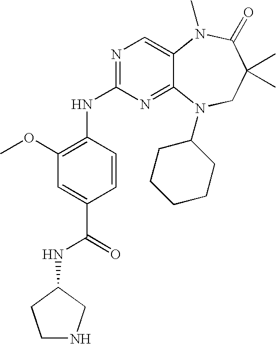

The title compound was synthesized from 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid and (S)-tert-butyl 3-aminopyrrolidine-1-carboxylate, followed by removal of the Boc with TFA in DCM, using an analogous procedure described for Compound 109. ¹H NMR (400 MHz, DMSO-d₆) δ ppm 1.09 (s, 6H) 1.13-1.27 (m, 2H) 1.30-1.49 (m, 2H) 1.50-1.79 (m, 7H) 1.80-1.91 (m, 2H) 1.91-2.03 (m, 1H) 2.60-2.69 (m, 1H) 2.69-2.82 (m, 1H) 2.84-3.03 (m, 2H) 3.18 (s, 3H) 3.40 (s, 2H) 3.95 (s, 3H) 4.31 (br. s., 1H) 4.62-4.84 (m, 1H) 7.47 (d, J=8.59 Hz, 2H) 7.68 (s, 1H) 7.97 (s, 1H) 8.22 (d, J=6.82 Hz, 1H) 8.36 (d, J=8.59 Hz, 1H) [M+H] C₂₈H₃₉N₇O₃ calc'd for, 522; found 522.

### Compound 116

### 4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide

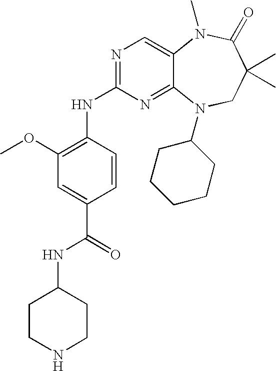

The title compound was synthesized from 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid and tert-butyl 4-aminopiperidine-1-carboxylate, followed by removal of the Boc with TFA in DCM, using an analogous procedure described for Compound 109. ¹H NMR (400 MHz, DMSO-d₆) δ ppm 1.09 (s, 6H) 1.14-1.28 (m, 2H) 1.33-1.51 (m, 4H) 1.57 (dd, J=11.87, 2.27 Hz, 2H) 1.74 (d, J=12.13 Hz, 6H) 1.86 (d, J=12.38 Hz, 2H) 2.54 (br. s., 1H) 2.97 (d, J=12.13 Hz, 2H) 3.18 (s, 3H) 3.36-3.45 (m, 3H) 3.83 (dt, J=7.58, 3.79 Hz, 1H) 3.95 (s, 3H) 4.71 (t, J=11.75 Hz, 1H) 7.48 (d, J=8.34 Hz, 2H) 7.67 (s, 1H) 7.97 (s, 1H) 8.13 (d, J=7.83 Hz, 1H) 8.36 (d, J=8.34 Hz, 1H). [M+H] C₂₉H₄₁N₇O₃ calc'd for, 536; found 536.

### Compound 117

### 4-(9-cyclohexyl-5,7,7-trimethyl-6-oxo-6,7,8,9-tetrahydro-5H-pyrimido[4,5-b][1,4]diazepin-2-ylamino)-N-(4-cyclopentylpiperazin-1-yl)-3-methoxybenzamide

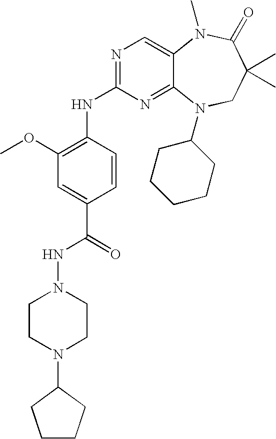

The title compound was synthesized from 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid and 4-cyclopentylpiperazin-1-amine, using an analogous procedure described for Compound 109. ¹H NMR (400 MHz, DMSO-d₆) δ ppm 1.02-1.14 (m, 8H) 1.16-1.41 (m, 4H) 1.49-1.77 (m, 14H) 1.78-1.90 (m, 4H) 1.94-2.12 (m, 4H) 3.16 (s, 3H) 3.48-3.56 (m, 3H) 3.96 (br. s., 3H) 4.68 (br. s., 1H) 7.42-7.57 (m, 2H) 8.01 (d, J=4.04 Hz, 1H) 8.16 (br. s., 1H) 9.32 (br. s., 1H) 9.89 (br. s., 1H). [M+H] C₃₃H₄₈N₈O₃ calc'd for, 605; found 605.

### Compound 118

### N-(azetidin-3-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide

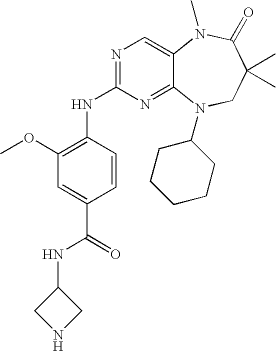

The title compound was synthesized from 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid and tert-butyl 3-aminoazetidine-1-carboxylate, followed by removal of the Boc with TFA in DCM, using an analogous procedure described for Compound 109. ¹H NMR (400 MHz, DMSO-d₆) δ ppm 1.05-1.12 (m, 10H) 1.13-1.27 (m, 2H) 1.31-1.49 (m, 2H) 1.51-1.65 (m, 2H) 1.64-1.78 (m, 2H) 1.80-1.93 (m, 2H) 3.18 (s, 3H) 3.38-3.44 (m, 2H) 3.55-3.69 (m, 2H) 3.93 (s, 3H) 4.62-4.77 (m, 1H) 7.50 (d, J=8.59 Hz, 2H) 7.69 (s, 1H) 7.97 (s, 1H) 8.38 (d, J=8.34 Hz, 1H) 8.75 (d, J=6.82 Hz, 1H) [M+H] C₂₇H₃₇N₇O₃ calc'd for, 508; found 508.

### Compound 119

### N-(azepan-4-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide

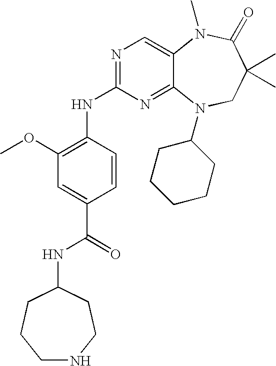

The title compound was synthesized using 4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid and tert-butyl 4-aminoazepane-1-carboxylate, by removal of the Boc with TFA in DCM, using an analogous procedure described for Compound 109. ¹H NMR (400 MHz, DMSO-d₆) δ ppm 1.04-1.13 (m, 8H) 1.14-1.28 (m, 2H) 1.32-1.50 (m, 4H) 1.48-2.04 (m, 9H) 2.83 (ddd, J=6.38, 3.09, 2.91 Hz, 2H) 2.88-3.01 (m, 2H) 3.18 (s, 3H) 3.37-3.44 (m, 2H) 3.95 (s, 3H) 4.06 (br. s., 1H) 4.71 (br. s., 1H) 7.41-7.52 (m, 2H) 7.68 (s, 1H) 7.97 (s, 1H) 8.21 (d, J=7.83 Hz, 1H) 8.36 (d, J=8.34 Hz, 1H) [M+H] C₃₀H₄₃N₇O₃ calc'd for, 550; found 550.

### Compound 120

### 4-(9-cyclopentyl-5,7,7-trimethyl-6-oxo-6,7,8,9-tetrahydro-5H-pyrimido[4,5-b][1,4]diazepin-2-ylamino)-3-hydroxybenzoic Acid

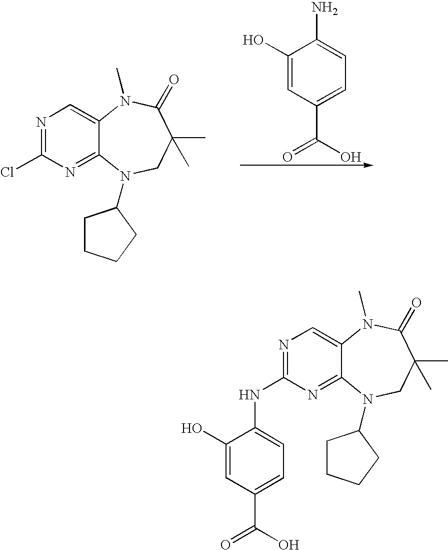

The title compound was synthesized using an analogous procedure similar to that described in the preparation of 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid, except that 4-amino-3-hydroxybenzoic acid was used. [M+H] calc'd for C₂₂H₂₇N₅O₄, 426; found 426.

### Compound 121

### 4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide

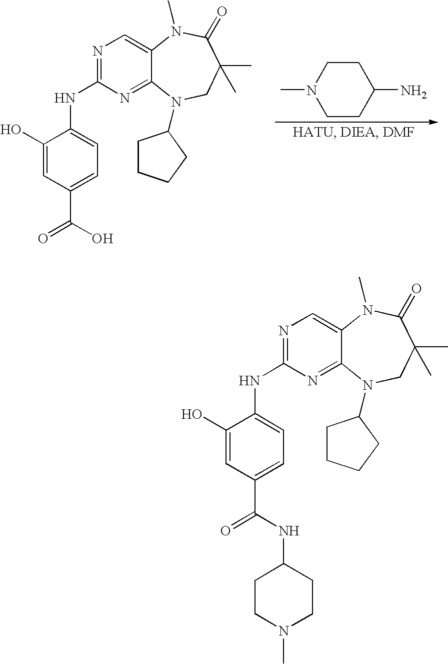

The title compound was synthesized using an analogous procedure similar to that described in preparation of Compound 86. ¹H NMR (400 MHz, MeOD) δ ppm 1.19 (d, J=0.19 Hz, 6H) 1.57-1.90 (m, 8H) 1.91-2.04 (m, 4H) 2.21 (br. s, 2H) 2.33 (d, J=0.25 Hz, 3H) 2.95 (br. s., 2H) 3.28 (dd, J=1.17, 0.16 Hz, 2H) 3.35 (d, J=1.39 Hz, 1H) 3.44 (d, J=0.32 Hz, 2H) 3.82-3.93 (m, 1H) 5.28-5.42 (m, 1H) 7.33 (qd, J=2.15, 1.39 Hz, 2H) 7.90 (dd, J=1.23, 0.16 Hz, 1H) 8.23 (dt, J=8.97, 0.66 Hz, 1H) [M+H] calc'd for C₂₈H₃₉N₇O₃, 522; found 522.

### Compound 122

### 4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide

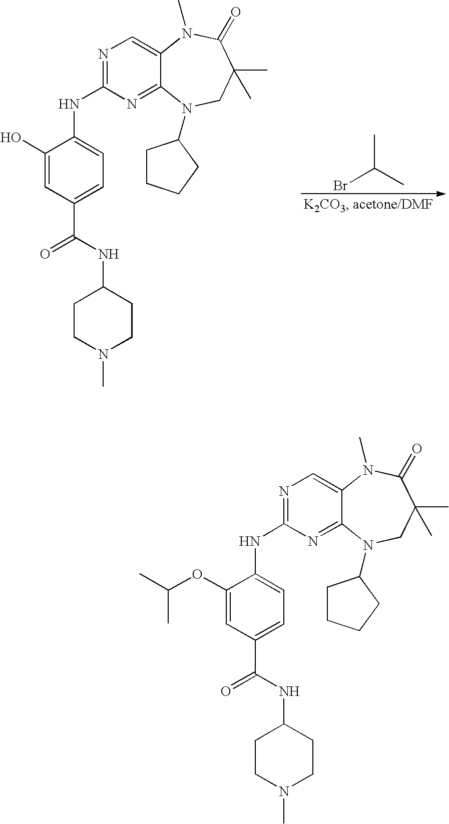

To a solution of 4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide (30 mg, 0.0576 mmole) in 200 uL/50 uL of acetone, anhydrous and DMF, anhydrous, was added K₂CO₃ (16 mg, 0.115 mmole) and 2-bromopropane (6.5 uL, 0.0691 mmole). The reaction was stirred at r.t. overnight and was purified by prep HPLC. The TFA salt was then converted to free base, which was finally crystallized from ether to give 8 mg white crystal (25%). ¹H NMR (400 MHz, CHLOROFORM-d) δ ppm 0.87 (br. s., 2H) 1.41 (br. s., 6H) 1.48-2.09 (m, 8H) 2.23 (br. s., 4H) 2.87 (br. s, 6H) 3.24 (d, J=10.67 Hz, 4H) 3.44 (br. s., 2H) 3.70 (d, J=10.86 Hz, 6H) 4.67-4.84 (m, 1H) 6.53 (t, J=8.27 Hz, 1H) 7.38-7.51 (m, 1H) 7.88-8.07 (m, 1H) 10.09 (br. s., 1H). [M+H] calc'd for C₃₁H₄₅N₇O₃, 564; found 564.

### Compound 123

### 4-(9-cyclopentyl-5,7,7-trimethyl-6-oxo-6,7,8,9-tetrahydro-5H-pyrimido[4,5-b][1,4]diazepin-2-ylamino)-3-ethoxy-N-(1-methylpiperidin-4-yl)benzamide

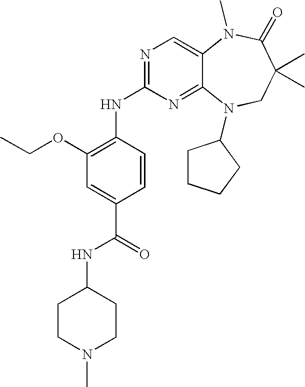

The title compound was synthesized using an analogous procedure same as that described in connection with Compound 122 except that bromoethane was used. ¹H NMR (400 MHz, MeOD) δ ppm 1.24 (s, 6H) 1.40 (d, J=19.07 Hz, 2H) 1.60-1.77 (m, 4H) 1.83 (br. s., 2H) 1.97 (d, J=5.87 Hz, 2H) 2.06-2.24 (m, 4H) 3.10 (d, J=5.37 Hz, 2H) 3.21 (d, J=8.65 Hz, 2H) 3.37-3.71 (m, 10H) 3.98 (s, 1H) 4.21 (d, J=5.05 Hz, 1H) 5.23-5.39 (m, 1H) 7.40 (dd, J=8.40, 1.58 Hz, 1H) 7.46 (s, 1H) 7.86 (s, 1H) 7.92 (d, J=8.46 Hz, 1H). [M+H] calc'd for C₃₀H₄₃N₇O₃, 550; found 550.

### Compound 124

### 4-(9-cyclopentyl-5,7,7-trimethyl-6-oxo-6,7,8,9-tetrahydro-5H-pyrimido[4,5-b][1,4]diazepin-2-ylamino)-N-(1-methylpiperidin-4-yl)-3-propoxybenzamide

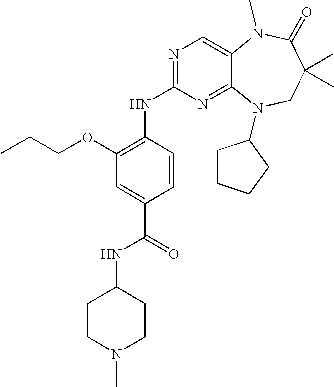

The title compound was synthesized using an analogous procedure same as that described in connection with Compound 122 except that 1-bromopropane was used. ¹H NMR (400 MHz, CHLOROFORM-d) δ ppm 0.76-0.91 (m, 6H) 1.03-1.42 (m, 8H) 1.65 (dd, J=12.73, 5.84 Hz, 8H) 1.77-2.00 (m, 6H) 2.08 (d, J=11.68 Hz, 2H) 3.33 (d, J=6.51 Hz, 2H) 3.92-4.23 (m, 4H) 4.64 (dt, J=12.09, 6.02 Hz, 1H) 5.21 (quin, J=8.62 Hz, 1H) 6.33 (d, J=7.70 Hz, 1H) 7.16-7.26 (m, 1H) 7.34 (s, 1H) 7.45 (dd, J=5.65, 3.32 Hz, 1H) 7.63 (dd, J=5.68, 3.28 Hz, 1H) 7.78 (s, 1H) 8.42 (d, J=8.46 Hz, 1H). [M+H] calc'd for C₃₁H₄₅N₇O₃, 564; found 564.

### Compound 125

### 4-(9-cyclopentyl-5,7,7-trimethyl-6-oxo-6,7,8,9-tetrahydro-5H-pyrimido[4,5-b][1,4]diazepin-2-ylamino)-3-(2,2-difluoroethoxy)-N-(1-methylpiperidin-4-yl)benzamide

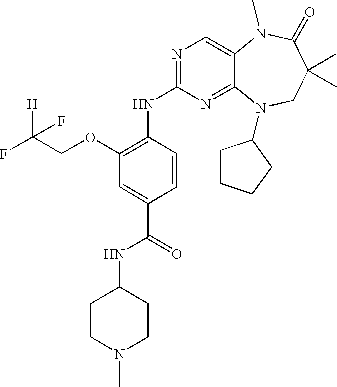

The titled compound was synthesized using an analogous procedure same as that described in connection with Compound 122 except that 1,1-difluoro-2-iodoethane was used, with the exception that 2,2-difluoroethanol was used. ¹H NMR (400 MHz, DMSO-d₆) δ ppm 1.09 (s, 6H) 1.47-2.03 (m, 16H) 2.17 (s, 3H) 2.78 (d, J=11.62 Hz, 2H) 3.19 (s, 3H) 3.73 (d, J=7.58 Hz, 1H) 4.48 (td, J=14.59, 3.41 Hz, 2H) 5.14 (t, J=8.34 Hz, 1H) 6.53 (t, J=3.28 Hz, 1H) 7.49-7.61 (m, 2H) 7.79 (s, 0H) 7.99 (s, 1H) 8.08 (d, J=7.83 Hz, 1H) 8.37 (d, J=8.34 Hz, 1H). [M+H] calc'd for C₃₀H₄₁F₂N₇O₃, 572; found 572.

### Compound 126

### 4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic Acid

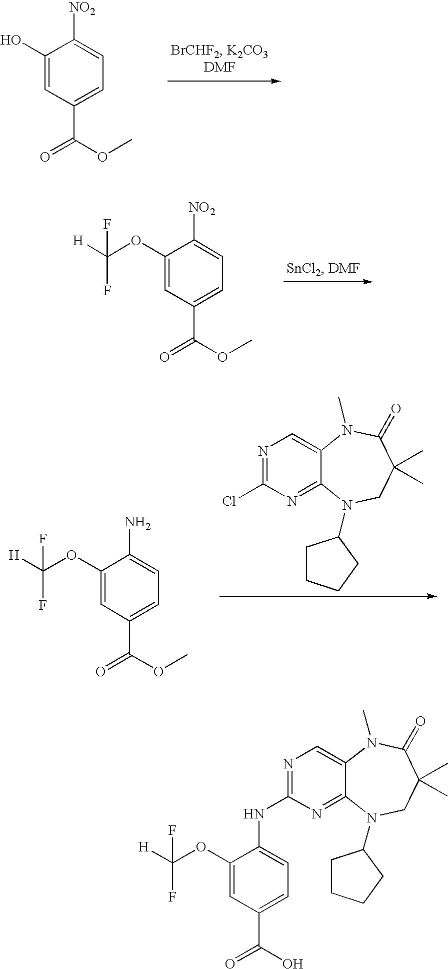

Methyl 3-(difluoromethoxy)-4-nitrobenzoate: To a solution of methyl 3-hydroxy-4-nitrobenzoate (1.0 g, 5.08 mmole), which was dissolved in DMF, anhydrous (50 ml) and chilled at 0° C., was added K₂CO₃ (1.05 g, 7.61 mmole). It was added, drop wise, iododifluoromethane (1.08 g, 6.09 mmole). The mixture was left at the ice bath and stirred overnight. Afterwards, the whole was poured into icy H₂O and was extracted with EtOAce. The organic layers were collected, dried over Na₂SO₄, and dried in vacuo. The resulting yellow solid (1.12 g, 89%), was carried onto the next step without any further purification. ¹H NMR (400 MHz, DMSO-d₆) δ ppm 0.00 (s, 1H) 3.93 (d, J=0.13 Hz, 3H) 7.27-7.72 (m, 1H) 7.94-7.99 (m, J=1.09, 1.09, 0.79, 0.57 Hz, 1H) 8.02 (dt, J=8.38, 0.89 Hz, 1H) 8.21 (d, J=8.40 Hz, 1H).

Methyl 4-amino-3-(difluoromethoxy)benzoate: To amount of 1.12 g (4.53 mmole) of methyl 3-(difluoromethoxy)-4-nitrobenzoate, which was dissolved in 50 ml THF, anhydrous, was added 3.07 g (13.59 mmole) of SnCl₂, at 0° C., and was stirred at r.t. overnight. The resulting mixture was poured into icy H₂O and was washed with EtOAce (2×300 ml). The organic layers were collected, dried over Na₂SO₄, and evaporated in vacuo to yield 900 mg (93%) of yellow solid. ¹H NMR (400 MHz, DMSO-d₆) δ ppm 3.80 (s, 3H) 7.15 (d, J=6.38 Hz, 2H) 7.55 (d, J=0.95 Hz, 1H) 7.76 (dd, J=8.53, 1.83 Hz, 1H) 8.82 (d, J=1.26 Hz, 1H) 8.97 (s, 1H). [M+H] calc'd for C₉H₉F₂NO₃, 218; found 218.

4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic acid: The title compound was synthesized using an analogous procedure same as that described in connection with 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid. [M+H] calc'd for C₂₃H₂₇F₂N₅O₄, 476; found 476.

### Compound 127

### 4-(9-Cyclopentyl-5,7,7-trimethyl-6-oxo-6,7,8,9-tetrahydro-5H-pyrimido[4,5-b][1,4]diazepin-2-ylamino)-3-(difluoromethoxy)-N-(1-methylpiperidin-4-yl)benzamide

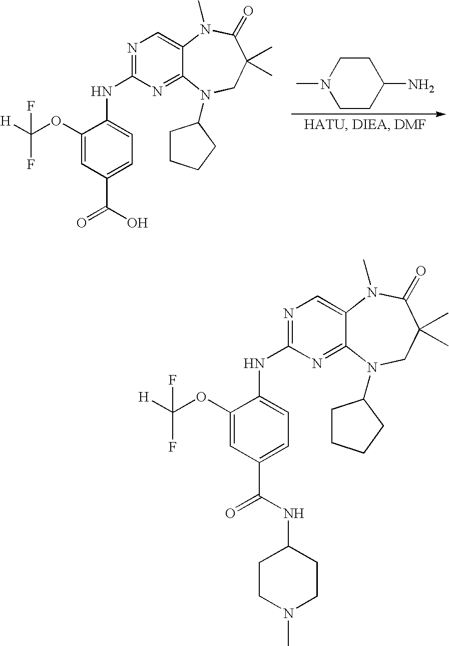

The title compound was synthesized using an analogous procedure same as that described in connection with Compound 10. ¹H NMR (400 MHz, MeOD) ppm 1.19 (s, 6H) 1.57-2.06 (m, 10H) 2.16 (d, J=12.00 Hz, 2H) 2.76 (s, 2H) 2.91-3.03 (m, 2H) 3.28 (s, 3H) 3.33-3.49 (m, 4H) 4.04-4.16 (m, 1H) 5.30 (quin, J=8.43 Hz, 1H) 6.72-7.15 (m, 1H) 7.71-7.77 (m, 2H) 7.94 (s, 1H) 8.55 (d, J=9.16 Hz, 1H). [M+H] calc'd for C₂₉H₃₉F₂N₇O₃, 572; found 572.

### Compound 128

### 4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic Acid

### Compound 129

### 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic Acid

### Compound 130

### 4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic Acid

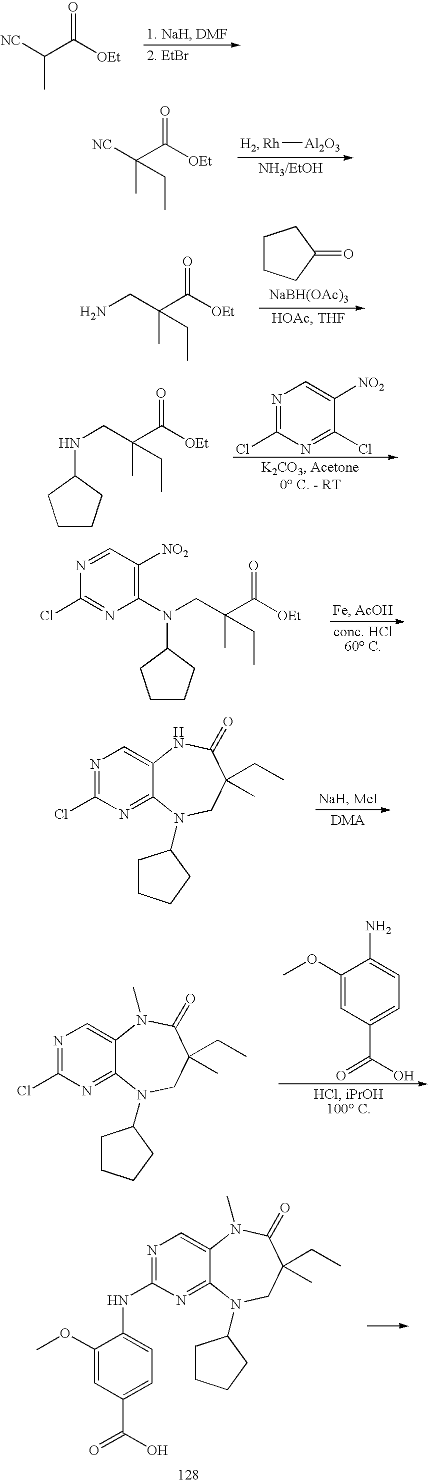

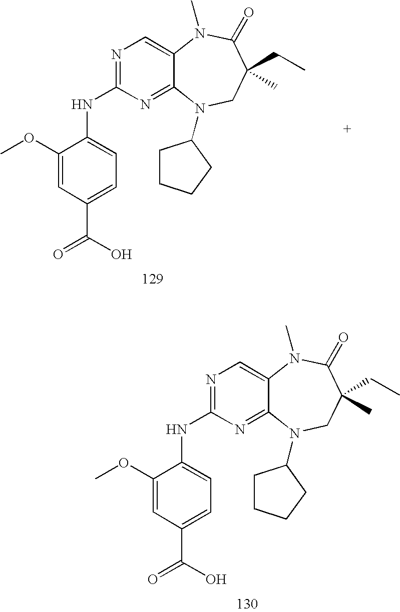

Ethyl 2-cyano-2-ethyl-2-methylbutanoate. The title compound was synthesized using an analogous procedure to that described in connection with ethyl 3-amino-2,2-dimethylpropanoate except that ethylbromide was used. ¹H NMR (400 MHz, CHLOROFORM-d) δ ppm 1.07 (t, J=7.4 Hz, 3H) 1.32 (t, J=7.2 Hz, 3H) 1.57 (s, 3H) 1.81 (dd, J=13.8, 7.4 Hz, 1H) 1.94-2.04 (m, 1H) 4.26 (qd, J=7.1, 1.5 Hz, 2H).

4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide: The title compound was synthesized using an analogous procedure to that described in connection with Compound 109 from the corresponding intermediate ethyl 3-amino-2,2-dimethylpropanoate and the final compound was purified by reverse phase HPLC as the TFA salt.

Ethyl 3-amino-2-ethyl-2-methylpropanoate. A solution of ethyl 2-cyano-2-ethyl 2-methylbutanoate in 1% ammonia in ethanol was hydrogenated with 5% rhodium on alumina for 20 h, after which, another portion of rhodium on alumina was added and the mixture was again hydrogenated for 20 h. When the reaction was done, the reaction mixture was filtered through celite. The filtrate was concentrated and dried to give the product, ethyl 3-amino-2-ethyl-2-methylpropanoate.

Ethyl 2-((cyclopentylamino)methyl)-2-ethyl-2-methylbutanoate. Ethyl 3-amino-2-ethyl-2-methyl-propanoate was treated with cyclopentanone (1.2 equivalent) in the presence of sodium triacetoxyborohydride (1.5 equivalent) and glacial acetic acid (15 mL) in tetrahydrofuran (150 ml) for 18 h at rt. The reaction mixture was carefully diluted with saturated aqueous NaHCO₃. The mixture was stirred for 1 hour at room temperature. The whole was extracted with ethyl acetate. The organic layer was dried over Na₂SO₄ and concentrated in vacuo to obtain ethyl 2-((cyclopentylamino)methyl)-2-ethyl-2-methylbutanoate as light yellow liquid.

Ethyl 3-((2-chloro-5-nitropyrimidin-4-yl)(cyclopentyl)amino)-2-ethyl-2-methylpropanoate: To a solution of 2,4-dichloro-5-nitropyrimidine (1.2 equivalent) in anhydrous acetone at 0° C., was added dropwise a solution of ethyl 2-((cyclopentylamino)methyl)-2-ethyl-2-methylbutanoate (1 equivalent) in acetone over 10 min. After which, potassium carbonate (2 equivalent) was added and the whole was stirred at rt for 18 h. After evaporation in vacuo, the residue was partitioned between ethyl acetate and water. The organic layer was washed with NaHCO₃, brine and water, dried over Na₂SO₄ and concentrated in vacuo. The residue was purified on silica gel chromatography (ethyl acetate:hexane=1:10 to 1:5) and recrystallized from ether/EtOAc to obtain ethyl 3-((2-chloro-5-nitropyrimidin-4-yl)(cyclopentyl)amino)-2-ethyl-2-methylpropanoate.

2-Chloro-9-cyclopentyl-7-ethyl-7-methyl-8,9-dihydro-5H-pyrimido[4,5-b][1,4]diazepin-6(7H)-one: To a suspension of ethyl 3-((2-chloro-5-nitropyrimidin-4-yl)(cyclopentyl)amino)-2-ethyl-2-methylpropanoate reduced iron (2 equivalent) in acetic acid (20 ml) was added dropwise concentrated hydrochloric acid (5 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2-chloro-9-cyclopentyl-7-ethyl-7-methyl-8,9-dihydro-5H-pyrimido[4,5-b][1,4]diazepin-6(7H)-one (in 50-60% yield) as white solid.

2-Chloro-9-cyclopentyl-7-ethyl-5,7-dimethyl-8,9-dihydro-5H-pyrimido[4,5-b][1,4]diazepin-6(7H)-one: To a solution of 2-Chloro-9-cyclopentyl-7-ethyl-7-methyl-8,9-dihydro-5H-pyrimido[4,5-b][1,4]diazepin-6(7H)-one in DMA was added sodium hydride (60% dispersion in mineral oil, (1.3 equivalent) at 0° C., followed by the dropwise addition of methyl iodide (1.3 equivalent). The reaction mixture was warmed up to rt and stirred for 1 h. The whole was poured into ice-water, extracted with ethyl acetate. The organic layer was washed with brine and dried over Na₂SO₄. The solution was concentrated in vacuo followed by precipitation from ether/EtOA to afford Compound 13-6 (70-79% yield) as white solid.

4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid: A mixture of 2-chloro-9-cyclopentyl-7-ethyl-5,7-dimethyl-8,9-dihydro-5H-pyrimido[4,5-b][1,4]diazepin-6(7H)-one, 4-amino-3-methoxybenzoic acid (1 equivalent), isopropanol (30 ml), and concentrated hydrochloric acid (20 drops) was stirred at 100° C. for 20 h. Solid was filtered to give 4-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid as white solid.

Compound 129: 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid and Compound 130: 4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid.

4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid and 4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid: 4-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid was further separated into two stereo isomers 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid and 4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid. The absolute configuration was determined by co-crystal structure after conversion of the carboxylic acid into amide.

### Compound 131

### 4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

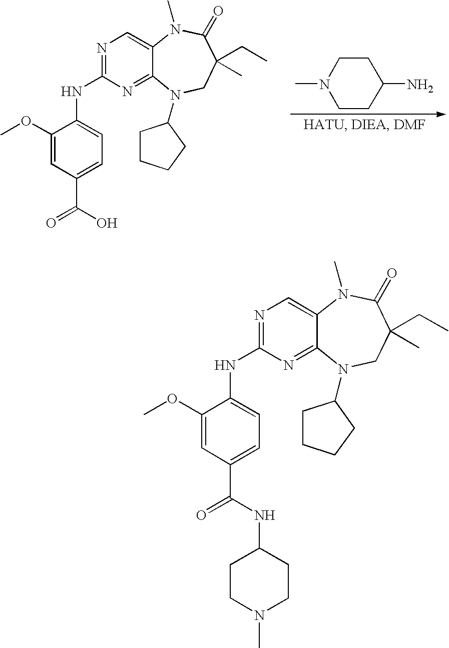

To a mixture of compound 4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid, DIEA (106 μL, 0.3 mmol) in 2.0 mL of anhydrous DMF was added HATU (114 mg, 0.3 mmol). The reaction mixture was stirred for 30 min. The reaction mixture was then diluted with ethyl acetate, washed with water and brine. The organic layer dried over Na₂SO₄ followed by HPLC purification. The TFA salt was then converted to free base, which was finally crystallized from ether to give title compound. ¹H NMR (400 MHz, DMSO-d₆) δ ppm 0.76 (t, J=7.4 Hz, 3H) 1.14 (s, 3H) 1.35 (d, J=6.6 Hz, 1H) 1.55 (t, J=6.8 Hz, 3H) 1.49-1.63 (m, 1H) 1.68-1.90 (m, 3H) 1.74 (d, J=9.4 Hz, 3H) 2.04 (d, J=12.4 Hz, 2H) 2.81 (d, J=5.0 Hz, 1H) 2.78 (d, J=4.5 Hz, 2H) 3.06-3.16 (m, 1H) 3.11 (d, J=12.6 Hz, 1H) 3.17 (s, 3H) 3.36 (d, J=14.6 Hz, 1H) 3.48 (d, J=11.6 Hz, 2H) 3.69 (d, J=14.9 Hz, 1H) 3.94 (s, 3H) 3.97-4.07 (m, 1H) 5.11 (d, J=7.8 Hz, 1H) 7.51-7.58 (m, 2H) 8.05 (s, 1H) 8.08 (d, J=8.6 Hz, 1H) 8.45 (d, J=7.6 Hz, 1H) 9.12 (br. s., 1H) 9.63 (br. s., 1H). [M+H] calc'd for C₃₀H₄₃N₇O₃, 550; found, 550.

### Compound 132

### (S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide

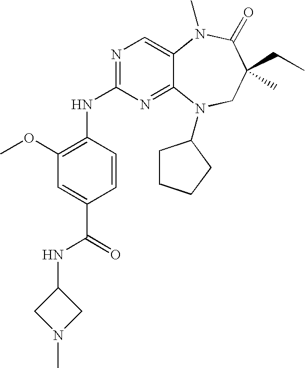

The title compound was synthesized from 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for Compound 131 except that 1-methylazetidin-3-amine was used and the final compound was purified by reverse phase HPLC and basified to give the free base. ¹H NMR (400 MHz, DMSO-d₆) δ ppm 0.74 (t, J=7.33 Hz, 3H) 1.12 (s, 3H) 1.29 (dd, J=14.02, 7.45 Hz, 2H) 1.40-2.02 (m, 12H) 2.33 (br. s., 3H) 3.18 (s, 3H) 3.56 (d, J=14.15 Hz, 3H) 3.95 (s, 3H) 4.40-4.50 (m, 1H) 5.16 (t, J=8.34 Hz, 1H) 7.48 (d, J=1.52 Hz, 1H) 7.71 (s, 1H) 8.00 (s, 1H) 8.38 (d, J=8.34 Hz, 1H) 8.65 (d, J=6.82 Hz, 1H) [M+H] calc'd for C₂₈H₃₉N₇O₃, 522; found 522.

### Compound 133

### (S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide

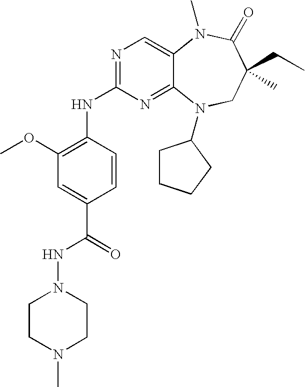

The title compound was synthesized from 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for Compound 131 except that 4-methylpiperazin-1-amine was used and the final compound was purified by reverse phase HPLC and basified to give the free base. ¹H NMR (400 MHz, DMSO-d₆) δ ppm 0.73 (t, J=7.45 Hz, 3H) 1.11 (s, 3H) 1.20-2.01 (m, 12H) 2.10-2.37 (m, 6H) 2.93 (br. s., 3H) 3.18 (s, 3H) 3.43-3.62 (m, 2H) 3.93 (s, 3H) 5.03-5.20 (m, 1H) 6.95 (dd, J=8.34, 1.77 Hz, 1H) 7.35-7.44 (m, 1H) 7.63-7.75 (m, 1H) 7.95-8.05 (m, 1H) 8.23-8.42 (m, 1H) 9.25-9.40 (m, 1H) [M+H] calc'd for C₂₉H₄₂N₈O₃, 551; found 551.

### Compound 134

### 4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide

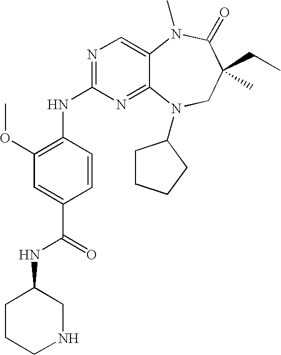

The title compound was synthesized from 4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for Compound 131 except that (S)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.45 Hz, 3H) 1.11 (s, 3H) 1.17-2.04 (m, 16H) 2.31-2.42 (m, 2H) 2.79 (d, J=2.02 Hz, 1H) 2.90-3.03 (m, 1H) 3.18 (s, 3H) 3.26 (d, J=13.89 Hz, 1H) 3.56 (d, J=13.89 Hz, 1H) 3.80 (t, J=13.89 Hz, 1H) 3.94 (s, 3H) 5.16 (quin, J=8.40 Hz, 1H) 7.46 (d, J=1.77 Hz, 1H) 7.68 (s, 1H) 7.98 (d, J=8.34 Hz, 2H) 8.36 (d, J=8.34 Hz, 1H). [M+H] C₂₉H₄₁N₇O₃ calc'd for, 536; found 536.

### Compound 135

### 4-((R)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide

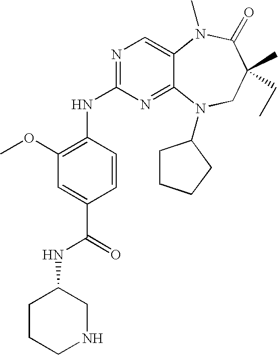

The title compound was synthesized from 4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for Compound 131 except that (R)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.45 Hz, 3H) 1.11 (s, 3H) 1.17-2.04 (m, 16H) 2.31-2.42 (m, 2H) 2.79 (d, J=2.02 Hz, 1H) 2.90-3.03 (m, 1H) 3.18 (s, 3H) 3.26 (d, J=13.89 Hz, 1H) 3.56 (d, J=13.89 Hz, 1H) 3.80 (t, J=13.89 Hz, 1H) 3.94 (s, 3H) 5.16 (quin, J=8.40 Hz, 1H) 7.46 (d, J=1.77 Hz, 1H) 7.68 (s, 1H) 7.98 (d, J=8.34 Hz, 2H) 8.36 (d, J=8.34 Hz, 1H). [M+H] C₂₉H₄₁N₇O₃ calc'd for, 536; found 536.

### Compound 136

### 4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic Acid

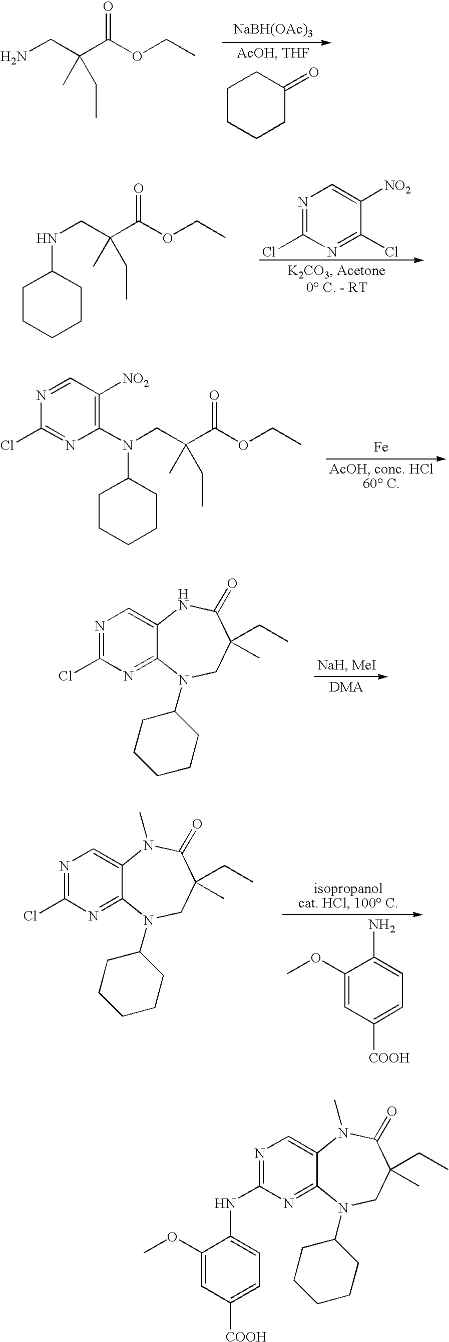

Ethyl 2-((cyclohexylamino)methyl)-2-ethyl-2-methylbutanoate. Ethyl 3-amino-2-ethyl-2-methyl-propanoate was treated with cyclohexanone (1.2 equivalent) in the presence of sodium triacetoxyborohydride (1.5 equivalent) and glacial acetic acid (15 mL) in tetrahydrofuran (150 ml) for 18 h at rt. The reaction mixture was carefully diluted with saturated aqueous NaHCO₃. The mixture was stirred for 1 hour at room temperature. The whole was extracted with ethyl acetate. The organic layer was dried over Na₂SO₄ and concentrated in vacuo to obtain ethyl 2-((cyclohexylamino)methyl)-2-ethyl-2-methylbutanoate as light yellow liquid.

Ethyl 3-((2-chloro-5-nitropyrimidin-4-yl)(cyclohexyl)amino)-2-ethyl-2-methylpropanoate: To a solution of 2,4-dichloro-5-nitropyrimidine (1.2 equivalent) in anhydrous acetone at 0° C., was added dropwise a solution of ethyl 2-((cyclohexylamino)methyl)-2-ethyl-2-methylbutanoate (1 equivalent) in acetone over 10 min. After which, potassium carbonate (2 equivalent) was added and the whole was stirred at rt for 18 h. After evaporation in vacuo, the residue was partitioned between ethyl acetate and water. The organic layer was washed with NaHCO₃, brine and water, dried over Na₂SO₄ and concentrated in vacuo. The residue was purified by silica gel chromatography (ethyl acetate:hexane=1:10 to 1:5) and recrystallized from ether/EtOAc to obtain ethyl 3-((2-chloro-5-nitropyrimidin-4-yl)(cyclohexyl)amino)-2-ethyl-2-methylpropanoate.

2-Chloro-9-cyclohexyl-7-ethyl-7-methyl-8,9-dihydro-5H-pyrimido[4,5-b][1,4]diazepin-6(7H)-one: To a suspension of ethyl 3-((2-chloro-5-nitropyrimidin-4-yl)(cyclohexyl)amino)-2-ethyl-2-methylpropanoate reduced iron (2 equivalent) in acetic acid (20 ml) was added dropwise concentrated hydrochloric acid (5 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2-chloro-9-cyclohexyl-7-ethyl-7-methyl-8,9-dihydro-5H-pyrimido[4,5-b][1,4]diazepin-6(7H)-one (in 50% yield) as white solid.

2-Chloro-9-cyclohexyl-7-ethyl-5,7-dimethyl-8,9-dihydro-5H-pyrimido[4,5-b][1,4]diazepin-6(7H)-one: To a solution of 2-Chloro-9-cyclohexyl-7-ethyl-7-methyl-8,9-dihydro-5H-pyrimido[4,5-b][1,4]diazepin-6(7H)-one in DMA was added sodium hydride (60% dispersion in mineral oil, (1.3 equivalent) at 0° C., followed by the dropwise addition of methyl iodide (1.3 equivalent). The reaction mixture was warmed up to rt and stirred for 1 h. The whole was poured into ice-water, extracted with ethyl acetate. The organic layer was washed with brine and dried over Na₂SO₄. The solution was concentrated in vacuo followed by precipitation from ether/EtOA to afford Compound 13-6 (70% yield) as white solid.

4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid: A mixture of 2-chloro-9-cyclohexyl-7-ethyl-5,7-dimethyl-8,9-dihydro-5H-pyrimido[4,5-b][1,4]diazepin-6(7H)-one, 4-amino-3-methoxybenzoic acid (1 equivalent), isopropanol (30 ml), and concentrated hydrochloric acid (20 drops) was stirred at 100° C. for 20 h. Solid was filtered to give 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid as white solid.

### Compound 137

### 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic Acid

### Compound 138

### 4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic Acid

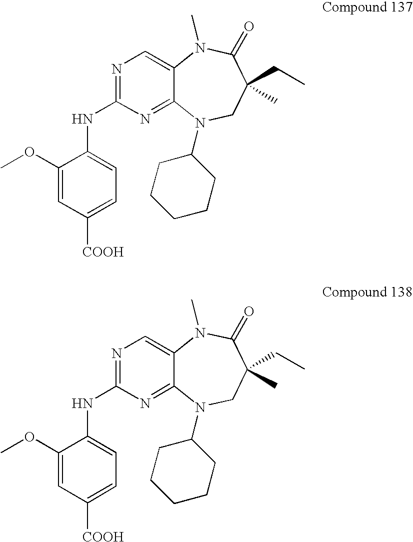

4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid can be further separated to two stereo isomer 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid and 4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid. The absolute configuration was determined by co-crystal structure after conversion of the carboxylic acid into amide.

### Compound 139

### 4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

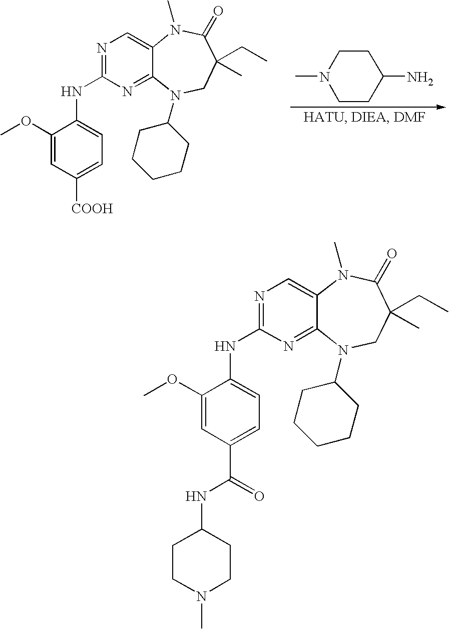

The title compound was synthesized from 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for Compound 131 and the final compound was purified by reverse phase HPLC and basified to give the free base. ¹H NMR (400 MHz, DMSO-d₆) δ ppm 0.77 (t, J=7.39 Hz, 3H) 0.83-0.92 (m, 2H) 1.16-1.43 (m, 8H) 1.49-1.77 (m, 4H) 1.84 (d, J=11.94 Hz, 1H) 1.99 (s, 3H) 2.72 (br. s., 3H) 3.02-3.12 (m, 2H) 3.17 (s, 3H) 3.34-3.50 (m, 2H) 3.57-3.69 (m, 2H) 3.96 (s, 3H) 4.11-4.16 (m, 1H) 4.61-4.73 (m, 1H) 7.55 (d, J=8.59 Hz, 1H) 7.65 (s, 1H) 7.69 (d, J=7.01 Hz, 1H) 8.10 (s, 2H) 8.55 (br. s., 1H). [M+H] calc'd for C₃₁H₄₅N₇O₃, 564; found 564.

### Compound 140

### 4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide

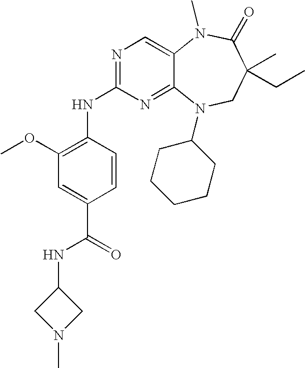

The title compound was synthesized from 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that 1-methylazetidin-3-amine was used followed by removal of boc using TFA in DCM and the final compound was purified by reverse phase HPLC and basified to give the free base. ¹H NMR (400 MHz, DMSO-d₆) δ ppm 0.74 (t, J=7.48 Hz, 3H) 1.11 (s, 3H) 1.18-1.68 (m, 8H) 1.78-1.95 (m, 4H) 2.27 (s, 3H) 2.99 (t, J=7.48 Hz, 2H) 3.17 (s, 3H) 3.39-3.67 (m, 4H) 3.95 (s, 3H) 4.36-4.51 (m, 1H) 4.60-4.83 (m, 1H) 7.41-7.57 (m, 2H) 7.69 (s, 1H) 7.98 (s, 1H) 8.37 (d, J=8.46 Hz, 1H) 8.65 (d, J=6.76 Hz, 1H). [M+H] calc'd for C₂₉H₄₁N₇O₃, 536; found 536.

### Compound 141

### 4-(9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

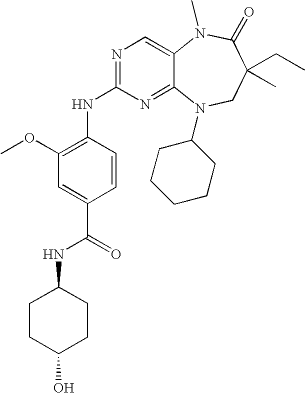

The title compound was synthesized from 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (1R,4R)-4-aminocyclohexanol was used and the final compound was purified by reverse phase HPLC and basified to give the free base. ¹H NMR (400 MHz, DMSO-d₆) δ ppm 0.75 (t, J=7.26 Hz, 3H) 1.11 (s, 3H) 1.14-1.93 (m, 19H) 3.17 (s, 3H) 3.32-3.53 (m, 4H) 3.66-3.82 (m, 1H) 3.94 (s, 3H) 4.54 (br. s., 1H) 4.70 (s, 1H) 7.46 (d, J=8.40 Hz, 2H) 7.65 (s, 1H) 7.97 (s, 1H) 8.01 (d, J=7.71 Hz, 1H) 8.34 (d, J=8.46 Hz, 1H) [M+H] calc'd for C₃₁H₄₄N₆O₄, 565; found 565.

### Compound 142

### 4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide

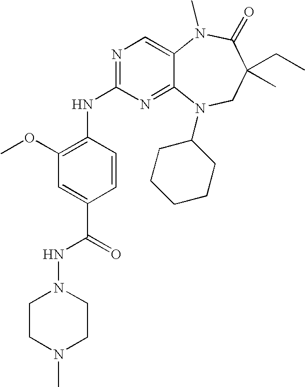

The title compound was synthesized from 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that 4-methylpiperazin-1-amine was used and the final compound was purified by reverse phase HPLC and basified to give the free base. ¹H NMR (400 MHz, DMSO-d₆) δ ppm 0.75 (t, J=7.45 Hz, 3H) 0.80-0.92 (m, 4H) 1.11 (s, 3H) 1.16-1.93 (m, 14H) 3.17 (s, 3H) 3.32-3.54 (m, 2H) 3.94 (s, 3H) 4.09-4.17 (m, 2H) 4.63-4.76 (m, 1H) 7.40-7.47 (m, 2H) 7.63-7.75 (m, 2H) 7.97 (s, 1H) 8.38 (d, J=8.34 Hz, 1H) [M+H] calc'd for C₃₀H₄₄N₈O₃, 565; found 565.

### Compound 143

### 4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide

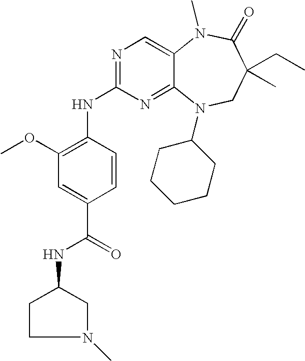

The title compound was synthesized from 4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (S)-1-methylpyrrolidin-3-amine was used and the final compound was purified by reverse phase HPLC and basified to give the free base. ¹H NMR (400 MHz, DMSO-d₆) δ ppm 0.75 (t, J=7.36 Hz, 3H) 1.11 (s, 3H) 1.13-1.95 (m, 16H) 2.11-2.22 (m, 1H) 2.27 (s, 3H) 2.31-2.42 (m, 1H) 2.58-2.71 (m, 2H) 3.17 (s, 3H) 3.32-3.52 (m, 2H) 3.95 (s, 3H) 7.45-7.56 (m, 2H) 7.66 (s, 1H) 7.97 (s, 1H) 8.36 (d, J=8.40 Hz, 2H). [M+H] calc'd for C₃₀H₄₃N₇O₃, 550; found 550.

### Compound 144

### (S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide

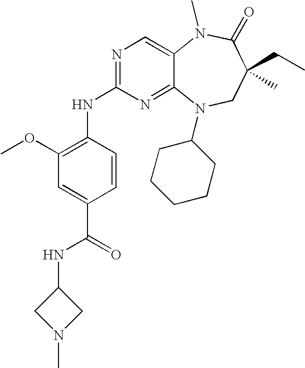

The title compound was synthesized from 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that 1-methylazetidin-3-amine was used and the final compound was purified by reverse phase HPLC and basified to give the free base. ¹H NMR (400 MHz, DMSO-d₆) δ ppm 0.75 (t, J=7.39 Hz, 3H) 1.11 (s, 3H) 1.16-1.93 (m, 16H) 2.86 (s, 3H) 3.17 (s, 3H) 3.32-3.54 (m, 2H) 3.97 (s, 3H) 4.62-4.79 (m, 2H) 7.53 (d, J=9.28 Hz, 1H) 7.60 (s, 1H) 7.71 (s, 1H) 7.98 (s, 1H) 8.41 (d, J=8.34 Hz, 1H) 9.13 (br. s., 1H) [M+H] calc'd for C₂₉H₄₁N₇O₃, 536; found 536.

### Compound 145

### (R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide

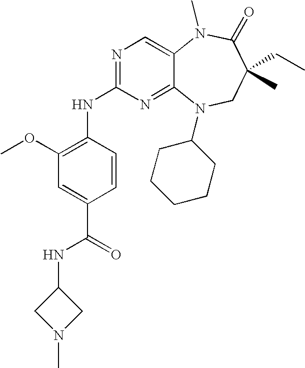

The title compound was synthesized from 4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that 1-methylazetidin-3-amine was used and the final compound was purified by reverse phase HPLC and basified to give the free base. ¹H NMR (400 MHz, DMSO-d₆) δ ppm 0.75 (t, J=7.39 Hz, 3H) 1.11 (s, 3H) 1.16-1.93 (m, 16H) 2.86 (s, 3H) 3.17 (s, 3H) 3.32-3.54 (m, 2H) 3.97 (s, 3H) 4.62-4.79 (m, 2H) 7.53 (d, J=9.28 Hz, 1H) 7.60 (s, 1H) 7.71 (s, 1H) 7.98 (s, 1H) 8.41 (d, J=8.34 Hz, 1H) 9.13 (br. s., 1H) [M+H] calc'd for C₂₉H₄₁N₇O₃, 536; found 536.

### Compound 146

### 4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

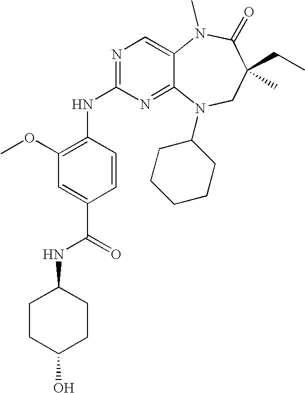

The title compound was synthesized from 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (1R,4R)-4-aminocyclohexanol was used and the final compound was purified by reverse phase HPLC and basified to give the free base. ¹H NMR (400 MHz, DMSO-d₆) δ ppm 0.70-0.78 (m, 3H) 1.11 (s, 3H) 1.14-1.92 (m, 20H) 3.17 (s, 3H) 3.32-3.55 (m, 3H) 3.66-3.83 (m, 1H) 3.94 (s, 3H) 4.54 (br. s., 1H) 4.67 (d, J=8.59 Hz, 1H) 7.42-7.52 (m, 2H) 7.65 (s, 1H) 7.96 (d, J=0.25 Hz, 2H) 8.34 (d, J=8.40 Hz, 1H) [M+H] calc'd for C₃₁H₄₄N₆O₄, 565; found 565.

### Compound 147

### 4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

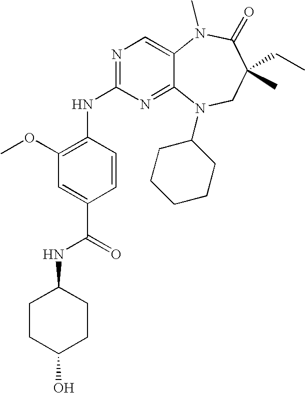

The title compound was synthesized from 4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (1R,4R)-4-aminocyclohexanol was used and the final compound was purified by reverse phase HPLC and basified to give the free base. ¹H NMR (400 MHz, DMSO-d₆) δ ppm 0.70-0.78 (m, 3H) 1.11 (s, 3H) 1.14-1.92 (m, 20H) 3.17 (s, 3H) 3.32-3.55 (m, 3H) 3.66-3.83 (m, 1H) 3.94 (s, 3H) 4.54 (br. s., 1H) 4.67 (d, J=8.59 Hz, 1H) 7.42-7.52 (m, 2H) 7.65 (s, 1H) 7.96 (d, J=0.25 Hz, 2H) 8.34 (d, J=8.40 Hz, 1H) [M+H] calc'd for C₃₁H₄₄N₆O₄, 565; found 565.

### Compound 148

### 4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide

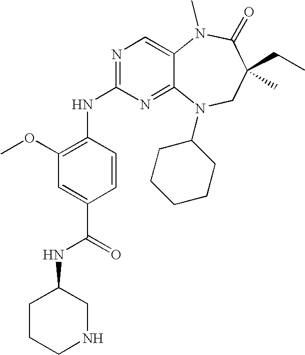

The title compound was synthesized from 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (S)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.33 Hz, 3H) 1.03-1.14 (m, 5H) 1.16-2.01 (m, 17H) 2.94 (d, J=1.01 Hz, 1H) 3.10 (d, J=11.87 Hz, 1H) 3.17 (s, 3H) 3.39 (d, J=1.52 Hz, 1H) 3.46-3.55 (m, 1H) 3.95 (br. s., 4H) 4.69 (t, J=12.63 Hz, 1H) 7.47 (d, J=8.84 Hz, 2H) 7.69 (s, 1H) 7.97 (s, 1H) 8.11 (d, J=7.33 Hz, 1H) 8.37 (d, J=8.34 Hz, 1H). [M+H] C₃₀H₄₃N₇O₃ calc'd for, 550; found 550.

### Compound 149

### 4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide

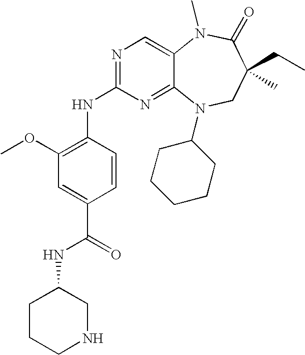

The title compound was synthesized from 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (R)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.33 Hz, 3H) 1.03-1.14 (m, 5H) 1.16-2.01 (m, 17H) 2.94 (d, J=1.01 Hz, 1H) 3.10 (d, J=11.87 Hz, 1H) 3.17 (s, 3H) 3.39 (d, J=1.52 Hz, 1H) 3.46-3.55 (m, 1H) 3.95 (br. s., 4H) 4.69 (t, J=12.63 Hz, 1H) 7.47 (d, J=8.84 Hz, 2H) 7.69 (s, 1H) 7.97 (s, 1H) 8.11 (d, J=7.33 Hz, 1H) 8.37 (d, J=8.34 Hz, 1H). [M+H] C₃₀H₄₃N₇O₃ calc'd for, 550; found 550.

### Compound 150

### 4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide

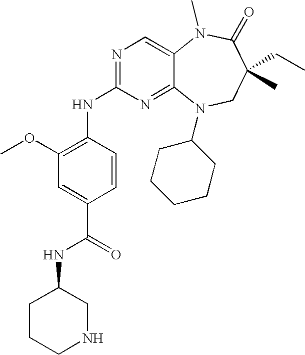

The title compound was synthesized from 4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (S)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.33 Hz, 3H) 1.03-1.14 (m, 5H) 1.16-2.01 (m, 17H) 2.94 (d, J=1.01 Hz, 1H) 3.10 (d, J=11.87 Hz, 1H) 3.17 (s, 3H) 3.39 (d, J=1.52 Hz, 1H) 3.46-3.55 (m, 1H) 3.95 (br. s., 4H) 4.69 (t, J=12.63 Hz, 1H) 7.47 (d, J=8.84 Hz, 2H) 7.69 (s, 1H) 7.97 (s, 1H) 8.11 (d, J=7.33 Hz, 1H) 8.37 (d, J=8.34 Hz, 1H). [M+H] C₃₀H₄₃N₇O₃ calc'd for, 550; found 550.

### Compound 151

### 4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide

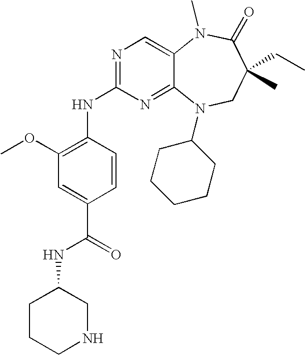

The title compound was synthesized from 4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid using an analogous procedure for the Compound 139 except that (R)-tert-butyl 3-aminopiperidine-1-carboxylate was used followed by removal of boc using TFA in DCM and the final compound was purified by reverse phase HPLC and basified to give the free base. ¹H NMR (400 MHz, DMSO-d₆) δ ppm 0.74 (t, J=7.33 Hz, 3H) 1.03-1.14 (m, 5H) 1.16-2.01 (m, 17H) 2.94 (d, J=1.01 Hz, 1H) 3.10 (d, J=11.87 Hz, 1H) 3.17 (s, 3H) 3.39 (d, J=1.52 Hz, 1H) 3.46-3.55 (m, 1H) 3.95 (br. s., 4H) 4.69 (t, J=12.63 Hz, 1H) 7.47 (d, J=8.84 Hz, 2H) 7.69 (s, 1H) 7.97 (s, 1H) 8.11 (d, J=7.33 Hz, 1H) 8.37 (d, J=8.34 Hz, 1H). [M+H] C₃₀H₄₃N₇O₃ calc'd for, 550; found 550.

### Compound 152

### 4-(9-Cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide

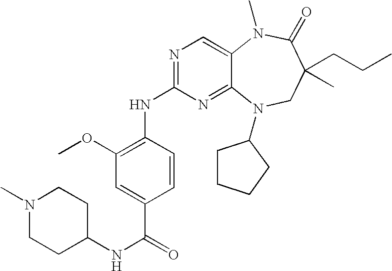

The title compound was synthesized using an analogous procedure to that described in connection with Compound 131 except that 1-bromopropane instead of iodoethane was used and the final compound was purified by reverse phase HPLC as the TFA salt. ¹H NMR (400 MHz, DMSO-d₆) δ ppm 0.77 (m, 3H) 1.16 (m, 4H) 1.31 (d, J=16.2 Hz, 2H) 1.44 (m, 1H) 1.56 (m, 3H) 1.74 (m, 6H) 2.02 (m, 3H) 2.79 (m, 3H) 3.36 (m, 1H) 3.47 (m, 2H) 3.66 (m, 1H) 3.94 (m, 5H) 5.11 (m, 2H) 7.56 (br. s., 2H) 8.04 (m, 2H) 8.44 (m, 1H) 9.09 (br. s., 1H) 9.53 (br. s., 1H). [M+H] calc'd for C₃₁H₄₅N₇O₃, 564; found, 564.

### Compound 153

### 4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide

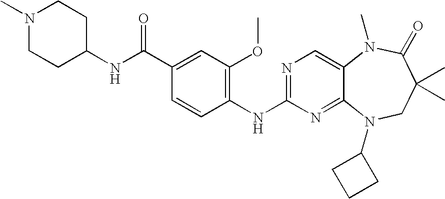

The title compound was synthesized using an analogous procedure to that described in connection with Compound 86 except that cyclobutanone instead of cyclopentanone was used and the final compound was purified by reverse phase HPLC followed by neutralization with base. ¹H NMR (400 MHz, DMSO-d₆) δ ppm 1.10 (s, 6H), 1.54-1.65 (m, 2H), 1.66-1.80 (m, 4H), 1.94 (t, J=10.86 Hz, 2H), 2.11-2.25 (m, 7H), 2.79 (d, J=11.62 Hz, 2H), 3.19 (s, 3H), 3.50 (s, 2H), 3.67-3.81 (m, 1H), 3.94 (s, 3H), 4.92-5.09 (m, 1H), 7.46-7.54 (m, 2H), 7.69 (s, 1H), 8.02 (s, 1H), 8.10 (d, J=7.83 Hz, 1H), 8.43 (d, J=8.34 Hz, 1H), [M+H] calc'd for C₂₈H₃₉N₇O₃ 522; found, 522

### Compound 154

### 4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide

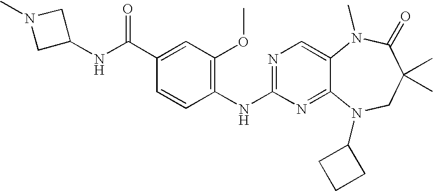

The title compound was synthesized using an analogous procedure to that described in connection with Compound 153 except that 1-methyl-3-amineazetidine instead of 1-methyl-4-aminopiperidine was used and the final compound was purified by reverse phase HPLC followed by neutralization with base. ¹H NMR (400 MHz, DMSO-d₆) δ ppm 1.09 (s, 6H), 1.65-1.75 (m, 2H), 2.12-2.22 (m, 4H), 2.46 (s, 3H), 3.18 (s, 3H), 3.50 (m, 4H), 3.80 (t, J=7.83 Hz, 2H), 3.95 (s, 3H), 4.50-4.56 (m, 1H), 5.00 (m, 1H), 7.49-7.56 (m, 2H), 7.72 (s, 1H), 8.02 (s, 1H), 8.45 (d, J=8.59 Hz, 1H), 8.75 (d, J=6.57 Hz, 1H), [M+H] calc'd for C₂₆H₃₅N₇O₃ 494; found, 494.

### Compound 155

### 4-(9-isopropyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide

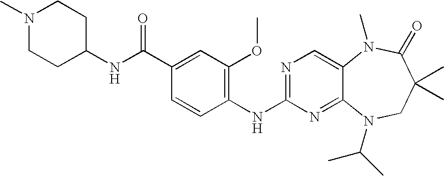

The title compound was synthesized using an analogous procedure to that described in connection with Compound 109 except that acetone instead of cyclopentanone was used and the final compound was purified by reverse phase HPLC followed by neutralization with base. ¹H NMR (400 MHz, CHLOROFORM-d) δ ppm 1.25-1.28 (m, 12H), 2.18-2.27 (m, 4H), 2.89 (d, J=4.29 Hz, 3H), 2.96-3.03 (m, 2H), 3.26 (s, 3H), 3.47 (s, 2H), 3.65 (d, J=12.13 Hz, 2H), 3.90 (s, 3H), 4.25-4.41 (m, 1H), 5.13-5.27 (m, 1H), 7.35-7.48 (m, 3H), 7.76 (s, 1H), 7.96 (d, J=8.34 Hz, 1H), 9.58 (s, 1H), 10.57 (br. s., 1H), [M+H] calc'd for C₂₇H₃₉N₇O₃ 510; found, 510.

### Compound 156

### 4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

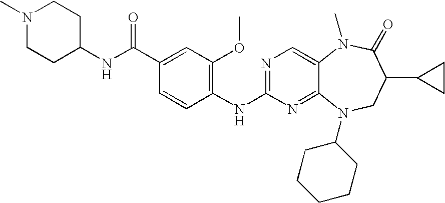

The title compound was synthesized using an analogous procedure to that described in connection with Compound 109 except that methyl 3-(cyclohexylamino)-2-cyclopropylpropanoate instead of methyl 3-(cyclohexylamino)propanoate was used and the final compound was purified by reverse phase HPLC followed by neutralization with base. ¹H NMR (400 MHz, CHLOROFORM-d) δ ppm 0.09-0.19 (m, 2H), 0.45-0.59 (m, 1H), 0.64-0.76 (m, 1H), 1.05-1.20 (m, 2H), 1.34-1.48 (m, 1H), 1.48-1.65 (m, 5H), 1.72-2.08 (m, 7H), 2.14 (t, J=10.86 Hz, 2H), 2.29 (s, 3H), 2.55 (br.s, 1H), 2.82 (d, J=11.62 Hz, 2H), 3.28 (s, 3H), 3.54-3.75 (m, 2H), 3.89-4.04 (m, 4H), 4.48 (m, 1H), 6.08 (d, J=7.83 Hz, 1H), 7.25 (m, 1H), 7.37 (d, J=1.77 Hz, 1H), 7.67 (s, 1H), 7.87 (s, 1H), 8.47 (d, J=8.59 Hz, 1H) [M+H] calc'd for C₃₁H₄₃N₇O₃ 562; found, 562.

### Compound 157

### 4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide

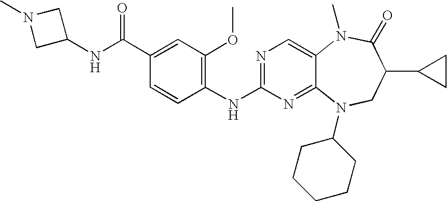

The title compound was synthesized using an analogous procedure to that described in connection with Compound 156 except that 3-amino-1-methylazetine instead of 4-amino-1-methylpiperidine was used and the final compound was purified by reverse phase HPLC followed by neutralization with base. ¹H NMR (400 MHz, CHLOROFORM-d) δ ppm 0.08-0.16 (m, 2H), 0.53-0.68 (m, 2H), 1.01-1.11 (m, 1H), 1.34-1.64 (m, 5H), 1.71-1.95 (m, 5H), 2.35 (s, 3H), 2.56 (br.s., 1H), 3.11-3.20 (m, 2H), 3.27 (s, 3H), 3.53-3.74 (m, 4H), 3.92 (s, 3H), 4.40-4.53 (m, 1H), 4.59-4.75 (m, 1H), 6.89 (d, J=7.58 Hz, 1H), 7.33 (dd, J=8.59, 1.77 Hz, 1H), 7.40 (d, J=1.77 Hz, 1H), 7.68 (s, 1H), 7.87 (s, 1H), 8.48 (d, J=8.34 Hz, 1H), [M+H] calc'd for C₂₉H₃₉N₇O₃ 534; found, 534.

### Compound 158

### 4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

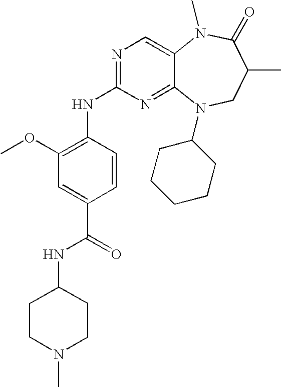

The title compound was synthesized using an analogous procedure to that described in connection with Compound 16 except that cyclohexaone instead of cyclopentanone was used and the final compound was purified by reverse phase HPLC followed by neutralization with base. ¹H NMR (400 MHz, DMSO-d₆) δ ppm 1.02-1.11 (m, 7H) 1.13-1.41 (m, 4H) 1.52-1.78 (m, 6H) 1.71-1.75 (m, 2H) 1.99 (s, 3H) 2.61-2.83 (m, 4H) 3.18 (s, 3H) 3.45-3.57 (m, 2H) 3.96 (s, 3H) 4.30-4.44 (m, 1H) 7.54 (d, J=9.16 Hz, 1H) 8.15 (br. s., 2H) 8.50 (br. s., 1H). [M+H] calc'd for C₂₉H₄₁N₇O₃, 536; found 536.

### Compound 159

### 4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide

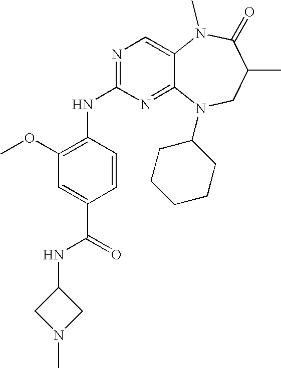

The title compound was synthesized using an analogous procedure to that described in connection with Compound 158 except that 3-amino-1-methylazetine instead of 4-amino-1-methylpiperidine was used and the final compound was purified by reverse phase HPLC followed by neutralization with base. ¹H NMR (400 MHz, DMSO-d₆) δ ppm 1.03 (s, 3H) 1.11-1.54 (m, 8H) 1.60-2.01 (m, 4H) 2.33 (s, 3H) 2.70-2.98 (m, 2H) 3.18 (s, 3H) 3.36-3.51 (m, 2H) 3.58-3.69 (m, 2H) 3.96 (s, 3H) 4.45 (q, J=6.91 Hz, 1H) 7.42-7.53 (m, 2H) 7.74 (s, 1H) 8.06 (s, 1H) 8.41 (d, J=8.34 Hz, 1H) 8.68 (d, J=6.82 Hz, 1H). [M+H] calc'd for C₂₇H₃₇N₇O₃, 508; found 508.

### Compound 160

### 4-(9-cyclohexyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

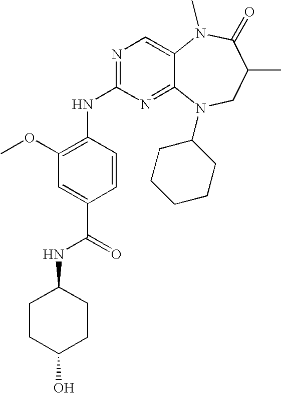

The title compound was synthesized using an analogous procedure to that described in connection with Compound 158 except that (1R,4R)-4-aminocyclohexanol of instead of 4-amino-1-methylpiperidine was used and the final compound was purified by reverse phase HPLC followed by neutralization with base. ¹H NMR (400 MHz, DMSO-d₆) δ ppm 1.03 (d, J=6.76 Hz, 3H) 1.08-1.63 (m, 8H) 1.63-1.97 (m, 11H) 2.77-2.89 (m, 1H) 3.18 (s, 3H) 3.30-3.49 (m, 4H) 3.67-3.79 (m, 1H) 4.27-4.36 (m, 1H) 4.49-4.56 (m, 1H) 7.42-7.52 (m, 3H) 7.70 (s, 1H) 8.01 (d, J=7.52 Hz, 1H) 8.05 (s, 1H) 8.37 (d, J=8.40 Hz, 1H).) [M+H] calc'd for C₂₉H₄₀N₆O₄, 537; found 537.

### Compound 161

### 4-(9-cyclohexyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide

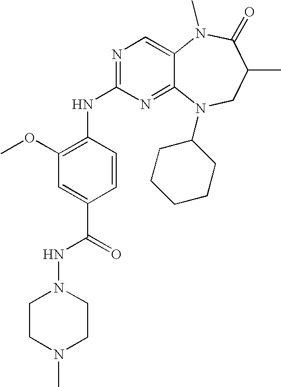

The title compound was synthesized using an analogous procedure to that described in connection with Compound 158 except that 4-methylpiperazin-1-amine of instead of 4-amino-1-methylpiperidine was used and the final compound was purified by reverse phase HPLC followed by neutralization with base. ¹H NMR (400 MHz, DMSO-d₆) δ ppm 0.75-0. (m, 3H) 1.11 (m, 6H) 1.16-1.93 (m, 14H) 3.17 (s, 3H) 3.32-3.54 (m, 2H) 3.94 (s, 3H) 4.09-4.17 (m, 2H) 4.63-4.76 (m, 1H) 7.40-7.47 (m, 2H) 7.63-7.75 (m, 2H) 7.97 (s, 1H) 8.38 (d, J=8.34 Hz, 1H) [M+H] calc'd for C₂₈H₄₀N₈O₃, 537; found 537.

### Compound 162

### 4-(9-cyclohexyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide

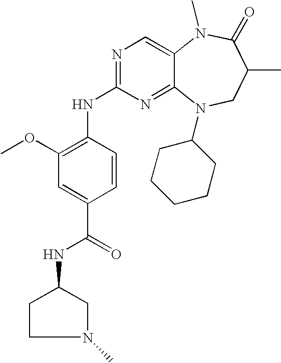

The title compound was synthesized using an analogous procedure to that described in connection with Compound 158 except that (R)-1-methylpyrrolidin-3-amine of instead of 4-amino-1-methylpiperidine was used and the final compound was purified by reverse phase HPLC followed by neutralization with base. ¹H NMR (400 MHz, DMSO-d₆) δ ppm 1.02-1.04 (s, 3H) 1.13-1.95 (m, 14H) 2.11-2.22 (m, 1H) 2.27 (s, 3H) 2.31-2.42 (m, 1H) 2.58-2.71 (m, 3H) 3.17 (s, 3H) 3.32-3.52 (m, 2H) 3.95 (s, 3H) 7.45-7.56 (m, 2H) 7.66 (s, 1H) 7.97 (s, 1H) 8.36 (d, J=8.40 Hz, 2H). [M+H] calc'd for C₂₈H₃₉N₇O₃, 522; found 522.

### Compound 163

### 4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide

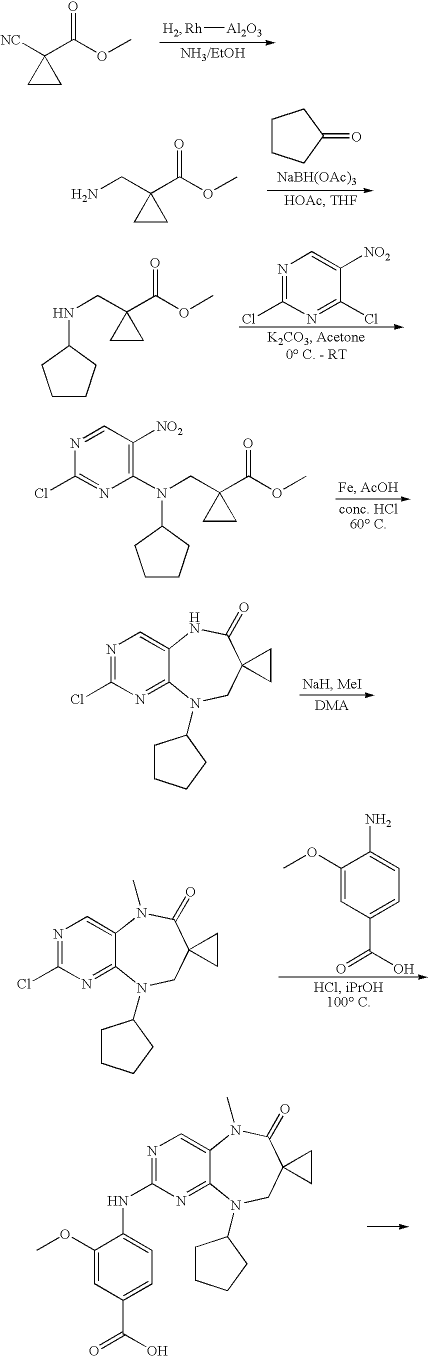

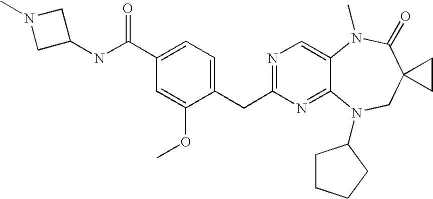

Methyl 1-cyanocyclopropanecarboxylate. To a solution of sodium ethoxide (21 wt % in EtOH, 5.79 mL, 15.5 mmol) in EtOH (25 mL), was added ethyl cyanoacetate (1.12 mL, 10.5 mmol), followed shortly thereafter by 1,1-dibromopropane (1.12 mL, 10 mmol). The reaction mixture was refluxed for 3 hrs. It was then concentrated, and diluted to ethyl acetate. The organic layer was washed with NaHCO₃, brine, water, dried over Na₂SO₄ and concentrated in vacuo to obtain ethyl 1-cyanocyclobutanecarboxylate (>70% yield) as a red liquid. It was used directly for next step reaction.

Methyl 1-(aminomethyl)cyclopropanecarboxylate. A solution of compound ethyl 1-cyanocyclopropanecarboxylate (2 g, 13 mmol) in 1% ammonia in ethanol (20 mL) was hydrogenated with 5% rhodium on alumina (0.8 g) over 3 days. The reaction mixture was filtered through celite. The filtrate was concentrated and dried to give the product, compound 3 (1.5 g, 75%) as light yellow liquid. [M+H] calc'd for C₆H₁₁ClNO₂, 130; found, 130.

Methyl 1-((cyclopentylamino)methyl)cyclopropanecarboxylate. Ethyl 1-(aminomethyl)cyclopropanecarboxylate (1.5 g, 9.6 mmol) was treated with cyclopentanone (1.02 ml, 11.5 mmol) in the presence of sodium triacetoxyborohydride (3 g, 14.4 mmol) and glacial acetic acid (3 mL) in tetrahydrofuran (100 ml) for 18 hours at room temperature. The reaction mixture was concentrated and carefully diluted with saturated aqueous NaHCO₃. The whole was extracted with ethyl acetate (200 ml×3). The organic layer was dried over Na₂SO₄ and concentrated in vacuo to obtain ethyl 1-((cyclopentylamino)methyl)cyclobutanecarboxylate (1.67 g, 77% yield) as light yellow liquid. [M+H] calc'd for C₁₁H₁₉ClNO₂, 198; found, 198.

Methyl 1-(((2-chloro-5-nitropyrimidin-4-l)(cyclopentyl)amino)methyl)cyclopropanecarboxylate. To a solution of 2,4-dichloro-5-nitropyrimidine (1.46 g, 7.5 mmol) in acetone (25 ml) at 0° C., was added ethyl 1-((cyclopentylamino)methyl)cyclobutanecarboxylate (1.67 g, 7.42 mmol) in acetone (5 ml) dropwise, followed by potassium carbonate (2.07 g, 15 mmol) and the whole was stirred at room temperature for 18 hours. After evaporation in vacuo, the residue was partitioned between ethyl acetate (200 ml) and water (200 ml). The organic layer was washed with NaHCO₃, brine and water, dried over Na₂SO₄ and concentrated in vacuo. This mixture was used for next step reaction without further purification. [M+H] calc'd for C₁₅H₁₉ClN₄O₄, 355; found, 355.

2′-Chloro-9′-cyclopentyl-8′,9′-dihydrospiro[cyclopropane-1,7′-pyrimido[5,4-b][1,4]diazepin]-6′(5′H)-one. To a suspension of methyl 1-(((2-chloro-5-nitropyrimidin-4-l)(cyclopentyl)amino)methyl)cyclopropanecarboxylate reduced iron (2 equivalent) in acetic acid (20 ml) was added dropwise concentrated hydrochloric acid (5 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2′-chloro-9′-cyclopentyl-8′,9′-dihydrospiro[cyclopropane-1,7′-pyrimido[5,4-b][1,4]diazepin]-6′(5′H)-one (in ˜50% yield) as white solid. [M+H] calc'd for C₁₄H₁₇ClN₄O, 293; found, 293.

2′-Chloro-9′-cyclopentyl-5′-methyl-8′,9′-dihydrospiro[cyclopropane-1,7′-pyrimido[5,4-b][1,4]diazepin]-6′(5′H)-one. To a solution of 2′-chloro-9′-cyclopentyl-8′,9′-dihydrospiro[cyclopropane-1,7′-pyrimido[5,4-b][1,4]diazepin]-6′(5′H)-one in DMA was added sodium hydride (60% dispersion in mineral oil, (1.3 equivalent) at 0° C., followed by the dropwise addition of methyl iodide (1.3 equivalent). The reaction mixture was warmed up to rt and stirred for 1 h. The whole was poured into ice-water, extracted with ethyl acetate. The organic layer was washed with brine and dried over Na₂SO₄. The solution was concentrated in vacuo followed by precipitation from ether/EtOA to afford 2′-chloro-9′-cyclopentyl-5′-methyl-8′,9′-dihydrospiro[cyclopropane-1,7′-pyrimido[5,4-b][1,4]diazepin]-6′(5′H)-one (70% yield) as white solid. [M+H] calc'd for C₁₅H₁₉ClN₄O, 307; found, 307.

4-(9′-Cyclopentyl-5′-methyl-6′-oxo-5′,6′,8′,9′-tetrahydrospiro[cyclopropane-1,7′-pyrimido[5,4-b][1,4]diazepine]-2′-ylamino)-3-methoxybenzoic acid. A mixture of 2′-chloro-9′-cyclopentyl-5′-methyl-8′,9′-dihydrospiro[cyclopropane-1,7′-pyrimido[5,4-b][1,4]diazepin]-6′(5′H)-one, 4-amino-3-methoxybenzoic acid (1 equivalent), isopropanol (30 ml), and concentrated hydrochloric acid (20 drops) was stirred at 100° C. for 20 h. Solid was filtered to give 4-(9′-cyclopentyl-5′-methyl-6′-oxo-5′,6′,8′,9′-tetrahydrospiro[cyclopropane-1,7′-pyrimido[5,4-b][1,4]diazepine]-2′-ylamino)-3-methoxybenzoic acid as white solid. ¹H NMR (400 MHz, DMSO-d₆) δ ppm 0.71-0.85 (m, 2H) 0.97-1.09 (m, 2H) 1.43-1.62 (m, 4H) 1.62-1.77 (m, 2H) 1.77-1.93 (m, 2H) 3.16 (s, 3H) 3.65 (br. s., 2H) 3.80 (s, 1H) 3.95 (s, 3H) 4.78-4.97 (m, 1H) 7.57-7.62 (m, 2H) 8.06 (s, 1H) 8.14 (d, J=8.84 Hz, 1H) 9.44 (br. s., 1H). [M+H] calc'd for C₂₃H₂₇ClN₅O₄, 438; found, 438.

4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide. The title compound was synthesized using an analogous procedure described for Compound 33 except that 1-methylazetidin-3-amine was used and the final compound was purified by reverse phase HPLC followed by neutralization with base. ¹H NMR (400 MHz, DMSO-d₆) δ ppm 0.69 (d, J=11.12, 0.25 Hz, 2H) 0.91 (dd, J=11.12, 4.80 Hz, 2H) 1.42-1.79 (m, 6H) 1.90 (br. s., 2H) 2.27 (s, 3H) 2.99 (t, J=7.20 Hz, 2H) 3.18 (s, 3H) 3.48 (s, 2H) 3.56 (t, J=7.20 Hz, 2H) 3.96 (s, 3H) 4.43 (q, J=6.82 Hz, 1H) 4.86 (t, J=8.59 Hz, 1H) 7.48 (d, J=1.77 Hz, 1H) 7.51 (s, 1H) 7.69 (s, 1H) 7.99 (s, 1H) 8.41 (d, J=8.34 Hz, 1H) 8.59 (d, J=6.82 Hz, 1H). [M+H] calc'd for C₂₇H₃₅N₇O₃ 506; found, 506.

### Compound 164

### (S)-4-(9′-cyclopentyl-5′-methyl-6′-oxo-5′,6′,8′,9′-tetrahydrospiro-[cyclopropane-1,7′-pyrimido[4,5-b][1,4]diazepine]-2′-ylamino)-3-methoxy-N-(1-methylpiperidin-3-yl)benzamide

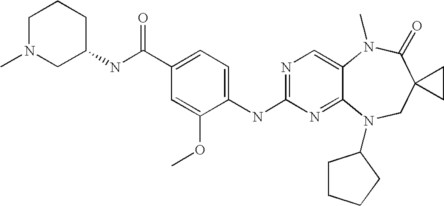

The title compound was synthesized using an analogous procedure described for Compound 33 except that (S)-1-methylpiperidin-3-amine was used and the final compound was purified by reverse phase HPLC followed by neutralization with base. ¹H NMR (400 MHz, DMSO-d₆) δ ppm 0.63-0.71 (m, 2H) 0.85-0.94 (m, 2H) 1.31 (dd, J=11.62, 3.79 Hz, 1H) 1.42-1.99 (m, 13H) 2.19 (s, 3H) 2.68 (d, J=8.59 Hz, 1H) 2.83 (d, J=9.60 Hz, 1H) 3.17 (s, 3H) 3.48 (s, 2H) 3.95 (s, 4H) 4.85 (t, J=8.59 Hz, 1H) 7.46 (s, 1H) 7.49 (s, 1H) 7.69 (s, 1H) 7.99 (s, 1H) 8.04 (d, J=7.83 Hz, 1H) 8.40 (d, 1H). [M+H] calc'd for C₂₉H₃₉N₇O₃ 534; found, 534.

### Compound 165

### (R)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide

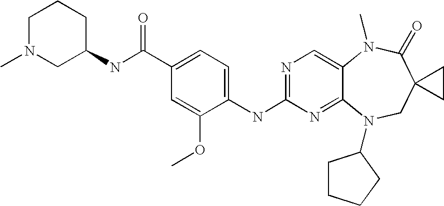

The title compound was synthesized using an analogous procedure described for Compound 33 except that (R)-1-methylpiperidin-3-amine was used and the final compound was purified by reverse phase HPLC followed by neutralization with base ¹H NMR (400 MHz, CHLOROFORM-d) □ ppm 0.61-0.72 (m, 2H) 0.84-0.95 (m, 2H) 1.22-1.39 (m, 1H) 1.41-1.97 (m, 12H) 2.17 (s, 3H) 2.60-2.73 (m, 2H) 2.80 (d, J=14.91 Hz, 1H) 3.16 (s, 3H) 3.47 (s, 2H) 3.94 (s, 4H) 4.81-4.88 (m, 1H) 7.45 (s, 1H) 7.49 (s, 1H) 7.67 (s, 1H) 7.95-8.01 (m, 2H) 8.38 (d, J=8.08 Hz, 1H). [M+H] calc'd for C₂₉H₃₉N₇O₃ 534; found, 534.

### Compound 166

### (S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide

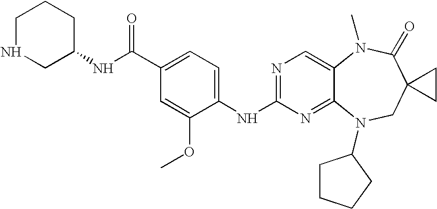

The title compound was synthesized using an analogous procedure described for Compound 33 except that (S)-tert-butyl 3-aminopiperidine-1-carboxylate followed by removal of Boc using TFA in DCM and the final compound was purified by reverse phase HPLC followed by neutralization with base. ¹H NMR (400 MHz, CHLOROFORM-d) δ ppm 0.64-0.71 (m, 2H) 0.86-0.96 (m, 2H) 1.32-1.77 (m, 9H) 1.79-1.97 (m, 3H) 2.29-2.46 (m, 3H) 2.81 (d, J=11.62 Hz, 1H) 2.97 (d, J=14.65 Hz, 1H) 3.17 (s, 3H) 3.48 (s, 2H) 3.74-3.89 (m, 1H) 3.95 (s, 3H) 4.85 (t, J=8.84 Hz, 1H) 7.45 (s, 1H) 7.49 (s, 1H) 7.68 (s, 1H) 7.95 (d, J=7.33 Hz, 1H) 7.99 (s, 1H) 8.39 (d, J=8.34 Hz, 1H). [M+H] calc'd for C₂₇₉H₃₇N₇O₃ 520; found, 520.

### Compound 167

### (S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpyrrolidin-3-yl)benzamide

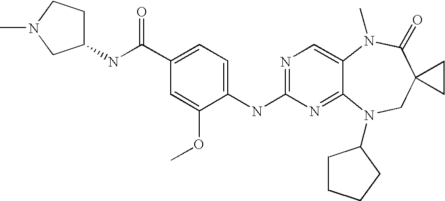

The title compound was synthesized using an analogous procedure described for Compound 33 except that (S)-1-methylpyrrolidin-3-amine and the final compound was purified by reverse phase HPLC followed by neutralization with base. ¹H NMR (400 MHz, CHLOROFORM-d) δ ppm 0.64-0.72 (m, 2H) 0.85-0.98 (m, 2H) 1.41-1.97 (m, 10H) 2.10-2.26 (m, 1H) 2.31 (s, 3H) 2.64-2.80 (m, 3H) 3.18 (s, 3H) 3.48 (s, 2H) 3.96 (s, 3H) 4.36-4.49 (m, 1H) 4.79-4.93 (m, 1H) 7.48 (s, 1H) 7.52 (s, 1H) 7.68 (s, 1H) 7.99 (s, 1H) 8.33 (d, J=7.07 Hz, 1H) 8.40 (d, J=8.34 Hz, 1H). [M+H] calc'd for C₂₇H₃₇N₇O₃ 520; found, 520.

### Compound 168

### 4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide

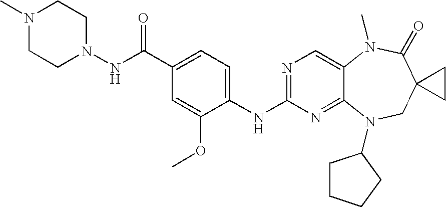

The title compound was synthesized using an analogous procedure described for Compound 33 except that 4-methylpiperazin-1-amine was used and the final compound was purified by reverse phase HPLC followed by neutralization with base. ¹H NMR (400 MHz, DMSO-d₆) δ ppm 0.62-0.71 (m, 2H) 0.85-0.94 (m, 2H) 1.42-1.77 (m, 6H) 1.88 (br. s., 2H) 2.21 (s, 3H) 2.35-2.49 (m, 4H) 2.93 (t, J=4.55 Hz, 4H) 3.16 (s, 3H) 3.47 (s, 2H) 3.94 (s, 3H) 4.84 (t, J=8.59 Hz, 1H) 7.40 (s, 1H) 7.42 (s, 1H) 7.69 (s, 1H) 7.98 (s, 1H) 8.39 (d, J=8.08 Hz, 1H) 9.34 (s, 1H). [M+H] calc'd for C₂₈H₃₈N₈O₃ 535; found, 535.

### Compound 169

### 4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-4-yl)benzamide

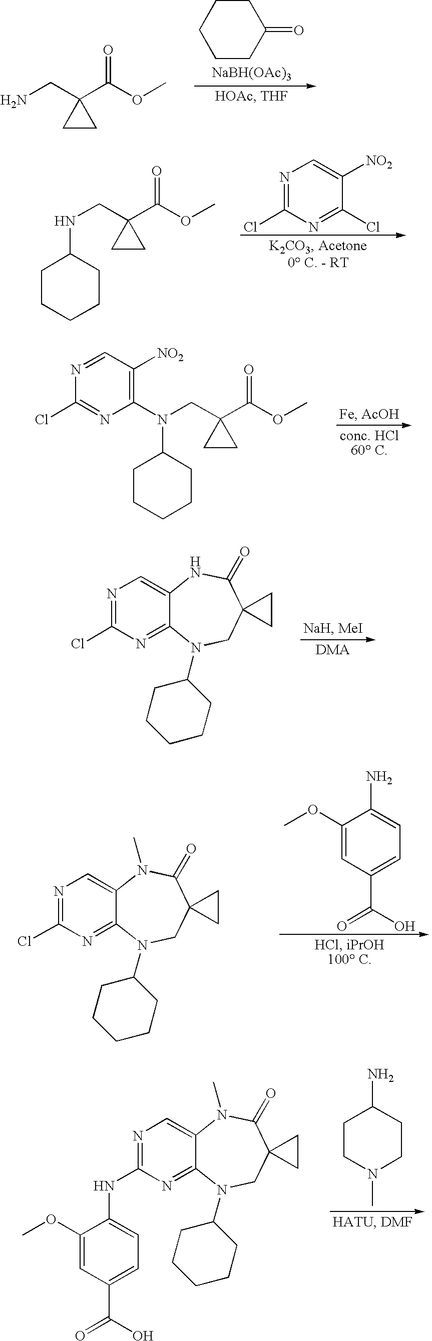

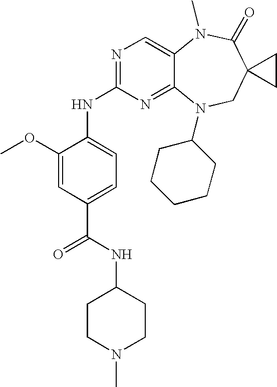

The title compound was synthesized using an analogous procedure described for Compound 33. except that cyclohexanone instead of cyclopentanone was used for the reductive amination and the final compound was purified by reverse phase HPLC followed by neutralization with base. ¹H NMR (400 MHz, DMSO-d₆) δ ppm 0.63-0.71 (m, 2H) 0.88-0.95 (m, 2H) 1.06-1.23 (m, 1H) 1.31-1.88 (m, 13H) 1.89-2.03 (m, 2H) 2.18 (s, 3H) 2.80 (d, J=11.62 Hz, 2H) 3.15 (s, 3H) 3.50 (s, 2H) 3.67-3.82 (m, 1H) 3.95 (s, 3H) 4.43 (br. s., 1H) 7.46 (d, J=9.09 Hz, 1H) 7.50 (s, 1H) 7.67 (s, 1H) 7.95 (s, 1H) 8.12 (d, J=7.83 Hz, 1H) 8.39 (d, 1H). [M+H] calc'd for C₃₀H₄₁N₇O₃ 548; found, 548.

### Compound 170

### 4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide

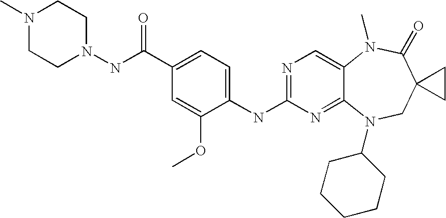

The title compound was synthesized using an analogous procedure described for Compound 169 except that 4-methylpiperazin-1-amine was used and the final compound was purified by reverse phase HPLC followed by neutralization with base. ¹H NMR (400 MHz, DMSO-d₆) δ ppm 0.65-0.73 (m, 2H) 0.87-0.95 (m, 2H) 1.06-1.23 (m, 1H) 1.32-1.54 (m, 4H) 1.63-1.88 (m, 5H) 2.21 (br. s, 3H) 2.34-2.48 (m, 4H) 2.95 (br. s., 4H) 3.15 (s, 3H) 3.50 (s, 2H) 3.94 (s, 3H) 4.37-4.49 (m, 1H) 7.38-7.45 (m, 2H) 7.67 (s, 1H) 7.95 (s, 1H) 8.39 (d, J=8.34 Hz, 1H) 9.38 (s, 1H). [M+H] calc'd for C₂₉H₄₁N₈O₃ 549; found, 549.

### Compound 171

### (S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)-benzamide

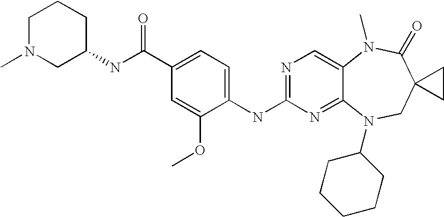

The title compound was synthesized using an analogous procedure described for Compound 169 except that (S)-1-methylpiperidin-3-amine was used and the final compound was purified by reverse phase HPLC followed by neutralization with base. ¹H NMR (400 MHz, DMSO-d₆) δ ppm 0.66-0.72 (m, 2H) 0.88-0.95 (m, 2H) 1.07-1.90 (m, 16H) 2.18 (s, 3H) 2.68 (d, J=10.11 Hz, 1H) 2.83 (d, J=9.09 Hz, 1H) 3.16 (s, 3H) 3.50 (s, 2H) 3.95 (s, 4H) 4.37-4.51 (m, 1H) 7.47 (d, J=8.59 Hz, 1H) 7.50 (s, 1H) 7.68 (s, 1H) 7.96 (s, 1H) 8.05 (d, J=8.08 Hz, 1H) 8.39 (d, J=8.34 Hz, 1H). [M+H] calc'd for C₃₀H₄₁N₇O₃ 548; found, 548.

### Compound 172

### (R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide

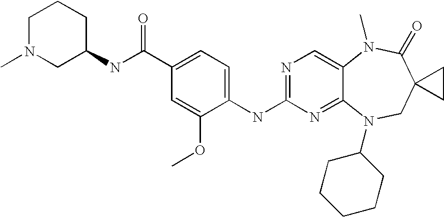

The title compound was synthesized using an analogous procedure described for Compound 169 except that (R)-1-methylpiperidin-3-amine was used and the final compound was purified by reverse phase HPLC followed by neutralization with base. ¹H NMR (400 MHz, DMSO-d₆) δ ppm 0.65-0.71 (m, 2H) 0.89-0.94 (m, 2H) 1.04-1.90 (m, 16H) 2.17 (s, 3H) 2.67 (d, J=10.61 Hz, 1H) 2.82 (d, J=10.36 Hz, 1H) 3.15 (s, 3H) 3.50 (s, 2H) 3.95 (s, 4H) 4.38-4.49 (m, 1H) 7.46 (dd, J=8.34, 1.77 Hz, 1H) 7.50 (d, J=1.77 Hz, 1H) 7.67 (s, 1H) 7.95 (s, 1H) 8.05 (d, J=8.08 Hz, 1H) 8.39 (d, 1H). [M+H] calc'd for C₃₀H₄₁N₇O₃ 548; found, 548.

### Compound 173

### 4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide

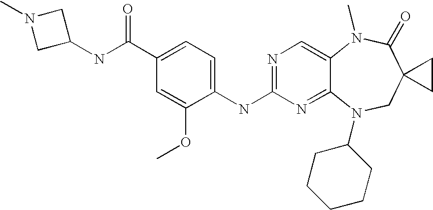

The title compound was synthesized using an analogous procedure described for Compound 169 except that 1-methylazetidin-3-amine was used and the final compound was purified by reverse phase HPLC followed by neutralization with base. ¹H NMR (400 MHz, DMSO-d₆) δ ppm 0.66-0.71 (m, 2H) 0.89-0.94 (m, 2H) 1.07-1.22 (m, 1H) 1.32-1.55 (m, 4H) 1.61-1.90 (m, 5H) 2.26 (s, 3H) 2.99 (t, J=7.20 Hz, 2H) 3.15 (s, 3H) 3.50 (s, 2H) 3.56 (t, J=7.20 Hz, 2H) 3.96 (s, 3H) 4.36-4.50 (m, 2H) 7.48 (dd, J=8.46, 1.64 Hz, 1H) 7.52 (d, J=1.77 Hz, 1H) 7.68 (s, 1H) 7.95 (s, 1H) 8.40 (d, J=8.34 Hz, 1H) 8.64 (d, 1H). [M+H] calc'd for C₂₈H₃₇N₇O₃ 520; found, 520.

### Compound 174

### (S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide

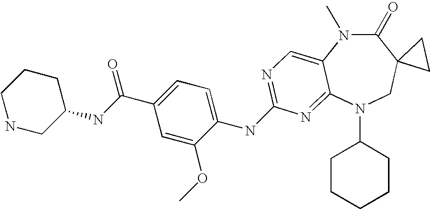

The title compound was synthesized using an analogous procedure described for Compound 169 except that (S)-tert-butyl 3-aminopiperidine-1-carboxylate followed by removal of Boc using TFA in DCM and the final compound was purified by reverse phase HPLC followed by neutralization with base. ¹H NMR (400 MHz, DMSO-d₆) δ ppm 0.64-0.73 (m, 2H) 0.87-0.95 (m, 2H) 1.06-1.21 (m, 2H) 1.32-1.59 (m, 6H) 1.61-1.94 (m, 7H) 2.51-2.58 (m, 2H) 2.93 (d, J=11.87 Hz, 1H) 3.08 (d, J=11.62 Hz, 1H) 3.15 (s, 3H) 3.49 (s, 2H) 3.86-3.98 (m, 4H) 4.37-4.49 (m, 1H) 7.46 (dd, J=8.34, 1.77 Hz, 1H) 7.49 (d, J=1.77 Hz, 1H) 7.68 (s, 1H) 7.95 (s, 1H) 8.10 (d, J=7.83 Hz, 1H) 8.39 (d, J=8.34 Hz, 1H). [M+H] calc'd for C₂₉H₃₉N₇O₃ 534; found, 534.

### Compound 175

### (R)-4-(9′-cyclohexyl-5′-methyl-6′-oxo-5′,6′,8′,9′-tetrahydrospiro [cyclopropane-1,7′-pyrimido[4,5-b][1,4]diazepine]-2′-ylamino)-3-methoxy-N-(piperidin-3-yl)benzamide

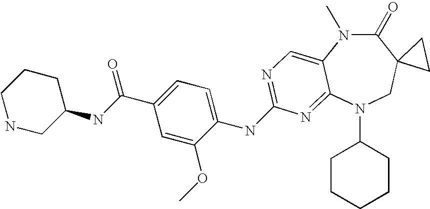

The title compound was synthesized using an analogous procedure described for Compound 169 except that (R)-tert-butyl 3-aminopiperidine-1-carboxylate followed by removal of Boc using TFA in DCM and the final compound was purified by reverse phase HPLC followed by neutralization with base. ¹H NMR (400 MHz, DMSO-d₆) δ ppm 0.65-0.70 (m, 2H) 0.89-0.94 (m, 2H) 1.06-1.21 (m, 2H) 1.30-1.58 (m, 6H) 1.62-1.92 (m, 6H) 2.51-2.56 (m, 3H) 2.93 (d, J=11.87 Hz, 1H) 3.08 (dd, J=12.00, 3.66 Hz, 1H) 3.15 (s, 3H) 3.49 (s, 2H) 3.85-3.97 (m, 4H) 4.37-4.51 (m, 1H) 7.46 (dd, J=8.46, 1.64 Hz, 1H) 7.49 (d, J=1.77 Hz, 1H) 7.68 (s, 1H) 7.95 (s, 1H) 8.10 (d, J=7.83 Hz, 1H) 8.39 (d, J=8.34 Hz, 1H). [M+H] calc'd for C₂₉H₃₉N₇O₃ 534; found, 534.

### Compound 176

### 4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-N-(1-ethylpiperidin-4-yl)-3-methoxybenzamide

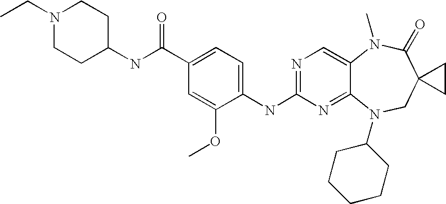

The title compound was synthesized using an analogous procedure described for Compound 169 except that 1-ethylpiperidine-4-amine was used and the final compound was purified by reverse phase HPLC followed by neutralization with base. ¹H NMR (400 MHz, DMSO-d₆) δ ppm 0.65-0.71 (m, 2H) 0.89-0.95 (m, 2H) 1.01-1.20 (m, 9H) 1.32-1.56 (m, 4H) 1.57-1.92 (m, 8H) 2.75-3.10 (m, 2H) 3.15 (s, 3H) 3.50 (s, 2H) 3.82 (br. s., 1H) 3.95 (s, 3H) 4.37-4.50 (m, 1H) 7.47 (d, J=8.34 Hz, 1H) 7.52 (d, J=1.52 Hz, 1H) 7.67 (s, 1H) 7.95 (s, 1H) 8.17-8.25 (m, 1H) 8.39 (d, J=8.59 Hz, 1H). [M+H] calc'd for C₃₁H₄₃N₇O₃ 562; found, 562.

### Compound 177

### 3-methoxy-4-(5′-methyl-9′-(2-methylcyclopentyl)-6′-oxo-5′,6′,8′,9′-tetrahydrospiro[cyclopropane-1,7′-pyrimido[4,5-b][1,4]diazepine]-2′-ylamino)-N-(1-methylpiperidin-4-yl)benzamide

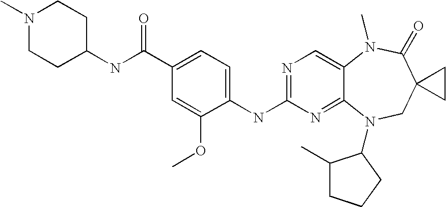

The title compound was synthesized using an analogous procedure described for Compound 33 except that 1-amino-2-methylcyclopentane was used for the reductive amination and the final compound was purified by reverse phase HPLC followed by neutralization with base. ¹H NMR (400 MHz, DMSO-d₆) δ ppm 0.53-2.08 (m, 20H) 2.19 (br. s., 3H) 2.73-2.89 (m, 2H) 3.12-3.21 (m, 3H) 3.46-3.63 (m, 2H) 3.66-3.82 (m, 1H) 3.93 (s, 3H) 4.41-4.67 (m, 1H) 7.42-7.50 (m, 2H) 7.64-7.71 (m, 1H) 7.95-8.03 (m, 1H) 8.09 (d, J=8.59 Hz, 1H) 8.37 (d, 1H). [M+H] calc'd for C₃₀H₄₁N₇O₃ 548; found, 548.

### Compound 178

### 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic Acid

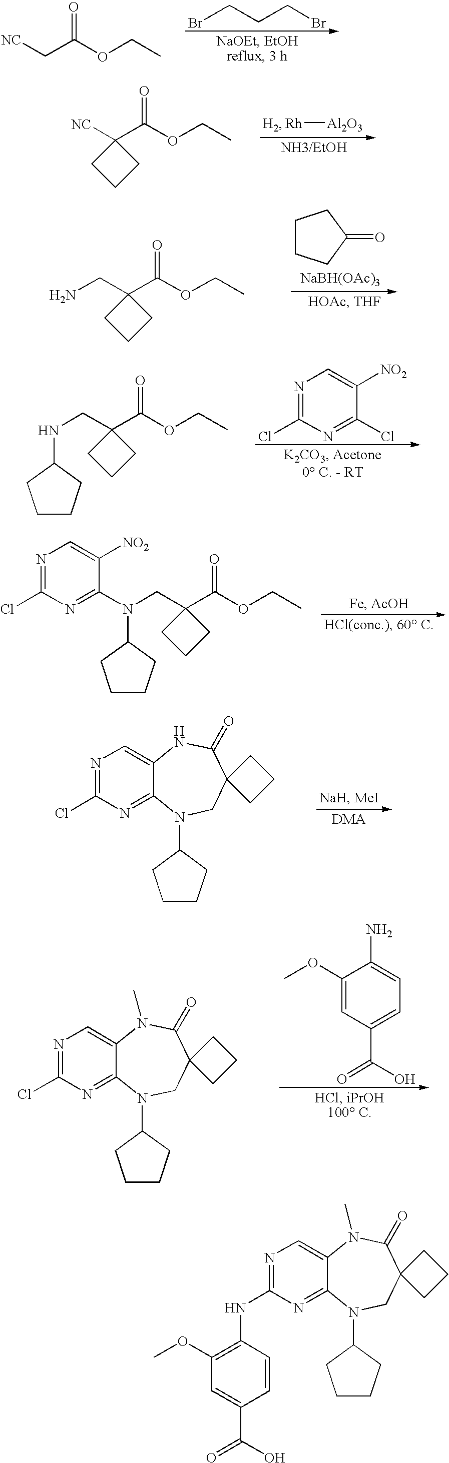

Ethyl 1-cyanocyclobutanecarboxylate. To a solution of sodium ethoxide (21 wt % in EtOH, 5.79 mL, 15.5 mmol) in EtOH (25 mL), was added ethyl cyanoacetate (1.12 mL, 10.5 mmol), followed shortly thereafter by 1,1-dibromopropane (1.12 mL, 10 mmol). The reaction mixture was refluxed for 3 hrs. It was then concentrated, and diluted to ethyl acetate. The organic layer was washed with NaHCO₃, brine, water, dried over Na₂SO₄ and concentrated in vacuo to obtain ethyl 1-cyanocyclobutanecarboxylate (1.17 g, 74% yield) as a red liquid. It was used directly for next step reaction. ¹H NMR (400 MHz, CHLOROFORM-d) δ 1.32 (t, J=7.2 Hz, 3H) 2.10-2.32 (m, 2H) 2.59-2.76 (m, 4H) 4.28 (q, J=7.1 Hz, 3H).

Ethyl 1-(aminomethyl)cyclobutanecarboxylate. A solution of compound ethyl 1-cyanocyclobutanecarboxylate (2 g, 13 mmol) in 1% ammonia in ethanol (20 mL) was hydrogenated with 5% rhodium on alumina (0.8 g) over the weekend (3 d). The reaction mixture was filtered through celite. The filtrate was concentrated and dried to give the product, compound 3 (1.5 g, 75%) as light yellow liquid. ¹H NMR (400 MHz, CHLOROFORM-d) δ 1.29 (t, J=7.1 Hz, 3H) 1.93-1.96 (m, 2H) 2.33-2.49 (m, 4H) 3.07 (s, 2H) 4.18 (q, J=7.2 Hz, 2H).

Ethyl 1-((cyclopentylamino)methyl)cyclobutanecarboxylate. Ethyl 1-(aminomethyl)cyclobutanecarboxylate (1.5 g, 9.6 mmol) was treated with cyclopentanone (1.02 ml, 11.5 mmol) in the presence of sodium triacetoxyborohydride (3 g, 14.4 mmol) and glacial acetic acid (3 mL) in tetrahydrofuran (100 ml) for 18 hours at room temperature. The reaction mixture was concentrated and carefully diluted with saturated aqueous NaHCO₃. The whole was extracted with ethyl acetate (200 ml×3). The organic layer was dried over Na₂SO₄ and concentrated in vacuo to obtain ethyl 1-((cyclopentylamino)methyl)cyclobutanecarboxylate (1.67 g, 77% yield) as light yellow liquid. ¹H NMR (400 MHz, CHLOROFORM-d) δ 1.27 (t, J=7.1 Hz, 3H), 1.35-1.57 (m, 6H), 1.77 (m, 2H), 2.38-2.43 (m, 4H), 2.91 (d, J=8.0 Hz, 2H), 4.13 (q, J=7.2 Hz, 2H). [M+H] calc'd for C₁₃H₂₃NO₂, 226; found, 226.

Ethyl 1-(((2-chloro-5-nitropyrimidin-4-l)(cyclopentyl)amino)methyl)cyclobutanecarboxylate. To a solution of 2,4-dichloro-5-nitropyrimidine (1.46 g, 7.5 mmol) in acetone (25 ml) at 0° C., was added ethyl 1-((cyclopentylamino)methyl)cyclobutanecarboxylate (1.67 g, 7.42 mmol) in acetone (5 ml) dropwise, followed by potassium carbonate (2.07 g, 15 mmol) and the whole was stirred at room temperature for 18 hours. After evaporation in vacuo, the residue was partitioned between ethyl acetate (200 ml) and water (200 ml). The organic layer was washed with NaHCO₃, brine and water, dried over Na₂SO₄ and concentrated in vacuo. This mixture was used for next step reaction without further purification. [M+H] calc'd for C₁₇H₂₃ClN₄O₄, 383; found, 383.

2′-chloro-9′-cyclopentyl-8′,9′-dihydrospiro[cyclobutane-1,7′-pyrimido[4,5-b][1,4]diazepin]-6′(5′H)-one. To a suspension of ethyl 1-(((2-chloro-5-nitropyrimidin-4-l)(cyclopentyl)amino)methyl)cyclobutanecarboxylate, reduced iron (2 equivalent) in acetic acid (20 ml) was added dropwise concentrated hydrochloric acid (5 ml) at 0° C. The reaction mixture was stirred at 60° C. for 4 h. It was then concentrated in vacuo, diluted to EtOAc, basified with 10% NaOH solution at 0° C. The whole was filtered through celite, washed with EtOAc. The filtrate was then separated. The organic layer was dried over Na₂SO₄. The solution was concentrated in vacuo followed by precipitation from ether to afford 2′-chloro-9′-cyclopentyl-8′,9′-dihydrospiro[cyclobutane-1,7′-pyrimido[4,5-b][1,4]diazepin]-6′(5′H)-one as white solid. [M+H] calc'd for C₁₅H₁₉ClNO₄, 307; found, 307.

4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid. A mixture of 2′-chloro-9′-cyclopentyl-8′,9′-dihydrospiro[cyclobutane-1,7′-pyrimido[4,5-b][1,4]diazepin]-6′(5′H)-one, 4-amino-3-methoxybenzoic acid (1 equivalent), isopropanol (30 ml), and concentrated hydrochloric acid (20 drops) was stirred at 100° C. for 20 h. Solid was filtered to give 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid as white solid. [M+H] calc'd for C₂₄H₂₉ClN₅O₄, 452; found, 452.

### Compound 179

### 4-(9′-Cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(1-methylpiperidin-4-yl)benzamide

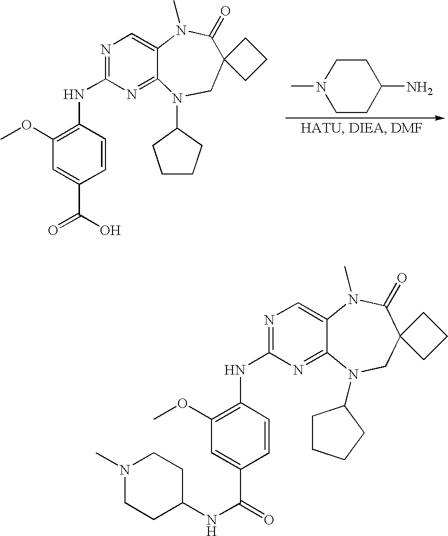

The title compound was synthesized using an analogous procedure to that described in connection with Compound 33 from 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid except that ethyl 1-((cyclopentylamino)methyl)cyclobutanecarboxylate, prepared from ethyl 2-cyanoacetate, was used and the final compound was purified by reverse phase HPLC followed by neutralization with base. ¹H NMR (400 MHz, DMSO-d₆) δ ppm 1.58 (br. s., 1H) 1.61-1.81 (m, 9H) 1.84-1.97 (m, 3H) 2.04 (br. s., 2H) 2.32 (dd, J=3.8, 1.8 Hz, 2H) 2.81 (d, J=5.0 Hz, 1H) 2.79 (d, J=4.8 Hz, 2H) 3.05-3.21 (m, 6H) 3.72 (s, 3H) 3.94 (s, 3H) 4.02 (td, J=7.9, 3.9 Hz, 1H) 4.85 (q, J=8.4 Hz, 1H) 7.53 (d, J=2.3 Hz, 1H) 7.49-7.55 (m, 1H) 8.07 (s, 1H) 8.17 (d, J=8.3 Hz, 1H) 8.40 (d, J=7.3 Hz, 1H) 8.75 (br. s., 1H) 9.36 (br. s., 1H). [M+H] calc'd for C₃₀H₄₁N₇O₃, 548; found, 548.

### Compound 180

### 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(4-methylpiperazin-1-yl)benzamide

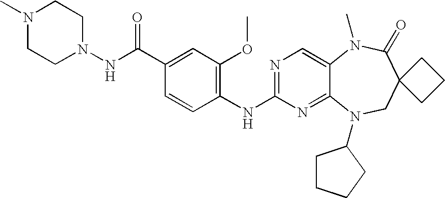

The title compound was synthesized using an analogous procedure to that described in connection with Compound 179 from 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid except that ethyl 4-methylpiperazin-1-amine was used and the final compound was purified by reverse phase HPLC followed by neutralization with base. ¹H NMR (400 MHz, CHLOROFORM-d) δ ppm 1.61-1.85 (m, 10H), 2.03-2.15 (m, 2H), 2.33 (s, 3H), 2.37-2.49 (m, 2H), 2.65 (br. s., 4H), 3.00 (br. s., 4H), 3.27 (s, 3H), 3.65 (s, 2H), 3.94 (s, 3H), 4.83-5.00 (m, 1H), 6.94 (br. s., 1H), 7.25 (d, J=7.33 Hz, 1H), 7.38 (br. s., 1H), 7.66 (s, 1H), 7.88 (s, 1H), 8.49 (d, J=8.34 Hz, 1H), [M+H] calc'd for C₂₉H₄₀N₈O₃ 549; found, 549.

### Compound 181

### (R)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide

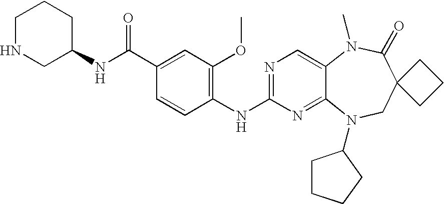

The title compound was synthesized using an analogous procedure to that described in connection with Compound 179 from 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid except that (R)-tert-butyl 3-aminopiperidine-1-carboxylate followed by removal of Boc using TFA in DCM and the final compound was purified by reverse phase HPLC followed by neutralization with base. ¹H NMR (400 MHz, CHLOROFORM-d) δ ppm 1.61-1.84 (m, 14H), 2.08 (br. s., 2H), 2.32-2.50 (m, 2H), 2.64-2.89 (m, 4H), 3.13 (d, J=11.87 Hz, 1H), 3.27 (s, 3H), 3.64 (s, 2H), 3.94 (s, 3H), 4.16 (br. s., 1H), 4.81-5.02 (m, 1H), 6.80 (d, J=7.07 Hz, 1H), 7.33 (d, J=8.34 Hz, 1H), 7.44 (s, 1H), 7.64 (s, 1H), 7.87 (s, 1H), 8.48 (d, J=9.85 Hz, 1H), [M+H] calc'd for C₂₉H₃₉N₇O₃ 534; found, 534.

### Compound 182

### (S)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide

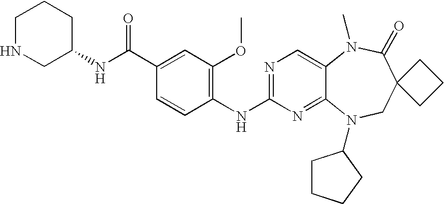

The title compound was synthesized using an analogous procedure to that described in connection with Compound 179 from 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid except that (S)-tert-butyl 3-aminopiperidine-1-carboxylate followed by removal of Boc using TFA in DCM and the final compound was purified by reverse phase HPLC followed by neutralization with base. ¹H NMR (400 MHz, CHLOROFORM-d) δ ppm 1.63-1.88 (m, 14H), 2.05-2.24 (m, 5H), 2.35-2.53 (m, 2H), 2.71-2.92 (m, 3H), 3.08-3.20 (m, 1H), 3.29 (s, 3H), 3.66 (s, 2H), 3.97 (s, 3H), 4.17 (br. s., 1H), 4.90-4.94 (m, 1H), 6.69 (d, J=7.33 Hz, 1H), 7.32 (dd, J=8.46, 1.89 Hz, 1H), 7.45 (d, J=1.77 Hz, 1H), 7.65 (s, 1H), 7.89 (s, 1H), 8.50 (d, J=8.59 Hz, 1H), [M+H] calc'd for C₂₉H₃₉N₇O₃ 534; found, 534.

### Compound 183

### 4-(7-(Cyanomethyl)-9-cyclopentyl-5-methyl-6-oxo-6,7,8,9-tetrahydro-5H-pyrimido[4,5-b][1,4]diazepin-2-ylamino)-3-methoxybenzoic Acid

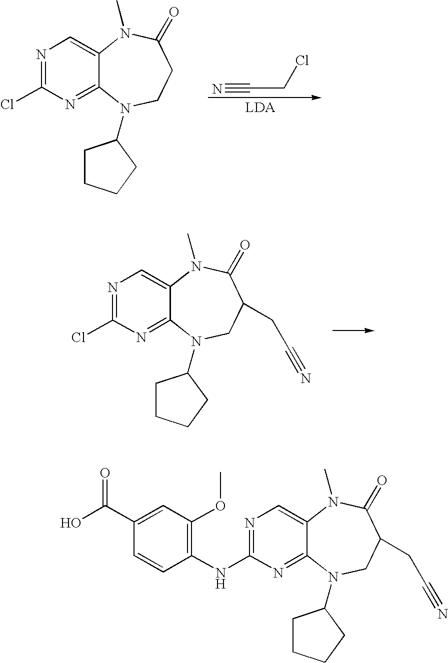

2-(2-Chloro-9-cyclopentyl-5-methyl-6-oxo-6,7,8,9-tetrahydro-5H-pyrimido[4,5-b][1,4]diazepin-7-yl)acetonitrile. The 2-chloro-9-cyclopentyl-5-methyl-8,9-dihydro-5H-pyrimido[4,5-b][1,4]diazepin-6(7H)-one was deprotonated with LDA (2 equiv), in THF (0.1-0.5M) at −78 C, stirred for 10 min-2 h at −78 C and the resulting anion quenched with 2-chloroacetonitrile (an electrophile) to generate compound 2-(2-chloro-9-cyclopentyl-5-methyl-6-oxo-6,7,8,9-tetrahydro-5H-pyrimido[4,5-b][1,4]diazepin-7-yl)acetonitrile. [M+H] calc'd for C₁₅H₁₈ClN₅O 320; found, 320.

4-(7-(cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid. 2-(2-chloro-9-cyclopentyl-5-methyl-6-oxo-6,7,8,9-tetrahydro-5H-pyrimido[4,5-b][1,4]diazepin-7-yl)acetonitrile reacted with 4-amino-3-methoxybenzoic acid to furnish acid 4-(7-(cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid using the analog procedure describe for Compound 178. [M+H] calc'd for C₂₃H₂₆N₆O₄ 451; found, 451.

### Compound 184

### 4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

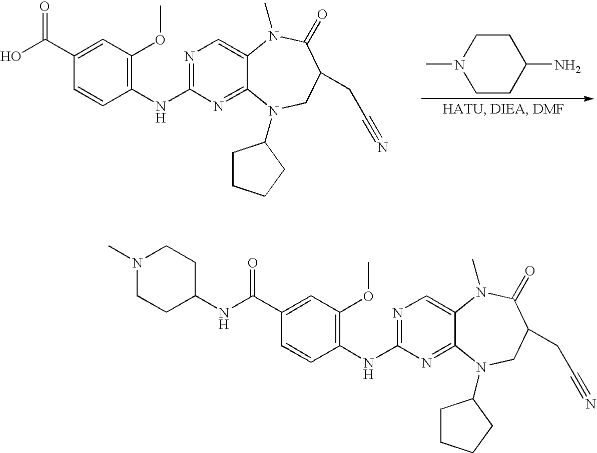

The title compound was synthesized using an analogous procedure to that described in connection with Compound 11 from 4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid and 1-methylpiperidin-4-amine, was used and the final compound was purified by reverse phase HPLC followed by neutralization with base. ¹H NMR (400 MHz, DMSO-d₆) δ ppm 1.50-1.67 (m, 3H) 1.67-1.91 (m, 6H) 1.91-2.11 (m, 3H) 2.65-2.83 (m, 5H) 3.03-3.18 (m, 2H) 3.22 (s, 3H) 3.24-3:40 (m, 1H) 3.49 (app. d, J=13.14 Hz, 3H) 3.61 (dd, J=12.88, 10.86 Hz, 1H) 3.95 (s, 3H) 3.98-4.08 (m, 1H) 4.77-4.88 (m, 1H) 7.46-7.54 (m, 2H) 8.16 (s, 1H) 8.22-8.38 (br s, 1H) 8.27 (d, J=8.34 Hz, 1H) 8.35 (d, J=7.58 Hz, 1H) 9.41 (br s, 1H). [M+H] calc'd for C₂₉H₃₈₉N₈O₃ 558; found, 558.

### Compound 185

### 4-(7-(2-amino-2-oxoethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

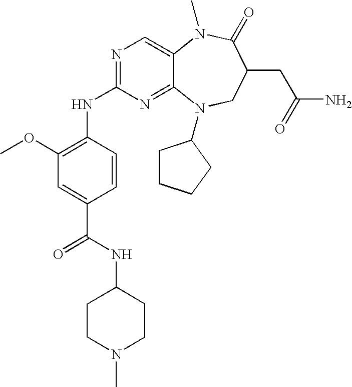

The title compound was prepared from 4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide using NaOH (aq., 1M) in MeOH (0.1-0.5M) at 100 C for 3 h and the final compound was purified by reverse phase HPLC followed by neutralization with base. ¹H NMR (400 MHz, DMSO-d₆) δ ppm 1.48-2.09 (m, 12H) 2.20 (dd, J=16.17, 7.83 Hz, 1H) 2.56 (dd, J=15.92, 6.32 Hz, 1H) 2.73-2.83 (m, 3H) 3.02-3.17 (m, 3H) 3.19 (s, 3H) 3.39-3.57 (m, 4H) 3.95 (s, 3H) 3.97-4.08 (m, 1H) 4.76-4.91 (m, 1H) 6.89 (br. s., 1H) 7.42 (br. s., 1H) 7.47-7.57 (m, 2H) 8.15 (s, 1H) 8.24 (d, J=8.34 Hz, 1H) 8.39 (d, J=7.33 Hz, 1H) 8.51 (br. s., 1H) 9.43 (br. s., 1H). [M+H] calc'd for C₂₉H₃₈N₈O₃ 565; found, 565.

### Compound 186

### 4-(7-allyl-9-cyclopentyl-5-methyl-6-oxo-6,7,8,9-tetrahydro-5H-pyrimido[4,5-b][1,4]diazepin-2-ylamino)-3-methoxybenzoic Acid

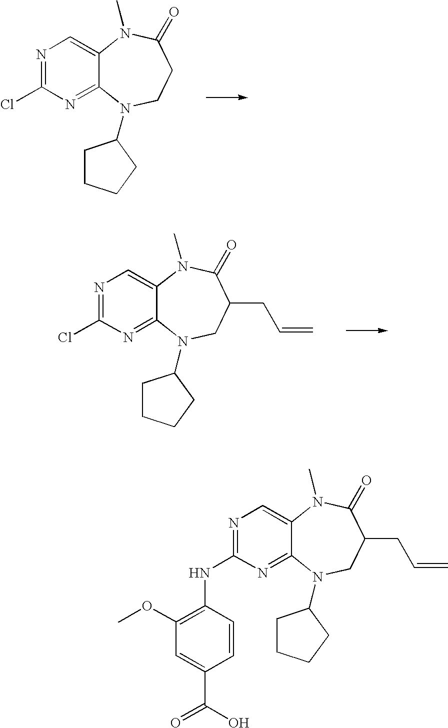

7-allyl-2-chloro-9-cyclopentyl-5-methyl-8,9-dihydro-5H-pyrimido[4,5-b][1,4]diazepin-6(7H)-one. 2-chloro-9-cyclopentyl-5-methyl-8,9-dihydro-5H-pyrimido[4,5-b][1,4]diazepin-6(7H)-one (5.62 g, 20.0 mmol) was dissolved in THF (90 mL) and added to a cooled (−78° C.) stirred solution of freshly prepared LDA (0.745M, 54.0 mL, 20.2 mmol) over the period of 10 min via cannula. The resulting yellow reaction mixture was stirred at −78° C. for 30 min and treated slowly with allyl bromide (7.00 mL, 80.8 mmol). The resulting mixture was stirred at −78° C. for 2 h and at room temperature for 2 h. It was quenched with ammonium chloride (sat. aq., 100 mL) and water (30 mL). The mixture was extracted with ethyl acetate (2×100 mL) and the combined organic extracts were washed with brine (50 mL), dried (MgSO₄), filtered and concentrated in vacuo. Column chromatography on silica gel (ethyl acetate), afforded the title compound as a yellowish solid. (4.09 g, 64%). m/z 321.3.

4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid. 7-Allyl-2-chloro-9-cyclopentyl-5-methyl-8,9-dihydro-5H-pyrimido[4,5-b][1,4]diazepin-6(7H)-one (642 mg, 2.00 mmol) and 4-amino-3-methoxybenzoic acid (368 mg, 2.20 mmol) were dissolved in dioxane (20 mL) and treated with p-toluenesulfonic acid monohydrate (300 mg, 1.60 mmol). The heterogeneous reaction mixture was stirred in a closed vial at 100° C. for 3 days and cooled to room temperature. The precipitate was filtered, washed with ethyl acetate (3×10 mL) and dried in vacuum to afford the title compound as a brown solid (0.812 g, 90%), which was used in the next step without further purification. m/z 452.4.

### Compound 187

### 4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

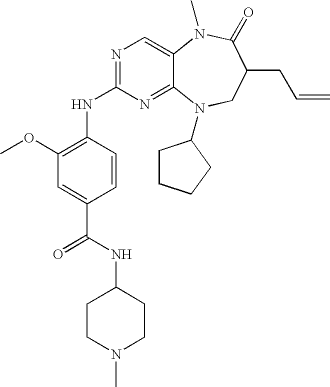

The title compound was synthesized using an analogous procedure to that described in connection with Compound 32 except that 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid instead of 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid was used and the final compound was purified by reverse phase HPLC and basified to give the free base. ¹H NMR (400 MHz, DMSO-d₆) δ ppm 1.37-1.51 (m, 1H) 1.54-1.84 (m, 10H) 1.88-2.12 (m, 4H) 2.17 (s, 3H) 2.37-2.47 (m, 1H) 2.73-2.85 (m, 3H) 3.19 (s, 3H) 3.36-3.42 (m, 2H) 3.66-3.80 (m, 1H) 3.94 (s, 3H) 4.77-4.88 (m, 1H) 5.03 (d, J=10.36 Hz, 1H) 5.08 (d, J=17.18 Hz, 1H) 5.71-5.87 (m, 1H) 7.43-7.52 (m, 2H) 7.75 (s, 1H) 8.05-8.14 (m, 2H) 8.37 (d, J=8.08 Hz, 1H). [M+H] calc'd for C₃₀H₄₁N₇O₃ 548; found, 548.

### Compound 188

### (S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

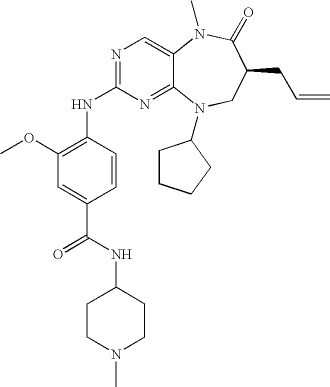

The enantiomers of Compound 187 were separated using SFC (ChiralPak OJ-H (5 um, 10×250 mm), 15% Ethanol buffered with NH4OAc (10 mM) in supercritical CO₂). The absolute configuration was determined by co-crystal of the title compound with PLK1 enzyme. ¹H NMR (400 MHz, DMSO-d₆) δ ppm 1.37-1.51 (m, 1H) 1.54-1.84 (m, 10H) 1.88-2.12 (m, 4H) 2.17 (s, 3H) 2.37-2.47 (m, 1H) 2.73-2.85 (m, 3H) 3.19 (s, 3H) 3.36-3.42 (m, 2H) 3.66-3.80 (m, 1H) 3.94 (s, 3H) 4.77-4.88 (m, 1H) 5.03 (d, J=10.36 Hz, 1H) 5.08 (d, J=17.18 Hz, 1H) 5.71-5.87 (m, 1H) 7.43-7.52 (m, 2H) 7.75 (s, 1H) 8.05-8.14 (m, 2H) 8.37 (d, J=8.08 Hz, 1H). [M+H] calc'd for C₃₀H₄₁N₇O₃ 548; found, 548.

### Compound 189

### (R)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

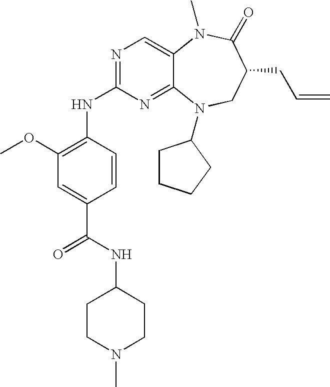

The enantiomers of Compound 187 were separated using SFC (ChiralPak OJ-H (5 um, 10×250 mm), 15% Ethanol buffered with NH4OAc (10 mM) in supercritical CO₂). The absolute configuration was determined based on the co-crystal of the Compound 188 with PLK1 enzyme. ¹H NMR (400 MHz, DMSO-d₆) δ ppm 1.37-1.51 (m, 1H) 1.54-1.84 (m, 10H) 1.88-2.12 (m, 4H) 2.17 (s, 3H) 2.37-2.47 (m, 1H) 2.73-2.85 (m, 3H) 3.19 (s, 3H) 3.36-3.42 (m, 2H) 3.66-3.80 (m, 1H) 3.94 (s, 3H) 4.77-4.88 (m, 1H) 5.03 (d, J=10.36 Hz, 1H) 5.08 (d, J=17.18 Hz, 1H) 5.71-5.87 (m, 1H) 7.43-7.52 (m, 2H) 7.75 (s, 1H) 8.05-8.14 (m, 2H) 8.37 (d, J=8.08 Hz, 1H). [M+H] calc'd for C₃₀H₄₁N₇O₃ 548; found, 548.

### Compound 190

### 4-(9-cyclopentyl-5-methyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide

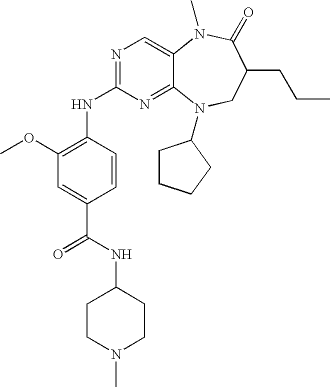

The title compound was prepared from hydrationation of 4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide using pd/C as catalyst. ¹H NMR (400 MHz, DMSO-d₆) δ ppm 0.85 (t, J=7.20 Hz, 3H) 1.19-1.33 (m, 3H) 1.50-1.63 (m, 3H) 1.64-1.82 (m, 6H) 1.96-2.09 (m, 3H) 2.69-2.83 (m, 4H) 3.01-3.16 (m, 2H) 3.19 (s, 3H) 3.31-3.54 (m, 4H) 3.95 (s, 3H) 3.99-4.08 (m, 1H) 4.74-4.89 (m, 1H) 7.47-7.55 (m, 2H) 8.12 (s, 1H) 8.23 (d, J=8.08 Hz, 1H) 8.38 (d, J=7.33 Hz, 1H) 8.53 (br. s., 1H) 9.36 (br. s., 1H). [M+H] calc'd for C₃₀H₄₃N₇O₃ 550; found, 550.

### Compound 191

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide

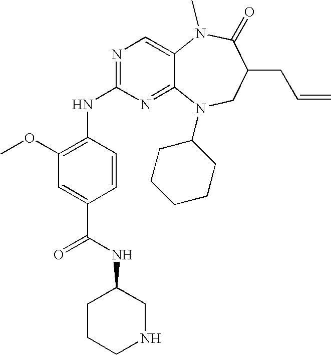

The title compound was prepared from 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-tert-butyl 3-aminopiperidine-1-carboxylate, using an analogous procedure to that described in connection with Compound 187 followed by removal of Boc using TFA in DCM. the final compound was purified by reverse phase HPLC and basified to give the free base. ¹H NMR (400 MHz, DMSO-d₆) δ ppm 1.06-1.23 (m, 1H) 1.26-1.77 (m, 9H) 1.77-1.91 (m, 4H) 1.99-2.10 (m, 1H) 2.33 (br. s., 1H) 2.34-2.48 (m, 3H) 2.72-2.85 (m, 2H) 2.97 (dd, J=11.75, 3.41 Hz, 1H) 3.19 (s, 3H) 3.23-3.36 (m, 1H) 3.53 (dd, J=12.76, 1.64 Hz, 1H) 3.74-3.86 (m, 1H) 3.95 (s, 3H) 4.31-4.44 (m, 1H) 5.04 (d, J=10.11 Hz, 1H) 5.09 (dd, J=17.18, 1.52 Hz, 1H) 5.74-5.88 (m, 1H) 7.46 (dd, J=8.34, 1.77 Hz, 1H) 7.49 (d, J=1.77 Hz, 1H) 7.72 (s, 1H) 7.96 (d, J=7.83 Hz, 1H) 8.07 (s, 1H) 8.37 (d, J=8.59 Hz, 1H). [M+H] calc'd for C₃₀H₄₁N₇O₃ 548; found, 548.

### Compound 192

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-piperidin-3-yl)benzamide

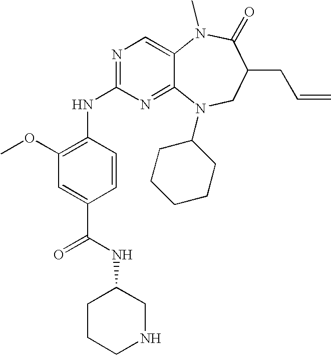

The title compound was prepared from 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-tert-butyl 3-aminopiperidine-1-carboxylate, using an analogous procedure to that described in connection with Compound 187 followed by removal of Boc using TFA in DCM. the final compound was purified by reverse phase HPLC and basified to give the free base. ¹H NMR (400 MHz, DMSO-d₆) δ ppm 1.06-1.77 (m, 10H) 1.88 (br. s., 4H) 1.98-2.11 (m, 1H) 2.25 (br. s., 1H) 2.35-2.46 (m, 3H) 2.72-2.87 (m, 2H) 2.96 (br. s., 1H) 3.19 (s, 3H) 3.53 (d, J=12.13 Hz, 1H) 3.74-3.87 (m, 1H) 3.96 (s, 3H) 4.32-4.44 (m, 1H) 5.04 (d, J=10.11 Hz, 1H) 5.09 (d, J=17.43 Hz, 1H) 5.73-5.89 (m, 1H) 7.42-7.52 (m, 2H) 7.72 (s, 1H) 7.96 (d, J=7.83 Hz, 1H) 8.07 (s, 1H) 8.37 (d, J=8.59 Hz, 1H). [M+H] calc'd for C₃₀H₄₁N₇O₃ 548; found, 548.

### Compound 193

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide

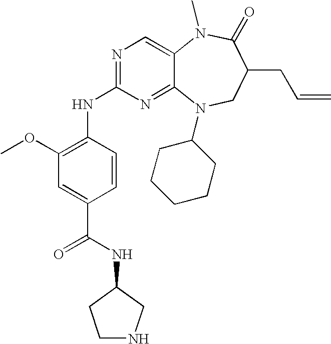

The title compound was prepared from 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-tert-butyl 3-aminopyrrolidine-1-carboxylate, using an analogous procedure to that described in connection with Compound 187 followed by removal of Boc using TFA in DCM. The final compound was purified by reverse phase HPLC and basified to give the free base. ¹H NMR (400 MHz, DMSO-d₆) δ ppm 1.06-2.12 (m, 13H) 2.37-2.48 (m, 1H) 2.65 (dd, J=11.24, 4.93 Hz, 1H) 2.71-2.82 (m, 2H) 2.85-3.02 (m, 2H) 3.19 (s, 3H) 3.24-3.36 (m, 2H) 3.53 (dd, J=12.76, 1.64 Hz, 1H) 3.95 (s, 3H) 4.23-4.45 (m, 2H) 5.04 (d, J=10.11 Hz, 1H) 5.09 (dd, J=17.18, 1.52 Hz, 1H) 5.73-5.88 (m, 1H) 7.41-7.48 (m, 1H) 7.50 (d, J=1.77 Hz, 1H) 7.72 (s, 1H) 8.07 (s, 1H) 8.17 (d, J=6.82 Hz, 1H) 8.37 (d, J=8.34 Hz, 1H). [M+H] calc'd for C₂₉H₃₉N₇O₃ 534; found, 534.

### Compound 194

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide

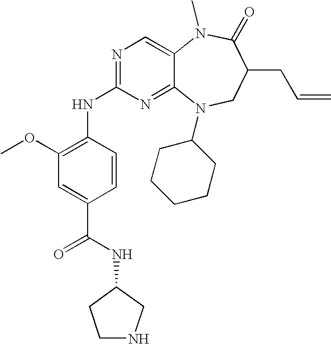

The title compound was prepared from 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-tert-butyl 3-aminopyrrolidine-1-carboxylate, using an analogous procedure to that described in connection with Compound 187 followed by removal of Boc using TFA in DCM. The final compound was purified by reverse phase HPLC and basified to give the free base. ¹H NMR (400 MHz, DMSO-d₆) δ ppm 1.07-1.22 (m, 1H) 1.26-1.41 (m, 1H) 1.41-1.77 (m, 5H) 1.87 (br. s., 3H) 1.93-2.10 (m, 2H) 2.38-2.47 (m, 1H) 2.65 (dd, J=11.12, 5.05 Hz, 1H) 2.71-2.81 (m, 2H) 2.86-2.95 (m, 1H) 2.98 (dd, J=11.12, 6.82 Hz, 1H) 3.19 (s, 3H) 3.25-3.34 (m, 2H) 3.49-3.57 (m, 1H) 3.95 (s, 3H) 4.24-4.34 (m, 1H) 4.34-4.44 (m, 1H) 5.04 (d, J=10.11 Hz, 1H) 5.09 (dd, J=17.31, 1.64 Hz, 1H) 5.74-5.88 (m, 1H) 7.46 (dd, J=8.34, 1.77 Hz, 1H) 7.50 (d, J=1.77 Hz, 1H) 7.72 (s, 1H) 8.07 (s, 1H) 8.17 (d, J=7.07 Hz, 1H) 8.37 (d, J=8.34 Hz, 1H). [M+H] calc'd for C₂₉H₃₉N₇O₃ 534; found, 534.

### Compound 195

### (S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide

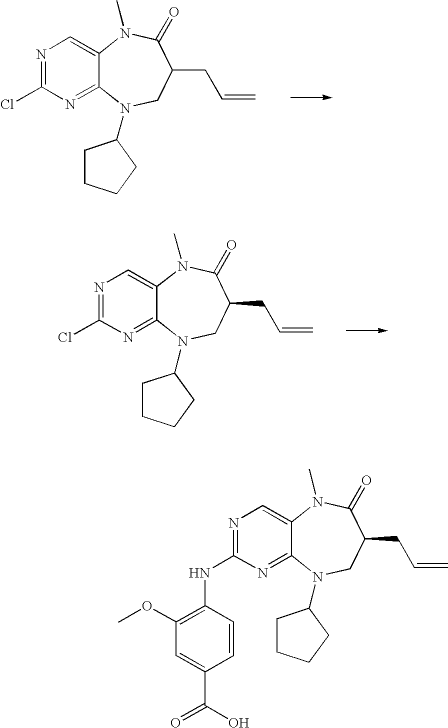

(S)-7-allyl-2-chloro-9-cyclopentyl-5-methyl-8,9-dihydro-5H-pyrimido[4,5-b][1,4]diazepin-6(7H)-one. The enantiomers of this compound were separated using SFC (ChiralPak AD-H (5 um, 10×250 mm), 30% Ethanol buffered with NH₄OAc (10 mM) in supercritical CO₂). The absolute configuration was determined by co-crystal after the conversion of the title compound to the Compound 188 with PLK1 enzyme.

(S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid. The intermediate acid was prepared using the same condition described for the 4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid.

### Compound 196

### (S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide

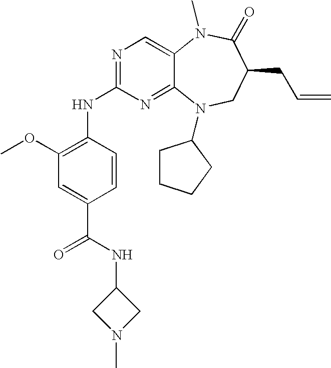

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and 1-methylazetidin-3-amine, using an analogous procedure to that described in connection with Compound 188. The final compound was purified by reverse phase HPLC and basified to give the free base. ¹H NMR (400 MHz, DMSO-d₆) δ ppm 1.39-1.86 (m, 7H) 1.99-2.12 (m, 2H) 2.28 (s, 3H) 2.38-2.48 (m, 1H) 2.71-2.83 (m, 1H) 3.01 (t, J=7.07 Hz, 2H) 3.19 (s, 3H) 3.40 (d, J=6.32 Hz, 2H) 3.57 (t, J=7.20 Hz, 2H) 3.94 (s, 3H) 4.35-4.50 (m, 1H) 4.74-4.91 (m, 1H) 5.03 (d, J=10.11 Hz, 1H) 5.08 (d, J=17.18 Hz, 1H) 5.72-5.87 (m, 1H) 7.44-7.55 (m, 2H) 7.76 (s, 1H) 8.10 (s, 1H) 8.39 (d, J=8.34 Hz, 1H) 8.60 (d, J=6.82 Hz, 1H). [M+H] calc'd for C₂₈H₃₇N₇O₃ 520; found, 520.

### Compound 197

### 4-((S)-7-allyl-9-cyclopentyl-5-methyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide

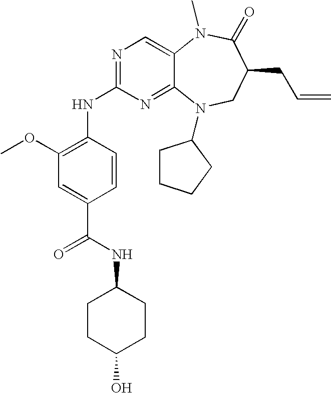

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (1R,4R)-4-aminocyclohexanol, using an analogous procedure to that described in connection with Compound 188. The final compound was purified by reverse phase HPLC and basified to give the free base. ¹H NMR (400 MHz, DMSO-d₆) δ ppm 1.17-1.31 (m, 2H) 1.32-1.52 (m, 3H) 1.52-1.76 (m, 5H) 1.76-1.92 (m, 5H) 1.98-2.12 (m, 2H) 2.38-2.48 (m, 1H) 2.73-2.84 (m, 1H) 3.19 (s, 3H) 3.33-3.49 (m, 3H) 3.66-3.79 (m, 1H) 3.94 (s, 3H) 4.53 (d, J=4.55 Hz, 1H) 4.75-4.90 (m, 1H) 5.03 (d, J=10.36 Hz, 1H) 5.08 (d, J=17.18 Hz, 1H) 5.72-5.87 (m, 1H) 7.40-7.51 (m, 2H) 7.73 (s, 1H) 7.99 (d, J=7.83 Hz, 1H) 8.09 (s, 1H) 8.37 (d, J=8.08 Hz, 1H). [M+H] calc'd for C₃₀₉H₄₀₉N₆O₃ 549; found, 549.

### Compound 198

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide

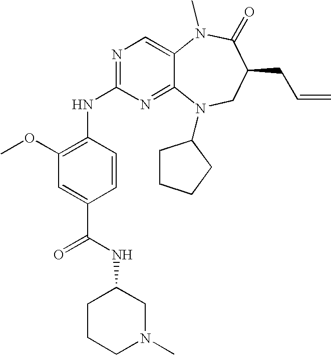

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-1-methylpiperidin-3-amine, using an analogous procedure to that described in connection with Compound 188. The final compound was purified by reverse phase HPLC and basified to give the free base. ¹H NMR (400 MHz, DMSO-d₆) δ ppm 1.24-1.38 (m, 1H) 1.39-1.93 (m, 12H) 1.97-2.13 (m, 2H) 2.19 (s, 3H) 2.38-2.48 (m, 1H) 2.61-2.72 (m, 1H) 2.72-2.88 (m, 2H) 3.19 (s, 3H) 3.40 (d, J=6.57 Hz, 2H) 3.94 (s, 4H) 4.77-4.90 (m, 1H) 5.03 (d, J=10.11 Hz, 1H) 5.08 (dd, J=17.05, 1.14 Hz, 1H) 5.72-5.86 (m, 1H) 7.43-7.51 (m, 2H) 7.74 (s, 1H) 8.00 (d, J=8.08 Hz, 1H) 8.09 (s, 1H) 8.38 (d, J=8.34 Hz, 1H). [M+H] calc'd for C₃₀H₄₁N₇O₃ 548; found, 548.

### Compound 199

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide

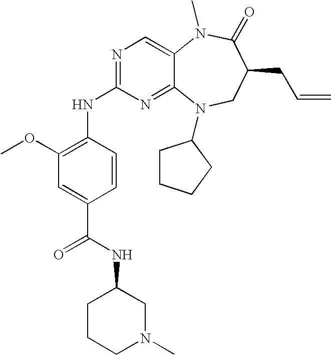

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-1-methylpiperidin-3-amine, using an analogous procedure to that described in connection with Compound 188. The final compound was purified by reverse phase HPLC and basified to give the free base. ¹H NMR (400 MHz, DMSO-d₆) δ ppm 1.24-1.39 (m, 1H) 1.39-1.93 (m, 12H) 1.97-2.14 (m, 2H) 2.19 (s, 3H) 2.36-2.48 (m, 1H) 2.61-2.72 (m, 1H) 2.72-2.88 (m, 2H) 3.19 (s, 3H) 3.40 (d, J=6.57 Hz, 2H) 3.86-4.03 (m, 4H) 4.77-4.89 (m, 1H) 5.03 (d, J=10.11 Hz, 1H) 5.05-5.13 (m, 1H) 5.73-5.86 (m, 1H) 7.42-7.51 (m, 2H) 7.74 (s, 1H) 8.00 (d, J=8.08 Hz, 1H) 8.09 (s, 1H) 8.38 (d, J=8.34 Hz, 1H). [M+H] calc'd for C₃₀H₄₁N₇O₃ 548; found, 548.

### Compound 200

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-piperidin-3-yl)benzamide

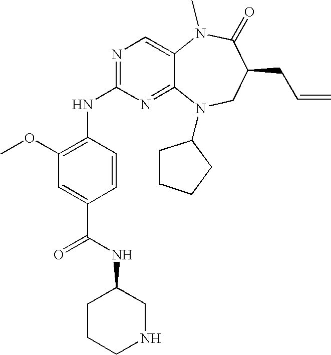

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-tert-butyl 3-aminopiperidine-1-carboxylate, using an analogous procedure to that described in connection with Compound 188 followed by removal of Boc using TFA in DCM. the final compound was purified by reverse phase HPLC and basified to give the free base. ¹H NMR (400 MHz, DMSO-d₆) δ ppm 1.38-1.85 (m, 9H) 1.85-1.97 (m, 2H) 1.97-2.13 (m, 2H) 2.38-2.48 (m, 1H) 2.72-2.91 (m, 3H) 3.19 (s, 3H) 3.21 (m., 1H) 3.28-3.47 (m, 4H) 3.94 (s, 3H) 4.10-4.23 (m, 1H) 4.78-4.90 (m, 1H) 5.03 (d, J=10.11 Hz, 1H) 5.09 (d, J=16.93 Hz, 1H) 5.72-5.86 (m, 1H) 7.47-7.56 (m, 2H) 7.95 (br. s., 1H) 8.11 (s, 1H) 8.38 (t, J=8.34 Hz, 2H). [M+H] calc'd for C₂₉H₃₉N₇O₃ 534; found, 534.

### Compound 201

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-piperidin-3-yl)benzamide

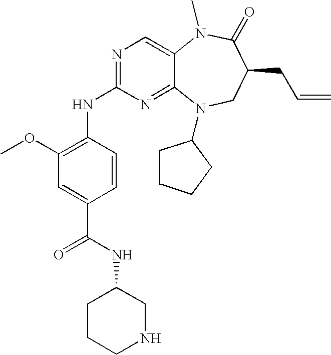

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-tert-butyl 3-aminopiperidine-1-carboxylate, using an analogous procedure to that described in connection with Compound 188 followed by removal of Boc using TFA in DCM. the final compound was purified by reverse phase HPLC and basified to give the free base. ¹H NMR (400 MHz, DMSO-d₆) δ ppm 1.36-1.91 (m, 11H) 2.00-2.13 (m, 2H) 2.34-2.47 (m, 3H) 2.73-2.86 (m, 2H) 2.97 (dd, J=11.62, 3.03 Hz, 1H) 3.19 (s, 3H) 3.37-3.43 (m, 2H) 3.76-3.87 (m, 1H) 3.94 (s, 3H) 4.78-4.90 (m, 1H) 5.03 (d, J=10.11 Hz, 1H) 5.09 (dd, J=17.43, 1.52 Hz, 1H) 5.73-5.86 (m, 1H) 7.44-7.50 (m, 2H) 7.75 (s, 1H) 7.99 (d, J=8.08 Hz, 1H) 8.10 (s, 1H) 8.37 (d, J=8.84 Hz, 1H). [M+H] calc'd for C₂₉H₃₉N₇O₃ 534; found, 534.

### Compound 202

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide

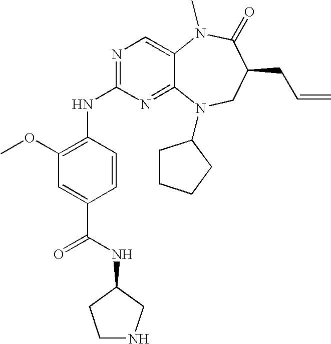

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-tert-butyl 3-aminopyrrolidine-1-carboxylate, using an analogous procedure to that described in connection with Compound 188 followed by removal of Boc using TFA in DCM. the final compound was purified by reverse phase HPLC and basified to give the free base. ¹H NMR (400 MHz, DMSO-d₆) δ ppm 1.45-1.79 (m, 7H) 1.79-1.89 (m, 1H) 1.95-2.13 (m, 3H) 2.42-2.48 (m, 1H) 2.64-2.73 (m, 1H) 2.73-2.83 (m, 2H) 2.90-3.04 (m, 3H) 3.21 (s, 3H) 3.37-3.47 (m, 2H) 3.96 (s, 3H) 4.27-4.38 (m, 1H) 4.78-4.90 (m, 1H) 5.03 (dd, J=10.11, 1.01 Hz, 1H) 5.08 (dd, J=17.31, 1.39 Hz, 1H) 5.76-5.88 (m, 1H) 7.44-7.52 (m, 2H) 7.67 (br. s., 1H) 7.95 (br. s., 1H) 8.08 (s, 1H) 8.36 (d, J=8.59 Hz, 1H). [M+H] calc'd for C₂₈H₃₇N₇O₃ 520; found, 520.

### Compound 203

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide

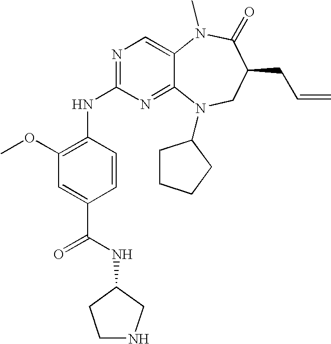

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-tert-butyl 3-aminopyrrolidine-1-carboxylate, using an analogous procedure to that described in connection with Compound 188 followed by removal of Boc using TFA in DCM. the final compound was purified by reverse phase HPLC and basified to give the free base. ¹H NMR (400 MHz, DMSO-d₆) δ ppm 1.46-1.78 (m, 7H) 1.79-1.89 (m, 1H) 1.95-2.13 (m, 3H) 2.42-2.48 (m, 1H) 2.70 (dd, J=11.12, 4.80 Hz, 1H) 2.74-2.83 (m, 2H) 2.89-3.04 (m, 3H) 3.21 (s, 3H) 3.37-3.47 (m, 2H) 3.96 (s, 3H) 4.27-4.38 (m, 1H) 4.76-4.89 (m, 1H) 5.03 (d, J=10.36 Hz, 1H) 5.08 (dd, J=17.05, 1.39 Hz, 1H) 5.74-5.90 (m, 1H) 7.44-7.52 (m, 2H) 7.66 (s, 1H) 7.89-7.97 (m, 1H) 8.08 (s, 1H) 8.35 (d, J=8.34 Hz, 1H). [M+H] calc'd for C₂₈H₃₇N₇O₃ 520; found, 520.

### Compound 204

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide

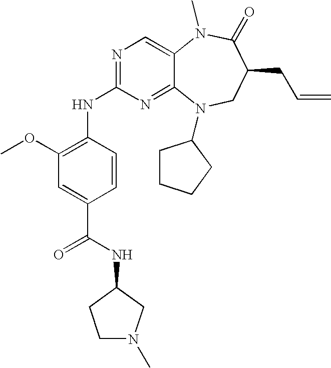

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (R)-1-methylpyrrolidin-3-amine, using an analogous procedure to that described in connection with Compound 188. The final compound was purified by reverse phase HPLC and basified to give the free base. ¹H NMR (400 MHz, DMSO-d₆) δ ppm 1.38-1.86 (m, 8H) 1.96-2.12 (m, 3H) 2.12-2.24 (m, 1H) 2.30 (s, 3H) 2.38-2.48 (m, 2H) 2.63-2.84 (m, 3H) 3.19 (s, 3H) 3.37-3.44 (m, 2H) 3.94 (s, 3H) 4.36-4.48 (m, 1H) 4.76-4.89 (m, 1H) 5.03 (d, J=10.11 Hz, 1H) 5.08 (d, J=17.43 Hz, 1H) 5.72-5.87 (m, 1H) 7.46-7.54 (m, 2H) 7.75 (s, 1H) 8.10 (s, 1H) 8.33-8.41 (m, 2H). [M+H] calc'd for C₂₉H₃₉N₇O₃ 534; found, 534.

### Compound 205

### 4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide

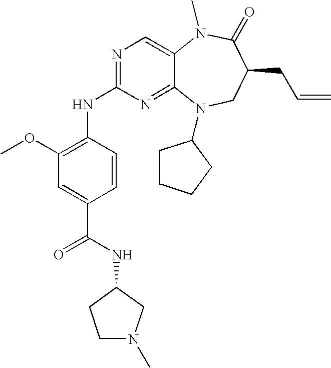

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and (S)-1-methylpyrrolidin-3-amine, using an analogous procedure to that described in connection with Compound 188. The final compound was purified by reverse phase HPLC and basified to give the free base. ¹H NMR (400 MHz, DMSO-d₆) δ ppm 1.37-1.86 (m, 8H) 1.97-2.23 (m, 3H) 2.28 (s, 3H) 2.35-2.48 (m, 3H) 2.60-2.73 (m, 2H) 2.74-2.84 (m, 1H) 3.19 (s, 3H) 3.38-3.45 (m, 2H) 3.94 (s, 3H) 4.34-4.48 (m, 1H) 4.76-4.89 (m, 1H) 5.03 (d, J=10.11 Hz, 1H) 5.08 (d, J=17.18 Hz, 1H) 5.72-5.88 (m, 1H) 7.46-7.56 (m, 2H) 7.75 (s, 1H) 8.10 (s, 1H) 8.32-8.42 (m, 2H). [M+H] calc'd for C₂₉H₃₉N₇O₃ 534; found, 534.

### Compound 206

### (S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide

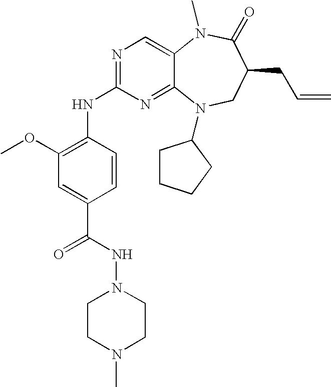

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and 4-methylpiperazin-1-amine, using an analogous procedure to that described in connection with Compound 188. The final compound was purified by reverse phase HPLC and basified to give the free base. ¹H NMR (400 MHz, DMSO-d₆) δ ppm 1.35-1.87 (m, 8H) 2.04 (dd, J=15.16, 7.33 Hz, 2H) 2.19 (s, 3H) 2.30-2.47 (m, 4H) 2.71-2.84 (m, 1H) 2.92 (t, J=4.55 Hz, 4H) 3.19 (s, 3H) 3.36-3.45 (m, 2H) 3.93 (s, 3H) 4.75-4.90 (m, 1H) 5.03 (d, J=10.11 Hz, 1H) 5.08 (d, J=16.93 Hz, 1H) 5.72-5.87 (m, 1H) 7.37-7.47 (m, 2H) 7.76 (s, 1H) 8.10 (s, 1H) 8.38 (d, J=8.59 Hz, 1H) 9.32 (s, 1H). [M+H] calc'd for C₂₉H₄₀N₈O₃ 549; found, 549.

### Compound 207

### (S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide

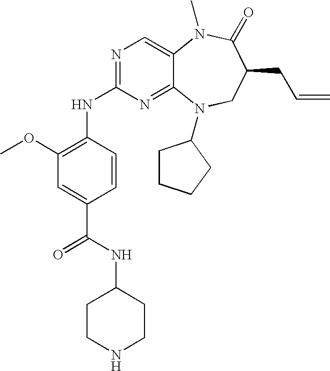

The title compound was prepared from (S)-4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid and tert-butyl 4-aminopiperidine-1-carboxylate, using an analogous procedure to that described in connection with Compound 188 followed by removal of Boc using TFA in DCM. the final compound was purified by reverse phase HPLC and basified to give the free base. ¹H NMR (400 MHz, DMSO-d₆) δ ppm 1.33-1.86 (m, 12H) 1.99-2.13 (m, 2H) 2.39-2.56 (m, 2H) 2.72-2.83 (m, 1H) 2.96 (br d, J=12.38 Hz, 2H) 3.19 (s, 3H) 3.40 (d, J=6.57 Hz, 2H) 3.76-3.89 (m, 1H) 3.94 (s, 3H) 4.76-4.90 (m, 1H) 5.03 (d, J=10.11 Hz, 1H) 5.08 (d, J=17.43 Hz, 1H) 5.72-5.87 (m, 1H) 7.45-7.52 (m, 2H) 7.74 (s, 1H) 8.06 (d, J=8.08 Hz, 1H) 8.09 (s, 1H) 8.37 (d, J=8.34 Hz, 1H). [M+H] calc'd for C₂₉H₃₉N₇O₃ 534; found, 534.

### Compound 208

### 4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic Acid

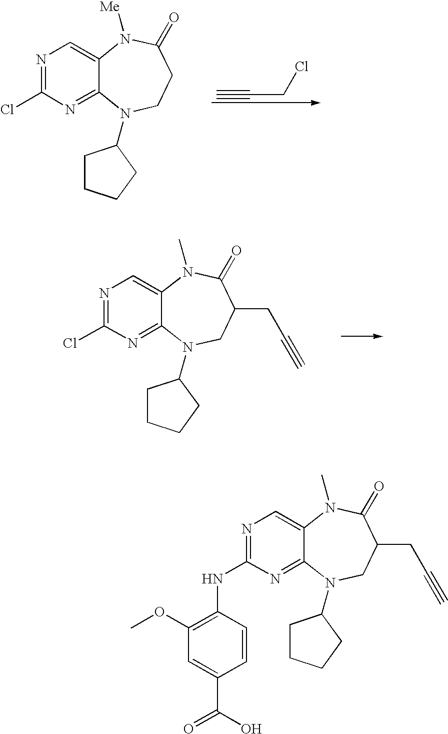

2-Chloro-9-cyclopentyl-5-methyl-7-(prop-2-ynyl)-8,9-dihydro-5H-pyrimido[4,5-b][1,4]diazepin-6(7H)-one. This intermediate was synthesized as described in Scheme 17 where propargyl chloride was used as the electrophile. [M+H] calc'd for C₁₆H₁₉ClNO₄ 319; found, 319.

4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid. 7-allyl-2-chloro-9-cyclopentyl-5-methyl-8,9-dihydro-5H-pyrimido[4,5-b][1,4]diazepin-6(7H)-one and 4-amino-3-methoxybenzoic acid (1.1 equivalent) were dissolved in dioxane and treated with p-toluenesulfonic acid monohydrate. The heterogeneous reaction mixture was stirred in a closed vial at 100° C. for 3 days and cooled to room temperature. The precipitate was filtered, washed with ethyl acetate (3×10 mL) and dried in vacuum to afford the title compound as a brown solid, which was used in the next step without further purification. [M+H] calc'd for C₂₄H₂₇N₅O₄ 450; found, 450.

### Compound 209

### 4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

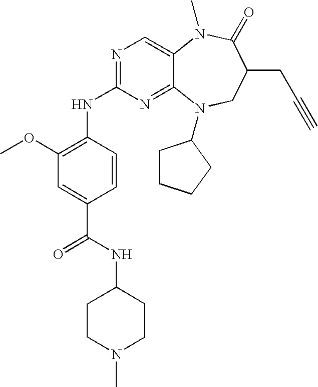

The title compound was synthesized using an analogous procedure to that described in connection with Compound 188 and the final compound was purified by reverse phase HPLC and basified to give the free base. ¹H NMR (400 MHz, DMSO-d₆) δ ppm 1.48-1.87 (m, 11H) 1.91-2.15 (m, 3H) 2.20 (br. s., 3H) 2.26-2.36 (m, 1H) 2.51-2.55 (m, 1H) 2.75-2.94 (m, 4H) 3.20 (s, 3H) 3.44-3.53 (m, 1H) 3.64 (dd, J=12.38, 2.02 Hz, 1H) 3.76 (br. s., 1H) 3.95 (s, 3H) 4.88 (t, J=8.08 Hz, 1H) 7.44-7.51 (m, 2H) 7.76 (s, 1H) 8.08 (d, J=7.58 Hz, 1H) 8.10 (s, 1H) 8.37 (d, J=8.34 Hz, 1H). [M+H] calc'd for C₃₀H₃₉N₇O₃ 546; found, 546.

### Compound 210

### 4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide

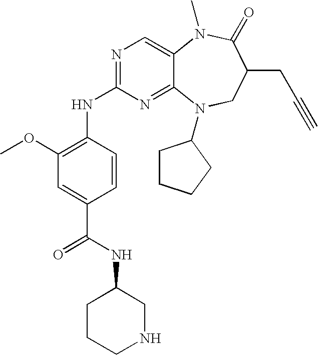

The title compound was prepared from 4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid and (R)-tert-butyl 3-aminopiperidine-1-carboxylate, using an analogous procedure to that described in connection with Compound 209 followed by removal of Boc using TFA in DCM. The final compound was purified by reverse phase HPLC and basified to give the free base. ¹H NMR (400 MHz, DMSO-d₆) δ ppm 1.33-1.90 (m, 11H) 2.03-2.16 (m, 1H) 2.16-2.45 (m, 4H) 2.75-2.84 (m, 1H) 2.85-3.00 (m, 3H) 3.19 (s, 3H) 3.43-3.54 (m, 1H) 3.64 (dd, J=12.51, 1.64 Hz, 1H) 3.73-3.87 (m, 1H) 3.94 (s, 3H) 4.88 (t, J=8.21 Hz, 1H) 7.43-7.52 (m, 2H) 7.78 (s, 1H) 7.98 (d, J=8.08 Hz, 1H) 8.11 (s, 1H) 8.37 (d, J=8.84 Hz, 1H). [M+H] calc'd for C₂₉H₃₇N₇O₃ 532; found, 532.

### Compound 211

### 4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(piperidin-4-yl)benzamide

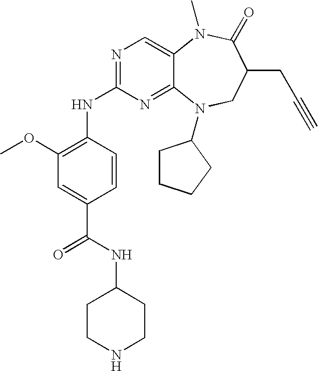

The title compound was prepared from 4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid and tert-butyl 4-aminopiperidine-1-carboxylate, using an analogous procedure to that described in connection with Compound 209 followed by removal of Boc using TFA in DCM. The final compound was purified by reverse phase HPLC and basified to give the free base. ¹H NMR (400 MHz, DMSO-d₆) δ ppm 1.32-1.87 (m, 11H) 2.09 (dd, J=10.11, 5.56 Hz, 1H) 2.25-2.36 (m, 1H) 2.42-2.56 (m, 2H) 2.84-3.01 (m, 4H) 3.19 (s, 3H) 3.43-3.55 (m, 1H) 3.64 (dd, J=12.38, 1.77 Hz, 1H) 3.75-3.89 (m, 1H) 3.94 (s, 3H) 4.78-4.97 (m, 1H) 7.44-7.55 (m, 2H) 7.77 (s, 1H) 8.06-8.14 (m, 2H) 8.37 (d, J=8.34 Hz, 1H). [M+H] calc'd for C₂₉H₃₇N₇O₃ 532; found, 532.

### Compound 212

### (S)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide

### Compound 213

### (R)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide

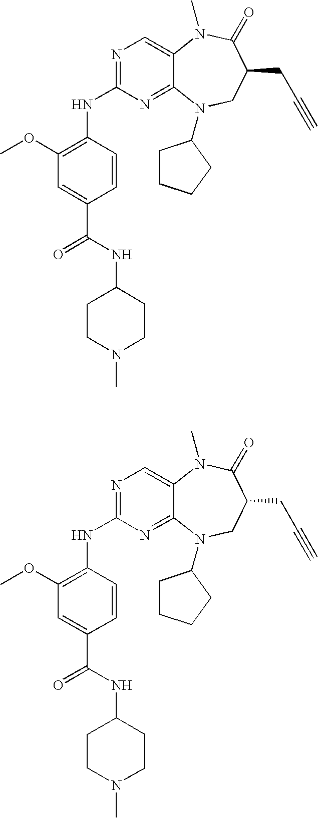

The title compounds were obtained by separation of Compound 209 using SFC (ChiralPak AD-H (5 um, 10×250 mm), 40% 2-Propanol buffered with NH₄OAc (10 mM) in supercritical CO₂). For both enantiomers: ¹H NMR (400 MHz, DMSO-d₆) δ ppm 1.48-1.87 (m, 11H) 1.91-2.15 (m, 3H) 2.20 (br. s., 3H) 2.26-2.36 (m, 1H) 2.51-2.55 (m, 1H) 2.75-2.94 (m, 4H) 3.20 (s, 3H) 3.44-3.53 (m, 1H) 3.64 (dd, J=12.38, 2.02 Hz, 1H) 3.76 (br. s., 1H) 3.95 (s, 3H) 4.88 (t, J=8.08 Hz, 1H) 7.44-7.51 (m, 2H) 7.76 (s, 1H) 8.08 (d, J=7.58 Hz, 1H) 8.10 (s, 1H) 8.37 (d, J=8.34 Hz, 1H). [M+H] calc'd for C₃₀H₃₉N₇O₃ 546; found, 546.

### Compound 214

### 4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic Acid

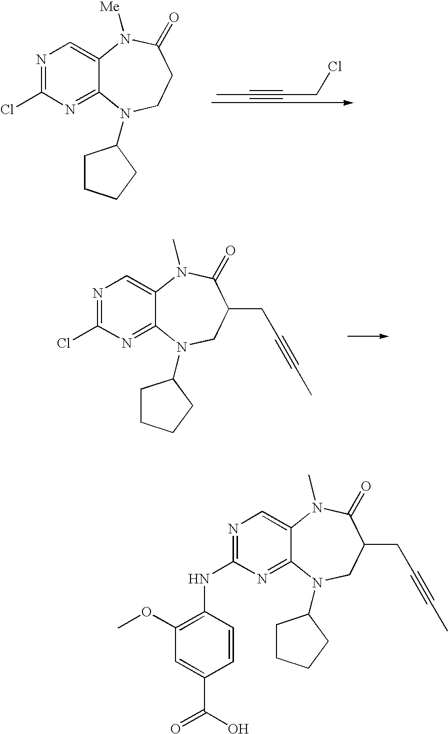

7-(but-2-ynyl)-2-chloro-9-cyclopentyl-5-methyl-8,9-dihydro-5H-pyrimido[4,5-b][1,4]diazepin-6(7H)-one. The compound was synthesized in the same manner as described in making 2-Chloro-9-cyclopentyl-5-methyl-7-(prop-2-ynyl)-8,9-dihydro-5H-pyrimido[4,5-b][1,4]diazepin-6(7H)-one except that 1-chlorobut-2-yne was used as electrophile.

7-(but-2-ynyl)-2-chloro-9-cyclopentyl-5-methyl-8,9-dihydro-5H-pyrimido[4,5-b][1,4]diazepin-6(7H)-one (3.51 g, 12.5 mmol) was dissolved in THF (20 mL) and added to a cooled (−78° C.) stirred solution of freshly prepared LDA (0.745M, 33.6 mL, 25.0 mmol) over the period of 10 min via cannula. The resulting yellow reaction mixture was stirred at −78° C. for 30 min and treated slowly with 1-bromobut-2-yne (5.00 g, 37.6 mmol). The resulting mixture was stirred at −78° C. for 2 h and at room temperature for 2 h. It was quenched with ammonium chloride (sat. aq., 50 mL) and water (15 mL). The mixture was extracted with ethyl acetate (2×50 mL) and the combined organic extracts were washed with brine (30 mL), dried (MgSO₄), filtered and concentrated in vacuo. Column chromatography on silica gel (ethyl acetate), afforded the title compound as a yellowish solid. (1.80 g, 43%). [M+H] calc'd for C₁₇H₂₁ClNO₄ 333; found, 333.

4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic acid. 7-(but-2-ynyl)-2-chloro-9-cyclopentyl-5-methyl-8,9-dihydro-5H-pyrimido[4,5-b][1,4]diazepin-6(7H)-one and 4-amino-3-methoxybenzoic acid (1.1 equivalent) were dissolved in dioxane and treated with p-toluenesulfonic acid monohydrate. The heterogeneous reaction mixture was stirred in a closed vial at 100° C. for 3 days and cooled to room temperature. The precipitate was filtered, washed with ethyl acetate (3×10 mL) and dried in vacuum to afford the title compound as a brown solid, which was used in the next step without further purification. [M+H] calc'd for C₂₅H₂₉N₅O₄ 464; found, 464.

### Compound 215

### 4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

### Compound 216

### (R)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

### Compound 217

### (S)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

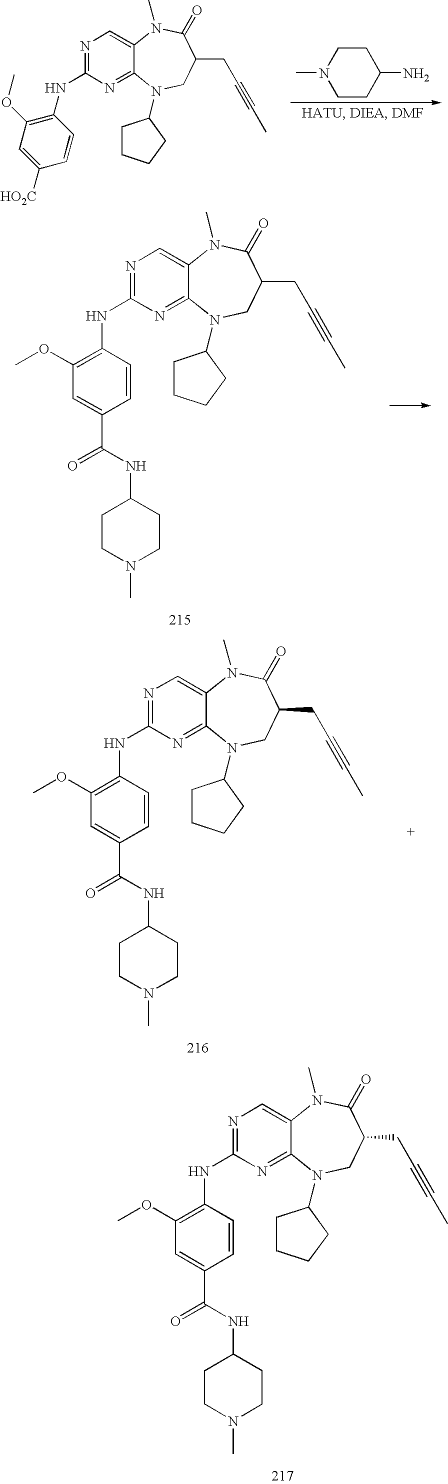

The mixture of the enantiomers (Compound 215) was synthesized using an analogous procedure to that described in connection with Compound 188 except that and the final compound was purified by reverse phase HPLC and basified to give the free base. The enantiomers were separated using SFC (ChiralPak OD-H (5 um, 10×250 mm), 16% MeOH buffered with NH₄OAc (10 mM) in supercritical CO₂).

The enantiomer of 4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide which shows shorter retention time: ¹H NMR (400 MHz, DMSO-d₆) δ ppm 1.47-1.87 (m, 14H) 1.89-2.02 (m, 2H) 2.06-2.16 (m, 1H) 2.16-2.21 (s, 3H) 2.21-2.29 (m, 1H) 2.38-2.48 (m, 1H) 2.75-2.85 (m, 3H) 3.19 (s, 3H) 3.46 (dd, J=12.38, 10.86 Hz, 1H) 3.66 (d, J=11.12 Hz, 1H) 3.69-3.81 (m, 1H) 3.94 (s, 3H) 4.87-4.99 (m, 1H) 7.45-7.52 (m, 2H) 7.77 (s, 1H) 8.05-8.15 (m, 2H) 8.37 (d, 1H). [M+H] calc'd for C₃₁H₄₁N₇O₃ 560; found, 560.

The enantiomer of 4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide which shows longer retention time: ¹H NMR (400 MHz, DMSO-d₆) δ ppm 1.47-1.87 (m, 14H) 1.89-2.02 (m, 2H) 2.06-2.16 (m, 1H) 2.16-2.21 (s, 3H) 2.21-2.29 (m, 1H) 2.38-2.48 (m, 1H) 2.75-2.85 (m, 3H) 3.19 (s, 3H) 3.46 (dd, J=12.38, 10.86 Hz, 1H) 3.66 (d, J=11.12 Hz, 1H) 3.69-3.81 (m, 1H) 3.94 (s, 3H) 4.87-4.99 (m, 1H) 7.45-7.52 (m, 2H) 7.77 (s, 1H) 8.05-8.15 (m, 2H) 8.37 (d, 1H). [M+H] calc'd for C₃₁H₄₁N₇O₃ 560; found, 560.

### Compound 218

### (Z)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

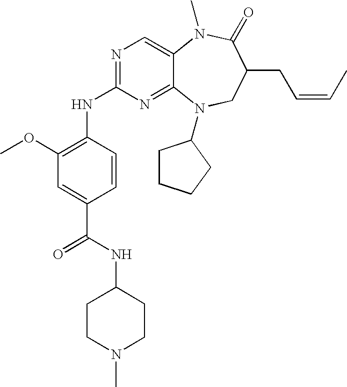

The title compound was obtained as described in the same manner in preparation of Compound 188 except that 1-bromo-2-butyne was used as electrophile, followed by hydrogenation using Lindnar's catalyst in acetone (1 atm H₂) and the final compound was purified by reverse phase HPLC and basified to give the free base. ¹H NMR (400 MHz, DMSO-d₆) δ ppm 1.37-1.87 (m, 14H) 1.87-2.38 (m, 8H) 2.63-2.77 (m, 1H) 2.77-2.97 (m, 2H) 3.19 (s, 3H) 3.36-3.50 (m, 2H) 3.68-3.84 (m, 1H) 3.94 (s, 3H) 4.75-4.93 (m, 1H) 5.29-5.44 (m, 1H) 5.44-5.58 (m, 1H) 7.42-7.50 (m, 2H) 7.75 (s, 1H) 8.09 (s, 1H) 8.12 (d, J=7.58 Hz, 1H) 8.37 (d, J=8.59 Hz, 1H). [M+H] calc'd for C₃₁H₄₃N₇O₃ 562; found, 562.

### Compound 219

### 4-(9-cyclopentyl-5-methyl-6-oxo-7-(pent-2-ynyl)-6,7,8,9-tetrahydro-5H-pyrimido[4,5-b][1,4]diazepin-2-ylamino)-3-methoxy-N-(1-methylpiperidin-4-yl)benzamide

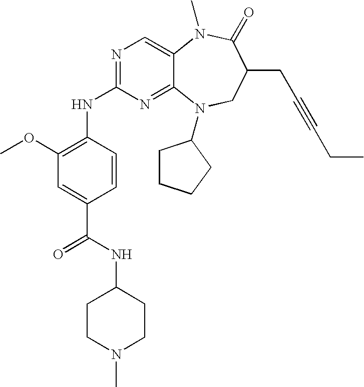

The title compound was obtained as described in the same manner in preparation of Compound 188 except that 1-bromo-2-pentyne was used as electrophile and the final compound was purified by reverse phase HPLC and basified to give the free base. ¹H NMR (400 MHz, CHLOROFORM-d) δ ppm 1.02 (t, J=7.45 Hz, 3H) 1.47-1.88 (m, 12H) 1.89-2.03 (m, 2H) 2.06-2.16 (m, 3H) 2.15-2.36 (m, 4H) 2.80 (dd, J=9.35, 3.54 Hz, 3H) 3.19 (s, 3H) 3.42-3.52 (m, 1H) 3.65-3.80 (m, 2H) 3.94 (s, 3H) 4.90-5.00 (m, 1H) 7.45-7.51 (m, 2H) 7.77 (s, 1H) 8.09 (s, 1H) 8.11 (d, J=8.08 Hz, 1H) 8.37 (d, J=8.84 Hz, 1H). [M+H] calc'd for C₃₂H₄₃N₇O₃ 574; found, 574.

### Compound 220

### (E)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

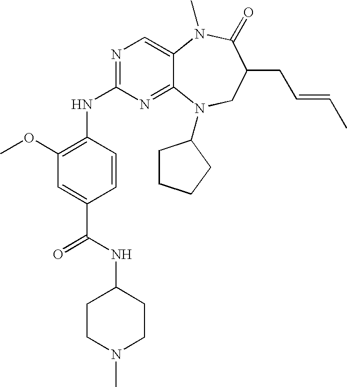

The title compound was obtained as described in the same manner in preparation of Compound 188 from 2-chloro-9-cyclopentyl-5-methyl-8,9-dihydro-5H-pyrimido[4,5-b][1,4]diazepin-6(7H)-one except that trans-1-chloro-2-butene was used as electrophile and the final compound was purified by reverse phase HPLC and basified to give the free base. ¹H NMR (400 MHz, DMSO-d₆) δ ppm 1.36-1.51 (m, 1H) 1.51-1.87 (m, 14H) 1.88-2.13 (m, 3H) 2.19 (s, 3H) 2.36 (d, J=14.40 Hz, 1H) 2.64-2.75 (m, 1H) 2.81 (d, J=10.36 Hz, 2H) 3.20 (s, 3H) 3.36-3.46 (m, 2H) 3.67-3.83 (m, 1H) 3.95 (s, 3H) 4.78-4.96 (m, 1H) 5.31-5.45 (m, 1H) 5.45-5.56 (m, 1H) 7.44-7.52 (m, 2H) 7.75 (s, 1H) 8.07-8.15 (m, 2H) 8.38 (d, J=8.34 Hz, 1H). [M+H] calc'd for C₃₁H₄₃N₇O₃ 562; found, 562.

### Compound 221

### 4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

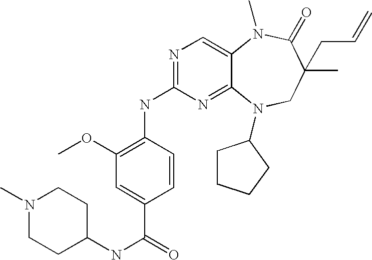

The title compound was obtained as described in the same manner in preparation of Compound 188 from 2-chloro-9-cyclopentyl-5,7-dimethyl-8,9-dihydro-5H-pyrimido[4,5-b][1,4]diazepin-6(7H)-one, using trans-1-chloro-2-butene as electrophile and the final compound was purified by reverse phase HPLC then basified to give the free base. ¹H NMR (400 MHz, CHLOROFORM-d) δ ppm 1.19 (d, J=5.05 Hz, 3H) 1.42-1.84 (m, 8H) 1.87-2.51 (m, 11H) 2.93 (br. s., 2H) 3.30 (d, J=5.56 Hz, 2H) 3.41 (dd, 2H) 3.98 (d, J=5.56 Hz, 2H) 4.05 (br. s., 2H) 5.00 (d, J=17.18 Hz, 1H) 5.08 (d, 1H) 5.31 (t, J=8.84 Hz, 1H) 5.67-5.86 (m, 1H) 5.96 (br. s., 1H) 7.18-7.30 (m, 2H) 7.41 (d, J=3.28 Hz, 1H) 7.63 (d, J=4.29 Hz, 1H) 7.85 (d, J=5.56 Hz, 1H) 8.49 (dd, J=8.59, 5.31 Hz, 1H). [M+H] calc'd for C₃₁H₄₃N₇O₃ 562; found, 562.

### Compound 222

### (S)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

### Compound 223

### (R)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

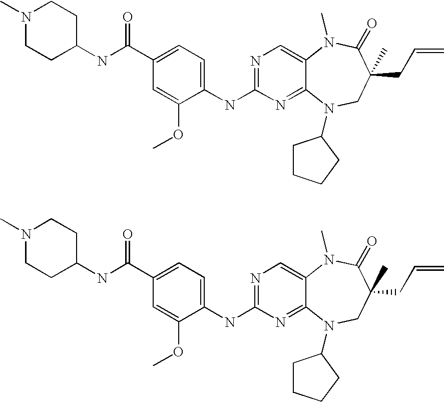

The enantiomers of Compound 221 were separated using SFC (ChiralPak OD-H (5 um, 10×250 mm), 16% Methanol in supercritical CO₂).

The enantiomer of 4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide which shows shorter retention time: ¹H NMR (400 MHz, DMSO-d₆) δ ppm 1.09 (s, 3H) 1.48-2.05 (m, 12H) 2.13 (dd, J=13.89, 7.33 Hz, 1H) 2.27 (dd, J=13.77, 7.20 Hz, 1H) 2.68 (s, 3H) 3.05 (br. s., 2H) 3.19 (s, 3H) 3.33-3.53 (m, 4H) 3.95 (s, 3H) 4.03 (br. s., 1H) 4.97 (d, J=14.91 Hz, 1H) 5.04 (d, J=12.38 Hz, 1H) 5.12-5.28 (m, 1H) 5.62-5.82 (m, 1H) 7.45-7.60 (m, 2H) 7.70 (s, 1H) 7.99 (s, 1H) 8.37 (d, J=8.34 Hz, 2H). [M+H] calc'd for C₃₁H₄₃N₇O₃ 562; found, 562.

The enantiomer of 4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide which shows longer retention time: ¹H NMR (400 MHz, DMSO-d₆) δ ppm 1.09 (s, 3H) 1.48-2.05 (m, 12H) 2.13 (dd, J=13.89, 7.33 Hz, 1H) 2.27 (dd, J=13.77, 7.20 Hz, 1H) 2.68 (s, 3H) 3.05 (br. s., 2H) 3.19 (s, 3H) 3.33-3.53 (m, 4H) 3.95 (s, 3H) 4.03 (br. s., 1H) 4.97 (d, J=14.91 Hz, 1H) 5.04 (d, J=12.38 Hz, 1H) 5.12-5.28 (m, 1H) 5.62-5.82 (m, 1H) 7.45-7.60 (m, 2H) 7.70 (s, 1H) 7.99 (s, 1H) 8.37 (d, J=8.34 Hz, 2H). [M+H] calc'd for C₃₁H₄₃N₇O₃ 562; found, 562.

### Compound 224

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

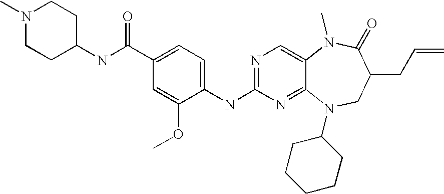

The title compound was synthesized using an analogous procedure to that described in connection with Compound 187 except that cyclohexanone instead of cyclopentanone was used in the reductive amination and the final compound was purified by reverse phase HPLC and basified to give the free base. ¹H NMR (400 MHz, DMSO-d₆) δ ppm 2.00 (s, 18H) 2.18 (s, 3H) 2.38-2.49 (m, 1H) 2.72-2.86 (m, 3H) 3.20 (s, 3H) 3.54 (d, J=13.64 Hz, 1H) 3.68-3.82 (m, 1H) 3.96 (s, 3H) 4.31-4.46 (m, 1H) 5.05 (d, J=9.85 Hz, 1H) 5.10 (d, J=17.18 Hz, 1H) 5.74-5.93 (m, 1H) 7.48 (d, J=8.59 Hz, 1H) 7.52 (s, 1H) 7.73 (s, 1H) 8.08 (s, 1H) 8.11 (d, J=7.83 Hz, 1H) 8.38 (d, J=8.34 Hz, 1H). [M+H] calc'd for C₃₁H₄₃N₇O₃ 562; found, 562.

### Compound 225

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide

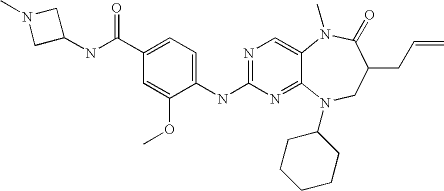

The title compound was synthesized using an analogous procedure to that described in connection with Compound 224 except that 3-amino-1-methylazetine instead of 4-amino-1-methylpiperidine was used and the final compound was purified by reverse phase HPLC and basified to give the free base. ¹H NMR (400 MHz, DMSO-d₆) δ ppm 1.06-1.94 (m, 11H) 1.99-2.09 (m, 1H) 2.26 (s, 3H) 2.38-2.47 (m, 1H) 2.77 (dd, J=8.21, 2.15 Hz, 1H) 2.99 (t, J=6.82 Hz, 2H) 3.18 (s, 3H) 3.55 (t, J=7.20 Hz, 3H) 3.95 (s, 3H) 4.31-4.49 (m, 2H) 5.04 (d, J=10.36 Hz, 1H) 5.09 (d, J=17.18 Hz, 1H) 5.80 (ddd, J=17.24, 9.92, 5.68 Hz, 1H) 7.47 (dd, J=8.46, 1.64 Hz, 1H) 7.52 (s, 1H) 7.73 (s, 1H) 8.07 (s, 1H) 8.38 (d, J=8.59 Hz, 1H) 8.62 (d, J=6.82 Hz, 1H). [M+H] calc'd for C₂₉H₃₉N₇O₃ 534; found, 534.

### Compound 226

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide

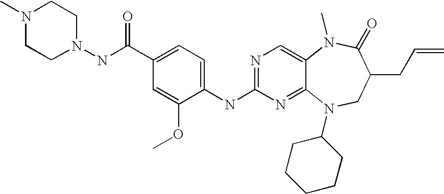

The title compound was synthesized using an analogous procedure to that described in connection with Compound 224 except that 4-methylpiperazin-1-amine instead of 4-amino-1-methylpiperidine was used and the final compound was purified by reverse phase HPLC and basified to give the free base. ¹H NMR (400 MHz, DMSO-d₆) δ ppm 1.04-1.24 (m, 1H) 1.27-1.42 (m, 1H) 1.43-1.64 (m, 3H) 1.64-1.78 (m, 2H) 1.77-1.97 (m, 3H) 1.98-2.11 (m, 1H) 2.19 (s, 3H) 2.28-2.48 (m, 4H) 2.70-2.84 (m, 1H) 2.95 (br. s., 4H) 3.19 (s, 3H) 3.53 (d, J=12.88 Hz, 1H) 3.95 (s, 3H) 4.26-4.47 (m, 1H) 4.99-5.14 (m, 2H) 5.71-5.91 (m, 1H) 7.36-7.50 (m, 2H) 7.73 (s, 1H) 8.07 (s, 1H) 8.38 (d, J=8.34 Hz, 1H) 9.35 (s, 1H). [M+H] calc'd for C₃₀H₄₁N₈O₃ 563; found, 563.

### Compound 227

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide

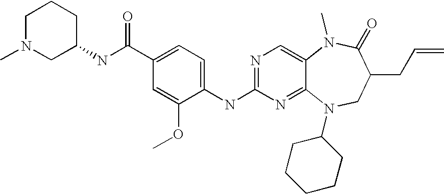

The title compound was synthesized using an analogous procedure to that described in connection with Compound 224 except that (S)-1-methylpiperidin-3-amine instead of 4-amino-1-methylpiperidine was used and the final compound was purified by reverse phase HPLC and basified to give the free base. ¹H NMR (400 MHz, DMSO-d₆) δ ppm 1.06-1.94 (m, 17H) 1.99-2.10 (m, 1H) 2.17 (s, 3H) 2.37-2.47 (m, 1H) 2.61-2.70 (m, 1H) 2.72-2.85 (m, 2H) 3.18 (s, 3H) 3.53 (dd, J=12.63, 1.52 Hz, 1H) ¹H NMR 3.95 (s, 4H) 4.38 (t, J=11.24 Hz, 1H) 5.04 (d, J=10.11 Hz, 1H) 5.09 (dd, J=17.31, 1.64 Hz, 1H) 5.72-5.89 (m, J=17.18, 7.89, 2.24, 2.24 Hz, 1H) 7.45 (dd, J=8.59, 1.77 Hz, 1H) 7.49 (d, J=1.77 Hz, 1H) 7.72 (s, 1H) 8.01 (d, J=8.08 Hz, 1H) 8.06 (s, 1H) 8.36 (d, J=8.34 Hz, 1H). [M+H] calc'd for C₃₁H₄₃N₇O₃ 562; found, 562.

### Compound 228

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide

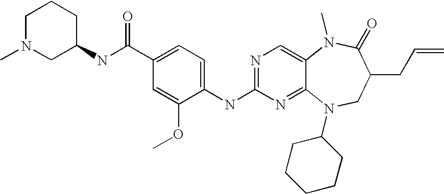

The title compound was synthesized using an analogous procedure to that described in connection with Compound 224 except that (R)-1-methylpiperidin-3-amine instead of 4-amino-1-methylpiperidine was used and the final compound was purified by reverse phase HPLC and basified to give the free base. ¹H NMR (400 MHz, DMSO-d₆) δ ppm 1.06-1.95 (m, 17H) 1.97-2.09 (m, 1H) 2.18 (s, 3H) 2.43 (ddd, J=14.27, 5.81, 5.68 Hz, 1H) 2.62-2.71 (m, 1H) 2.71-2.86 (m, 2H) 3.19 (s, 3H) 3.53 (dd, J=12.76, 1.64 Hz, 1H) 3.95 (s, 4H) 4.38 (br. s., 1H) 5.04 (d, J=10.11 Hz, 1H) 5.09 (dd, J=17.18, 1.52 Hz, 1H) 5.70-5.94 (m, J=17.21, 7.86, 2.24, 2.24 Hz, 1H) 7.46 (dd, J=8.46, 1.64 Hz, 1H) 7.50 (s, 1H) 7.72 (s, 1H) 8.01 (d, J=7.83 Hz, 1H) 8.07 (s, 1H) 8.37 (d, J=8.34 Hz, 1H). [M+H] calc'd for C₃₁H₄₃N₇O₃ 562; found, 562.

### Compound 229

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide

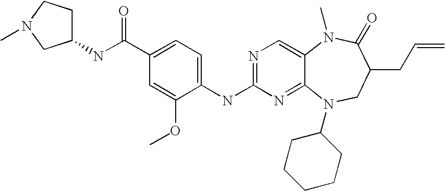

The title compound was synthesized using an analogous procedure to that described in connection with Compound 224 except that (S)-1-methylpyrrolidin-3-amine instead of 4-amino-1-methylpiperidine was used and the final compound was purified by reverse phase HPLC and basified to give the free base. ¹H NMR (400 MHz, DMSO-d₆) δ ppm 1.03-1.96 (m, 12H) 1.97-2.11 (m, 1H) 2.12-2.26 (m, 1H) 2.33 (s, 3H) 2.36-2.47 (m, 2H) 2.52-2.63 (m, 1H) 2.65-2.86 (m, 3H) 3.19 (s, 3H) 3.53 (d, J=11.37 Hz, 1H) 3.95 (s, 3H) 4.30-4.49 (m, 2H) 5.04 (d, J=8.34 Hz, 1H) 5.09 (d, J=17.43 Hz, 1H) 5.72-5.92 (m, 1H) 7.49 (d, J=8.59 Hz, 1H) 7.54 (s, 1H) 7.72 (s, 1H) 8.07 (s, 1H) 8.33-8.42 (m, 2H). [M+H] calc'd for C₃₀H₄₁N₇O₃ 548; found, 548.

### Compound 230

### 4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide

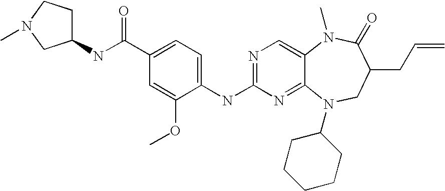

The title compound was synthesized using an analogous procedure to that described in connection with Compound 224 except that (R)-1-methylpyrrolidin-3-amine instead of 4-amino-1-methylpiperidine was used and the final compound was purified by reverse phase HPLC and basified to give the free base. ¹H NMR (400 MHz, DMSO-d₆) δ ppm 1.03-1.96 (m, 12H) 1.97-2.11 (m, 1H) 2.12-2.26 (m, 1H) 2.33 (s, 3H) 2.36-2.47 (m, 2H) 2.52-2.63 (m, 1H) 2.65-2.86 (m, 3H) 3.19 (s, 3H) 3.53 (d, J=11.37 Hz, 1H) 3.95 (s, 3H) 4.30-4.49 (m, 2H) 5.04 (d, J=8.34 Hz, 1H) 5.09 (d, J=17.43 Hz, 1H) 5.72-5.92 (m, 1H) 7.49 (d, J=8.59 Hz, 1H) 7.54 (s, 1H) 7.72 (s, 1H) 8.07 (s, 1H) 8.33-8.42 (m, 2H). [M+H] calc'd for C₃₀H₄₁N₇O₃ 548; found, 548.

### Compound 231

### 4-(9-cyclopentyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

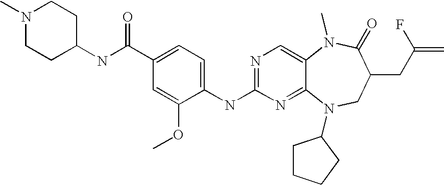

The title compound was obtained as described in the same manner in preparation of Compound 188 from 2-chloro-9-cyclopentyl-5-methyl-8,9-dihydro-5H-pyrimido[4,5-b][1,4]diazepin-6(7H)-one except that 3-bromo-2-fluoroprop-1-ene was used as electrophile and the final compound was purified by reverse phase HPLC and basified to give the free base. ¹H NMR (400 MHz, CHLOROFORM-d) δ ppm 1.45-1.91 (m, 12H), 2.04-2.26 (m, 5H), 2.33 (s, 3H), 2.63 (dd, J=9.09, 3.54 Hz, 1H), 2.82-2.92 (m, 2H), 3.31 (s, 3H), 3.72 (m, 2H), 3.96-4.04 (m, 4H), 4.53 (br.s, 1H), 4.91-4.96 (m, 1H), 5.93 (d, J=7.83 Hz, 1H), 7.24 (dd, J=8.34, 1.77 Hz, 1H), 7.43 (d, J=2.02 Hz, 1H), 7.70 (s, 1H), 7.98 (s, 1H), 8.50 (d, J=8.59 Hz, 1H). [M+H] calc'd for C₃₀H₄₀FN₇O₃ 566; found, 566

### Compound 232

### 4-(9-Cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide

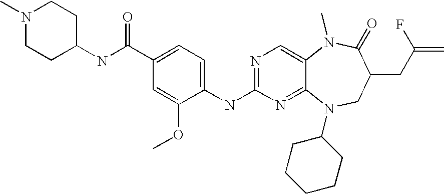

The title compound was obtained as described in the same manner in preparation of Compound 224 from 2-chloro-9-cyclohexyl-5-methyl-8,9-dihydro-5H-pyrimido[4,5-b][1,4]diazepin-6(7H)-one except that 3-bromo-2-fluoroprop-1-ene was used as electrophile and the final compound was purified by reverse phase HPLC and basified to give the free base. ¹H NMR (400 MHz, CHLOROFORM-d) δ ppm 1.15-1.22 (m, 1H), 1.41-1.69 (m, 7H), 1.75-1.86 (m, 2H), 1.87-2.10 (m, 7H), 2.15-2.22 (m, 2H), 2.31 (s, 3H), 2.62 (d, J=11.12 Hz, 1H), 2.83 (d, J=12.13 Hz, 2H), 3.30 (s, 3H), 3.53-3.59 (m, 1H), 3.86 (d, J=12.88 Hz, 1H), 3.92-4.07 (m, 4H), 4.42 (br. s, 1H), 4.49-4.63 (m, 1H), 5.93 (d, J=7.83 Hz, 1H), 7.25 (dd, J=8.34, 1.77 Hz, 1H), 7.39 (d, J=1.77 Hz, 1H), 7.71 (s, 1H), 7.94 (s, 1H), 8.50 (d, J=8.34 Hz, 1H). [M+H] calc'd for C₃₁H₄₂FN₇O₃ 580; found, 580.

### Compound 233

### 4-(9-cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylazetidin-3-yl)benzamide

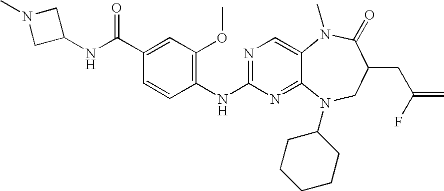

The title compound was obtained as described in the same manner in preparation of Compound 232 except that 3-amino-1methylazetine was used and the final compound was purified by reverse phase HPLC and basified to give the free base. ¹H NMR (400 MHz, CHLOROFORM-d) δ ppm 1.13-1.24 (m, 1H), 1.42-1.69 (m, 6H), 1.76-2.04 (m, 6H), 2.38 (S, 3H), 2.61-2.64 (m, 1H), 3.12-3.19 (m, 2H), 3.30 (s, 3H), 3.50-3.61 (m, 1H), 3.67 (t, J=7.71 Hz, 2H), 3.82-3.90 (m, 1H), 3.96 (s, 3H), 4.43-4.57 (br. s., 1H), 4.51-4.63 (m, 1H), 4.64-4.77 (m, 1H), 6.60 (d, J=7.58 Hz, 1H), 7.32 (dd, J=8.46, 1.1.77 Hz, 1H), 7.42 (d, J=1.77 Hz, 1H), 7.74 (s, 1H), 7.95 (s, 1H), 8.51 (d, J=8.59 Hz, 1H). [M+H] calc'd for C₂₉H₃₈FN₇O₃ 552; found, 552.

### Compound 234

### 4-(9-cyclopentyl-7-methoxy-5,7-dimethyl-6-oxo-6,7,8,9-tetrahydro-5H-pyrimido[4,5-b][1,4]diazepin-2-ylamino)-3-methoxybenzoic Acid

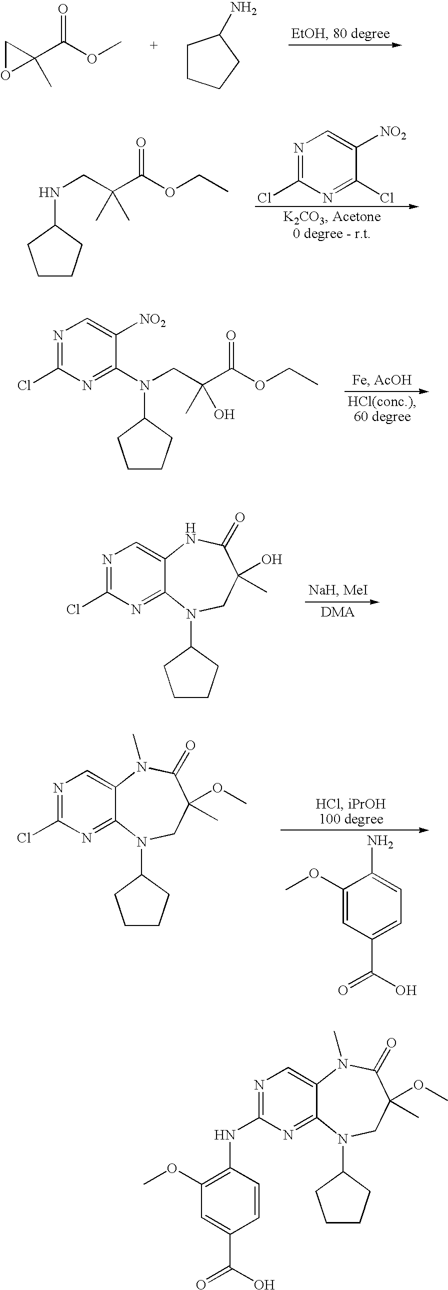

Ethyl 3-(cyclopentylamino)-2,2-dimethylpropanoate. To an amount of 10 g (0.0861 mole) of methyl 2-methyl glycidate, was added 150 ml of EtOH and 13 mL (0.129 mole) cyclopentylamine. The mixture was heated to 80 C overnight. Solvent was evaporated in vacuo and the resulting yellow oil (17 g, 92%) was carried onto the next step without any purification. [M+H] calc'd for C₁₂H₂₃NO₂, 214; found 214.

Ethyl 3-((2-chloro-5-nitropyrimidin-4-yl)(cyclopentyl)amino)-2-hydroxy-2-methylpropanoate. The title compound was synthesized using an analogous procedure same as that described in connection with ethyl 3-((2-chloro-5-nitropyrimidin-4-yl)(cyclopentyl)amino)-2,2-dimethyl propanoate. [M+H] calc'd for C₁₅H₂₁ClN₄O₅, 373; found 373.

2-Chloro-9-cyclopentyl-7-hydroxy-7-methyl-8,9-dihydro-5H-pyrimido[4,5-b][1,4]diazepin-6(7H)-one. The titled compound was synthesized using an analogous procedure same as that described in connection with 2-chloro-9-cyclopentyl-7,7-dimethyl-8,9-dihydro-5H-pyrimido[4,5-b][1,4]diazepin-6(7H)-one. [M+H] calc'd for C₁₃H₁₇ClN₄O₂, 297; found 297.

2-Chloro-9-cyclopentyl-7-methoxy-5,7-dimethyl-8,9-dihydro-5H-pyrimido[4,5-b][1,4]diazepin-6(7H)-one. The title compound was synthesized using an analogous procedure same as that described in connection with 2-chloro-9-cyclopentyl-5,7,7-trimethyl-8,9-dihydro-5H-pyrimido[4,5b][1,4]diazepin-6(7H)-one, except that 3.0 eq of iodomethane and sodium hydride were used. [M+H] calc'd for C₁₅H₂₁ClN₄O₂, 325; found 325.

4-(9-Cyclopentyl-7-methoxy-5,7-dimethyl-6-oxo-6,7,8,9-tetrahydro-5H-pyrimido[4,5-b][1,4]diazepin-2-ylamino)-3-methoxybenzoic acid. The title compound was synthesized using an analogous procedure same as that described in connection with 4-(9-cyclopentyl-5,7,7-trimethyl-6-oxo-6,7,8,9-tetrahydro-5H-pyrimido[4,5-b][1,4]diazepin-2-ylamino)-3-methoxybenzoic acid. [M+H] calc'd for C₂₃H₂₉N₅O₅, 456; found 456.

### Compound 235

### 4-(9-cyclopentyl-7-methoxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

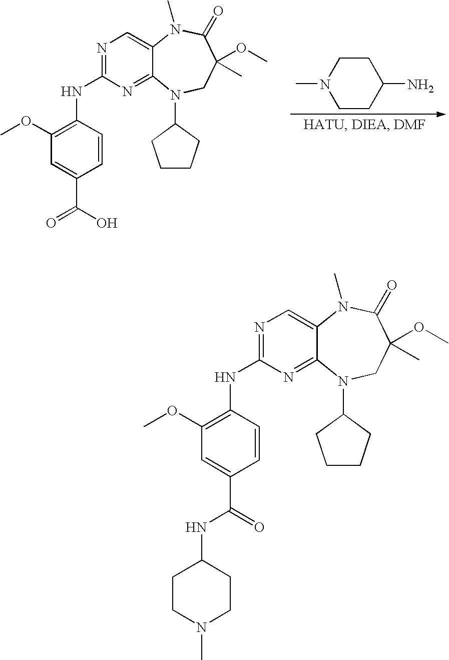

The title compound was synthesized using an analogous procedure same as that described in connection with Compound 86 and the final compound was purified by reverse phase HPLC and basified to give the free base. ¹H NMR (400 MHz, MeOD) δ ppm 1.46 (s, 3H) 1.49-2.12 (m, 10H) 2.26 (d, J=14.08 Hz, 2H) 2.90 (s, 3H) 3.10 (s, 3H) 3.11-3.25 (m, 2H) 3.40 (s, 3H) 3.61 (d, J=14.78 Hz, 2H) 3.91 (d, J=14.78 Hz, 2H) 4.01 (s, 3H) 4.16 (t, J=11.62 Hz, 1H) 5.09 (t, J=8.56 Hz, 1H) 7.54 (d, J=9.54 Hz, 1H) 7.61 (s, 1H) 7.94 (s, 1H) 8.08 (d, J=8.40 Hz, 1H). [M+H] calc'd for C₂₉H₄₁N₇O₄, 552; found 552.

### Compound 236

### 4-(9-cyclopentyl-7-hydroxy-5,7-dimethyl-6-oxo-6,7,8,9-tetrahydro-5H-pyrimido[4,5-b][1,4]diazepin-2-ylamino)-3-methoxybenzoic Acid

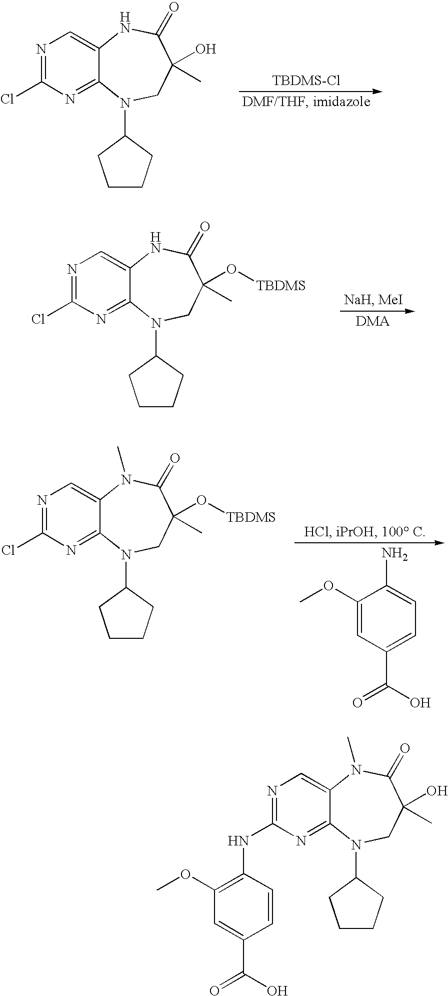

7-(tert-Butyldimethylsilyloxy)-2-chloro-9-cyclopentyl-7-methyl-8,9-dihydro-5H-pyrimido[4,5-b][1,4]diazepin-6(7H)-one. To a solution of 2-chloro-9-cyclopentyl-7-hydroxy-7-methyl-8,9-dihydro-5H-pyrimido[4,5-b][1,4]diazepin-6(7H)-one (62 mg, 0.2095 mmole), which was dissolved in DMF/THF (300 uL/100 uL), was added TBDMS-Cl (176 mg, 1.047 mmole) and imidazole (71.3 mg, 1.0473 mmole). The mixture was stirred at r.t. for 3 days. Afterwards, it was diluted with EtoAce (5 mL) and was washed with brine (3×5 mL). The organic layer was dried over Na₂SO₄ and evaporated in vacuo to yield a yellow solid (87 mg, 98%). [M+H] calc'd for C₁₉H₃₁ClN₄O₂Si, 411; found 411.

7-(tert-Butyldimethylsilyloxy)-2-chloro-9-cyclopentyl-5,7-dimethyl-8,9-dihydro-5H-pyrimido[4,5-b][1,4]diazepin-6(7H)-one. The title compound was synthesized using an analogous procedure same as that described in connection with 2-chloro-9-cyclopentyl-7-methoxy-5,7-dimethyl-8,9-dihydro-5H-pyrimido[4,5-b][1,4]diazepin-6(7H)-one. [M+H] calc'd for C₂₀H₃₃ClN₄O₂Si, 425; found 425.

4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxybenzoic acid. The title compound was synthesized using an analogous procedure same as that described in connection with Compound 236. [M+H] calc'd for C₂₂H₂₇N₅O₅, 442; found 442.

### Compound 237

### 4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

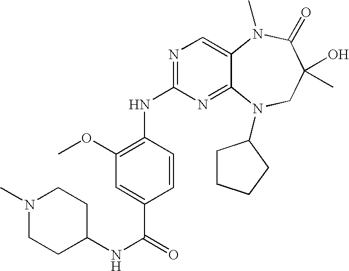

The title compound was synthesized using an analogous procedure same as that described in connection with Compound 235 and the final compound was purified by reverse phase HPLC and basified to give the free base. ¹H NMR (400 MHz, MeOD) δ ppm 1.59 (s, 3H) 1.63-2.04 (m, 6H) 2.11-2.30 (m, 4H) 2.90 (s, 2H) 3.12-3.25 (m, 2H) 3.41 (s, 3H) 3.51-3.54 (m, 1H) 3.49-3.71 (m, 4H) 3.98 (s, 3H) 4.01 (s, 3H) 4.16 (tt, J=11.85, 4.07 Hz, 1H) 7.46-7.57 (m, 2H) 8.30 (s, 1H) 8.46-8.53 (m, J=10.71, 8.64, 0.62, 0.62 Hz, 1H). [M+H] calc'd for C₂₈H₃₉N₇O₄, 538; found 538.

### Compound 238

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic Acid

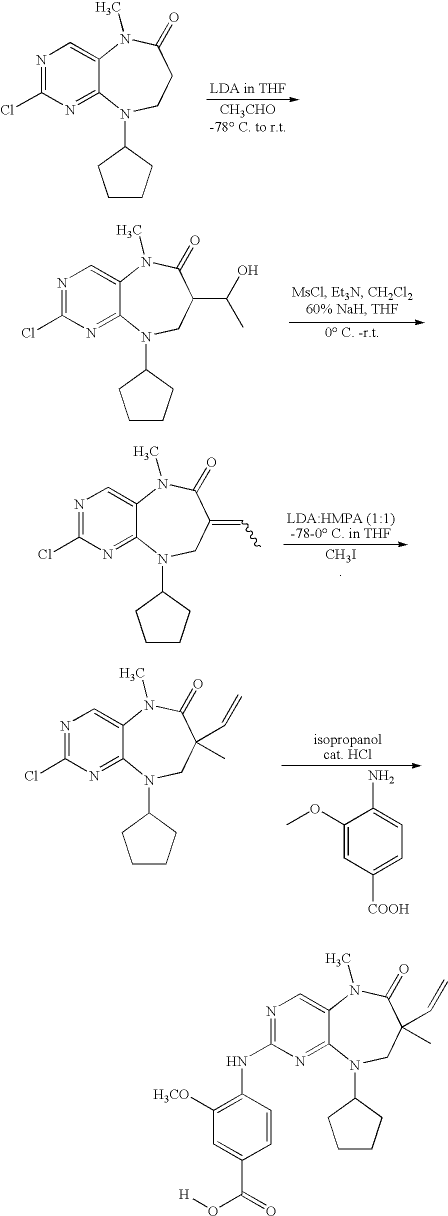

2-Chloro-9-cyclopentyl-7-(1-hydroxyethyl)-5-methyl-8,9-dihydro-5H-pyrimido[4,5-b][1,4]diazepin-6(7H)-one: In a round bottom flask, 2-chloro-9-cyclopentyl-8,9-dihydro-5H-pyrimido[4,5-b][1,4]diazepin-6(7H)-one (0.840 g, 3.0 mmol) was dissolved in dry tetrahydrofuran (12 mL), cooled to −78° C., then added 2.0 M lithium diisopropyl amide in tetrahydrofuran (6.0 mmol) drop wise. After 30 minutes, acetaldehyde (1.056 g, 24.0 mmol) in 6.0 mL tetrahydrofuran was added slowly drop wise to it, continued stirring for 60 minutes. After disappearing the starting material, cooled the reaction flask to −78° C., then quenched with sat. ammonium chloride solution (3 mL). At room temperature, the resultant reaction mixture was taken into ethyl acetate (50 mL), washed with 0.1 N HCl, ammonium chloride solution and finally with water, dried over sodium sulphate and evaporated. Purified the product using column chromatography with hexane:ethyl acetate mixtures to give the 2-chloro-9-cyclopentyl-7-(1-hydroxyethyl)-5-methyl-8,9-dihydro-5H-pyrimido[4,5-b][1,4]diazepin-6(7H)-one (0.842 g, 86%). [M+H] calculated for C₁₅H₂₂ClN₄O₂, 325; found 325.

2-Chloro-9-cyclopentyl-7-ethylidene-5-methyl-8,9-dihydro-5H-pyrimido[4,5-b][1,4]diazepin-6(7H)-one: In a round bottom flask, 2-chloro-9-cyclopentyl-7-(1-hydroxyethyl)-5-methyl-8,9-dihydro-5H-pyrimido[4,5-b][1,4]diazepin-6(7H)-one (1.60 g, 4.94 mmol) was dissolved in dichloromethane (15 mL) and cooled to 0° C. Then added triethylamine (1.39 mL, 9.88 mmol) to it, after 5 minutes added methanesulfonyl chloride (0.46 mL, 5.93 mmol) drop wise, continued stirring at room temperature for 14 hrs. Then after completion, the reaction mixture was taken into dichloromethane, washed with ice cold water (50 mL), dried over sodium sulfate and evaporated. The resultant O-methane sulfonyl compound was dissolved in dry tetrahydrofuran and cooled to 0° C., and then added 60% sodium hydride in mineral oil (0.218 g, 5.47 mmol) portion wise slowly, continued at room temperature for another 30 minutes. After completing the reaction, the reaction mixture was slowly added to ice cold water (50 mL) and extracted with ethyl acetate (2×50 mL), ethyl acetate layer was dried evaporated and purified using column chromatography to yield 2-chloro-9-cyclopentyl-7-ethylidene-5-methyl-8,9-dihydro-5H-pyrimido[4,5-b][1,4]diazepin-6(7H)-one (1.38 g, 94%). ¹H NMR (400 MHz, DMSO-d₆) δ ppm 1.12 (d, J=6.06 Hz, 3H) 1.42-2.10 (m, 8H) 3.18 (s, 3H) 3.42-4.05 (m, 3H) 4.75 (d, J=6.06 Hz, 1H) 4.85 (quin, J=8.39 Hz, 1H) 8.06-8.23 (m, 1H). [M+H] calculated for C₁₅H₂₀ClN₄O, 307; found 307.

2-Chloro-9-cyclopentyl-5,7-dimethyl-7-vinyl-8,9-dihydro-5H-pyrimido[4,5-b][1,4]diazepin-6(7H)-one: 2-chloro-9-cyclopentyl-7-ethylidene-5-methyl-8,9-dihydro-5H-pyrimido[4,5-b][1,4]diazepin-6(7H)-one (1.01 g, 3.30 mmol) in dry tetrahydrofuran (10 mL) was added to 1:1 molar 2 M lithium diisopropyl amide in tetrahydrofuran and hexamethyl phosphoramide (HMPA) (3.96 mmol each) in tetrahydrofuran (5 mL) at −78° C. After 20 minutes, methyl iodide (617.6 μL, 2.46 mmol) was added dropwise to it. Continued the reaction at −78° C. for 30 minutes, and then elevated the temperature to 0° C. for 30 minutes. After disappearing the starting material, cooled the reaction flask to −78° C., then quenched with sat. ammonium chloride solution (6 mL). At room temperature, the resultant reaction mixture was taken into ethyl acetate (70 mL), washed with ammonium chloride solution and finally with water, dried over sodium sulphate and evaporated. Purified the product using column chromatography with hexane:ethyl acetate mixtures to give 2-chloro-9-cyclopentyl-7-ethylidene-5-methyl-8,9-dihydro-5H-pyrimido[4,5-b][1,4]diazepin-6(7H)-one (0.212 g, 21%). ¹H NMR (400 MHz, CHLOROFORM-d) δ ppm 1.36 (s, 3H) 1.43-1.59 (m, 1H) 1.64-1.85 (m, 6H) 1.97-2.22 (m, 1H) 3.35 (s, 3H) 3.39 (d, J=14.15 Hz, 1H) 3.75 (d, J=14.15 Hz, 1H) 4.93 (d, J=17.68 Hz, 1H) 5.04 (d, J=10.86 Hz, 1H) 5.13 (m, 1H) 5.64 (dd, J=17.56, 10.74 Hz, 1H) 7.93 (br. s., 1H). [M+H] calculated for C₁₆H₂₂ClN₄O, 321; found 321.

4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic acid. The title compound was synthesized using an analogous procedure same as that described in connection with Compound 235. [M+H] calculated for C₂₄H₂₉ClN₅O₄ 452; found 452.

### Compound 239

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide

### Compound 240

### (R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide

### Compound 241

### (S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide

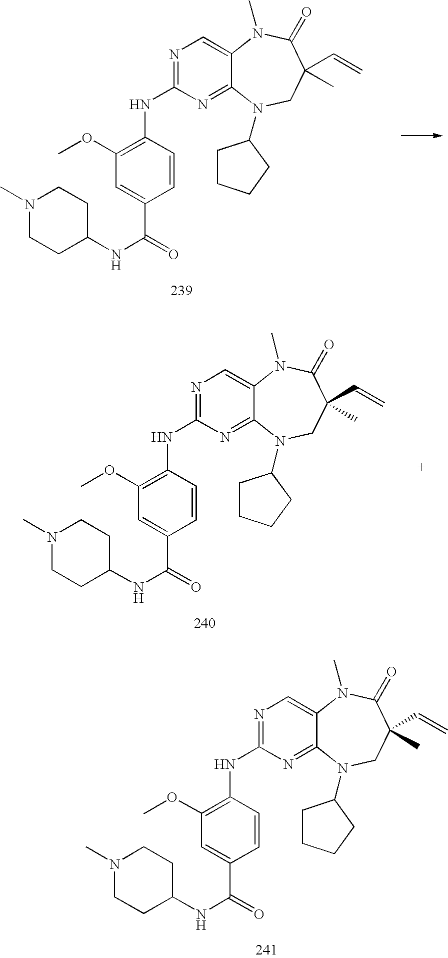

Compound 240 was synthesized using an analogous procedure same as that described in connection with Compound 86 and the final compound was purified by reverse phase HPLC and basified to give the free base. The enantiomers of this compound were separated using Gilson SFC (ChiralPak AD-H (5 um, 10×250 mm), 14% 2-propanol with 10 mM NH₄OAc in supercritical CO₂).

The enantiomer of 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide which shows shorter retention time: ¹H NMR (400 MHz, DMSO-d₆) δ ppm 1.23 (br. s., 3H) 1.49-2.03 (m, 12H) 2.17 (s, 3H) 2.77 (br. d., 2H) 3.23 (s, 3H) 3.42 (d, J=13.64 Hz, 2H) 3.66 (d, J=13.39 Hz, 2H) 3.70 (m, 1H) 3.94 (s, 3H) 4.81 (d, 1H) 4.90 (d, J=10.61 Hz, 1H) 5.69 (m, 1H) 7.47 (d, J=8.0 Hz, 1H), 7.49 (br. s., 1H) 7.65 (s, 1H) 7.93 (s, 1H) 8.11 (d, J=7.83 Hz, 1H)) 8.36 (d, J=8.0 Hz, 1H). MS (ES) [M+H] calc'd for C₃₀H₄₂N₇O₃, 548; found, 548.

The enantiomer of 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide which shows longer retention time: ¹H NMR (400 MHz, DMSO-d₆) δ ppm 1.23 (br. s., 3H) 1.49-2.03 (m, 12H) 2.17 (s, 3H) 2.77 (br. d., 2H) 3.23 (s, 3H) 3.42 (d, J=13.64 Hz, 2H) 3.66 (d, J=13.39 Hz, 2H) 3.70 (m, 1H) 3.94 (s, 3H) 4.81 (d, 1H) 4.90 (d, J=10.61 Hz, 1H) 5.69 (m, 1H) 7.47 (d, J=8.0 Hz, 1H), 7.49 (br. s., 1H) 7.65 (s, 1H) 7.93 (s, 1H) 8.11 (d, J=7.83 Hz, 1H)) 8.36 (d, J=8.0 Hz, 1H). MS (ES) [M+H] calc'd for C₃₀H₄₂N₇O₃, 548; found, 548.

### Compound 242

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide

### Compound 243

### (R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide

### Compound 244

### (S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide

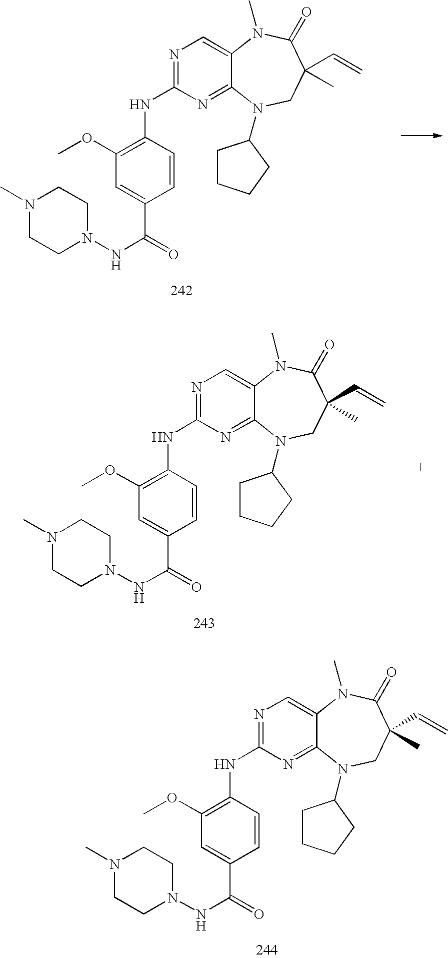

Compound 243 was synthesized using an analogous procedure to that described in connection with Compounds 239-241 except that 1-amino 4-methylpiperazine was used and the final compound was purified by reverse phase HPLC and basified to give the free base. The enantiomers of this compound were separated using Gilson SFC (ChiralPak AS-H (5 um, 21×250 mm), 25% 2-propanol with 10 mM NH₄OAc in supercritical CO₂).

The enantiomer of 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide which shows shorter retention time. ¹H NMR (400 MHz, DMSO-d₆) δ ppm 1.22 (s, 3H) 1.51-1.82 (m, 8H) 2.18 (s, 3H) 2.42 (m, 4H) 2.92 (t, J=4.55 Hz, 4H) 3.22 (s, 3H) 3.30-3.55 (m, 1H) 3.55-3.70 (m, 1H) 3.93 (s, 3H) 4.77-5.02 (m, 3H) 5.68 (dd, J=17.56, 10.74 Hz, 1H) 7.40 (d, J=8.0 Hz, 1H), 7.41 (br. s., 1H) 7.65 (s, 1H) 7.92 (s, 1H) 8.35 (d, J=8.0 Hz, 1H) 9.32 (s, 1H). [M+H] calculated for C₂₉H₄₁N₈O₃, 549; found 549.

The enantiomer of 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide which shows longer retention time. ¹H NMR (400 MHz, DMSO-d₆) δ ppm 1.22 (s, 3H) 1.51-1.82 (m, 8H) 2.18 (s, 3H) 2.42 (m, 4H) 2.92 (t, J=4.55 Hz, 4H) 3.22 (s, 3H) 3.30-3.55 (m, 1H) 3.55-3.70 (m, 1H) 3.93 (s, 3H) 4.77-5.02 (m, 3H) 5.68 (dd, J=17.56, 10.74 Hz, 1H) 7.40 (d, J=8.0 Hz, 1H), 7.41 (br. s., 1H) 7.65 (s, 1H) 7.92 (s, 1H) 8.35 (d, J=8.0 Hz, 1H) 9.32 (s, 1H). [M+H] calculated for C₂₉H₄₁N₈O₃, 549; found 549.

### Compound 245

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide

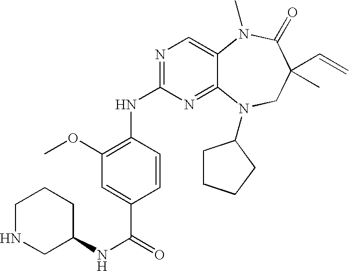

The title compound was synthesized using an analogous procedure same as that described in connection with Compound 239 except that (R)-1-Boc-3-aminopiperidine was used. Further, after washing with water (10 ml), t-butoxycarbonyl (Boc) protection group was removed using 40% TFA in dichloromethane (6 ml) and purified the product using preparative HPLC followed by neutralization to give the free base. ¹H NMR (400 MHz, DMSO-d₆) δ ppm 1.22 (br. s., 3H) 1.35-2.10 (m, 12H) 2.42 (m, 2H) 2.83 (d, J=8.3 Hz, 1H) 2.98 (d, J=8.3 Hz, 1H) 3.23 (br. s., 3H) 3.36 (d, J=13.80 Hz, 1H) 3.66 (d, J=13.89 Hz, 1H) 3.80 (m, 1H) 3.94 (br. s., 3H) 4.77-5.02 (m, 3H) 5.68 (dd, J=17.56, 10.74 Hz, 1H) 7.47 (d, J=8.0 Hz, 1H), 7.48 (br. s., 1H) 7.65 (s, 1H) 7.93 (s, 1H) 8.11 (d, J=7.83 Hz, 1H)) 8.35 (d, J=8.0 Hz, 1H). [M+H] calculated for C₂₉H₄₁N₈O₃, 534; found 534.

### Compound 246

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((S)-piperidin-3-yl)benzamide

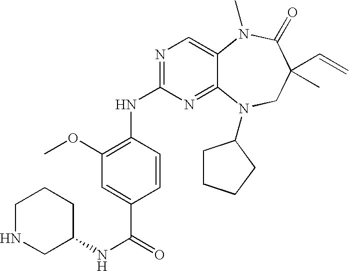

The title compound was synthesized using an analogous procedure same as that described in connection with Compound 239 except that (S)-1-Boc-3-aminopiperidine was used. Further, after washing with water (10 ml), t-butoxycarbonyl (Boc) protection group was removed using 40% TFA in dichloromethane (6 ml) and purified the product using preparative HPLC followed by neutralization to give the free base. ¹H NMR (400 MHz, DMSO-d₆) δ ppm 1.22 (br. s., 3H) 1.35-2.10 (m, 12H) 2.42 (m, 2H) 2.83 (d, J=8.3 Hz, 1H) 2.98 (d, J=8.3 Hz, 1H) 3.23 (br. s., 3H) 3.36 (d, J=13.80 Hz, 1H) 3.66 (d, J=13.89 Hz, 1H) 3.80 (m, 1H) 3.94 (br. s., 3H) 4.77-5.02 (m, 3H) 5.68 (dd, J=17.56, 10.74 Hz, 1H) 7.47 (d, J=8.0 Hz, 1H), 7.48 (br. s., 1H) 7.65 (s, 1H) 7.93 (s, 1H) 8.11 (d, J=7.83 Hz, 1H)) 8.35 (d, J=8.0 Hz, 1H). [M+H] calculated for C₂₉H₄₁N₈O₃, 534; found 534.

### Compound 247

### 4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide

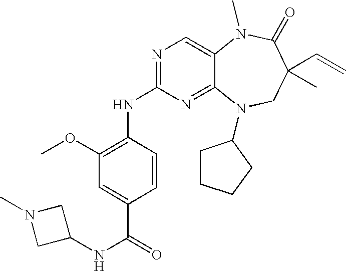

The title compound was synthesized using an analogous procedure same as that described in connection with Compound 240 except that methylazetidin-3-amine was used and the final compound was purified by reverse phase HPLC and basified to give the free base 1-bis-hydrochloride was used. ¹H NMR (400 MHz, DMSO-d₆) δ ppm 1.22 (s, 3H) 1.48-2.10 (m, 8H) 2.26 (s, 3H) 2.98 (t, J=6.82 Hz, 2H) 3.23 (s, 2H) 3.42 (d, J=13.89 Hz, 1H) 3.55 (t, J=6.95 Hz, 2H) 3.66 (d, J=13.89 Hz, 1H) 3.94 (s, 3H) 4.42 (m, 1H) 4.77-5.02 (m, 3H) 5.68 (dd, J=17.56, 10.74 Hz, 1H) 7.48 (d, J=8.0 Hz, 1H), 7.49 (br. s., 1H) 7.66 (s, 1H) 7.92 (s, 1H) 8.35 (d, J=8.0 Hz, 1H) 8.62 (d, J=7.83 Hz, 1H). [M+H] calculated for C₂₈H₃₈N₇O₃, 520; found 520.

### Compound 248

### (S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

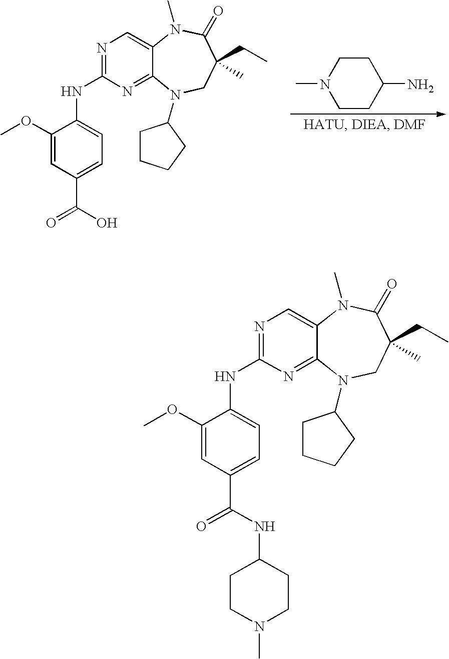

To a mixture of compound (S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid, DIEA (106 μL, 0.3 mmol) in 2.0 mL of anhydrous DMF was added HATU (114 mg, 0.3 mmol). The reaction mixture was stirred for 30 min. The reaction mixture was then diluted with ethyl acetate, washed with water and brine. The organic layer dried over Na₂SO₄ followed by HPLC purification. The TFA salt was then converted to free base, which was finally crystallized from ether to give title compound. ¹H NMR (400 MHz, DMSO-d₆) δ ppm 0.76 (t, J=7.4 Hz, 3H) 1.14 (s, 3H) 1.35 (d, J=6.6 Hz, 1H) 1.55 (t, J=6.8 Hz, 3H) 1.49-1.63 (m, 1H) 1.68-1.90 (m, 3H) 1.74 (d, J=9.4 Hz, 3H) 2.04 (d, J=12.4 Hz, 2H) 2.81 (d, J=5.0 Hz, 1H) 2.78 (d, J=4.5 Hz, 2H) 3.06-3.16 (m, 1H) 3.11 (d, J=12.6 Hz, 1H) 3.17 (s, 3H) 3.36 (d, J=14.6 Hz, 1H) 3.48 (d, J=11.6 Hz, 2H) 3.69 (d, J=14.9 Hz, 1H) 3.94 (s, 3H) 3.97-4.07 (m, 1H) 5.11 (d, J=7.8 Hz, 1H) 7.51-7.58 (m, 2H) 8.05 (s, 1H) 8.08 (d, J=8.6 Hz, 1H) 8.45 (d, J=7.6 Hz, 1H) 9.12 (br. s., 1H) 9.63 (br. s., 1H). [M+H] calc'd for C₃₀H₄₃N₇O₃, 550; found, 550.

### Compound 249

### (R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide

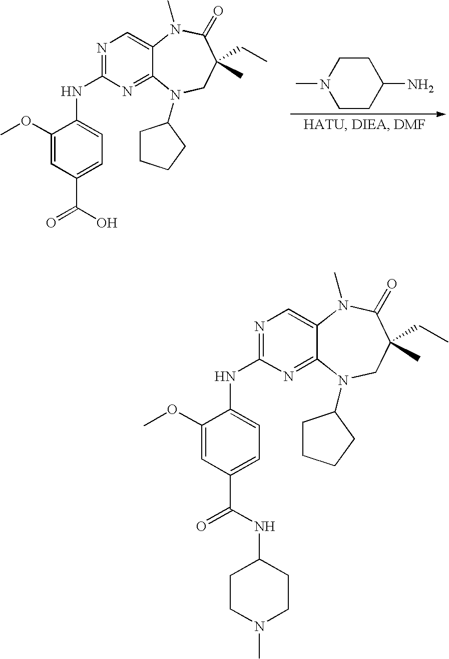

To a mixture of compound (R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid, DIEA (106 μL, 0.3 mmol) in 2.0 mL of anhydrous DMF was added HATU (114 mg, 0.3 mmol). The reaction mixture was stirred for 30 min. The reaction mixture was then diluted with ethyl acetate, washed with water and brine. The organic layer dried over Na₂SO₄ followed by HPLC purification. The TFA salt was then converted to free base, which was finally crystallized from ether to give title compound. ¹H NMR (400 MHz, DMSO-d₆) δ ppm 0.76 (t, J=7.4 Hz, 3H) 1.14 (s, 3H) 1.35 (d, J=6.6 Hz, 1H) 1.55 (t, J=6.8 Hz, 3H) 1.49-1.63 (m, 1H) 1.68-1.90 (m, 3H) 1.74 (d, J=9.4 Hz, 3H) 2.04 (d, J=12.4 Hz, 2H) 2.81 (d, J=5.0 Hz, 1H) 2.78 (d, J=4.5 Hz, 2H) 3.06-3.16 (m, 1H) 3.11 (d, J=12.6 Hz, 1H) 3.17 (s, 3H) 3.36 (d, J=14.6 Hz, 1H) 3.48 (d, J=11.6 Hz, 2H) 3.69 (d, J=14.9 Hz, 1H) 3.94 (s, 3H) 3.97-4.07 (m, 1H) 5.11 (d, J=7.8 Hz, 1H) 7.51-7.58 (m, 2H) 8.05 (s, 1H) 8.08 (d, J=8.6 Hz, 1H) 8.45 (d, J=7.6 Hz, 1H) 9.12 (br. s., 1H) 9.63 (br. s., 1H). [M+H] calc'd for C₃₀H₄₃N₇O₃, 550; found, 550.

In addition to the foregoing, the above reaction schemes, and variations thereof, have been used to prepare the following:

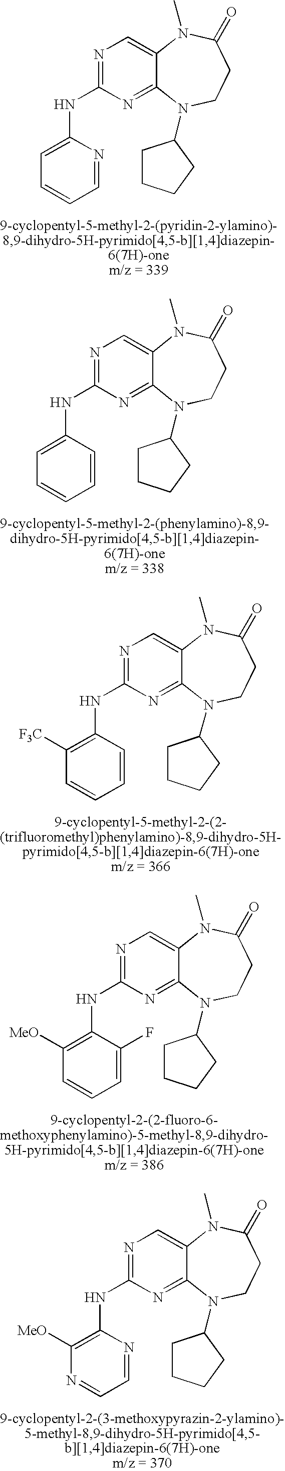

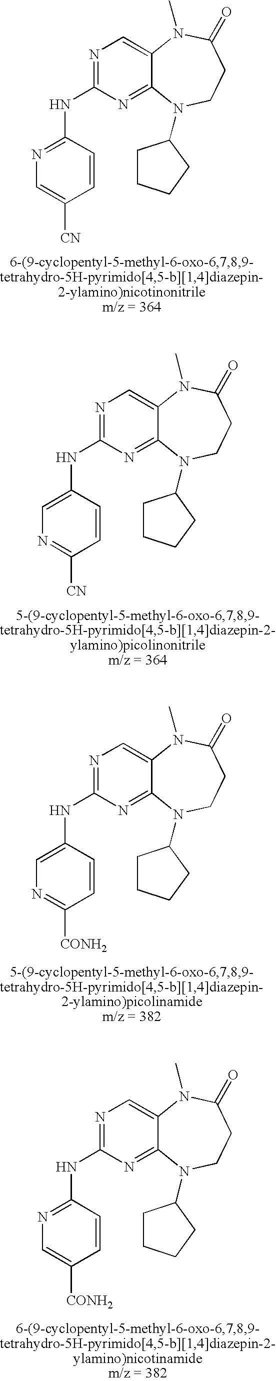

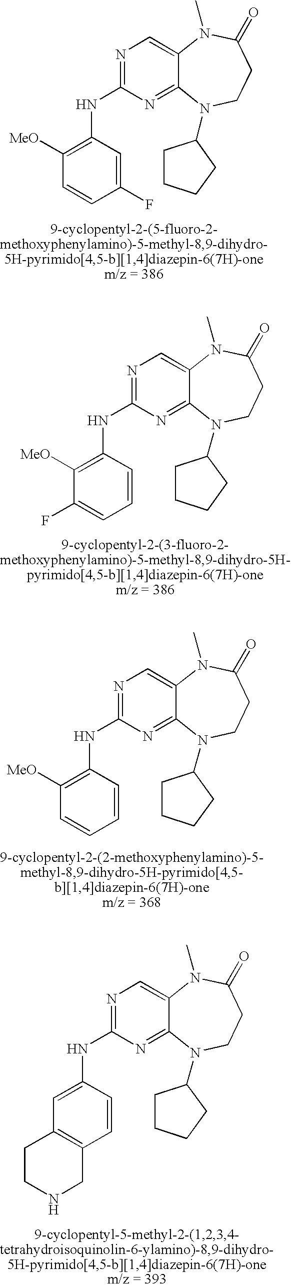

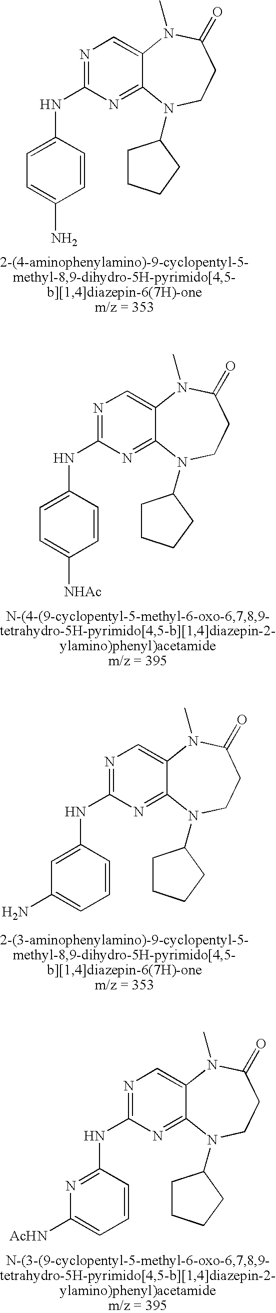

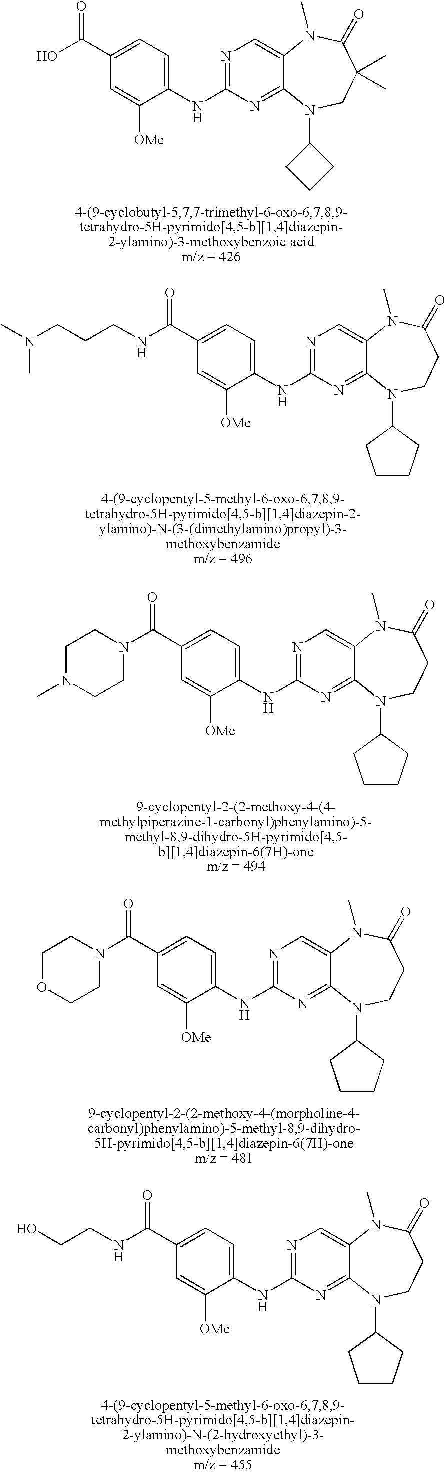

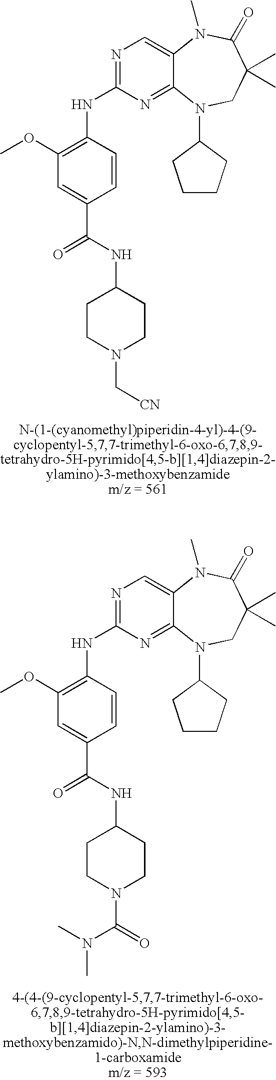

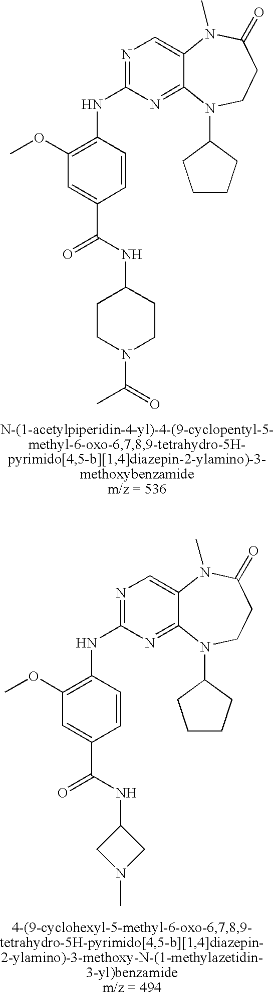

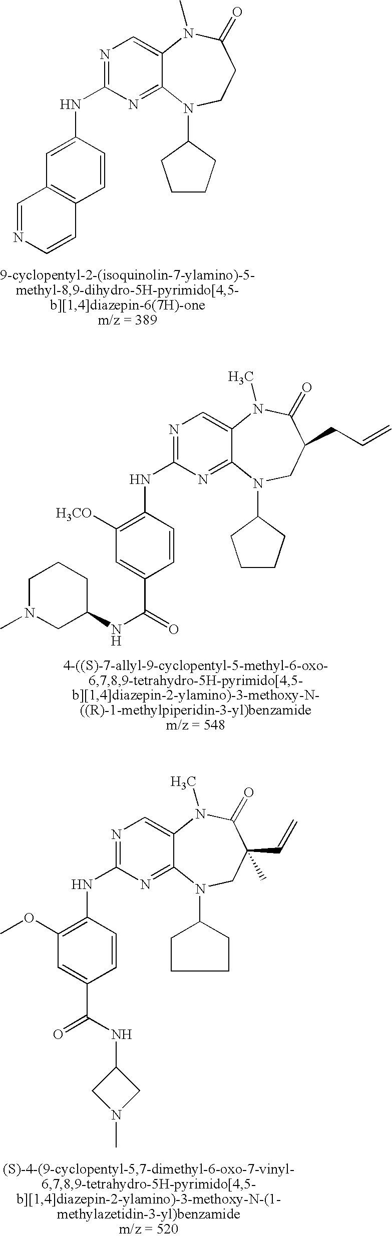

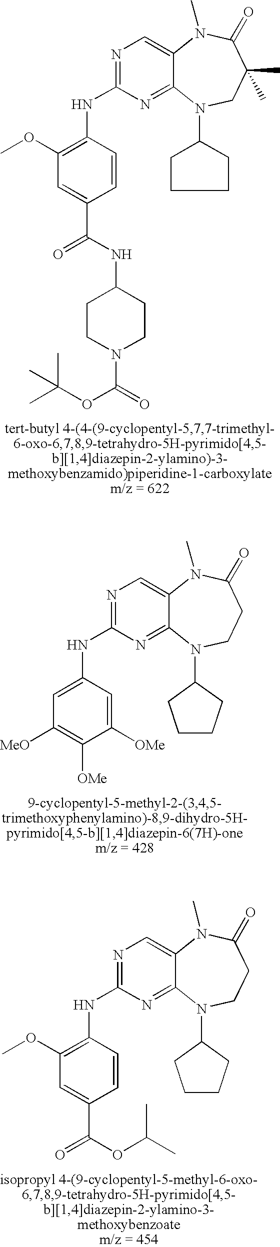

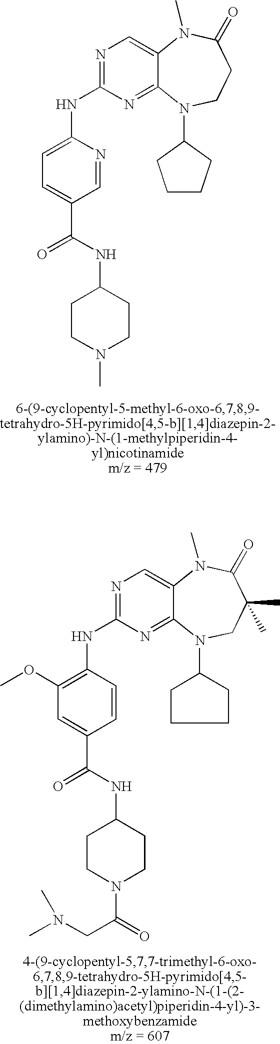

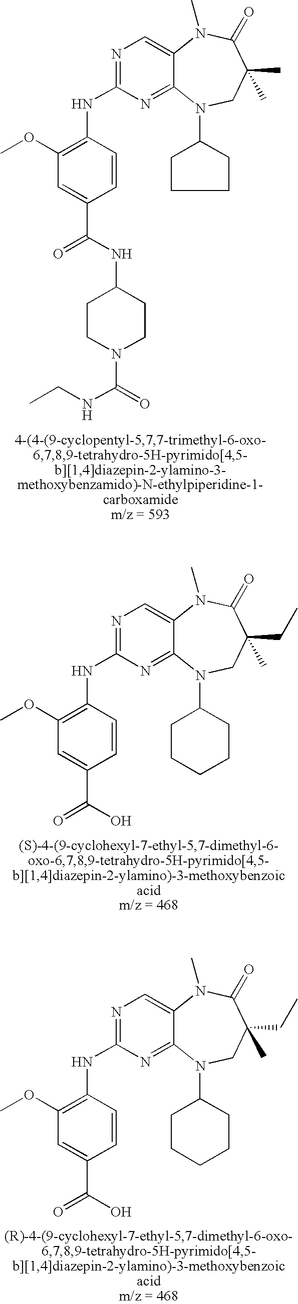

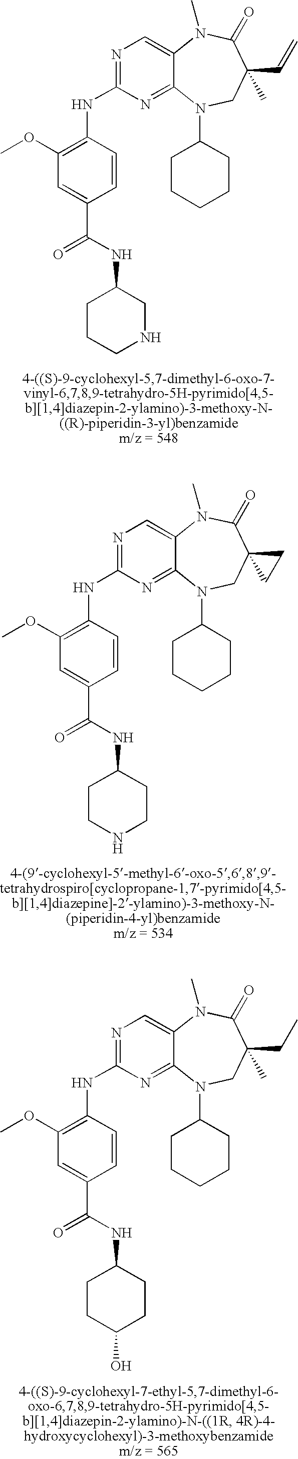

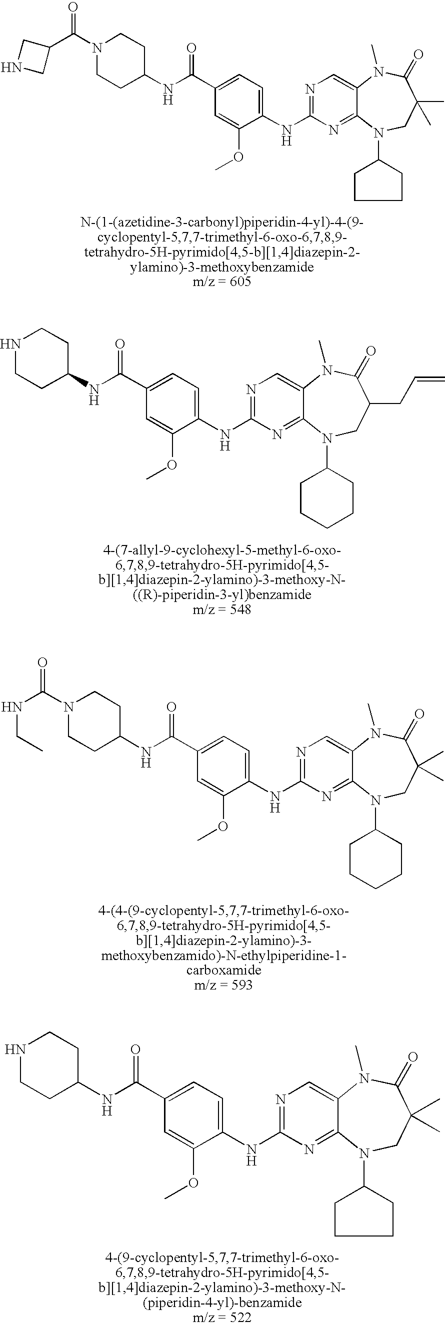

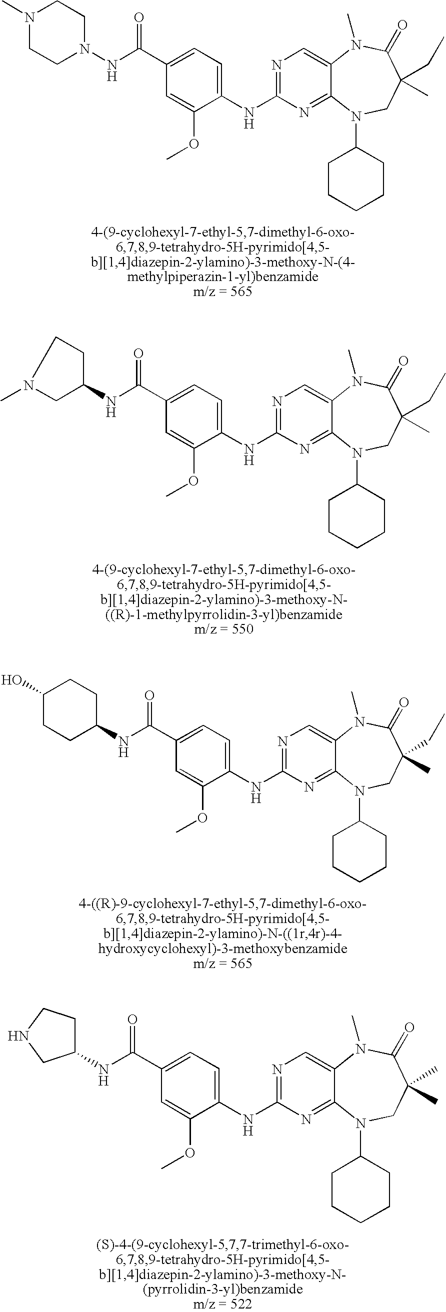

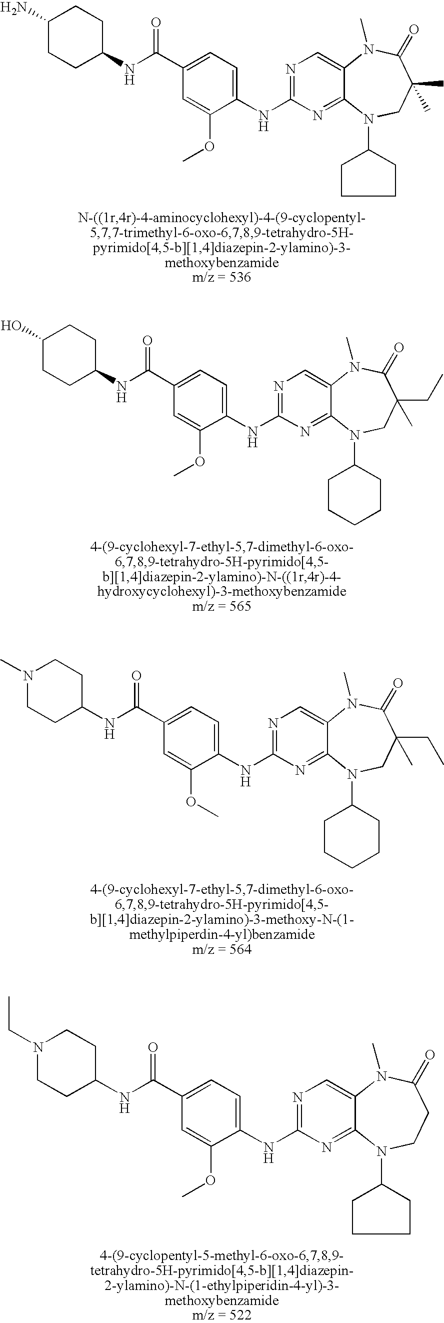

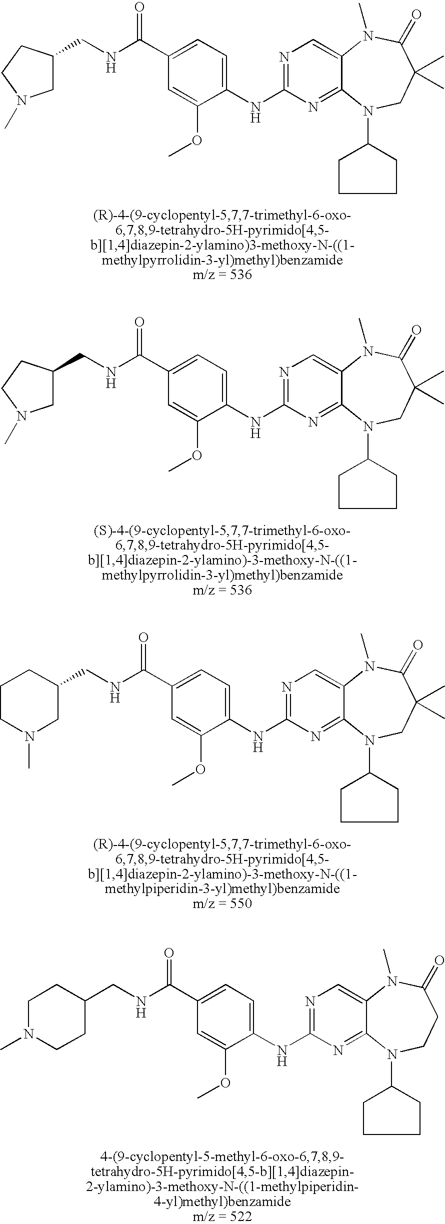

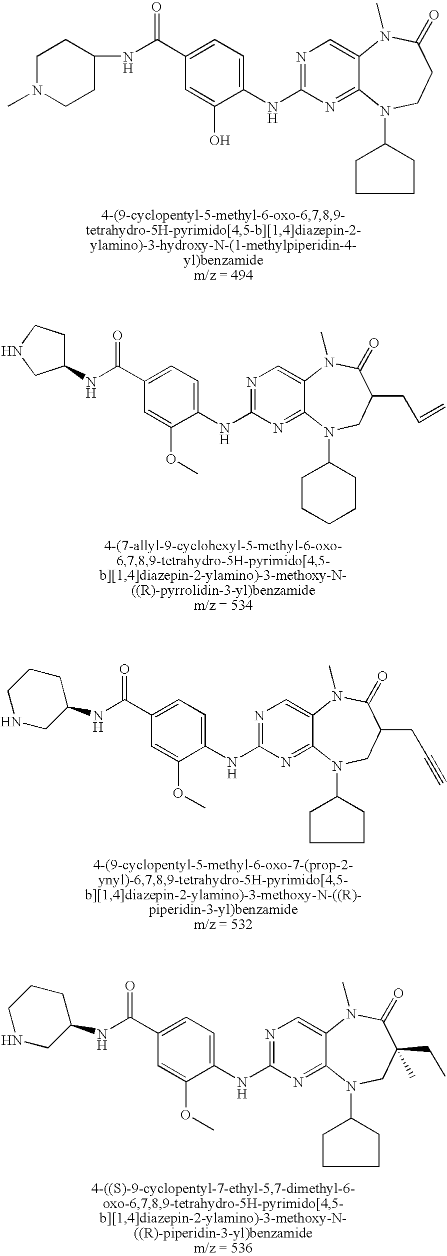

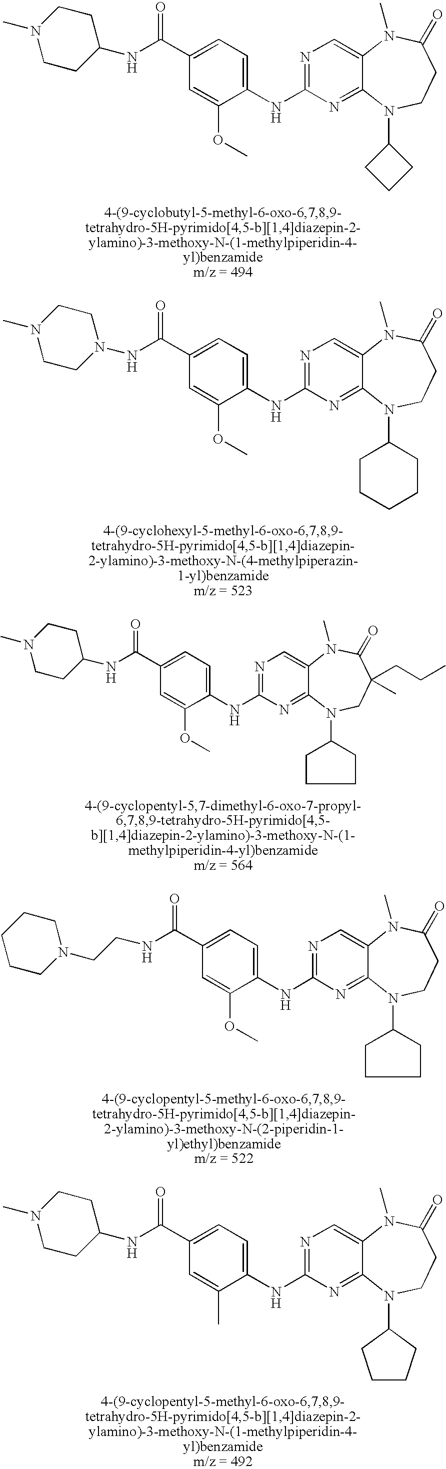

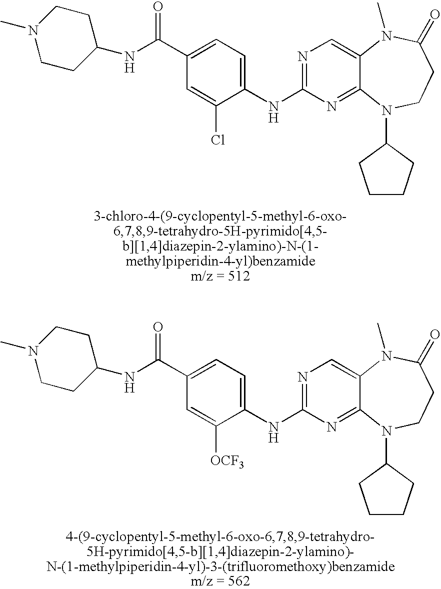

### Biological Testing

The activity of compounds as PLK inhibitors may be assayed in vitro, in vivo or in a cell line. Provided below is an in vitro enzymatic activity assay for activity against PLK1.

Purified PLK1 may be obtained as follows. cDNA encoding human PLK1 (SEQ ID No. 1, Accession Number: NM_{—}005030) was isolated by polymerase chain reaction (PCR) with primers (SEQ ID Nos. 2 and 3) and cloned into pcDNA4/His-Max-TOPO (Invitrogen, USA) according to the manufacturer's manual. PCR was performed using the vector as a template. In the PCR, primers containing sequences encoding a FLAG-tag (DYKDDDDK) in the amino-terminal region (SEQ ID No. 4) and vector sequence (SEQ ID No. 5) were used. After XbaI and XhoI digestion of the PCR product, the fragment was subcloned into pFASTBAC1 (Invitrogen, USA). Recombinant baculoviruses were prepared according to the procedure of the Bac-to-Bac baculovirus expression system (Invitrogen, USA). Sf21 cells were purchased from Invitrogen and grown in Sf-900 II SFM medium containing 10% fetal bovine serum, 50 μg/mL gentamicin and 0.1% pluronic F-68 (Invitrogen, USA) at 28° C. For preparation of PLK1 enzyme, Sf21 cells were infected with recombinant baculoviruses and cultured at 28° C. for 72 h. Cells were lysed and FLAG-tagged PLK1 protein (SEQ ID No. 6) was purified by affinity chromatography using anti-FLAG M2 affinity gel (Sigma, USA).

It should be noted that a variety of other expression systems and hosts are also suitable for the expression of PLK1, as would be readily appreciated by one of skill in the art.

The inhibition of PLK1 by the compounds was determined with the following assay that measures the phosphorylation of alpha casein by recombinant PLK1. Kinase reactions were performed at room temperature for 40 min in the kinase reaction buffer (25 mmol/L HEPES, pH 7.5, 10 mmol/L magnesium acetate, 1 mmol/L dithiothreitol) containing 50 ng PLK1 enzyme, 0.1 μCi [γ-³²P]ATP, 500 nmol/L ATP and 3 μg alpha casein (MP Biomedicals Inc., USA) in a final volume of 50 μL. The incubation was terminated by the addition of 10% trichloroacetic acid (Wako, Japan). Phosphorylated proteins were filtrated in GF/C filter plates (Packard, USA) with a Cell harvester (Packard, USA) and washed out free [γ-³²P]-ATP with 250 mmol/L phosphoric acid. Then, the plates were air dried for 60 min at 45° C., followed by the addition of 20 μL of MicroScint-O (Packard, USA). The radioactivity was counted by a Top-count scintillation counter (Packard, USA). The IC₅₀ values for test compounds were calculated by Prism 3.02 (GraphPad Software, USA).

The cell culture and proliferation assay may be carried out as follows: the HT29 human colorectal adenocarcinoma cell line (ATCC, USA) was maintained in Dulbecco's Modified Eagle's Medium (Invitrogen, USA) supplemented with 10% fetal bovine serum (JRH, USA). The cell proliferation assay was carried out with the Cell Titer-Glo luminescent cell viability assay (Promega, USA) according to the manufacture's instruction after the 72 hr treatment of the cells in the presence of the compounds. The cell viability was shown as a percentage of DMSO treated cells. The IC₅₀ values for the compounds were calculated by Prism 4 (GraphPad Software, USA).

For analysis of the cell cycle distribution and the phosphorylation of histone H3, the cells were harvested and fixed with ice-cold 70% ethanol after the 48 hr treatment of the cells in the presence of the compounds. The cells were washed twice with PBS containing 2% FCS (JRH), then incubated with Alexa Flour 647-conjugated anti-phospho-histone H3 antibody (Cell signaling, USA) and RNase (Invitrogen) for 30 min at room temperature. After washing twice with PBS containing 2% FCS, the cells were counterstained with propidium iodide. The cell cycle distribution and phosphorylation of histone H3 were analyzed using the FACSCalibur system (BD Bioscience, San Jose, Calif., USA).

The activity of compounds as PLK inhibitors can be assessed in vivo using BALB/cA Jcl-nu/nu mice bearing the HCT116 or the HT29 cells inoculated subcutaneously in axillary area. The growth retardation may be determined, for example, by caliper measurements of the tumor volume.

pIC₅₀ values may be calculated by non-linear curve fitting of the compound concentrations and fluorescence intensities to the standard pIC₅₀ equation. pIC₅₀ values for the cell proliferative assay of select compounds of the present invention are given in Table 1.

TABLE 1

| Compound | pIC₅₀ |
|---|---|
| 10 | 8.0-8.5 |
| 11 | 8.0-8.5 |
| 33 | 8.6-9.0 |
| 36 | 8.0-8.5 |
| 37 | 8.0-8.5 |
| 38 | 8.0-8.5 |
| 39 | 8.6-9.0 |
| 40 | <8.0 |
| 41 | <8.0 |
| 42 | 8.0-8.5 |
| 43 | <8.0 |
| 46 | 8.0-8.5 |
| 47 | 8.0-8.5 |
| 48 | <8.0 |
| 49 | <8.0 |
| 50 | <8.0 |
| 51 | <8.0 |
| 52 | <8.0 |
| 53 | <8.0 |
| 54 | 8.0-8.5 |
| 55 | <8.0 |
| 56 | <8.0 |
| 57 | <8.0 |
| 59 | <8.0 |
| 60 | <8.0 |
| 61 | <8.0 |
| 62 | <8.0 |
| 63 | <8.0 |
| 64 | <8.0 |
| 66 | <8.0 |
| 69 | 8.0-8.5 |
| 70 | 8.0-8.5 |
| 71 | 8.0-8.5 |
| 73 | <8.0 |
| 76 | <8.0 |
| 77 | <8.0 |
| 79 | <8.0 |
| 80 | 8.0-8.5 |
| 85 | 8.0-8.5 |
| 86 | 8.0-8.5 |
| 87 | 8.6-9.0 |
| 88 | <8.0 |
| 89 | 8.0-8.5 |
| 90 | 8.6-9.0 |
| 91 | 8.6-9.0 |
| 92 | 8.0-8.5 |
| 93 | 8.0-8.5 |
| 94 | 8.6-9.0 |
| 95 | 8.6-9.0 |
| 96 | 8.0-8.5 |
| 97 | 8.6-9.0 |
| 98 | 8.6-9.0 |
| 99 | >9.0 |
| 100 | 8.0-8.5 |
| 101 | 8.6-9.0 |
| 102 | >9.0 |
| 103 | 8.6-9.0 |
| 104 | >9.0 |
| 105 | 8.6-9.0 |
| 106 | >9.0 |
| 107 | 8.0-8.5 |
| 109 | 8.6-9.0 |
| 110 | 8.6-9.0 |
| 111 | 8.6-9.0 |
| 112 | 8.6-9.0 |
| 113 | >9.0 |
| 116 | 8.0-8.5 |
| 117 | 8.0-8.5 |
| 118 | 8.0-8.5 |
| 119 | 8.0-8.5 |
| 121 | 8.0-8.5 |
| 122 | <8.0 |
| 123 | >9.0 |
| 124 | <8.0 |
| 127 | <8.0 |
| 131 | 8.0-8.5 |
| 132 | 8.0-8.5 |
| 133 | <8.0 |
| 134 | 8.0-8.5 |
| 140 | 8.0-8.5 |
| 141 | 8.0-8.5 |
| 145 | 8.0-8.5 |
| 148 | 8.0-8.5 |
| 149 | 8.0-8.5 |
| 151 | 8.0-8.5 |
| 153 | 8.0-8.5 |
| 154 | 8.0-8.5 |
| 155 | 8.0-8.5 |
| 156 | <8.0 |
| 157 | <8.0 |
| 163 | 8.6-9.0 |
| 164 | 8.6-9.0 |
| 165 | 8.6-9.0 |
| 166 | 8.6-9.0 |
| 167 | 8.0-8.5 |
| 168 | 8.6-9.0 |
| 169 | 8.6-9.0 |
| 170 | >9.0 |
| 171 | >9.0 |
| 172 | 8.6-9.0 |
| 173 | 8.6-9.0 |
| 174 | 8.6-9.0 |
| 175 | >9.0 |
| 176 | <8.0 |
| 177 | >9.0 |
| 179 | >9.0 |
| 180 | >9.0 |
| 181 | 8.0-8.5 |
| 182 | 8.6-9.0 |
| 187 | 8.6-9.0 |
| 188 | <8.0 |
| 189 | 8.6-9.0 |
| 191 | 8.0-8.5 |
| 192 | <8.0 |
| 194 | >9.0 |
| 196 | >9.0 |
| 197 | >9.0 |
| 198 | >9.0 |
| 200 | 8.6-9.0 |
| 201 | 8.6-9.0 |
| 202 | 8.6-9.0 |
| 203 | 8.6-9.0 |
| 204 | 8.6-9.0 |
| 205 | >9.0 |
| 206 | >9.0 |
| 207 | 8.0-8.5 |
| 209 | 8.6-9.0 |
| 211 | 8.6-9.0 |
| 216 | 8.6-9.0 |
| 218 | >9.0 |
| 219 | 8.0-8.5 |
| 220 | 8.0-8.5 |
| 221 | 8.6-9.0 |
| 225 | 8.0-8.5 |
| 226 | 8.6-9.0 |
| 227 | <8.0 |
| 229 | 8.6-9.0 |
| 230 | <8.0 |
| 231 | <8.0 |
| 239 | 8.0-8.5 |
| 240 | 8.0-8.5 |
| 241 | <8.0 |
| 242 | <8.0 |
| 243 | >9.0 |
| 244 | <8.0 |
| 245 | 8.0-8.5 |
| 246 | 8.6-9.0 |
| 247 | 8.0-8.5 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A compound comprising: 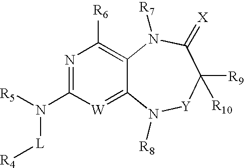
wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
L is absent or a linker providing 1, 2, 3, 4, 5 or 6 atom separation between R₄ and the nitrogen to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₄ is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₅ is hydrogen or a substituent convertible in vivo to hydrogen;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

2. The compound according to claim 1 comprising: 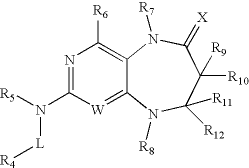
wherein
R₁₁ and R₁₂ are each independently selected from the group consisting of hydrogen, cyano, carbonyl, oxycarbonyl, aminocarbonyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₁ and R₁₂ are taken together with the atom to which they are bound to form a carbonyl or imino group,
or any two R₈, R₉, R₁₀, R₁₁ and R₁₂ are taken together to form a substituted or unsubstituted ring.

3. The compound according to claim 2 comprising:

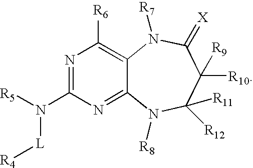

4. The compound according to claim 2 comprising:

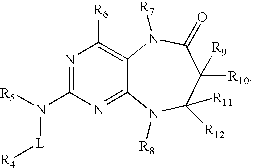

5. The compound according to claim 2 comprising: 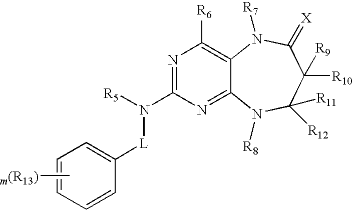
wherein
m is selected from the group consisting of 0, 1, 2, 3, 4 and 5; and
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring.

6. The compound according to claim 2 comprising: 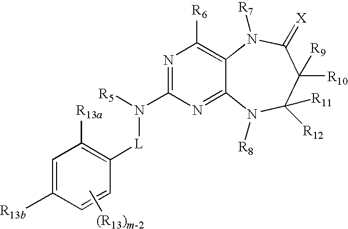
wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ and R_{13b} are each independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

7. The compound according to claim 2 comprising: 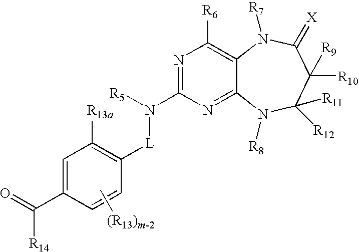
wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ is selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₁₄ is selected from the group consisting of hydrogen, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

8. The compound according to claim 2 comprising: 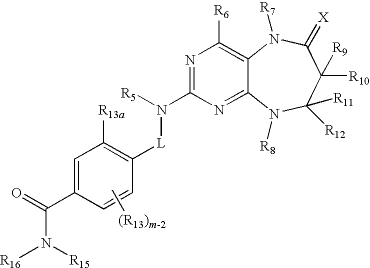
wherein
m is selected from the group consisting of 0, 1, 2, 3, 4 and 5;
each R₁₃ is independently selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or two R₁₃ are taken together to form a substituted or unsubstituted ring; and
R₁₃ₐ is selected from the group consisting of hydrogen, halo, nitro, cyano, thio, oxy, hydroxy, carbonyloxy, alkoxy, aryloxy, heteroaryloxy, carbamoyloxy, carbonyl, oxycarbonyl, aminocarbonyl, amino, (C₁₋₁₀)alkylamino, sulfonamido, carboxyamino, ureido, imino, sulfonyl, aminosulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₁₅ are R₁₆ are each independently selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form a substituted or unsubstituted ring.

9. The compound according to claim 1 comprising: 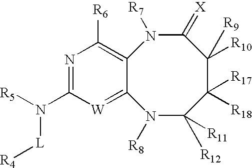
wherein
R₁₁ and R₁₂ are each independently selected from the group consisting of hydrogen, cyano, carbonyl, oxycarbonyl, aminocarbonyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₁₁ and R₁₂ are taken together with the atom to which they are bound to form a carbonyl or imino group; and
R₁₇ and R₁₈ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted,
or any two R₈, R₉, R₁₀, R₁₁, R₁₂, R₁₇ and R₁₈ are taken together to form a substituted or unsubstituted ring.

10. A process comprising:
reacting a compound comprising the formula 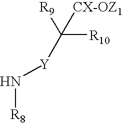
with a compound comprising the formula 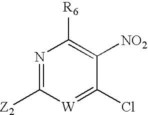
under conditions that form a first reaction product comprising the formula 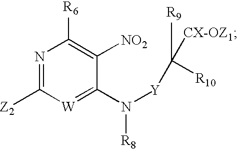
treating the first reaction product under conditions that form a second reaction product comprising the formula 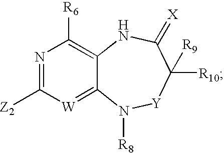
reacting the second reaction product with a compound comprising the formula
R₇-Z₃
under conditions that form a third reaction product comprising the formula 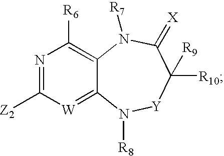
reacting the third reaction product with a compound comprising the formula
R₄-L-NR₅H
under conditions that form a compound comprising the formula 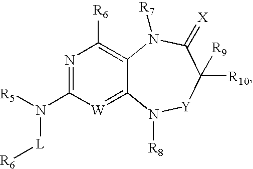
wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₁, Z₂ and Z₃ are each independently a leaving group;
L is absent or a linker providing 1, 2, 3, 4, 5 or 6 atom separation between R₄ and the nitrogen to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₄ is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₅ is hydrogen or a substituent convertible in vivo to hydrogen;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

11. A compound comprising: 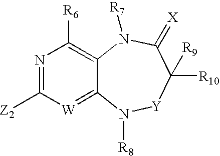
wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₂ is a leaving group;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₇ is selected from the group consisting of hydrogen, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₆ and R₇ are taken together to form a substituted or unsubstituted ring;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

12. A compound comprising: 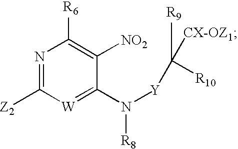
wherein
W is selected from the group consisting of CR₁ and N;
X is selected from the group consisting of NR₂₁, O and S;
Y is —(CR₂R₃)ₙ—;
n is selected from the group consisting of 1, 2, 3 and 4;
Z₁ and Z₂ are each independently a leaving group;
R₁ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₂ and R₃ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₆ is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₈ is selected from the group consisting of hydrogen, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
R₉ and R₁₀ are each independently selected from the group consisting of hydrogen, cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₉ and R₁₀ are taken together with the atom to which they are bound to form a carbonyl or imino group; and
R₂₁ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₁ and R₇ are taken together to form a substituted or unsubstituted ring,
or any two R₂, R₃, R₈, R₉ and R₁₀ are taken together to form a substituted or unsubstituted ring.

13. The compound or process according to any one of claims 1, 2 and 9-12, wherein W is —CH═.

14. The compound or process according to any one of claims 1-3 and 5-13, wherein X is O.

15. The compound or process according to any one of claims 1-10, 13 and 14, wherein L is a substituted or unsubstituted (C₁₋₃)alkyl.

16. The compound or process according to any one of claims 1-10, 13 and 14, wherein L is —CHR₁₉—; and R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

17. The compound or process according to any one of claims 1-10, 13 and 14, wherein L is absent.

18. The compound or process according to any one of claims 1, 2 and 9-17, wherein R₁ is selected from the group consisting of hydrogen and a substituted or unsubstituted (C₁₋₅)alkyl.

19. The compound or process according to any one of claims 1-4, 9, 10 and 13-18, wherein R₄ is a substituted or unsubstituted hetero(C₁₋₁₀)aryl.

20. The compound or process according to any one of claims 1-10 and 13-19, wherein R₅ is hydrogen.

21. The compound or process according to any one of claims 1-10 and 13-19, wherein R₅ is a substituent convertible in vivo to hydrogen.

22. The compound or process according to any one of claims 1-10 and 13-19, wherein R₅ is selected from the group consisting of hydrolyzable groups, groups having an oxycarbonyl group, amino acid residues, peptide residues, o-nitrophenylsulfenyl, trimethylsilyl, tetrahydro-pyranyl, diphenylphosphinyl, arylsulfonyl groups, methyl groups substituted with phenyl or benzyloxy, arylmethoxycarbonyl groups, and halogenoethoxycarbonyl groups.

23. The compound or process according to any one of claims 1-22, wherein R₆ is hydrogen.

24. The compound or process according to any one of claims 1-11 and 13-23, wherein R₇ is selected from the group consisting of hydrogen and a substituted or unsubstituted (C₁₋₃)alkyl.

25. The compound or process according to any one of claims 1-24, wherein R₈ is selected from the group consisting of (C₁₋₅)alkyl and (C₃₋₁₂)cycloalkyl, each substituted or unsubstituted.

26. The compound or process according to any one of claims 1-24, wherein R₈ is selected from the group consisting of isopropyl, cyclopropyl, cyclopentyl and cyclohexyl, each substituted or unsubstituted.

27. The compound or process according to any one of claims 1-24, wherein R₈ is a substituted or unsubstituted cyclohexyl.

28. The compound or process according to any one of claims 1-27, wherein R₉ is selected from the group consisting of hydrogen and a substituted or unsubstituted (C₁₋₃)alkyl.

29. The compound or process according to any one of claims 1-27, wherein R₉ is ethyl.

30. The compound or process according to any one of claims 1-27, wherein R₉ is propyl.

31. The compound or process according to any one of claims 1-27, wherein R₉ is —CH₂—CN.

32. The compound or process according to any one of claims 1-27, wherein R₉ is alkoxy.

33. The compound or process according to any one of claims 1-27, wherein R₉ is hydroxy.

34. The compound or process according to any one of claims 1-27, wherein R₉ has the formula 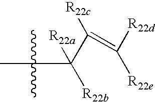
wherein
each R₂₂ₐ, R_{22b}, R_{22c}, R_{22d} and R₂₂ₑ are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

35. The compound or process according to any one of claims 1-27, wherein R₉ has the formula 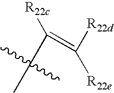
wherein
each R_{22c}, R_{22d} and R₂₂ₑ are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

36. The compound or process according to any one of claims 1-27, wherein R₉ has the formula 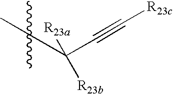
wherein
each R₂₃ₐ, R_{23b} and R_{23c} are individually selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

37. The compound or process according to any one of claims 1-27, wherein R₉ has the formula 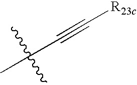
wherein
R_{23c} is selected from the group consisting of hydrogen, cyano, thio, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

38. The compound or process according to any one of claims 1-37, wherein R₁₀ is selected from the group consisting of hydrogen and a substituted or unsubstituted (C₁₋₃)alkyl.

39. The compound or process according to any one of claims 1-37, wherein R₁₀ is methyl.

40. The compound or process according to any one of claims 2-9 and 13-39, wherein R₁₁ is selected from the group consisting of hydrogen and a substituted or unsubstituted (C₁₋₃)alkyl.

41. The compound or process according to any one of claims 2-9 and 13-40, wherein R₁₂ is selected from the group consisting of hydrogen and a substituted or unsubstituted (C₁₋₃)alkyl.

42. The compound or process according to any one of claims 5-8 and 13-41, wherein at least one R₁₃ is a substituted or unsubstituted alkoxy.

43. The compound or process according to any one of claims 5-8 and 13-41, wherein at least one R₁₃ comprises —C(O)NR₁₅R₁₆, wherein R₁₅ and R₁₆ are each independently selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

44. The compound or process according to any one of claims 5-8 and 13-41, wherein at least one R₁₃ comprises —C(O)OR₂₀, wherein R₂₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

45. The compound or process according to any one of claims 5-8 and 13-44, wherein m is 2.

46. The compound or process according to any one of claims 6-8 and 13-45, wherein R₁₃ₐ is a substituted or unsubstituted alkoxy.

47. The compound or process according to any one of claims 6 and 13-46, wherein R_{13b} comprises —C(O)NR₁₅R₁₆, wherein R₁₅ and R₁₆ are each independently selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

48. The compound or process according to any one of claims 6 and 13-46, wherein R_{13b} comprises —C(O)OR₂₀, wherein R₂₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

49. The compound or process according to any one of claims 7 and 13-48, wherein R₁₄ is hydroxy.

50. The compound or process according to any one of claims 35 and 48, wherein R₂₀ is hydroxy.

51. The compound or process according to any one of claims 9 and 13-50, wherein R₉ and R₁₇ are taken together to form a substituted or unsubstituted 3-, 4-, 5-, 6-, 7- or 8-membered ring.

52. The compound or process according to any one of claims 9 and 13-50, wherein R₉ and R₁₇ are taken together to form a substituted or unsubstituted (C₃₋₁₀)cycloalkyl ring.

53. The compound or process according to any one of claims 9 and 13-50, wherein R₉ and R₁₇ are taken together to form a substituted or unsubstituted ring selected from the group consisting of cyclopropane, cyclobutane, cyclopentane and cyclohexane.

54. The compound or process according to any one of claims 9 and 13-53, wherein the ring formed by R₉ and R₁₇ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

55. The compound or process according to any one of claims 2-9 and 13-54, wherein R₈ and R₁₁ are taken together to form a substituted or unsubstituted 5-, 6-, 7- or 8-membered ring.

56. The compound or process according to any one of claims 2-9 and 13-54, wherein R₅ and R₁₁ are taken together to form a substituted or unsubstituted pyrrolidine.

57. The compound or process according to any one of claims 2-9 and 13-56, wherein the ring formed by R₈ and R₁₁ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

58. The compound or process according to any one of claims 9 and 13-57, wherein R₁₁ and R₁₇ are taken together to form a substituted or unsubstituted 5-, 6-, 7- or 8-membered ring.

59. The compound or process according to any one of claims 9 and 13-57, wherein R₁₁ and R₁₇ are taken together to form a substituted or unsubstituted pyrrolidine.

60. The compound or process according to any one of claims 8 and 13-59, wherein R₁₅ is 1-methylpiperidin-4-yl.

61. The compound or process according to any one of claims 8 and 13-59, wherein R₁₅ is selected from the group consisting of hydrogen, oxy, hydroxy, carbonyloxy, alkoxy, aryloxy, heteroaryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₉₋₁₂)bicycloaryl and hetero(C₄₋₁₂)bicycloaryl, each substituted or unsubstituted.

62. The compound or process according to any one of claims 9 and 13-61, wherein the ring formed by R₁₁ and R₁₇ is substituted with one or more substituents independently selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

63. The compound or process according to any one of claims 1-62, wherein
R₉ is ethyl; and
R₁₀ is methyl.

64. The compound or process according to any one of claims 1-62, wherein
R₉ is selected from the group consisting of cyano, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, (C₁₋₁₀)azaalkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₁₀ is methyl.

65. The compound or process according to any one of claims 8 and 13-64, wherein
L is absent;
R₁₃ₐ is —OCH₃;
R₁₅ is 1-methylpiperidin-4-yl; and
R₁₆ is hydrogen.

66. The compound or process according to any one of claims 10 and 12-65, wherein Z₁ is halo.

67. The compound or process according to any one of claims 10-66, wherein Z₂ is halo.

68. The compound or process according to any one of claims 10 and 13-67, wherein Z₃ is halo.

69. The compound according to claim 1, selected from the group consisting of:
4-(9-cyclopentyl-5-methyl-6-oxo-6,7,8,9-tetrahydro-5H-pyrimido[4,5-b][1,4]diazepin-2-ylamino)-3-methoxybenzoic acid;
4-[(9-cyclopentyl-5-methyl-6-oxo-6,7,8,9-tetrahydro-5H-pyrimido[4,5-b][1,4]diazepin-2-yl)amino]-3-methoxy-N-methylbenzamide;
4-[(9-cyclopentyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopentyl-5,8-dimethyl-6-oxo-6,7,8,9-tetrahydro-5H-pyrimido[4,5-b][1,4]diazepin-2-ylamino)-3-methoxybenzoic acid;
4-[(9-cyclopentyl-5,8-dimethyl-6-oxo-6,7,8,9-tetrahydro-5H-pyrimido[4,5-b][1,4]diazepin-2-yl)amino]-3-methoxy-N-methylbenzamide;
4-{[(cis)-10-cyclopentyl-5-methyl-6-oxo-5,6,6a,7,8,9,9a,10-octahydrocyclopenta[e]pyrimido[5,4-b][1,4]diazepin-2-yl]amino}-3-methoxy-N-methylbenzamide;
4-(9-cyclopentyl-8-ethyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-{[(7aR)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
3-methoxy-4-{[(7aS)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
4-[(9-isopropyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopropyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-[(9-cyclohexyll-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-isobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-benzyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-(pentan-3-yl)-6,7,8,9-tetrahydro-5H-pyrimido[4,5-b][1,4]diazepin-2-ylamino)-N-(1-methylpiperidin-4-yl)benzamide;
4-(9-sec-butyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-allyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-phenyl-6,7,8,9-tetrahydro-5H-pyrimido[4,5-b][1,4]diazepin-2-ylamino)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(10-Cyclopentyl-5-methyl-6-oxo-5,6,7,8,9,10-hexahydro-pyrimido[4,5-b][1,4]diazocin-2-ylamino)-3-methoxy-N-(1-methyl-piperidin-4-yl)-benzamide;
(R)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
(S)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
4-(9-cyclopentyl-5-methyl-6-oxo-6,7,8,9-tetrahydro-5H-pyrimido[4,5-b][1,4]diazepin-2-ylamino)-3-methoxy N-(1-(methylsulfonyl)piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(3-(2-oxopyrrolidin-1-yl)propyl)benzamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
N-(2-acetamidoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxoimidazolidin-1-yl)ethyl)benzamide;
N-(2-aminoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxopyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(6-oxopiperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperazin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(methylamino)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-2-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-ylmethyl)benzamide;
4-(9-cyclopentyl-5-methyl-6-oxo-6,7,8,9-tetrahydro-5H-pyrimido[4,5-b][1,4]diazepin-2-ylamino)-N-cyclopropyl-3-methoxy-N-(methylpyridin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-(cyclopropanecarbonyl)pyridin-4-yl)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-N-((1-(cyclopropanecarbonyl)piperidin-4-yl)methyl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(1-isopropylpiperidin-4-yl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(dimethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(diethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(1,2,2,6,6-pentamethylpiperidin-4-yl)benzamide;
9-cyclopentyl-5-methyl-2-(pyridin-3-ylamino)-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
3-Methoxy-4-(5-methyl-6-oxo-9-(tetrahydro-2H-pyran-4-yl)-6,7,8,9-tetrahydro-5H-pyrimido[4,5-b][1,4]diazepin-2-ylamino)-N-(1-methylpiperidin-4-yl)benzamide;
4-(9-(4,4-Difluorocyclohexyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclopentyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(3-(dimethylamino)propyl)-3-methoxybenzamide;
N-(1-acetylpiperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-(methylsulfonyl)piperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(2-(dimethylamino)ethyl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclopentyl-5,7,7-trimethyl-6-oxo-6,7,8,9-tetrahydro-5H-pyrimido[4,5-b][1,4]diazepin-2-ylamino)-N,3-dimethoxybenzamide;
4-(9-cyclopentyl-5,7,7-trimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-morpholinobenzamide;
4-(9-cyclopentyl-5,7,7-trimethyl-6-oxo-6,7,8,9-tetrahydro-5H-pyrimido[4,5-b][1,4]diazepin-2-ylamino)-N-(4-(2-hydroxyethyl)piperazin-1-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(4-cyclopentylpiperazin-1-yl)-3-methoxybenzamide;
N-(azetidin-3-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
N-(azepan-4-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-hydroxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-ethoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-methylpiperidin-4-yl)-3-propoxybenzamide;
4-(9-cyclopentyl-5,7,7-trimethyl-6-oxo-6,7,8,9-tetrahydro-5H-pyrimido[4,5-b][1,4]diazepin-2-ylamino)-3-(2,2-difluoroethoxy)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic acid;
4-(9-Cyclopentyl-5,7,7-trimethyl-6-oxo-6,7,8,9-tetrahydro-5H-pyrimido[4,5-b][1,4]diazepin-2-ylamino)-3-(difluoromethoxy)-N-(1-methylpiperidin-4-yl)benzamide;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-isopropyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclopentyl-5′-methyl-6′-oxo-5′,6′,8′,9′-tetrahydrospiro-[cyclopropane-1,7′-pyrimido[4,5-b][1,4]diazepine]-2′-ylamino)-3-methoxy-N-(1-methylpiperidin-3-yl)benzamide;
(R)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)-benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-N-(1-ethylpiperidin-4-yl)-3-methoxybenzamide;
3-methoxy-4-(5′-methyl-9′-(2-methylcyclopentyl)-6′-oxo-5′,6′,8′,9′-tetrahydrospiro[cyclopropane-1,7′-pyrimido[4,5-b][1,4]diazepine]-2′-ylamino)-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid;
4-(9′-Cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(4-methylpiperazin-1-yl)benzamide;
(R)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
4-(7-(Cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-(2-amino-2-oxoethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid;
4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(piperidin-4-yl)benzamide;
(S)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(Z)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(pent-2-ynyl)-6,7,8,9-tetrahydro-5H-pyrimido[4,5-b][1,4]diazepin-2-ylamino)-3-methoxy-N-(1-methylpiperidin-4-yl)benzamide;
(E)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9-cyclopentyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxybenzoic acid;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-7-hydroxy-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic acid;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((S)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
(S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
9-cyclopentyl-5-methyl-2-(pyridin-2-ylamino)-8,9-dihydro-5H-pyrimido[4,5-b][1,4]diazepin-6(7H)-one;
9-cyclopentyl-5-methyl-2-(phenylamino)-8,9-dihydro-5H-pyrimido[4,5-b][1,4]diazepin-6(7H)-one;
9-cyclopentyl-5-methyl-2-(2-(trifluoromethyl)phenylamino)-8,9-dihydro-5H-pyrimido[4,5-b][1,4]diazepin-6(7H)-one;
9-cyclopentyl-2-(2-fluoro-6-methoxyphenylamino)-5-methyl-8,9-dihydro-5H-pyrimido[4,5-b][1,4]diazepin-6(7H)-one;
9-cyclopentyl-2-(3-methoxypyrazin-2-ylamino)-5-methyl-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)nicotinonitrile;
5-(9-cyclopentyl-5-methyl-6-oxo-6,7,8,9-tetrahydro-5H-pyrimido[4,5-b][1,4]diazepin-2-ylamino)picolinonitrile;
5-(9-cyclopentyl-5-methyl-6-oxo-6,7,8,9-tetrahydro-5H-pyrimido[4,5-b][1,4]diazepin-2-ylamino)picolinamide;
6-(9-cyclopentyl-5-methyl-6-oxo-6,7,8,9-tetrahydro-5H-pyrimido[4,5-b][1,4]diazepin-2-ylamino)nicotinamide;
9-cyclopentyl-2-(5-fluoro-2-methoxyphenylamino)-5-methyl-8,9-dihydro-5H-pyrimido[4,5-b][1,4]diazepin-6(7H)-one;
9-cyclopentyl-2-(3-fluoro-2-methoxyphenylamino)-5-methyl-8,9-dihydro-5H-pyrimido[4,5-b][1,4]diazepin-6(7H)-one;
9-cyclopentyl-2-(2-methoxyphenylamino)-5-methyl-8,9-dihydro-5H-pyrimido[4,5-b][1,4]diazepin-6(7H)-one;
9-cyclopentyl-5-methyl-2-(1,2,3,4-tetrahydroisoquinolin-6-ylamino)-8,9-dihydro-5H-pyrimido[4,5-b][1,4]diazepin-6(7H)-one;
2-(4-aminophenylamino)-9-cyclopentyl-5-methyl-8,9-dihydro-5H-pyrimido[4,5-b][1,4]diazepin-6(7H)-one;
N-(4-(9-cyclopentyl-5-methyl-6-oxo-6,7,8,9-tetrahydro-5H-pyrimido[4,5-b][1,4]diazepin-2-ylamino)phenyl)acetamide;
2-(3-aminophenylamino)-9-cyclopentyl-5-methyl-8,9-dihydro-5H-pyrimido[4,5-b][1,4]diazepin-6(7H)-one;
N-(3-(9-cyclopentyl-5-methyl-6-oxo-6,7,8,9-tetrahydro-5H-pyrimido[4,5-b][1,4]diazepin-2-ylamino)phenyl)acetamide;
4-(9-cyclobutyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclopentyl-5-methyl-6-oxo-6,7,8,9-tetrahydro-5H-pyrimido[4,5-b][1,4]diazepin-2-ylamino)-N-(3-(dimethylamino)propyl)-3-methoxybenzamide;
9-cyclopentyl-2-(2-methoxy-4-(4-methylpiperazine-1-carbonyl)phenylamino)-5-methyl-8,9-dihydro-5H-pyrimido[4,5-b][1,4]diazepin-6(7H)-one;
9-cyclopentyl-2-(2-methoxy-4-(morpholine-4-carbonyl)phenylamino)-5-methyl-8,9-dihydro-5H-pyrimido[4,5-b][1,4]diazepin-6(7H)-one;
4-(9-cyclopentyl-5-methyl-6-oxo-6,7,8,9-tetrahydro-5H-pyrimido[4,5-b][1,4]diazepin-2-ylamino)-N-(2-hydroxyethyl)-3-methoxybenzamide;
N-(1-(cyanomethyl)piperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N,N-dimethylpiperidine-1-carboxamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
9-cyclopentyl-2-(isoquinolin-7-ylamino)-5-methyl-8,9-dihydro-5H-pyrimido[4,5-b][1,4]diazepin-6(7H)-one;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
tert-butyl 4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)piperidine-1-carboxylate;
9-cyclopentyl-5-methyl-2-(3,4,5-trimethoxyphenylamino)-8,9-dihydro-5H-pyrimido[4,5-b][1,4]diazepin-6(7H)-one;
isopropyl 4-(9-cyclopentyl-5-methyl-6-oxo-6,7,8,9-tetrahydro-5H-pyrimido[4,5-b][1,4]diazepin-2-ylamino)-3-methoxybenzoate;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
4-(9-cyclopentyl-5,7,7-trimethyl-6-oxo-6,7,8,9-tetrahydro-5H-pyrimido[4,5-b][1,4]diazepin-2-ylamino)-N-(1-(2-(dimethylamino)acetyl)piperidin-4-yl)-3-methoxybenzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N-ethylpiperidine-1-carboxamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-((S)-9-cyclohexyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-4-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
N-(1-(azetidine-3-carbonyl)piperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(4-(9-cyclopentyl-5,7,7-trimethyl-6-oxo-6,7,8,9-tetrahydro-5H-pyrimido[4,5-b][1,4]diazepin-2-ylamino)-3-methoxybenzamido)-N-ethylpiperidine-1-carboxamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
N-((1r,4r)-4-aminocyclohexyl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1r,4r)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-((1-methylpyrrolidin-3-yl)methyl)benzamide;
(S)-4-(9-cyclopentyl-5,7,7-trimethyl-6-oxo-6,7,8,9-tetrahydro-5H-pyrimid[4,5-b][1,4]diazepin-2-ylamino)-3-methoxy-N-((1-methylpyrrolidin-3-yl)methyl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-((1-methylpiperidin-3-yl)methyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-((1-methylpiperidin-4-yl)methyl)benzamide;
4-(9-cyclopentyl-5-methyl-6-oxo-6,7,8,9-tetrahydro-5H-pyrimido[4,5-b][1,4]diazepin-2-ylamino)-3-hydroxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methyl-N-(1-methylpiperidin-4-yl)benzamide;
3-chloro-4-(9-cyclopentyl-5-methyl-6-oxo-6,7,8,9-tetrahydro-5H-pyrimido[4,5-b][1,4]diazepin-2-ylamino)-N-(1-methylpiperidin-4-yl)benzamide; and
4-(9-cyclopentyl-5-methyl-6-oxo-6,7,8,9-tetrahydro-5H-pyrimido[4,5-b][1,4]diazepin-2-ylamino)-N-(1-methylpiperidin-4-yl)-3-(trifluoromethoxy)benzamide.

70. The compound according to claim 1, selected from the group consisting of:
4-(9-cyclopentyl-5,8-dimethyl-6-oxo-6,7,8,9-tetrahydro-5H-pyrimido[4,5-b][1,4]diazepin-2-ylamino)-3-methoxybenzoic acid;
4-(9-cyclopentyl-8-ethyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-{[(7aR)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
3-methoxy-4-{[(7aS)-5-methyl-6-oxo-6,7,7a,8,9,10-hexahydro-5H-pyrimido[5,4-b]pyrrolo[1,2-d][1,4]diazepin-2-yl]amino}-N-(1-methylpiperidin-4-yl)benzamide;
4-[(9-isopropyl-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopropyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-[(9-cyclohexyll-5-methyl-6-oxo-6,7,8,9-tetrahydro-5H-pyrimido[4,5-b][1,4]diazepin-2-yl)amino]-3-methoxy-N-(1-methylpiperidin-4-yl)benzamide;
4-(9-isobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-benzyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-(pentan-3-yl)-6,7,8,9-tetrahydro-5H-pyrimido[4,5-b][1,4]diazepin-2-ylamino)-N-(1-methylpiperidin-4-yl)benzamide;
4-(9-sec-butyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-allyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
3-methoxy-4-(5-methyl-6-oxo-9-phenyl-6,7,8,9-tetrahydro-5H-pyrimido[4,5-b][1,4]diazepin-2-ylamino)-N-(1-methylpiperidin-4-yl)benzamide;
(S)-4-(9-Cyclopentyl-7-ethyl-5-methyl-6-oxo-6,7,8,9-tetrahydro-5H-pyrimido[4,5-b][1,4]diazepin-2-ylamino)-3-methoxy-N-(1-methyl-piperidin-4-yl)-benzamide;
4-(9-cyclopentyl-5-methyl-6-oxo-6,7,8,9-tetrahydro-5H-pyrimido[4,5-b][1,4]diazepin-2-ylamino)-3-methoxy N-(1-(methylsulfonyl)piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(3-(2-oxopyrrolidin-1-yl)propyl)benzamide;
N-(1-acetylpiperidin-4-yl)-4-(9-cyclopentyl-5-methyl-6-oxo-6,7,8,9-tetrahydro-5H-pyrimido[4,5-b][1,4]diazepin-2-ylamino)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(tetrahydro-2H-pyran-4-yl)benzamide;
N-(2-acetamidoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxoimidazolidin-1-yl)ethyl)benzamide;
N-(2-aminoethyl)-4-(9-cyclopentyl-5-methyl-6-oxo-6,7,8,9-tetrahydro-5H-pyrimido[4,5-b][1,4]diazepin-2-ylamino)-3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(2-oxopyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(6-oxopiperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(1-methylpiperidin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperazin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(pyrrolidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(piperidin-1-yl)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(2-(methylamino)ethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(piperidin-4-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-2-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(piperidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-yl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-2-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-3-ylmethyl)benzamide;
4-(9-cyclopentyl-5-methyl-6-oxo-6,7,8,9-tetrahydro-5H-pyrimido[4,5-b][1,4]diazepin-2-ylamino)-3-methoxy-N-(pyridin-4-ylmethyl)benzamide;
4-(9-cyclopentyl-5-methyl-6-oxo-6,7,8,9-tetrahydro-5H-pyrimido[4,5-b][1,4]diazepin-2-ylamino)-N-cyclopropyl-3-methoxy-N-(methylpyridin-4-yl)benzamide;
4-(9-cyclopentyl-5-methyl-6-oxo-6,7,8,9-tetrahydro-5H-pyrimido[4,5-b][1,4]diazepin-2-ylamino)-N-(1-(cyclopropanecarbonyl)pyridin-4-yl)-3-methoxybenzamide;
(S)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
(R)-4-(9-cyclopentyl-5-methyl-6-oxo-6,7,8,9-tetrahydro-5H-pyrimido[4,5-b][1,4]diazepin-2-ylamino)-3-methoxy-N-(pyrrolidin-3-ylmethyl)benzamide;
4-(9-cyclopentyl-5-methyl-6-oxo-6,7,8,9-tetrahydro-5H-pyrimido[4,5-b][1,4]diazepin-2-ylamino)-N-((1-(cyclopropanecarbonyl)piperidin-4-yl)methyl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(1-isopropylpiperidin-4-yl)-3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(dimethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-N-(4-(diethylamino)cyclohexyl) 3-methoxybenzamide;
4-(9-cyclopentyl-5-methyl-6-oxo-6,7,8,9-tetrahydro-5H-pyrimido[4,5-b][1,4]diazepin-2-ylamino)-3-methoxy-N-(1,2,2,6,6-pentamethylpiperidin-4-yl)benzamide;
9-cyclopentyl-5-methyl-2-(pyridin-3-ylamino)-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)-N-(1-methylpiperidin-4-yl)nicotinamide;
3-Methoxy-4-(5-methyl-6-oxo-9-(tetrahydro-2H-pyran-4-yl)-6,7,8,9-tetrahydro-5H-pyrimido[4,5-b][1,4]diazepin-2-ylamino)-N-(1-methylpiperidin-4-yl)benzamide;
4-(9-(4,4-Difluorocyclohexyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-N-(3-(dimethylamino)propyl)-3-methoxybenzamide;
N-(1-acetylpiperidin-4-yl)-4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-(methylsulfonyl)piperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(2-(dimethylamino)ethyl)-3-methoxybenzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpyrrolidin-3-yl)benzamide;
(R)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(1-methylpiperidin-3-yl)benzamide;
(S)-4-(9-cyclopentyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclopentyl-5,7,7-trimethyl-6-oxo-6,7,8,9-tetrahydro-5H-pyrimido[4,5-b][1,4]diazepin-2-ylamino)-N,3-dimethoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-morpholinobenzamide;
4-(9-cyclopentyl-5,7,7-trimethyl-6-oxo-6,7,8,9-tetrahydro-5H-pyrimido[4,5-b][1,4]diazepin-2-ylamino)-N-(4-(2-hydroxyethyl)piperazin-1-yl)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-methoxybenzamide;
4-(9-cyclohexyl-5,7,7-trimethyl-6-oxo-6,7,8,9-tetrahydro-5H-pyrimido[4,5-b][1,4]diazepin-2-ylamino)-3-methoxybenzoic acid;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(1-ethylpiperidin-4-yl)-3-methoxybenzamide;
(R)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
(S)-4-(9-cyclohexyl-5,7,7-trimethyl-6-oxo-6,7,8,9-tetrahydro-5H-pyrimido[4,5-b][1,4]diazepin-2-ylamino)-3-methoxy-N-(pyrrolidin-3-yl)benzamide;
4-(9-cyclohexyl-5,7,7-trimethyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclohexyl-5,7,7-trimethyl-6-oxo-6,7,8,9-tetrahydro-5H-pyrimido[4,5-b][1,4]diazepin-2-ylamino)-N-(4-cyclopentylpiperazin-1-yl)-3-methoxybenzamide;
N-(azetidin-3-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
N-(azepan-4-yl)-4-(9-cyclohexyl-5,7,7-trimethyl-6-oxo-6,7,8,9-tetrahydro-5H-pyrimido[4,5-b][1,4]diazepin-2-ylamino)-3-methoxybenzamide;
4-(9-cyclopentyl-5,7,7-trimethyl-6-oxo-6,7,8,9-tetrahydro-5H-pyrimido[4,5-b][1,4]diazepin-2-ylamino)-3-hydroxybenzoic acid;
4-(9-cyclopentyl-5,7,7-trimethyl-6-oxo-6,7,8,9-tetrahydro-5H-pyrimido[4,5-b][1,4]diazepin-2-ylamino)-3-isopropoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-ethoxy-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-N-(1-methylpiperidin-4-yl)-3-propoxybenzamide;
4-(9-cyclopentyl-5,7,7-trimethyl-6-oxo-6,7,8,9-tetrahydro-5H-pyrimido[4,5-b][1,4]diazepin-2-ylamino)-3-(2,2-difluoroethoxy)-N-(1-methylpiperidin-4-yl)benzamide;
4-(9-cyclopentyl-5,7,7-trimethyl-6-oxo-6,7,8,9-tetrahydro-5H-pyrimido[4,5-b][1,4]diazepin-2-ylamino)-3-(difluoromethoxy)benzoic acid;
4-(9-Cyclopentyl-5,7,7-trimethyl-6-oxo-6,7,8,9-tetrahydro-5H-pyrimido[4,5-b][1,4]diazepin-2-ylamino)-3-(difluoromethoxy)-N-(1-methylpiperidin-4-yl)benzamide;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclopentyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(9-cyclopentyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-((S)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclopentyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(S)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(R)-(9-cyclohexyl-7-ethyl-5,7-dimethyl-6-oxo-6,7,8,9-tetrahydro-5H-pyrimido[4,5-b][1,4]diazepin-2-ylamino)-3-methoxybenzoic acid;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
(S)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
(R)-4-(9-cyclohexyl-7-ethyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((R)-piperidin-3-yl)benzamide;
4-((R)-9-cyclohexyl-7-ethyl-5,7-dimethyl-6-oxo-6,7,8,9-tetrahydro-5H-pyrimido[4,5-b][1,4]diazepin-2-ylamino)-3-methoxy-N-((S)-piperidin-3-yl)benzamide;
4-(9-Cyclopentyl-5,7-dimethyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclobutyl-5,7,7-trimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-isopropyl-5,7,7-trimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-cyclopropyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclohexyl-5,7-dimethyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(9-cyclohexyl-5,7-dimethyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclopentyl-5′-methyl-6′-oxo-5′,6′,8′,9′-tetrahydrospiro-[cyclopropane-1,7′-pyrimido[4,5-b][1,4]diazepine]-2′-ylamino)-3-methoxy-N-(1-methylpiperidin-3-yl)benzamide;
(R)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpyrrolidin-3-yl)benzamide;
4-(9′-cyclopentyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(1-methylpiperidin-4-yl)benzamide;
4-(9′-cyclohexyl-5′-methyl-6′-oxo-5′,6′, 8′,9′-tetrahydrospiro[cyclopropane-1,7′-pyrimido[4,5-b][1,4]diazepine]-2′-ylamino)-3-methoxy-N-(4-methylpiperazin-1-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)-benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylpiperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(1-methylazetidin-3-yl)benzamide;
(S)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
(R)-4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-3-methoxy-N-(piperidin-3-yl)benzamide;
4-(9′-cyclohexyl-5′-methyl-6′-oxo-5′,6′,8′,9′-tetrahydrospiro[cyclopropane-1,7′-pyrimido[4,5-b][1,4]diazepine]-2′-ylamino)-N-(1-ethylpiperidin-4-yl)-3-methoxybenzamide;
3-methoxy-4-(5′-methyl-9′-(2-methylcyclopentyl)-6′-oxo-5′,6′,8′,9′-tetrahydrospiro[cyclopropane-1,7′-pyrimido[4,5-b][1,4]diazepine]-2′-ylamino)-N-(1-methylpiperidin-4-yl)benzamide;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxybenzoic acid;
4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(4-methylpiperazin-1-yl)benzamide;
(R)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
(S)-4-(9′-cyclopentyl-5′-methyl-6′-oxo-5′,6′,8′,9′-tetrahydrospiro[cyclobutane-1,7′-pyrimido[4,5-b][1,4]diazepine]-2′-ylamino)-3-methoxy-N-(piperidin-3-yl)benzamide;
4-(7-(Cyanomethyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(cyanomethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-(2-amino-2-oxoethyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5-methyl-6-oxo-6,7,8,9-tetrahydro-5H-pyrimido[4,5-b][1,4]diazepin-2-ylamino)-3-methoxybenzoic acid;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-propyl-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-N-((1R,4R)-4-hydroxycyclohexyl)-3-methoxybenzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-piperidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-pyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-((S)-7-allyl-9-cyclopentyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
(S)-4-(7-allyl-9-cyclopentyl-5-methyl-6-oxo-6,7,8,9-tetrahydro-5H-pyrimido[4,5-b][1,4]diazepin-2-ylamino)-3-methoxy-N-(piperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxybenzoic acid;
4-(7-allyl-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(piperidin-4-yl)benzamide;
(S)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5-methyl-6-oxo-7-(prop-2-ynyl)-6,7,8,9-tetrahydro-5H-pyrimido[4,5-b][1,4]diazepin-2-ylamino)-3-methoxy-N-(1-methylpiperidin-4-yl)benzamide;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxybenzoic acid;
4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-(but-2-ynyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
(Z)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5-methyl-6-oxo-7-(pent-2-ynyl)-6,7,8,9-tetrahydro-5H-pyrimido[4,5-b][1,4]diazepin-2-ylamino)-3-methoxy-N-(1-methylpiperidin-4-yl)benzamide;
(E)-4-(7-(but-2-enyl)-9-cyclopentyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(S)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(7-allyl-9-cyclopentyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-(4-methylpiperazin-1-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpiperidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((S)-1-methylpyrrolidin-3-yl)benzamide;
4-(7-allyl-9-cyclohexyl-5-methyl-6-oxo-6,7,8,9-tetrahydro-5H-pyrimido[4,5-b][1,4]diazepin-2-ylamino)-3-methoxy-N-((R)-1-methylpyrrolidin-3-yl)benzamide;
4-(9-cyclopentyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-Cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclohexyl-7-(2-fluoroallyl)-5-methyl-6-oxo-6,7,8,9-tetrahydro-5H-pyrimido[4,5-b][1,4]diazepin-2-ylamino)-3-methoxy-N-(1-methylazetidin-3-yl)benzamide;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxybenzoic acid;
4-(9-cyclopentyl-7-methoxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-7-hydroxy-5,7-dimethyl-6-oxo-6,7,8,9-tetrahydro-5H-pyrimido[4,5-b][1,4]diazepin-2-ylamino)-3-methoxybenzoic acid;
4-(9-Cyclopentyl-7-hydroxy-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxybenzoic acid;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylpiperidin-4-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(R)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
(S)-4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(4-methylpiperazin-1-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((R)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-((S)-piperidin-3-yl)benzamide;
4-(9-cyclopentyl-5,7-dimethyl-6-oxo-7-vinyl-6,7,8,9-tetrahydro-5H-pyrimido[4,5-b][1,4]diazepin-2-ylamino)-3-methoxy-N-(1-methylazetidin-3-yl)benzamide;
(S)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
(R)-4-(9-Cyclopentyl-7-ethyl-5,7-dimethyl-6-oxo-6,7,8,9-tetrahydro-5H-pyrimido[4,5-b][1,4]diazepin-2-ylamino)-3-methoxy-N-(1-methylpiperidin-4-yl)benzamide;
9-cyclopentyl-5-methyl-2-(pyridin-2-ylamino)-8,9-dihydro-5H-pyrimido[4,5-b][1,4]diazepin-6(7H)-one;
9-cyclopentyl-5-methyl-2-(phenylamino)-8,9-dihydro-5H-pyrimido[4,5-b][1,4]diazepin-6(7H)-one;
9-cyclopentyl-5-methyl-2-(2-(trifluoromethyl)phenylamino)-8,9-dihydro-5H-pyrimido[4,5-b][1,4]diazepin-6(7H)-one;
9-cyclopentyl-2-(2-fluoro-6-methoxyphenylamino)-5-methyl-8,9-dihydro-5H-pyrimido[4,5-b][1,4]diazepin-6(7H)-one;
9-cyclopentyl-2-(3-methoxypyrazin-2-ylamino)-5-methyl-8,9-dihydro-5H-pyrimido[4,5-b][1,4]diazepin-6(7H)-one;
6-(9-cyclopentyl-5-methyl-6-oxo-6,7,8,9-tetrahydro-5H-pyrimido[4,5-b][1,4]diazepin-2-ylamino)nicotinonitrile;
5-(9-cyclopentyl-5-methyl-6-oxo-6,7,8,9-tetrahydro-5H-pyrimido[4,5-b][1,4]diazepin-2-ylamino)picolinonitrile;
5-(9-cyclopentyl-5-methyl-6-oxo-6,7,8,9-tetrahydro-5H-pyrimido[4,5-b][1,4]diazepin-2-ylamino)picolinamide;
6-(9-cyclopentyl-5-methyl-6-oxo-6,7,8,9-tetrahydro-5H-pyrimido[4,5-b][1,4]diazepin-2-ylamino)nicotinamide;
9-cyclopentyl-2-(5-fluoro-2-methoxyphenylamino)-5-methyl-8,9-dihydro-5H-pyrimido[4,5-b][1,4]diazepin-6(7H)-one;
9-cyclopentyl-2-(3-fluoro-2-methoxyphenylamino)-5-methyl-8,9-dihydro-5H-pyrimido[4,5-b][1,4]diazepin-6(7H)-one;
9-cyclopentyl-5-methyl-2-(1,2,3,4-tetrahydroisoquinolin-6-ylamino)-8,9-dihydro-5H-pyrimido[4,5-b][1,4]diazepin-6(7H)-one;
2-(4-aminophenylamino)-9-cyclopentyl-5-methyl-8,9-dihydro-5H-pyrimido[4,5-b][1,4]diazepin-6(7H)-one;
N-(4-(9-cyclopentyl-5-methyl-6-oxo-6,7,8,9-tetrahydro-5H-pyrimido[4,5-b][1,4]diazepin-2-ylamino)phenyl)acetamide;
2-(3-aminophenylamino)-9-cyclopentyl-5-methyl-8,9-dihydro-5H-pyrimido[4,5-b][1,4]diazepin-6(7H)-one; and
N-(3-(9-cyclopentyl-5-methyl-6-oxo-6,7,8,9-tetrahydro-5H-pyrimido[4,5-b][1,4]diazepin-2-ylamino)phenyl)acetamide.

71. The compound according to any one of claims 1-9 and 13-70, wherein the compound is in the form of a pharmaceutically acceptable salt.

72. The compound or process according to any one of claims 1-9 and 13-71, wherein the compound is present in a mixture of stereoisomers.

73. The compound or process according to any one of claims 1-9 and 13-71, wherein the compound comprises a single stereoisomer.

74. A pharmaceutical composition comprising as an active ingredient a compound according to any one of claims 1-9 and 13-73.

75. The pharmaceutical composition according to claim 74, wherein the composition is a solid formulation adapted for oral administration.

76. The pharmaceutical composition according to claim 74, wherein the composition is a liquid formulation adapted for oral administration.

77. The pharmaceutical composition according to claim 74, wherein the composition is a tablet.

78. The pharmaceutical composition according to claim 74, wherein the composition is a liquid formulation adapted for parenteral administration.

79. A pharmaceutical composition comprising a compound according to any one of claims 1-9 and 13-73, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

80. A kit comprising:
a compound of any one of claims 1-9 and 13-73; and
instructions which comprise one or more forms of information selected from the group consisting of indicating a disease state for which the compound is to be administered, storage information for the compound, dosing information and instructions regarding how to administer the compound.

81. The kit of claim 80, wherein the kit comprises the compound in a multiple dose form.

82. An article of manufacture comprising:
a compound of any one of claims 1-9 and 13-73; and
packaging materials.

83. The article of manufacture of claim 82, wherein the packaging material comprises a container for housing the compound.

84. The article of manufacture of claim 83, wherein the container comprises a label indicating one or more members of the group consisting of a disease state for which the compound is to be administered, storage information, dosing information and/or instructions regarding how to administer the compound.

85. The article of manufacture of claim 83, wherein the article of manufacture comprises the compound in a multiple dose form.

86. A therapeutic method comprising administering a compound of any one of claims 1-9 and 13-73 to a subject.

87. A method of inhibiting a kinase comprising contacting the kinase with a compound of any one of claims 1-9 and 13-73.

88. A method of inhibiting a kinase comprising causing a compound of any one of claims 1-9 and 13-73 to be present in a subject in order to inhibit the kinase in vivo.

89. A method of inhibiting a kinase comprising administering a first compound to a subject that is converted in vivo to a second compound wherein the second compound inhibits the kinase in vivo, the second compound being a compound according to any one of claims 1-9 and 13-73.

90. A method of treating a disease state for which a kinase possesses activity that contributes to the pathology and/or symptomology of the disease state, the method comprising causing a compound of any one of claims 1-9 and 13-73 to be present in a subject in a therapeutically effective amount for the disease state.

91. A method of treating a disease state for which a kinase possesses activity that contributes to the pathology and/or symptomology of the disease state, the method comprising administering a compound of any one of claims 1-9 and 13-73 to a subject, wherein the compound is present in the subject in a therapeutically effective amount for the disease state.

92. A method of treating a disease state for which a kinase possesses activity that contributes to the pathology and/or symptomology of the disease state, the method comprising administering a first compound to a subject that is converted in vivo to a second compound wherein the second compound inhibits the kinase in vivo, the second compound being a compound according to any one of claims 1-9 and 13-73.

93. The method according to any one of claims 90-92, wherein the disease state is selected from the group consisting of hyperproliferative disorders; cancer; inflammatory diseases; auto-immune diseases; chemotherapy agent-induced alopecia and mucositis; cardiovascular diseases; viral, bacterial, fungal and/or parasitic infectious diseases; nephrological diseases; chronic and acute neurodegenerative diseases; skin diseases; bone diseases; and the protection of proliferating cells.

94. The method according to any one of claims 90-92, wherein the disease state is selected from the group consisting of solid tumors; leukemias; lymphomas; non-small cell lung cancers; esophageal carcinoma; psoriasis, alopecia; multiple sclerosis; colitis, arthritis, Alzheimer's disease, glomerulonephritis; wound healing; stenoses, arterioscleroses; restenoses; hypertrophy; cytomegalic infections, herpes, hepatitis B and C, Karposi's sarcoma; HIV diseases; disease caused by unicellular parasites; glomerulonephritis; Huntington's disease, amyotrophic lateral sclerosis, Parkinson's disease, AIDS dementia; ischemias of the brain and neurotraumas.

95. The method according to any one of claims 87-94, wherein the kinase is a protein tyrosine kinase.

96. The method according to any one of claims 87-94, wherein the kinase is a polo-like kinase.

97. The method according to claim 96, wherein the polo-like kinase is selected from the group consisting of PLK1, PLK2, PLK3 and PLK4.

98. The method according to any one of claims 87-94, wherein the kinase is a TTK.

99. The method according to any one of claims 87-94, wherein the kinase is a FAK.

100. The method according to any one of claims 87-94, wherein the kinase is a AIK.

101. A compound according to any one of claims 1-9 and 13-73 for use as a medicament.

102. Use of a compound according to any one of claims 1-9 and 13-73 in the manufacture of a medicament for inhibiting a polo-like kinase.

103. Use of a compound according to any one of claims 1-9 and 13-73 in the manufacture of a medicament for treating a disease state for which a polo-like kinase possess activity that contributes to the pathology and/or symptomology of the disease state.

104. Use of a compound according to any one of claims 1-9 and 13-73 in the manufacture of a medicament for treating hyperproliferative disorders; cancer; inflammatory diseases; auto-immune diseases; chemotherapy agent-induced alopecia and mucositis; cardiovascular diseases; viral, bacterial, fungal and/or parasitic infectious diseases; nephrological diseases; chronic and acute neurodegenerative diseases; skin diseases; bone diseases; and the protection of proliferating cells.


SEQUENCE LISTING (6 sequences)

SEQ ID NO: 1 - DNA, 1809 bp, Homo sapiens
atgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcc

SEQ ID NO: 2 - DNA, 27 bp, Artificial
  DNA sequence endoding PCR Primer
agtgctgcagtgactgcagggaagctg

SEQ ID NO: 3 - DNA, 29 bp, Artificial
  Synthetic Primer
ttaggaggccttgagacggttgctggccg

SEQ ID NO: 4 - DNA, 69 bp, Artificial
  Synthetic Primer
aaatctagagccaccatggactacaaggacgacgatgacaagagtgctgcagtgactgcagggaagctg

SEQ ID NO: 5 - DNA, 27 bp, Artificial
  Synthetic Primer
tggcaactagaaggcacagtcgaggct

SEQ ID NO: 6 - protein, 611 aa, Artificial
  Protein Sequence encoding FLAG-tagged PLK1
MDYKDDDDKSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS